(19) United States
(12) Patent Application Publication
(10) Pub. No.: US 2007/0155730 A1
(43) Pub. Date: Jul. 5, 2007

(54) Benzodiazepine And Benzopiperazine Analog Inhibitors Of Histone Deacetylase

(75) Inventors: Silvana Leit, Kirkland, QC (CA); Amal Wahhab, Laval, QC (CA); Martin Allan, Montreal, QC (CA); David Smil, Montreal, QC (CA); Pierre Tessier, Hawkesbury, ON (CA); Robert Deziel, Ville Mont-Royal, QC (CA); Yves Andre Chantigny, Pincourt, QC (CA)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: METHYLGENE, INC., Montreal, QC (CA)

(21) Appl. No.: 11/467,444

(22) Filed: Aug. 25, 2006

Related U.S. Application Data
(60) Provisional application No. 60/712,011, filed on Aug. 26, 2005.

(51) Int. Cl. A61K 31/5513; C07D 497/04; C07D 243/12; C07D 487/04

(52) U.S. Cl. 514/221; 540/502; 540/504

(57) ABSTRACT

This invention relates to compounds for the inhibition of histone deacetylase. More particularly, the invention provides for compounds of formula (I) 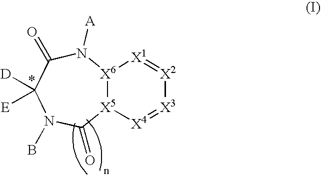
wherein A, B, D, E, X¹, X², X³, X⁴ and n are as defined in the specification.

This application claims the benefit of U.S. provisional application No. 60/712,011, filed Aug. 26, 2005.

### BACKGROUND OF THE INVENTION

(a) Field of the Invention

This invention relates to the inhibition of histone deacetylase. More particularly, the invention relates to compounds and methods for inhibiting histone deacetylase enzymatic activity.

(b) Description of Related Art

In eukaryotic cells, nuclear DNA associates with histones to form a compact complex called chromatin. The histones constitute a family of basic proteins which are generally highly conserved across eukaryotic species. The core histones, termed H2A, H₂B, H3, and H4, associate to form a protein core. DNA winds around this protein core, with the basic amino acids of the histones interacting with the negatively charged phosphate groups of the DNA. Approximately 146 base pairs of DNA wrap around a histone core to make up a nucleosome particle, the repeating structural motif of chromatin.

Csordas, Biochem. J., 265: 23-38 (1990) teaches that histones are subject to post-translational acetylation of the ε-amino groups of N-terminal lysine residues, a reaction that is catalyzed by histone acetyl transferase (HAT1). Acetylation neutralizes the positive charge of the lysine side chain, and is thought to impact chromatin structure. Indeed, Taunton et al., Science, 272: 408-411 (1996), teaches that access of transcription factors to chromatin templates is enhanced by histone hyperacetylation. Taunton et al. further teach that an enrichment in underacetylated histone H4 has been found in transcriptionally silent regions of the genome.

Histone acetylation is a reversible modification, with deacetylation being catalyzed by a family of enzymes termed histone deacetylases (HDACs). The molecular cloning of gene sequences encoding proteins with HDAC activity has established the existence of a set of discrete HDAC enzyme isoforms. Grozinger et al., Proc. Natl. Acad. Sci. USA, 96:4868-4873 (1999), teaches that HDACs may be divided into two classes, the first represented by yeast Rpd3-like proteins, and the second represented by yeast Hd1-like proteins. Grozinger et al. also teaches that the human HDAC-1, HDAC-2, and HDAC-3 proteins are members of the first class of HDACs, and discloses new proteins, named HDAC-4, HDAC-5, and HDAC-6, which are members of the second class of HDACs. Kao et al., Gene & Development 14:55-66 (2000), discloses an additional member of this second class, called HDAC-7. More recently, Hu, E. et al. J. Bio. Chem. 275:15254-13264 (2000) discloses the newest member of the first class of histone deacetylases, HDAC-8. Zhou et al., Proc. Natl. Acad. Sci. U.S.A., 98: 10572-10577 (2001) teaches the cloning and characterization of a new histone deacetylase, HDAC-9. Kao et al., J. Biol. Chem., 277:187-93 (2002) teaches the isolation and characterization of mammalian HDAC10, a novel histone deacetylase. Gao et al, J. Biol. Chem. (In press) teaches the cloning and functional characterization of HDAC11, a novel member of the human histone deacetylase family. Shore, Proc. Natl. Acad. Sci. U.S.A. 97: 14030-2 (2000) discloses another class of deacetylase activity, the Sir2 protein family. It has been unclear what roles these individual HDAC enzymes play.

Studies utilizing known HDAC inhibitors have established a link between acetylation and gene expression. Numerous studies have examined the relationship between HDAC and gene expression. Taunton et al., Science 272:408-411 (1996), discloses a human HDAC that is related to a yeast transcriptional regulator. Cress et al., J. Cell. Phys. 184:1-16 (2000), discloses that, in the context of human cancer, the role of HDAC is as a corepressor of transcription. Ng et al., TIBS 25: March (2000), discloses HDAC as a pervasive feature of transcriptional repressor systems. Magnaghi-Jaulin et al., Prog. Cell Cycle Res. 4:41-47 (2000), discloses HDAC as a transcriptional co-regulator important for cell cycle progression.

Richon et al., Proc. Natl. Acad. Sci. USA, 95: 3003-3007 (1998), discloses that HDAC activity is inhibited by trichostatin A (TSA), a natural product isolated from Streptomyces hygroscopicus, which has been shown to inhibit histone deacetylase activity and arrest cell cycle progression in cells in the G1 and G2 phases (Yoshida et al., J. Biol. Chem. 265: 17174-17179, 1990; Yoshida et al., Exp. Cell Res. 177: 122-131, 1988), and by a synthetic compound, suberoylanilide hydroxamic acid (SAHA). Yoshida and Beppu, Exper. Cell Res., 177: 122-131 (1988), teaches that TSA causes arrest of rat fibroblasts at the G₁ and G₂ phases of the cell cycle, implicating HDAC in cell cycle regulation. Indeed, Finnin et al., Nature, 401: 188-193 (1999), teaches that TSA and SAHA inhibit cell growth, induce terminal differentiation, and prevent the formation of tumors in mice. Suzuki et al., U.S. Pat. No. 6,174,905, EP 0847992, and JP 258863/96, disclose benzamide derivatives that induce cell differentiation and inhibit HDAC. WO 03/024448, WO 2004/069823, WO 00/71703, WO 01/38322, WO 01/70675, WO 2004/035525, WO 2005/030705, WO 2005/092899, among others, disclose additional compounds that serve as HDAC inhibitors. Other inhibitors of histone deacetylase activity, including trapoxin, depudecin, FR901228 (Fujisawa Pharmaceuticals), and butyrate, have been found to similarly inhibit cell cycle progression in cells (Taunton et al., Science 272: 408-411, 1996; Kijima et al., J. Biol. Chem. 268(30):22429-22435, 1993; Kwon et al., Proc. Natl. Acad. Sci. USA 95(7):3356-61, 1998).

It would be highly desirable to be provided with additional compounds and methods for inhibiting histone deacetylase enzymatic activity.

### SUMMARY OF THE INVENTION

The present invention provides compounds for the inhibition of histone deacetylase.

In a first aspect, the invention provides compounds, and N-oxides, hydrates, solvates, pharmaceutically acceptable salts, prodrugs and complexes thereof, that are useful as histone deacetylase inhibitors that have the formula (I): 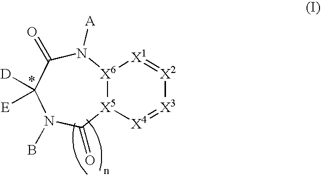
wherein A, B, D, E, X¹, X², X³, X⁴ and n are as defined below. The compounds are, therefore, also useful research tools for the study of the role of histone deacetylase in both normal and disease states.

In a second aspect, the invention provides compounds, and N-oxides, hydrates, solvates, pharmaceutically acceptable salts, prodrugs and complexes thereof, that are useful as histone deacetylase inhibitiors that have the formula (XVI): 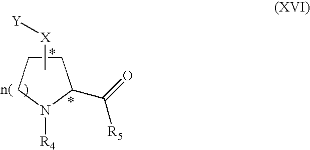
wherein X, Y, R⁴, R⁵ and n are as defined below. The compounds are, therefore, also useful research tools for the study of the role of histone deceatylase in both normal and disease states.

In a third aspect, the invention provides a composition comprising a compound according to any one of paragraphs [0009] to [0010], or as depicted in any of the tables herein together with a pharmaceutically acceptable carrier, diluent or excipient.

In a fourth aspect, the third aspect of the invention provides a method of inhibiting histone deacetylase, the method comprising contacting the histone deacetylase or a cell containing histone deacetylase with a compound according to any one of paragraphs [0009] to [0010] or as depicted in any of the tables herein, or with a composition according to paragraph [0011]. Because compounds of the invention inhibit histone deacetylase, they are useful research tools for the study of the role of histone deacetylase in biological processes.

The foregoing merely summarizes various aspects of the invention and is not intended to be limiting in nature. These aspects and other aspects and embodiments are described more fully below. The patent and scientific literature referred to herein establishes knowledge that is available to those with skill in the art. The issued patents, applications, and references that are cited herein are hereby incorporated by reference to the same extent as if each was specifically and individually indicated to be incorporated by reference. In the case of inconsistencies, the present disclosure will prevail.

Throughout the specification, preferred embodiments of one or more chemical substituents are identified. Also preferred are combinations of preferred embodiments. For example, the invention describes preferred embodiments of E in the compounds and describes preferred embodiments of group A. Thus, as an example, also contemplated as within the scope of the invention are compounds in which preferred examples of E are as described and in which preferred examples of group A are as described. Furthermore, compounds excluded from any one particular genus of compounds (e.g., through a proviso clause) are intended to be excluded from the scope of the invention entirely, including from other disclosed genera, unless expressly stated to the contrary.

### DETAILED DESCRIPTION OF THE INVENTION

The present invention provides compounds that are useful as inhibitors of histone deacetylase.

In one aspect of the present invention there is provided compounds of formula (I): 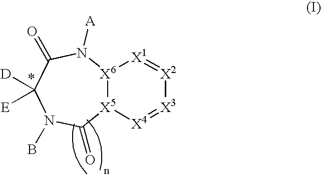
and N-oxides, hydrates, solvates, pharmaceutically acceptable salts, prodrugs and complexes thereof, wherein
n is 0 or 1;
X¹, X², X³ and X⁴ are independently selected from the group consisting of CH, C-Z and N, wherein no more than two of X¹, X², X³ and X⁴ are N and no more than one of X¹, X², X³ and X⁴ are C-Z;
X⁵-X⁶ is C═C;
or
X¹, X², X³ and X⁴ are absent, X⁵ is a covalent bond and X⁶ is independently selected from the group consisting of CH₂ and CH(Z), with the provisio that an N, O or S(O)₀₋₁ in Z is separated from the CH of X⁶ by at least —(CH₂)₂—;

Z is independently selected from the group consisting of halo, —CF₃, —NO₂, —CN, —(C₀-C₆)alkyl-OR¹, —(C₀-C₆)alkyl-N(R¹)₂, —(C₁-C₆)alkyl, —N(R¹)—C(O)—(C₁-C₆)alkyl, —N(R¹)—S(O)₂—(C₁-C₆)alkyl, —O—(C₂-C₆)alkyl-N(R¹)(R¹), —S—R¹, —(C₀-C₆)alkyl-C(O)—OR¹, —N(R¹)—C(O)—CF₃, —N(R¹)—(C₂-C₆)alkyl-N(R¹)(R¹), —(C₀-C₇)alkyl-W, —(C₂-C₇)alkenyl-W, —(C₂-C₇)alkynyl-W, —(C₀-C₅)alkyl-CH═CH—W, —C(O)—(C₁-C₇)alkyl-W, —(C₀-C₃)alkyl-N(R¹)—C(O)—(C₁-C₆)alkyl-W, —(C₀-C₃)alkyl-N(R¹)—C(S)—(C₁-C₆)alkyl-W, —C(O)—N(R¹)—(C₁-C₆)alkyl-W, —(C₀-C₃)alkyl-N(R¹)—(C₁-C₆)alkyl-W, —(C₀-C₃)alkyl-N(R¹)—C(O)—N(R¹)—(C₁-C₆)alkyl-W, —(C₀-C₃)alkyl-N(R¹)—C(O)—O—(C₁-C₆)alkyl-W, —S(O)₂—N(R¹)—(C₁-C₆)alkyl-W, —(C₀-C₃)alkyl-N(R¹)—S(O)₂—(C₁-C₆)alkyl-W, —C(O)—N(R¹)₂, —(C₀-C₃)alkyl-O—C(O)—N(R¹)—(C₁-C₆)alkyl-W, —(C₀-C₃)alkyl-O—(C₁-C₆)alkyl-W, —(C₀-C₃)alkyl-S—(C₁-C₆)alkyl-W, —N(R¹)—C(O)—OR¹, —S(O)₂—N(R¹)₂, —N(R¹)—S(O)₂R¹, —(C₀-C₇)alkyl-aryl-W, —(C₀-C₇)alkyl-heteroaryl-W, —(C₀-C₃)alkyl-O—(C₀-C₃)alkyl-aryl, —(C₀-C₃)alkyl-O—(C₀-C₃)alkyl-heteroaryl, -aryl, —(C₁-C₆)alkylaryl-, -heteroaryl, —(C₁-C₆)alkylheteroaryl-, —(C₁-C₈) heteroalkyl, —(C₃-C₆)cycloalkyl, —(C₃-C₆)heterocycloalkyl, —(C₀-C₃)alkyl-N(R¹)—(C₀-C₃)alkyl-aryl-(CH═CH)₀₋₁—W, —(C₀-C₃)alkyl-O—(C₀-C₃)alkyl-aryl-(CH═CH)₀₋₁—W, —(C₀-C₃)alkyl-S—(C₀-C₃)alkyl-aryl-(CH═CH)₀₋₁—W, —(C₀-C₃)alkyl-N(R¹)C(O)—O—(C₀-C₃)alkyl-aryl-(CH═CH)₀₋₁—W, —(C₀-C₃)alkyl-O—C(O)N(R¹)—(C₀-C₃)alkyl-aryl-(CH═CH)₀₋₁—W, —(C₀-C₃)alkyl-S(O)₂N(R¹)—(C₀-C₃)alkyl-aryl-(CH═CH)₀₋₁—W, —(C₀-C₃)alkyl-N(R¹)S(O)₂—(C₀-C₃)alkyl-aryl-(CH═CH)₀₋₁—W, —(C₀-C₃)alkyl-C(O)N(R¹)—(C₀-C₃)alkyl-aryl-(CH═CH)₀₋₁—W, —(C₀-C₃)alkyl-N(R¹)C(O)—(C₀-C₃)alkyl-aryl-(CH═CH)₀₋₁—W, —(C₀-C₃)alkyl-N(R¹)C(O)N(R¹)—(C₀-C₃)alkyl-aryl-(CH═CH)₀₋₁—W, —(C₀-C₃)alkyl-(CH═CH)—(C₀-C₃)alkyl-aryl-(CH═CH)₀₋₁—W, —(C₀-C₃)alkyl-N(R¹)—(C₀-C₃)alkyl-heteroaryl-(CH═CH)₀₋₁—W, —(C₀-C₃)alkyl-O—(C₀-C₃)alkyl-heteroaryl-(CH═CH)₀₋₁—W, —(C₀-C₃)alkyl-S—(C₀-C₃)alkyl-heteroaryl-(CH═CH)₀₋₁—W, —(C₀-C₃)alkyl-N(R¹)C(O)—O—(C₀-C₃)alkyl-heteroaryl-(CH═CH)₀₋₁—W, —(C₀-C₃)alkyl-OC(O)N(R¹)—(C₀-C₃)alkyl-heteroaryl-(CH═CH)₀₋₁—W, —(C₀-C₃)alkyl-S(O)₂N(R₁)—(C₀-C₃)alkyl-heteroaryl-(CH═CH)₀₋₁—W, —(C₀-C₃)alkyl-N(R¹)S(O)₂—(C₀-C₃)alkyl-heteroaryl-(CH═CH)₀₋₁—W, —(C₀-C₃)alkyl-C(O)N(R¹)—(C₀-C₃)alkyl-heteroaryl-(CH═CH)₀₋₁—W, —(C₀-C₃)alkyl-N(R¹)C(O)—(C₀-C₃)alkyl-heteroaryl-(CH═CH)₀₋₁—W, —(C₀-C₃)alkyl-N(R¹)C(O)N(R¹)—(C₀-C₃)alkyl-heteroaryl-(CH═CH)₀₋₁—W and —(C₀-C₃)alkyl-(CH═CH)—(C₀-C₃)alkyl-heteroaryl-(CH═CH)₀₋₁—W, —(C₀-C₅)alkyl-C≡C—W, —(C₀-C₃)alkyl-O—(C₀-C₃)alkyl-aryl-(C≡C)₀₋₁—W, —(C₀-C₃)alkyl-S—(C₀-C₃)alkyl-aryl-(C≡C)₀₋₁—W, —(C₀-C₃)alkyl-N(R¹)C(O)—O—(C₀-C₃)alkyl-aryl-(C≡C)₀₋₁—W, —(C₀-C₃)alkyl-O—C(O)N(R¹)—(C₀-C₃)alkyl-aryl-(C≡C)₀₋₁—W, —(C₀-C₃)alkyl-S(O)₂N(R¹)—(C₀-C₃)alkyl-aryl-(C≡C)₀₋₁—W, —(C₀-C₃)alkyl-N(R¹)S(O)₂—(C₀-C₃)alkyl-aryl-(C≡C)₀₋₁—W, —(C₀-C₃)alkyl-C(O)N(R¹)—(C₀-C₃)alkyl-aryl-(C≡C)₀₋₁—W, —(C₀-C₃)alkyl-N(R¹)C(O)—(C₀-C₃)alkyl-aryl-(C≡C)₀₋₁—W, —(C₀-C₃)alkyl-N(R¹)C(O)N(R¹)—(C₀-C₃)alkyl-aryl-(C≡C)₀₋₁—W, —(C₀-C₃)alkyl-(C≡C)—(C₀-C₃)alkyl-aryl-(CH═CH)₀₋₁—W, —(C₀-C₃)alkyl-(CH═CH)—(C₀-C₃)alkyl-aryl-(C≡C)₀₋₁—-W, —(C₀-C₃)alkyl-(C≡C)—(C₀-C₃)alkyl-aryl-(C≡C)₀₋₁—W, —(C₀-C₃)alkyl-N(R¹)—(C₀-C₃)alkyl-heteroaryl-(C≡C)₀₋₁—W, —(C₀-C₃)alkyl-O—(C₀-C₃)alkyl-heteroaryl-(C≡C)₀₋₁—W, —(C₀-C₃)alkyl-S—(C₀-C₃)alkyl-heteroaryl-(C≡C)₀₋₁—W, —(C₀-C₃)alkyl-N(R¹)C(O)—O—(C₀-C₃)alkyl-heteroaryl-(C≡C)₀₋₁—W, —(C₀-C₃)alkyl-OC(O)N(R¹)—(C₀-C₃)alkyl-heteroaryl-(C≡C)₀₋₁—W, —(C₀-C₃)alkyl-S(O)₂N(R₁)—(C₀-C₃)alkyl-heteroaryl-(C≡C)₀₋₁—W, —(C₀-C₃)alkyl-N(R¹)S(O)₂—(C₀-C₃)alkyl-heteroaryl-(C≡C)₀₋₁—W, —(C₀-C₃)alkyl-C(O)N(R¹)—(C₀-C₃)alkyl-heteroaryl-(C≡C)₀₋₁—W, —(C₀-C₃)alkyl-N(R¹)C(O)—(C₀-C₃)alkyl-heteroaryl-(C≡C)₀₋₁—W, —(C₀-C₃)alkyl-N(R¹)C(O)N(R¹)—(C₀-C₃)alkyl-heteroaryl-(C≡C)₀₋₁—W, —(C₀-C₃)alkyl-(C≡C)—(C₀-C₃)alkyl-heteroaryl-(CH═CH)₀₋₁—W, —(C₀-C₃)alkyl-(CH═CH)—(C₀-C₃)alkyl-heteroaryl-(C≡C)₀₋₁—W, —(C₀-C₃)alkyl-(C≡C)—(C₀-C₃)alkyl-heteroaryl-(C≡C)₀₋₁—W, (C₀-C₃)alkyl-C(O)—N(R¹)—(C₁-C₆)alkyl-W, (C₀-C₃)alkyl-C(S)—N(R¹)—(C₁-C₆)alkyl-W, —(C₀-C₃)alkyl-N(R₁)—C(O)—(C₁-C₆)alkyl-C(O)-aryl, —(C₀-C₃)alkyl-N(R₁)—C(O)—(C₁-C₆)alkyl-C(O)-heteroaryl, —(C₀-C₃)alkyl-N(R₁)—C(O)—(C₁-C₆)alkyl-C(O)—N(R₁)-aryl and —(C₀-C₃)alkyl-N(R₁)—C(O)—(C₁-C₆)alkyl-C(O)—N(R₁)-heteroaryl, wherein each of the aryl, heteroaryl, cycloalkyl and heterocyclyl moieties of the above-mentioned Z is optionally substituted with one or more substituents selected from the group consisting of oxo, —OH, —CN, —(C₁-C₆)alkyl, —(C₁-C₆)alkoxy, —NO₂, —N(R¹)₂, halo, —SH, mono- to per-halogenated —(C₁-C₆)alkyl, and —(C₂-C₄)alkyl-N(R¹)₂, wherein two R¹ groups, together with the nitrogen atom to which they are attached optionally form a heterocyclyl group;
R¹ is independently selected from the group consisting of —H, —(C₁-C₆)alkyl, —(C₁-C₆)heteroalkyl, —(C₃-C₆)cycloalkyl, heterocyclyl, —(C₀-C₆)alkyl-aryl, —(C₀-C₆)alkyl-heteroaryl and —(C₂-C₄)alkyl-N(R¹)₂, wherein each aryl, heteroaryl, cycloalkyl and heterocyclyl moiety of said —(C₃-C₆)cycloalkyl, heterocyclyl, —(C₀-C₆)alkyl-aryl and —(C₀-C₆)alkyl-heteroaryl is optionally substituted with one or more substituents selected from the group consisting of oxo, —OH, —CN, —(C₁-C₆)alkyl, —(C₁-C₆)alkoxy, —NO₂, —N(R¹)₂, halo, aryl, heteroaryl, mono- to per-halogenated-(C₁-C₆)alkyl and —(C₂-C₄)alkyl-N(R¹)₂, wherein two R¹ groups, together with the nitrogen atom to which they are attached optionally form a heterocyclyl group;
W is selected from the group consisting of —C(O)—NH—OH, —C(O)—C₁-C₄ alkyl, —C(O)—N(R¹)₂, —(C₁-C₆)alkyl-N(OH)—C(O)H—, —(C₁-C₆)alkyl-SR¹, —(C₁-C₆)alkyl-S—C(O)—(C₁-C₄)alkyl, —C(O)—OR¹, 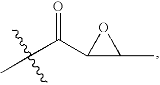
—C(O)—(C₁-C₄)alkyl-SH, —C(O)—(C₁-C₄)alkyl-S—C(O)R¹, —C(O)—(C₁-C₄)alkyl-5-heteroaryl, (C₁-C₆)alkyl-NH—C(O)—(C₁-C₆) alkyl-halo, —(C₁-C₆)alkyl-NH—C(O)—(C₁-C₆)alkyl-SH, —(C₁-C₆)alkyl-NH—C(O)—(C₁-C₆)alkyl-SC(O)R¹, —C(O)—NH—(C₂-C₆)alkyl-SH, —C(O)—N(R¹)—(C₀-C₆)alkyl-SR¹, —C(O)-cycloalkyl, —C(O)-heterocyclyl, —C(O)—N(R¹)-aryl-Q, —C(O)—N(R¹)— heteroaryl-Q, —C(O)-aryl, —C(O)-heteroaryl and —C(O)—(C₁-C₆) alkyl wherein the alkyl is optionally substituted with one or more substituents selected from the group consisting of halo, mono to per-halogenated-(C₁-C₆)alkyl, —C(O)-heteroaryl, —C(O)—NH-heteroaryl and —C(O)—NH-aryl, wherein each aryl and heteroaryl moiety of the afore-mentioned W group is optionally substituted with one or more substituents selected from the group consisting of —NH₂, —OH, —SH, —CN, —NO₂, —N(R¹)₂, halo, mono- to per-halogenated —(C₁-C₆)alkyl, aryl and heteroaryl;
E and D are independently selected from the group consisting of —H, —(C₁-C₆)alkyl, —(C₁-C₆)heteroalkyl, —(C₀-C₆)alkyl-(C₃-C₆)cycloalkyl, —(C₀-C₆) heteroalkyl-(C₃-C₆)cycloalkyl, —(C₀-C₆)alkyl-(C₃-C₆) heterocyclyl, —(C₀-C₆)heteroalkyl-(C₃-C₆)heterocyclyl, —(C₀-C₆)alkyl-aryl, —(C₀-C₆)alkyl-heteroaryl, —(C₀-C₆)alkyl-heteroaryl-(C₀-C₃)alkyl-aryl, —(C₀-C₆)alkyl-aryl-(C₀-C₃)alkyl-aryl, —(C₀-C₆)alkyl-heteroaryl-(C₀-C₃)alkyl-heteroaryl, —(C₀-C₆)alkyl-aryl-(C₀-C₃)alkyl-heteroaryl, heterocyclyl, —(C₁-C₆)alkyl-S—R¹, —(C₁-C₆)heteroalkyl-S—R¹, —(C₁-C₆)alkyl-O—R¹, —(C₁-C₆)heteroalkyl-O—R¹, —C₁-C₆ alkyl-W, —(C₁-C₆)heteroalkyl-W, —(C₁-C₆)alkyl-M-(C₁-C₃)alkyl-W, —(C₁-C₆)heteroalkyl-M-(C₁-C₃)alkyl-W, —(C₁-C₆)alkyl-N(R¹)₂, —(C₁-C₆)heteroalkyl-N(R¹)₂, —(C₁-C₆)alkyl-N(R¹)—C(O)—OR¹, —(C₀-C₆)alkyl-C(O)—O—(C₁-C₆)alkyl, —(C₀-C₆)alkyl-C(O)—O—(C₁-C₆) heteroalkyl, —(C₀-C₆)heteroalkyl-C(O)—O—(C₁-C₆)alkyl, —(C₀-C₆)heteroalkyl-C(O)—O—(C₁-C₆)heteroalkyl, —(C₀-C₆)alkyl-C(O)—O—(C₁-C₆)cycloalkyl, —(C₀-C₆) heteroalkyl-C(O)—O—(C₁-C₆)cycloalkyl, —(C₀-C₆)alkyl-C(O)—O—(C₁-C₆)heterocyclyl, —(C₀-C₆) heteroalkyl-C(O)—O—(C₁-C₆)heterocyclyl, —(C₀-C₆)alkyl-C(O)—N(R¹)₂, —(C₀-C₆)heteroalkyl-C(O)—N(R¹)₂ and —C(O)—N(R¹)—C₂-C₆alkyl-W, wherein each aryl, heteroaryl, cycloalkyl or heterocyclyl moiety is optionally substituted with one or more groups selected from R²; wherein
M is selected from the group consisting of CH₂, O, S, S(O), S(O)₂, and N(R¹); or
C and D together with the carbon atom to which they are attached form a (C₃-C₆)cycloalkyl, wherein the cycloalkyl is optionally substituted;
R² is independently selected from the group consisting of —H, —(C₁-C₆)alkyl, —(C₁-C₆)heteroalkyl, —(C₀-C₆)alkyl-OR¹, —(C₀-C₆)heteroalkyl-OR¹, —(C₀-C₆)alkyl-C(O)—OR¹, —(C₀-C₆)heteroalkyl-C(O)—OR¹, —CH═CH—C(O)—OR¹, —C≡C—C(O)—OR¹, —CH═CH—C(O)—N(R¹)₂, —C≡C—C(O)—N(R¹)₂, —N(R¹)—C(O)—CF₃, —C(O)—N(R¹)—CF₃, —N(R¹)—(C₁-C₆)alkyl-N(R¹)₂, —N(R¹)—(C₁-C₆)heteroalkyl-N(R¹)₂, —(C₀-C₆)alkyl-N(R¹)₂, —(C₀-C₆)heteroalkyl-N(R¹)₂, —N(R¹)—C(O)—(C₁-C₆)alkyl, —C(O)—N(R¹)—(C₁-C₆)alkyl, —N(R¹)—C(O)—(C₁-C₆)heteroalkyl, —C(O)—N(R¹)—(C₁-C₆)heteroalkyl, —N(R¹)—S(O)₂—(C₁-C₆)alkyl, —N(R¹)—S(O)₂—(C₁-C₆)heteroalkyl, —S(O)₂—N(R¹)—(C₁-C₆)alkyl, —S(O)₂—N(R¹)—(C₁-C₆)heteroalkyl, —O—(C₁-C₆)alkyl-N(R¹)₂, —O—(C₁-C₆)heteroalkyl-N(R¹)₂, —S—(C₁-C₆)alkyl-N(R¹)₂, —S—(C₁-C₆)heteroalkyl-N(R¹)₂, —S—R¹, —S(O)—(C₁-C₆)alkyl, —S(O)—(C₁-C₆)heteroalkyl, —S(O)₂—(C₁-C₆)alkyl, —S(O)₂—(C₁-C₆)heteroalkyl, —(C₃-C₆)cycloalkyl, heterocyclyl, halo, —CF₃, —OCF₃, —C(Ph)₃, —CN, —(C₁-C₆)alkylaryl, aryl, heteroaryl, —(C₁-C₆)alkylheteroaryl, —(C₁-C₆)heteroalkylaryl, —(C₁-C₆) heteroalkylheteroaryl, and —(C₁-C₆)alkyl substituted with a moiety selected from the group consisting of halo, —OH, —NO₂, —(C₀-C₆)alkyl-C(O)—N(R¹)₂ and —(C₀-C₆)heteroalkyl-C(O)—N(R¹)₂;
A and B are independently selected from the group consisting of —H, —(C₁-C₆)alkyl, —(C₁-C₆)heteroalkyl, —(C₃-C₆)cycloalkyl, heterocyclyl, —(C₀-C₆)alkyl-aryl, —(C₀-C₆)alkyl-heteroaryl, —(C₀-C₆)heteroalkyl-aryl, —(C₀-C₆)hetroalkyl-heteroaryl, —S(O)₂—(C₀-C₆)alkyl-aryl, —S(O)₂—(C₀-C₆)alkyl-heteroaryl, —S(O)₂—(C₀-C₆)heteroalkyl-aryl, —S(O)₂—(C₀-C₆)heteroalkyl-heteroaryl, —C(O)—(C₁-C₆)alkyl-aryl, —C(O)—(C₁-C₆)alkyl-heteroaryl, —C(O)—(C₁-C₆)heteoralkyl-aryl, —C(O)—(C₁-C₆)heteroalkyl-heteroaryl, —C(O)O—(C₀-C₆)alkyl-aryl, —C(O)O—(C₁-C₆)alkyl-heteroaryl, —C(O)O—(C₁-C₆)heteroalkyl-aryl, —C(O)O—(C₁-C₆)heteroalkyl-heteroaryl, —C(O)N(R¹)—(C₁-C₆)alkyl-aryl, —C(O)N(R¹)—(C₁-C₆)heteroalkyl-aryl, —C(O)N(R¹)—(C₁-C₆)alkyl-heteroaryl, —C(O)N(R¹)—(C₁-C₆)heteroalkyl-heteroaryl, —(C₂-C₆)alkyl-N(R¹)₂, —(C₂-C₆)heteroalkyl-N(R¹)₂, —(C₂-C₆)alkyl-O(R¹), —(C₂-C₆)heteroalkyl-O(R¹), —(C₁-C₇)alkyl-W, —(C₁-C₇)heteoralkyl-W, —(C₂-C₅)alkyl-(CH═CH)₀₋₁—W, —(C₂-C₅)heteroalkyl-(CH═CH)₀₋₁—W, —(C₂-C₅)alkyl-(C≡C)₀₋₁—W, —(C₂-C₅)heteroalkyl-C≡C—W, —C(O)—(C₁-C₇)alkyl-W, —C(O)—(C₁-C₇)heteroalkyl-W, —S(O)₂—(C₁-C₆)alkyl-W, —S(O)₂—(C₁-C₆)heteroalkyl-W, —(C₀-C₇)alkyl-aryl-(CH═CH)₀₋₁—W, —(C₀-C₇)heteroalkyl-aryl-(CH═CH)₀₋₁—W, —(C₀-C₇)alkyl-aryl-(C≡C)₀₋₁—W, —(C₀-C₇)heteroalkyl-aryl-(C≡C)₀₋₁—W, —(C₀-C₇)alkyl-heteroaryl-(CH═CH)₀₋₁—W, —(C₀-C₇)heteroalkyl-heteroaryl-(CH═CH)₀₋₁—W, —(C₀-C₇)alkyl-heteroaryl-(C≡C)₀₋₁—W, —(C₀-C₇)heteroalkyl-heteroaryl-(C≡C)₀₋₁—W, —(C₀-C₇)alkyl-aryl-(C₀-C₄)alkyl-W, —(C₀-C₇)heteroalkyl-aryl-(C₀-C₄)alkyl-W, —(C₀-C₇)alkyl-aryl-(C₀-C₄)heteroalkyl-W, —(C₀-C₇) heteroalkyl-aryl-(C₀-C₄)heteroalkyl-W, —(C₀-C₇)alkyl-heteroaryl-(C₀-C₄)alkyl-W, —(C₀-C₇) heteroalkyl-heteroaryl-(C₀-C₄)alkyl-W, —(C₀-C₇)alkyl-heteroaryl-(C₀-C₄) heteroalkyl-W, —(C₀-C₇) heteroalkyl-heteroaryl-(C₀-C₄) heteroalkyl-W, —S(O)₂—(C₁-C₆)alkyl-aryl-(C₀-C₄)alkyl-(CH═CH)₀₋₁—W, —S(O)₂—(C₁-C₆)heteroalkyl-aryl-(C₀-C₄)alkyl-(CH═CH)₀₋₁—W, —S(O)₂—(C₁-C₆)alkyl-aryl-(C₀-C₄) heteroalkyl-(CH═CH)₀₋₁—W, —S(O)₂—(C₁-C₆)heteroalkyl-aryl-(C₀-C₄)heteroalkyl-(CH═CH)₀₋₁—W, —S(O)₂—(C₁-C₆)alkyl-aryl-(C₀-C₄)alkyl-(C≡C)₀₋₁—W, —S(O)₂—(C₁-C₆)heteroalkyl-aryl-(C₀-C₄)alkyl-(C≡C)₀₋₁—W, —S(O)₂—(C₁-C₆)alkyl-aryl-(C₀-C₄)heteroalkyl-(C≡C)₀₋₁—W, —S(O)₂—(C₁-C₆)heteroalkyl-aryl-(C₀-C₄)heteroalkyl-(C≡C)₀₋₁—W, —S(O)₂—(C₁-C₆)alkyl-heteroaryl-(C₀-C₄)alkyl-(CH═CH)₀₋₁—W, —S(O)₂—(C₁-C₆)heteroalkyl-heteroaryl-(C₀-C₄)alkyl-(CH═CH)₀₋₁—W, —S(O)₂—(C₁-C₆)alkyl-heteroaryl-(C₀-C₄)heteroalkyl-(CH═CH)₀₋₁—W, —S(O)₂—(C₁-C₆)heteroalkyl-heteroaryl-(C₀-C₄)heteroalkyl-(CH═CH)₀₋₁—W, —S(O)₂—(C₁-C₆)alkyl-heteroaryl-(C₀-C₄)alkyl-(C≡C)₀₋₁—W, —S(O)₂—(C₁-C₆)heteroalkyl-heteroaryl-(C₀-C₄)alkyl-(C≡C)₀₋₁—W, —S(O)₂—(C₁-C₆)alkyl-heteroaryl-(C₀-C₄)heteroalkyl-(C≡C)₀₋₁—W, —S(O)₂—(C₁-C₆)heteroalkyl-heteroaryl-(C₀-C₄)heteroalkyl-(C≡C)₀₋₁—W, —C(O)—(C₁-C₆)alkyl-aryl-(C₀-C₄)alkyl-(CH═CH)₀₋₁—W, —C(O)—(C₁-C₆)heteroalkyl-aryl-(C₀-C₄)alkyl-(CH═CH)₀₋₁—W, —C(O)—(C₁-C₆)alkyl-aryl-(C₀-C₄)heteroalkyl-(CH═CH)₀₋₁—W, —C(O)—(C₁-C₆)heteroalkyl-aryl-(C₀-C₄) heteroalkyl-(CH═CH)₀₋₁—W, —C(O)—(C₁-C₆)alkyl-aryl-(C₀-C₄)alkyl-(C≡C)₀₋₁—W, —C(O)—(C₁-C₆)heteroalkyl-aryl-(C₀-C₄)alkyl-(C≡C)₀₋₁—W, —C(O)—(C₁-C₆)alkyl-aryl-(C₀-C₄) heteroalkyl-(C≡C)₀₋₁—W, —C(O)—(C₁-C₆) heteroalkyl-aryl-(C₀-C₄)heteroalkyl-(C≡C)₀₋₁—W, —C(O)—(C₁-C₆)alkyl-heteroaryl-(C₀-C₄)alkyl-(CH═CH)₀₋₁—W, —C(O)—(C₁-C₆)heteroalkyl-heteroaryl-(C₀-C₄)alkyl-(CH═CH)₀₋₁—W, —C(O)—(C₁-C₆)alkyl-heteroaryl-(C₀-C₄)heteroalkyl-(CH═CH)₀₋₁—W, —C(O)—(C₁-C₆)heteroalkyl-heteroaryl-(C₀-C₄)heteroalkyl-(CH═CH)₀₋₁—W, —C(O)—(C₁-C₆)alkyl-heteroaryl-(C₀-C₄)alkyl-(C≡C)₀₋₁—W, —C(O)—(C₁-C₆)heteroalkyl-heteroaryl-(C₀-C₄)alkyl-(C≡C)₀₋₁—W, —C(O)—(C₁-C₆)alkyl-heteroaryl-(C₀-C₄)heteroalkyl-(C≡C)₀₋₁—W, —C(O)—(C₁-C₆) heteroalkyl-heteroaryl-(C₀-C₄)heteroalkyl-(C≡C)₀₋₁—W, —C(O)O—(C₀-C₆)alkyl-aryl-(C₀-C₄)alkyl-(CH═CH)₀₋₁—W, —C(O)O—(C₀-C₆)heteroalkyl-aryl-(C₀-C₄)alkyl-(CH═CH)₀₋₁—W, —C(O)O—(C₀-C₆)alkyl-aryl-(C₀-C₄)heteroalkyl-(CH═CH)₀₋₁—W, —C(O)O—(C₀-C₆)heteroalkyl-aryl-(C₀-C₄) heteroalkyl-(CH═CH)₀₋₁—W, —C(O)O—(C₀-C₆)alkyl-aryl-(C₀-C₄)alkyl-(C≡C)₀₋₁—W, —C(O)O—(C₀-C₆)heteroalkyl-aryl-(C₀-C₄)alkyl-(C≡C)₀₋₁—W, —C(O)O—(C₀-C₆)alkyl-aryl-(C₀-C₄) heteroalkyl-(C≡C)₀₋₁—W, —C(O)O—(C₀-C₆)heteroalkyl-aryl-(C₀-C₄)heteroalkyl-(C≡C)₀₋₁—W, —C(O)O—(C₀-C₆)alkyl-heteroaryl-(C₀-C₄)alkyl-(CH═CH)₀₋₁—W, —C(O)O—(C₀-C₆)heteroalkyl-heteroaryl-(C₀-C₄)alkyl-(CH═CH)₀₋₁—W, —C(O)O—(C₁-C₆)alkyl-heteroaryl-(C₀-C₄)heteroalkyl-(CH═CH)₀₋₁—W, —C(O)O—(C₁-C₆)heteroalkyl-heteroaryl-(C₀-C₄)heteroalkyl-(CH═CH)₀₋₁—W, —C(O)O—(C₁-C₆)alkyl-heteroaryl-(C₀-C₄)alkyl-(C≡C)₀₋₁—W, —C(O)O—(C₁-C₆)heteroalkyl-heteroaryl-(C₀-C₄)alkyl-(C≡C)₀₋₁—W, —C(O)O—(C₁-C₆)alkyl-heteroaryl-(C₀-C₄)heteroalkyl-(C≡C)₀₋₁—W, —C(O)O—(C₁-C₆)heteroalkyl-heteroaryl-(C₀-C₄)heteroalkyl-(C≡C)₀₋₁—W, —C(O)N(R¹)—(C₀-C₆)alkyl-aryl-(C₀-C₄)alkyl-(CH═CH)₀₋₁—W, —C(O)N(R₁)—(C₀-C₆)heteroalkyl-aryl-(C₀-C₄)alkyl-(CH═CH)₀₋₁—W, —C(O)N(R₁)—(C₀-C₆)alkyl-aryl-(C₀-C₄)heteroalkyl-(CH═CH)₀₋₁—W, —C(O)N(R₁)—(C₁-C₆)heteroalkyl-aryl-(C₀-C₄)heteroalkyl-(CH═CH)₀₋₁—W, —C(O)N(R₁)—(C₁-C₆)alkyl-aryl-(C₀-C₄)alkyl-(C≡C)₀₋₁—W, —C(O)N(R₁)—(C₁-C₆)heteroalkyl-aryl-(C₀-C₄)alkyl-(C≡C)₀₋₁—W, —C(O)N(R₁)—(C₁-C₆)alkyl-aryl-(C₀-C₄)heteroalkyl-(C≡C)₀₋₁—W, —C(O)N(R₁)—(C₁-C₆)heteroalkyl-aryl-(C₀-C₄)heteroalkyl-(C≡C)₀₋₁—W, —C(O)N(R¹)—(C₁-C₆)alkyl-heteroaryl-(C₀-C₄)alkyl-(CH═CH)₀₋₁—W, —C(O)N(R¹)—(C₁-C₆)heteroalkyl-heteroaryl-(C₀-C₄)alkyl-(CH═CH)₀₋₁—W, —C(O)N(R¹)—(C₁-C₆)alkyl-heteroaryl-(C₀-C₄)heteroalkyl-(CH═CH)₀₋₁—W, —C(O)N(R¹)—(C₁-C₆)heteroalkyl-heteroaryl-(C₀-C₄)heteroalkyl-(CH═CH)₀₋₁—W, —C(O)N(R¹)—(C₁-C₆)alkyl-heteroaryl-(C₀-C₄)alkyl-(C≡C)₀₋₁—W, —C(O)N(R¹)—(C₁-C₆)heteroalkyl-heteroaryl-(C₀-C₄)alkyl-(C≡C)₀₋₁—W, —C(O)N(R¹)—(C₁-C₆)alkyl-heteroaryl-(C₀-C₄)heteroalkyl-(C≡C)₀₋₁—W and —C(O)N(R¹)—(C₁-C₆)heteroalkyl-heteroaryl-(C₀-C₄)heteroalkyl-(C≡C)₀₋₁—W;
   wherein each of the alkyl and heteroalkyl moieties is optionally substituted; and
   wherein each of the aryl, heteroaryl, cycloalkyl or heterocyclyl moieties is optionally substituted with one or more groups selected from R²; and the asterisk mark * indicates a chiral carbon atom, with the proviso that no more than two of Z, A, B, D and E end with the moiety W.

In a preferred embodiment of Formula (I) of the present invention, Embodiment A, n is 0.

In another preferred embodiment of Formula (I) of the present invention, Embodiment B, n is 1.

In another preferred embodiment of Formula (I) of the present invention, Embodiment C, X¹, X², X³ and X⁴ are independently selected from the group consisting of CH and C-Z, wherein no more than one of X¹, X², X³ and X⁴ are C-Z.

In another preferred embodiment of Formula (I) of the present invention, Embodiment D, X¹, X², X³ and X⁴ are independently selected from the group consisting of CH, N and C-Z, wherein no more than two of X¹, X², X³ and X⁴ are N and no more than one of X¹, X², X³ and X⁴ are C-Z, wherein Z is selected from the group consisting of —H, halo, —CF₃, —NO₂, —CN, —(C₀-C₆)alkyl-OR¹, —(C₀-C₆)alkyl-N(R¹)₂, —(C₁-C₆)alkyl, —N(R¹)—C(O)—(C₁-C₆)alkyl, —N(R¹)—S(O)₂—(C₁-C₆)alkyl, —O—(C₂-C₆)alkyl-N(R¹)(R¹), —S—R¹, —(C₀-C₆)alkyl-C(O)—OR¹, —N(R¹)—C(O)—CF₃ or —N(R¹)—(C₂-C₆)alkyl-N(R¹)(R¹), —(C₀-C₇)alkyl-W, —C(O)—N(R¹)—(C₁-C₆)alkyl-W, —(C₀-C₃)alkyl-N(R¹)—C(O)—(C₁-C₆)alkyl-W, —(C₀-C₃)alkyl-N(R¹)—C(O)—(C₁-C₆)alkyl-C(O)-aryl, —(C₀-C₃)alkyl-N(R¹)—C(O)—(C₁-C₆)alkyl-C(O)-heteroaryl, —(C₀-C₃)alkyl-N(R¹)—C(O)—(C₁-C₆)alkyl-C(O)—N(R¹)-aryl, —(C₀-C₃)alkyl-N(R¹)—C(O)—(C₁-C₆)alkyl-C(O)—N(R¹)-heteroaryl, —(C₀-C₇)alkyl-aryl-W, —(C₀-C₆)alkyl-OR¹, —N(R¹)—C(O)—OR¹, wherein each of the aryl, heteroaryl, cycloalkyl and heterocyclyl moieties of the above-mentioned Z is optionally substituted with one or more substituents selected from the group consisting of oxo, —OH, —CN, —(C₁-C₆)alkyl, —(C₁-C₆)alkoxy, —NO₂, —N(R¹)₂, halo, —SH, mono- to per-halogenated-(C₁-C₆)alkyl and —(C₂-C₄)alkyl-N(R¹)₂, wherein two R¹ groups, together with the nitrogen atom to which they are attached optionally form a heterocyclyl group.

In another preferred embodiment of Formula (I) of the present invention, Embodiment E, X¹, X², X³ and X⁴ are independently selected from the group consisting of CH, C-Z and N, wherein no more than two of X¹, X², X³ and X⁴ are N and no more than one of X¹, X², X³ and X⁴ are C-Z, wherein Z is selected from the group consisting of —F, —Cl, —Br, CF₃, NO₂, —CN, —OR¹, —NR¹R¹, —(CH₂)₀₋₄OR¹, —(CH₂)₀₋₄N(R¹)₂, —CH₂OH, —CH₃, —N(R¹)C(O)CH₃, —N(R¹)SO₂CH₃, —O(CH₂)₂₋₄N(R¹)(R¹), —SR¹, —(CH₂)₀₋₄C(O)OR¹, —N(R¹)C(O)CF₃ and —N(R¹)(CH₂)₂N(R¹)(R¹), wherein two R¹ groups, together with the nitrogen atom to which they are attached, optionally form a heterocyclyl group.

In another preferred embodiment of Formula (I) of the present invention, Embodiment F. X¹, X², X³ and X⁴ are independently selected from the group consisting of CH and C-Z, wherein only one of X¹, X², X³ and X⁴ are C-Z, and wherein Z is selected from the group consisting of —H, —(C₀-C₇)alkyl-W, —(C₀-C₅)alkyl-CH═CH—W, —(C₀-C₅)alkyl-C≡C—W, —C(O)—(C₁-C₇)alkyl-W, —(C₀-C₃)alkyl-N(R¹)—C(O)—(C₁-C₆)alkyl-W, —(C₀-C₃)alkyl-N(R¹)—C(S)—(C₁-C₆)alkyl-W, —C(O)—N(R¹)—(C₁-C₆)alkyl-W, —C(S)—N(R¹)—(C₁-C₆)alkyl-W, —(C₀-C₃)alkyl-N(R¹)—(C₁-C₆)alkyl-W, —(C₀-C₃)alkyl-N(R¹)—C(O)—N(R¹)—(C₁-C₆)alkyl-W, —(C₀-C₃)alkyl-N(R¹)—C(S)—N(R¹)—(C₁-C₆)alkyl-W, —(C₀-C₃)alkyl-N(R¹)—C(O)—O—(C₁-C₆)alkyl-W, —S(O)₂—N(R¹)—(C₁-C₆)alkyl-W, —(C₀-C₃)alkyl-N(R¹)—S(O)₂—(C₁-C₆)alkyl-W, —O—C(O)—N(R¹)₂, —(C₀-C₆)alkyl-O—C(O)—N(R¹)—(C₁-C₆)alkyl-W, —(C₀-C₃)alkyl-O—(C₁-C₆)alkyl-W, —(C₀-C₃)alkyl-S—(C₁-C₆)alkyl-W, —N(R¹)—C(O)—O—S(O)₂—N(R¹)₂, —N(R¹)—S(O)₂—R¹, —(C₀-C₇)alkyl-aryl-W, —(C₀-C₇)alkyl-heteroaryl-W, —(C₀-C₃)alkyl-O—(C₀-C₃)alkyl-aryl, —(C₀-C₃)alkyl-O—(C₀-C₃)alkyl-heteroaryl, -aryl, —(C₁-C₆)alkylaryl, -heteroaryl, —(C₁-C₆)alkylheteroaryl, —(C₁-C₈)heteroalkyl, —(C₃-C₆)cycloalkyl, —(C₃-C₆)heterocycloalkyl, —(C₀-C₃)alkyl-N(R¹)—C(O)—(C₁-C₆)alkyl-C(O)-aryl, —(C₀-C₃)alkyl-N(R¹)—C(O)—(C₁-C₆)alkyl-C(O)-heteroaryl, —(C₀-C₃)alkyl-N(R¹)—C(O)—(C₁-C₆)alkyl-C(O)—N(R¹)-aryl and —(C₀-C₃)alkyl-N(R¹)—C(O)—(C₁-C₆)alkyl-C(O)—N(R¹)-heteroaryl, wherein two R¹ groups, together with the nitrogen atom to which they are attached, optionally form a heterocyclyl group.

In another preferred embodiment of Formula (I) of the present invention, Embodiment G, R¹ is independently —(C₀-C₆)alkyl-aryl or —(C₁-C₄)alkyl.

In another preferred embodiment of Formula (I) of the present invention, Embodiment H, R¹ is independently selected from the group consisting of phenyl, benzyl, methyl, ethyl, t-butyl and i-propyl.

In another preferred embodiment of Formula (I) of the present invention, Embodiment I, Z is —(C₂-C₄)alkyl-N(R¹)₂, and the two R¹ groups, together with the nitrogen atom to which they are attached, optionally form a heterocyclyl selected from the group consisting of morpholinyl, piperazinyl, piperidinyl, pyrrolydinyl, and azetidinyl.

In another preferred embodiment of Formula (I) of the present invention, Embodiment J, W is selected from the group consisting of 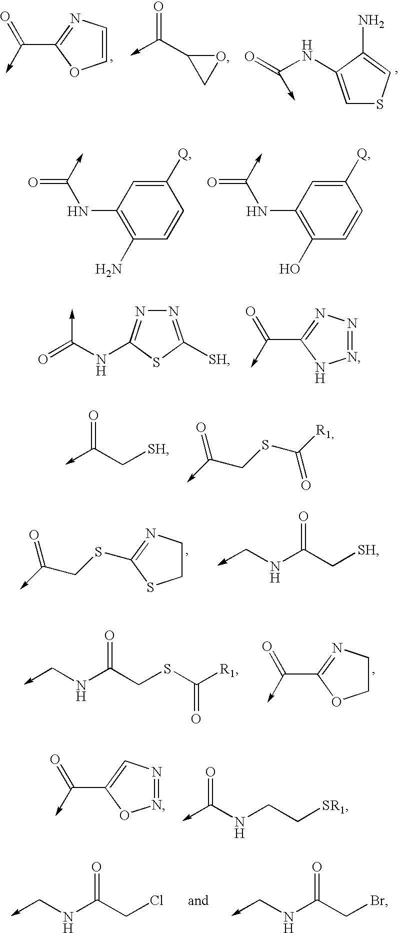
wherein Q is selected from the group consisting of —H, —(C₁-C₆)alkyl, —(C₀-C₆)alkyl-OR¹, heterocyclyl, —N(R¹)₂, halo, aryl and heteroaryl.

In another preferred embodiment of Formula (I) of the present invention, Embodiment K, W is selected from the group consisting of —C(O)—NH—OH, —COCF₃, —COCHF₂, —COCH₂F, —C(O)CH₃, —C(O)C₂H₅, —(CH₂)₁₋₆—N(OH)C(O)H and —CON(R¹)₂.

In another preferred embodiment of Formula (I) of the present invention, Embodiment L, Q is independently selected from the group consisting of heterocyclyl, aryl and heteroaryl.

In another preferred embodiment of Formula (I) of the present invention, Embodiment M, Q is independently selected from the group consisting of thiophenyl, furanyl, tetrazolyl, imidazolyl, pyridinyl, pyrimidinyl, pyrrolyl, thiazolyl, oxazolyl and isooxazolyl.

In another preferred embodiment of Formula (I) of the present invention, Embodiment N, E and D are independently selected from the group consisting of —H, —(C₁-C₆)alkyl, —(C₁-C₆)heteroalkyl, —(C₁-C₆)alkyl-OR¹, —(C₁-C₆)alkyl-C(O)—N(R¹)₂, —(C₁-C₆)alkyl-C(O)—O—(C₁-C₆)alkyl, 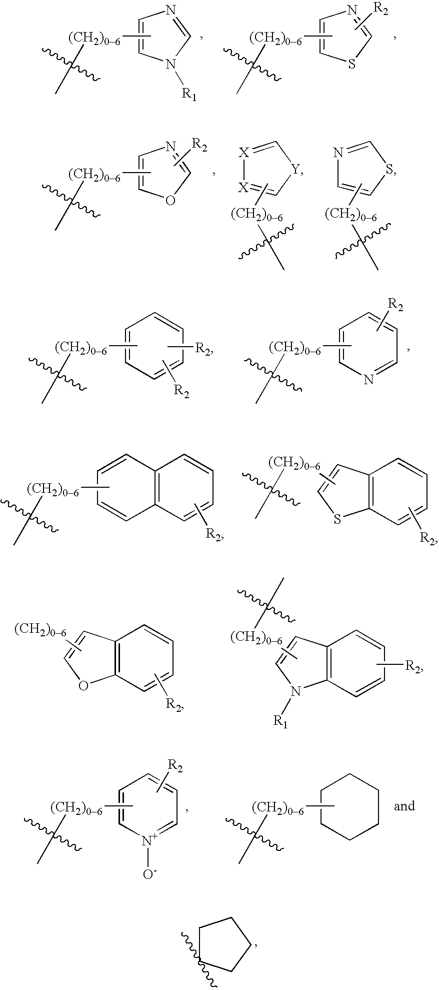
wherein Y is selected from the group consisting of —O—, —NR¹—, and —S—, and X is —CH— or —N—.

In another preferred embodiment of Formula (I) of the present invention, Embodiment O, E and D together with the carbon atom to which they are attached form a 3- to 6-membered cycloalkyl wherein the cycloalkyl is optionally substituted.

In another preferred embodiment of Formula (I) of the present invention, Embodiment P. R² is independently selected from the group consisting of —H, —CH₃, —OR₁, —(CH₂)₀₋₄N(R₁)₂, —F, —Cl, —Br, —OCF₃, —CF₃, —C(Ph)₃, N₂, alkyl, aryl, heteroaryl, SR₁ and —CN.

In another preferred embodiment of Formula (I) of the present invention, Embodiment Q. A and B are independently selected from the group consisting of —H, —(C₁-C₆)alkyl, heteroalkyl, —(C₃-C₆)cycloalkyl, heterocycle, —(C₀-C₃)alkyl-aryl, —(C₀-C₃)alkyl-heteroaryl, —(C H₂)₁₋₅—W, —S(O)₂—(CH₂)₀₋₅-aryl, —S(O)₂—(CH₂)₀₋₅-heteroaryl and —C(O)—R², wherein each of the alkyl and heteroalkyl moieties is optionally substituted; and wherein each of the aryl and heteroaryl moieties is optionally substituted with one or more moieties selected from the group consisting of —(C₀-C₆)alkyl-aryl, —(C₀-C₆)alkyl-heteroaryl, —(C₁-C₆)alkyl, halo, —OH, —O—(C₁-C₆)alkyl, —C(O)OH, —C(O)—NH—OH.

In another preferred embodiment of Formula (I) of the present invention, Embodiment R, A and B are independently selected from the group consisting of —H, 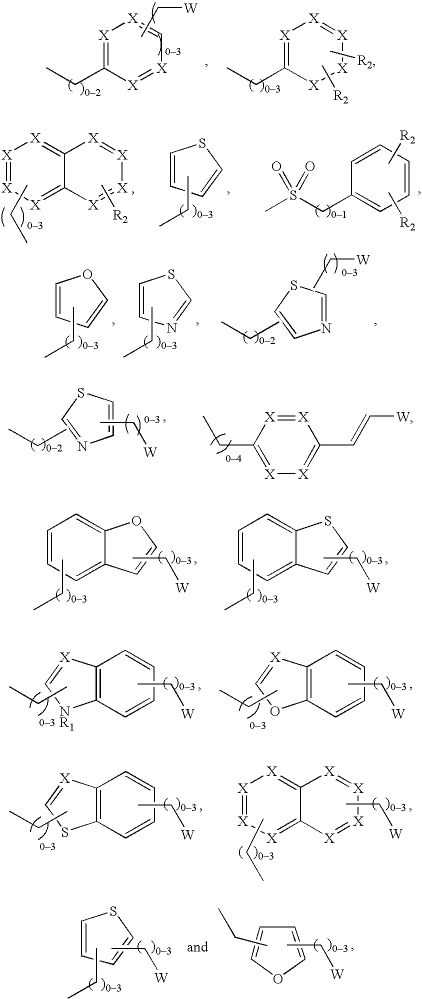
wherein X is —CH— or —N—.

In another preferred embodiment of Formula (I) of the present invention, Embodiment S,

X¹, X³ and X⁴ are CH;

X² is C-Z;

n is 0; and

A is —H,

with the proviso that one of E or D is H.

Another preferred embodiment of Formula (I) of the present invention, Embodiment T, provides compounds of Embodiment S according to the formula (II) 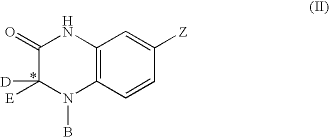
and N-oxides, hydrates, solvates, pharmaceutically acceptable salts, prodrugs and complexes thereof, wherein
Z is selected from the group consisting of —H, —C(O)—N(R¹)₂, —C(O)—N(R¹)—(C₁-C₆)alkyl-W, —(C₀-C₇)alkyl-W, —(C₂-C₇)alkenyl-W, —(C₀-C₃)alkyl-N(R¹)—C(O)—(C₁-C₆)alkyl-W and —(C₀-C₇)alkyl-aryl-W;
B is selected from the group consisting of —H, —S(O)₂—(C₀-C₆)alkyl-aryl, —(C₀-C₆)alkyl-aryl, —(C₀-C₆)alkyl-heteroaryl and —(C₀-C₇)alkyl-aryl-(CH═CH)₀₋₁—W; and
E and D are independently selected from the group consisting of —H, —(C₁-C₆)alkyl, —(C₁-C₆)heteroalkyl, —(C₀-C₆)alkyl-aryl, —(C₀-C₆)alkyl-heteroaryl, —(C₀-C₆)alkyl-W, —(C₀-C₆)alkyl-C(O)—N(R¹)₂, wherein each of the aryl and heteroaryl is optionally substituted with one or more groups selected from R²,
with the proviso that one of E and D is —H.

In a preferred embodiment of Embodiment T of the present invention, Embodiment U,

Z is selected from the group consisting of —C(O)—N(R¹)₂, —C(O)—N(R¹)—(C₁-C₆)alkyl-W; and B is —H.

Another preferred embodiment of Formula (I) of the present invention, Embodiment V, provides compounds according to the formula (III)

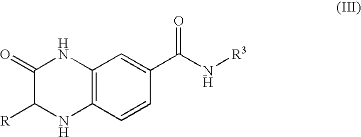

and N-oxides, hydrates, solvates, pharmaceutically acceptable salts, prodrugs and complexes thereof, wherein R and R³ are a combination selected from the group consisting of:

| R | R³ |
|---|---|
| 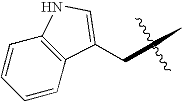 | 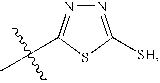 |
| 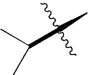 | 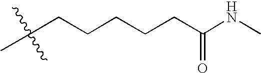 |
| 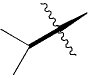 | —OH, |
| 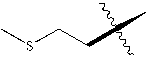 | —OH, |
| 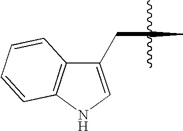 | —OH, |
| 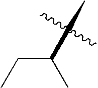 | —OH, |
| 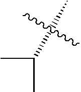 | —OH, |
| 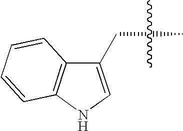 | —OH, |
| 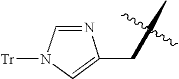 | —OH, |
| 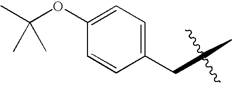 | —OH, |
| 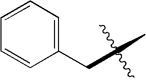 | —OH, |
| H | —OH, |
| 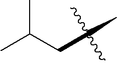 | —OH, |
| 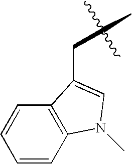 | —OH, |
| 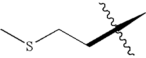 | 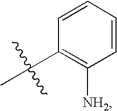 |
| 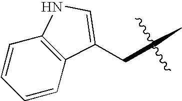 | 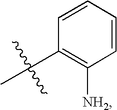 |
| 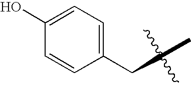 | —OH, |
| 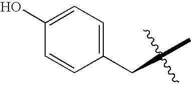 | 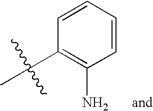 |
| 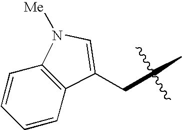 | —OH. |

In another preferred embodiment of Formula (I) of the present invention, Embodiment W,

Z is —C(O)—NH—OH;

B is selected from the group consisting of —S(O)₂—(C₀-C₆)alkyl-aryl, —(C₀-C₆)alkyl-aryl, —(C₀-C₆)alkyl-heteroaryl, each of which is optionally substituted and —(C₀-C₇)alkyl-aryl-(CH═CH)₀₋₁—W; and

E and D are independently selected from the group consisting of —H and —(C₁-C₆)alkyl, wherein the alkyl moiety is optionally substituted, with the proviso that one of C and D is —H.

Another preferred embodiment of Embodiment T of the present invention, Embodiment X, provides compounds according to the formula (IV)

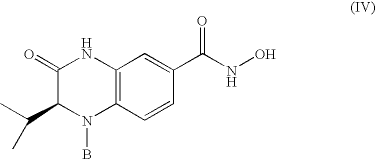

and N-oxides, hydrates, solvates, pharmaceutically acceptable salts, prodrugs and complexes thereof, wherein B is selected from the group consisting of

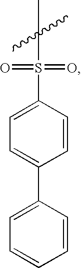

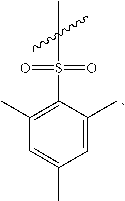

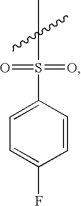

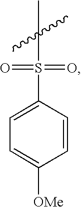

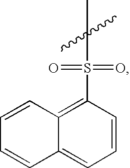

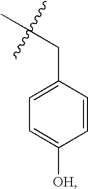

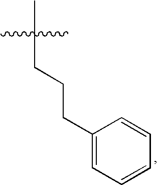

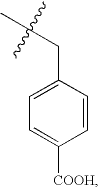

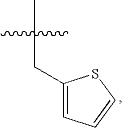

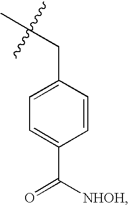

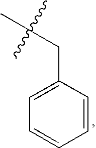

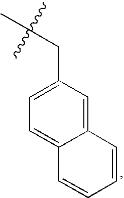

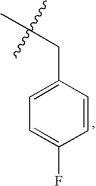

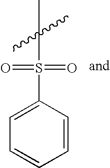

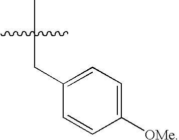

In another preferred embodiment of Formula (I) of the present invention, Embodiment Y,
n is 0;
X¹, X³ and X⁴ are CH;
X² is C-Z;
Z is —(C₀-C₃)alkyl-N(R¹)—C(O)—(C₁-C₆)alkyl-W;
W is selected from the group consisting of —C(O)—NH—OH, —C(O)-heteroaryl, —C(O)-aryl, —C(O)—OR¹, —C(O)—N(R¹)₂ and —C(O)-alkyl, wherein the aryl and heteroaryl moieties of said W are optionally substituted;
A is —H;
B is —H or —(C₀-C₆)alkyl-aryl, wherein the aryl moiety is optionally substituted with one or more groups selected from R²; and
E and D are independently selected from the group consisting of —H, —(C₁-C₆)alkyl and —(C₀-C₆)alkyl-heteroaryl, wherein the heteroaryl moiety is optionally substituted, with the proviso that at least one of E and D are —H.

A preferred embodiment of Embodiment X of the present invention, Embodiment Z, provides compounds according to the formula (V)

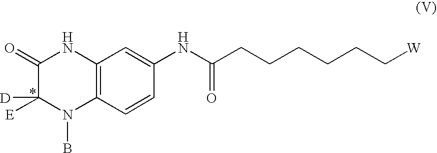

and N-oxides, hydrates, solvates, pharmaceutically acceptable salts, prodrugs and complexes thereof, wherein the combination of B, E, D and W is selected from the group consisting of

| B | E | D | W |
|---|---|---|---|
| H | 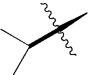 | H | 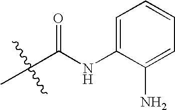 |
| H | 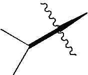 | H | 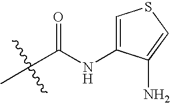 |
| H | 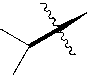 | H | 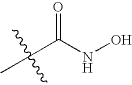 |
| H | H | 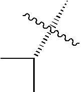 | 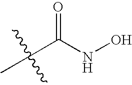 |
| H | H | 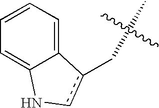 | 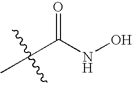 |
| 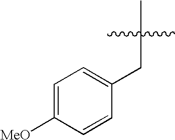 | H | 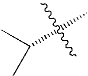 | 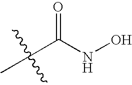 |
| H | 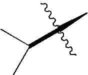 | H | 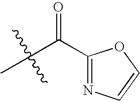 |
| H | 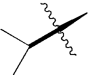 | H | 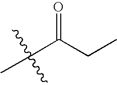 |

In another preferred embodiment of Formula (I) of the present invention, Embodiment AA,
n is 0;
X¹, X², X³ and X⁴ are CH;
A is H;
B is selected from the group consisting of —(C₀-C₆)alkyl-aryl and —(C₀-C₆)alkyl-aryl-(CH═CH)₀₋₁—W, wherein the W moiety is optionally meta or para to the —(C₀-C₆)alkyl moiety, and wherein the aryl moiety of each of the aforementioned B is optionally substituted with one or more substitutents selected from R²;
W is selected from the group consisting of —C(O)—NH—OH, —C(O)—NH-aryl, wherein the aryl is optionally substituted,
E and D are independently selected from the group consisting of —H, —(C₁-C₆)alkyl-M-(C₁-C₃)alkyl-W, —(C₀-C₆)alkyl-C(O)—N(R¹)₂, —(C₀-C₆)alkyl-heteroaryl, —(C₀-C₆)alkyl-aryl and —(C₁-C₆)alkyl-N(R¹)—C(O)—OR¹; and
R¹ is independently selected from the group consisting of —H and —(C₁-C₆)alkyl.

A preferred embodiment of Embodiment AA of the present invention, Embodiment BB, provides compounds according to the formula (VI)

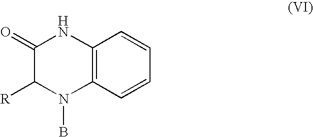

and N-oxides, hydrates, solvates, pharmaceutically acceptable salts, prodrugs and complexes thereof, wherein B and R are a combination selected from the group consisting of

| R | B |
|---|---|
| 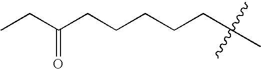 | 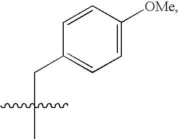 |
| 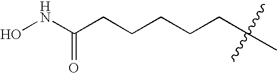 | 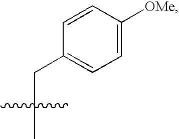 |
| 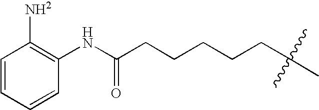 | 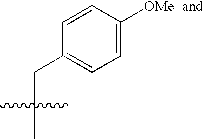 |
| 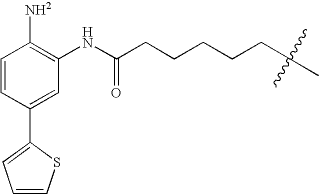 | 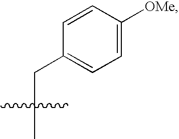 |

Another preferred embodiment of Embodiment AA of the present invention, Embodiment CC, provides compounds according to the formula (VII)

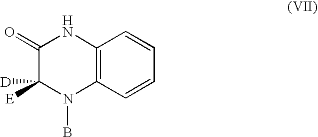

and N-oxides, hydrates, solvates, pharmaceutically acceptable salts, prodrugs and complexes thereof, wherein B, D and E are a combination selected from the group consisting of

| D | E | B |
|---|---|---|
| 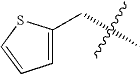 | H | 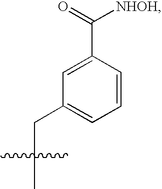 |
| 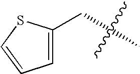 | H | 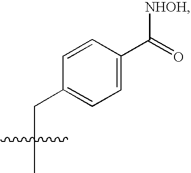 |
| H | 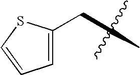 | 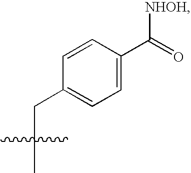 |
| 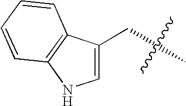 | H | 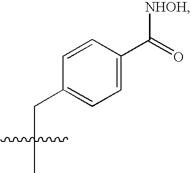 |
| H | 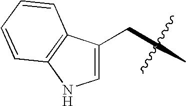 | 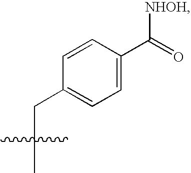 |
| H | 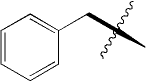 | 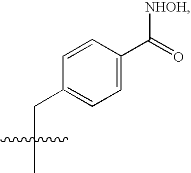 |
| 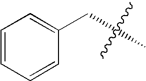 | H | 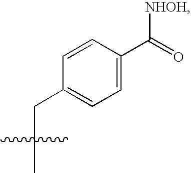 |
| H | H | 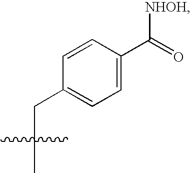 |
| 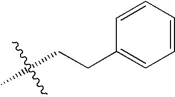 | H | 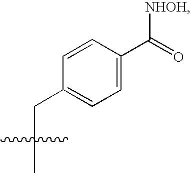 |
| 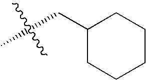 | H | 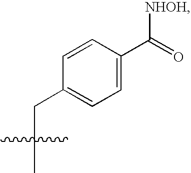 |
| 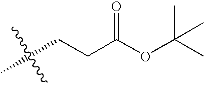 | H | 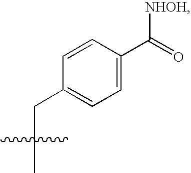 |
| 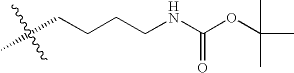 | H | 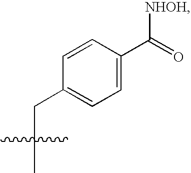 |
| 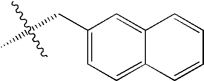 | H | 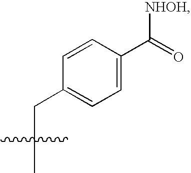 |
| 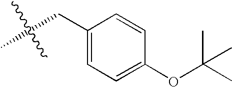 | H | 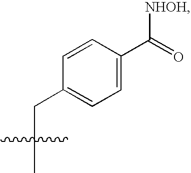 |
| 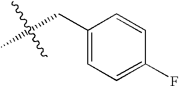 | H | 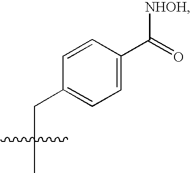 |
| 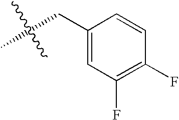 | H | 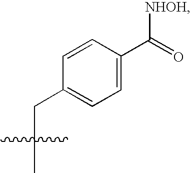 |
| 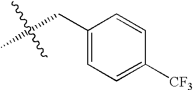 | H | 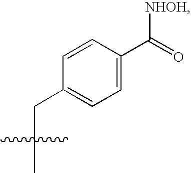 |
| 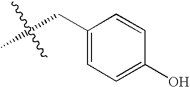 | H | 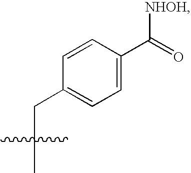 |
| H | 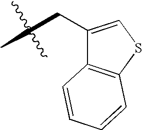 | 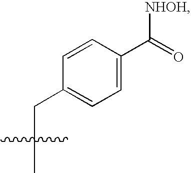 |
| 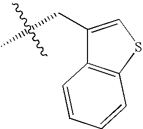 | H | 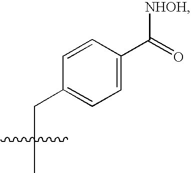 |
| 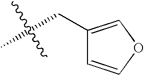 | H | 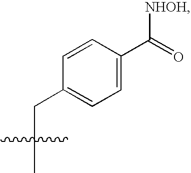 |
| 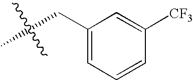 | H | 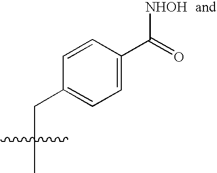 |
| H | 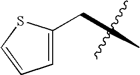 | 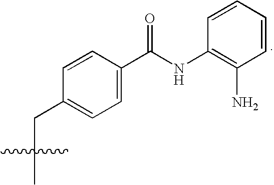 |

In another preferred embodiment of Formula (I) of the present invention, Embodiment DD,
n is 0;
X¹, X² and X³ are CH;
X⁴ is C-Z;
Z is —(C₀-C₇)alkyl-aryl-W;
W is —C(O)—N(R₁)₂;
A and B are —H; and
E and D are independently selected from the group consisting of —H and —(C₁-C₆)alkyl-heteroaryl, with the proviso that one of C and D is —H.

A preferred embodiment of Embodiment DD of the present invention, Embodiment EE, provides compounds according to the formula (VIII)

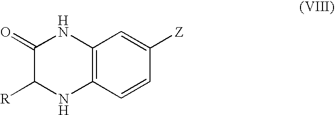

and N-oxides, hydrates, solvates, pharmaceutically acceptable salts, prodrugs and complexes thereof, wherein the combination of R and Z is selected from the group consisting of

| R | Z |
|---|---|
| 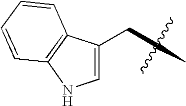 | 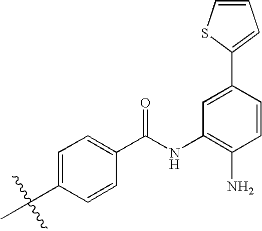 |

In another preferred embodiment of Formula (I) of the present invention, Embodiment FF,
n is 0;
X¹, X² and X³ are CH;
X⁴ is C-Z;
Z is —(C₀-C₇)alkyl-W or —(C₂-C₇)alkenyl-W;
W is —C(O)—NH—OH;
A and B are —H; and
E and D are independently selected from the group consisting of —H and —(C₁-C₆)alkyl-heteroaryl, with the proviso that one of C and D is —H.

A preferred embodiment of Embodiment FF of the present invention, Embodiment GG, provides compounds according to the formula (IX)

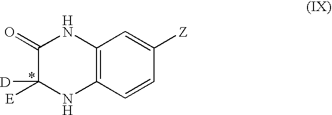

and N-oxides, hydrates, solvates, pharmaceutically acceptable salts, prodrugs and complexes thereof, wherein the combination of E, D and Z is selected from the group consisting of

| E | D | Z |
|---|---|---|
| 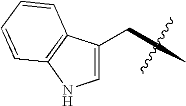 | H | 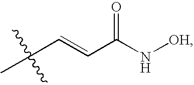 |
| 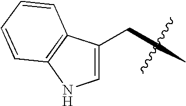 | H | 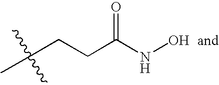 |
| H | 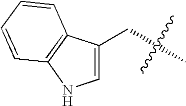 | 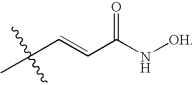 |

In another preferred embodiment of Formula (I) of the present invention, Embodiment HH,
n is 1;
X¹ and X⁴ are CH;
X² and X³ are C-Z;
Z is selected from the group consisting of —H, —(C₀-C₇)alkyl-W, —(C₀-C₆)alkyl-OR¹, —N(R¹)—C(O)—OR¹ and —(C₀-C₃)alkyl-N(R¹)—C(O)—(C₁-C₆)alkyl-W;
A is selected from the group consisting of —H and —(C₁-C₇)alkyl-W, —(C₀-C₆)alkyl-aryl, —(C₀-C₆)alkyl-heteroaryl, wherein the aryl and heteoraryl moiety are optionally substituted with one or more substituents selected from the group consisting of R²;
B is —H;
D and E are independently selected from the group consisting of —H, —(C₁-C₆)alkyl, —(C₀-C₆)alkyl-(C₃-C₆)cylcoalkyl, —(C₀-C₆)alkyl-aryl, —(C₁-C₆)alkyl-heteroaryl, —(C₁-C₆)alkyl-W, wherein each of the cylcoalkyl, aryl and heteroaryl moieties is optionally substituted with one or more groups selected from R²;
W is independently selected from the group consisting of —C(O)—NH—OH, —C(O)—OR¹, —C(O)—N(R¹)₂;
R¹ is independently selected from the group consisting of —H and —(C₀-C₆)-alkyl-aryl, —(C₀-C₆)alkyl-heteroaryl, —(C₁-C₆)-alkyl wherein each of the aryl and heteroaryl moieties is optionally substituted; and
R² is selected from the group consisting of —(C₀-C₆)alkyl substituted with halo, —(C₀-C₆)alkyl-OR₁, —(C₁-C₇)alkyl-W.

In a preferred embodiment of Embodiment HH of the present invention, Embodiment II,
X¹, X², X³ and X⁴ are CH;
A is selected from the group consisting of —(C₁-C₇)alkyl-W;
D and E are independently selected from the group consisting of —H, —(C₁-C₆)alkyl, —(C₀-C₆)alkyl-(C₃-C₆)cylcoalkyl, —(C₀-C₆)alkyl-aryl, —(C₁-C₆)alkyl-heteroaryl, wherein each of the cylcoalkyl, aryl and heteroaryl moieties is optionally substituted with one or more groups selected from R²;
W is independently selected from the group consisting of —C(O)—NH—OH, —C(O)—OR¹, —C(O)—N(R¹)₂;
R¹ is independently selected from the group consisting of —H, —(C₀-C₆)-alkyl-aryl and —(C₀-C₆)alkyl-heteroaryl, wherein each of the aryl and heteroaryl moieties is optionally substituted; and
R² is selected from the group consisting of —(C₀-C₆)alkyl-OR₁.

A preferred embodiment of Embodiment II of the present invention, Embodiment JJ, provides compounds according to the formula (X)

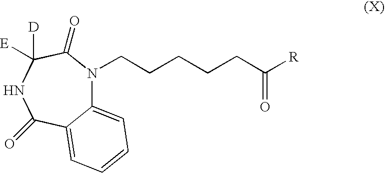

and N-oxides, hydrates, solvates, pharmaceutically acceptable salts, prodrugs and complexes thereof, wherein D, E and R are a combination selected from the group consisting of

| D | E | R |
|---|---|---|
| 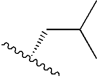 | H | 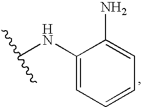 |
| H | 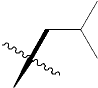 | 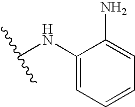 |
| 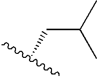 | H | 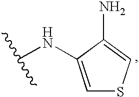 |
| H | 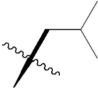 | 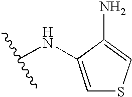 |
| 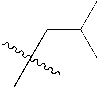 | H | 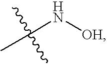 |
| H | H | 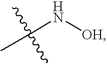 |
| 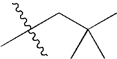 | H | 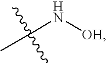 |
| 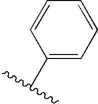 | H | 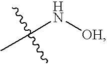 |
| H | 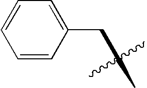 | —OH, |
| H | 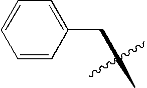 | —NH—OH |
| 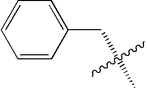 | H | 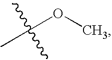 |
| 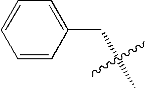 | H | —OH, |
| 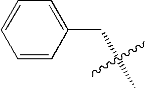 | H | —NH—OH, |
| H | 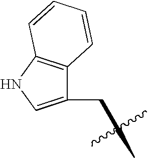 | 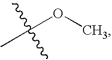 |
| H | 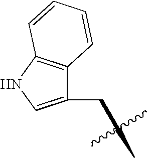 | —OH, |
| H | 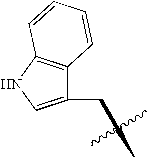 | —NH—OH, |
| 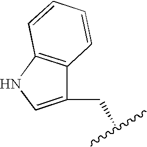 | H | —OH, |
| 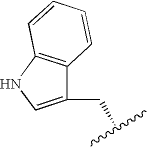 | H | —NH—OH, |
| 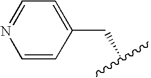 | H | —OH, |
| 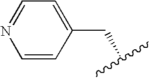 | H | —NH—OH, |
| H | 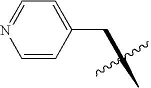 | —OH, |
| H | 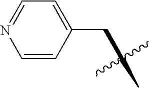 | —NH—OH, |
| H | 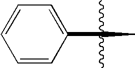 | —OH, |
| H | 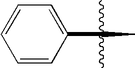 | —NH—OH, |
| 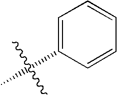 | H | —NH—OH, |
| 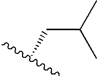 | H | —OH, |
| 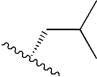 | H | —NH—OH, |
| H | 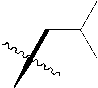 | —NH—OH, |
| H | 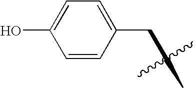 | —OH, |
| 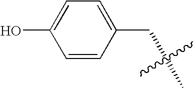 | H | —OH, |
| H | 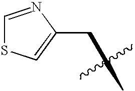 | —OH, |
| 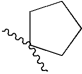 | | —NH—OH, |
| H | 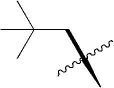 | —NH—OH, |
| 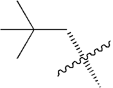 | H | —NH—OH, |
| H | 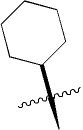 | —NH—OH, |
| 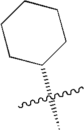 | H | —NH—OH, |
| H | 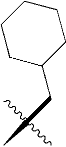 | —NH—OH, |
| 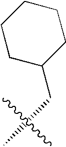 | H | —NH—OH, |
| H | 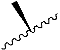 | —NH—OH and |
| 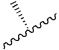 | H | —NH—OH. |

Another preferred embodiment of Embodiment II of the present invention, Embodiment KK, provides compounds according to the formula (XI)

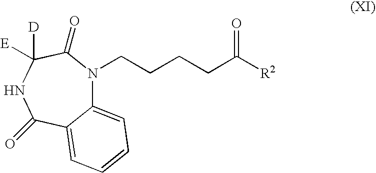

and N-oxides, hydrates, solvates, pharmaceutically acceptable salts, prodrugs and complexes thereof, wherein D, E and R are a combination selected from the group consisting of

| D | E | R |
|---|---|---|
| H | 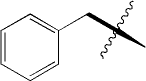 | 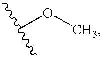 |
| H | 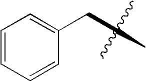 | —OH, |
| 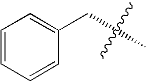 | H | 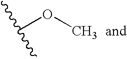 |
| 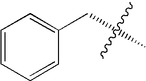 | H | —OH. |

In another preferred embodiment of Embodiment HH of the present invention, Embodiment LL,
X¹, X², X³ and X⁴ are CH;
A is selected from the group consisting of —H, —(C₀-C₆)alkyl-aryl, —(C₀-C₆)alkyl-heteroaryl, wherein the aryl and heteoraryl moiety are optionally substituted with one or more substituents selected from the group consisting of R²;
B is —H;
D and E are independently selected from the group consisting of —H, —(C₁-C₆)alkyl-W;
W is —C(O)—NH—OH; and
R² is selected from the group consisting of —(C₀-C₆)alkyl substituted with halo and —(C₀-C₆)alkyl-OR₁.

A preferred embodiment of Embodiment LL of the present invention, Embodiment MM, provides compounds according to the formula (XII)

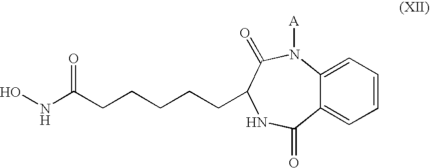

and N-oxides, hydrates, solvates, pharmaceutically acceptable salts, prodrugs and complexes thereof, wherein A is selected from the group consisting of

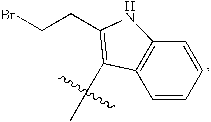

H

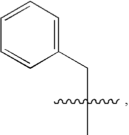

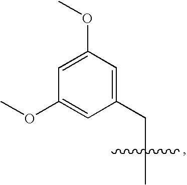

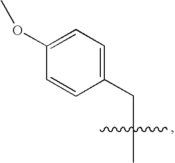

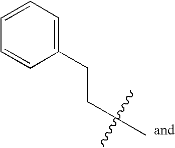

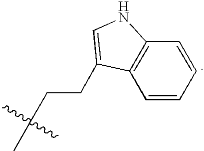

In another embodiment of Embodiment HH of the present invention, Embodiment NN,

X¹, X² and X⁴ are CH;

X³ is C-Z;

Z is —(C₀-C₆)alkyl-OR¹;

R¹ is —(C₀-C₆)alkyl-aryl;

A is —H;

D and E are independently selected from the group consisting of —H and —(C₁-C₆)alkyl-W; and

W is —C(O)—NH—OH and —C(O)—OR¹.

In a preferred embodiment of Embodiment NN of the present invention, Embodiment OO, provides compounds according to the formula (XIII)

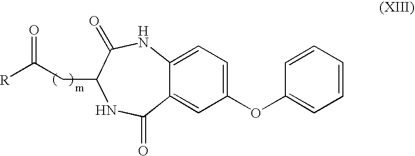

and N-oxides, hydrates, solvates, pharmaceutically acceptable salts, prodrugs and complexes thereof, wherein the combination of m and R is selected from the group consisting of

| m | R, |
|---|---|
| 1 | —NH—OH, |
| 2 | —NH—OH, |
| 5 | —NH—OH, |
| 1 | —OH, |
| 2 | —OH and |
| 4 | —OH. |

In another preferred embodiment of Embodiment HH of the present invention, Embodiment PP,
X¹, X² and X⁴ are CH;
X³ is C-Z;
Z is selected from the group consisting of —N(R¹)—C(O)—OR¹ and —(C₀-C₃)alkyl-N(R¹)—C(O)—(C₁-C₆)alkyl-W;
A and B are —H;
D and E are independently selected from the group consisting of —H, —(C₁-C₆)alkyl and —(C₁-C₆)alkyl-W;
W is independently selected from the group consisting of —C(O)—NH—OH and —C(O)—OR¹; and
R¹ is independently selected from the group consisting of —H and —(C₀-C₆)-alkyl-aryl, wherein the aryl moiety is optionally substituted.

A preferred embodiment of Embodiment PP of the present invention, Embodiment QQ, provides compounds according to the formula (XIV)

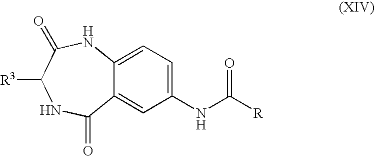

and N-oxides, hydrates, solvates, pharmaceutically acceptable salts, prodrugs and complexes thereof, wherein the combination of R³ and R is selected from the group consisting of

| R³ | R |
|---|---|
| 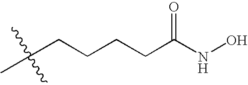 | 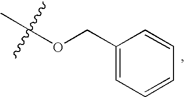 |
| 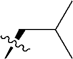 | 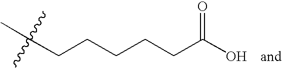 |
| 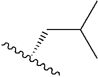 | 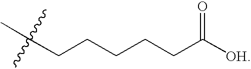 |

In another preferred embodiment of Embodiment HH of the present invention, Embodiment RR,
X¹, X³ and X⁴ are CH;
X² is C-Z;
Z is —(C₀-C₇)alkyl-W;
A and B are —H;
D and E are independently selected from the group consisting of —H, —(C₁-C₆)alkyl, —(C₀-C₆)alkyl-(C₃-C₆)cylcoalkyl, —(C₀-C₆)alkyl-aryl and —(C₁-C₆)alkyl-heteroaryl, wherein each of the cylcoalkyl, aryl and heteroaryl moieties is optionally substituted with one or more groups selected from R²; and
W is —C(O)—NH—OH.

Another preferred embodiment of Embodiment HH of the present invention, Embodiment SS, provides compounds according to the formula (XV) 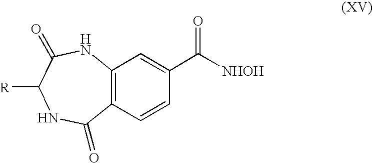
and N-oxides, hydrates, solvates, pharmaceutically acceptable salts, prodrugs and complexes thereof, wherein R is selected from the group consisting of

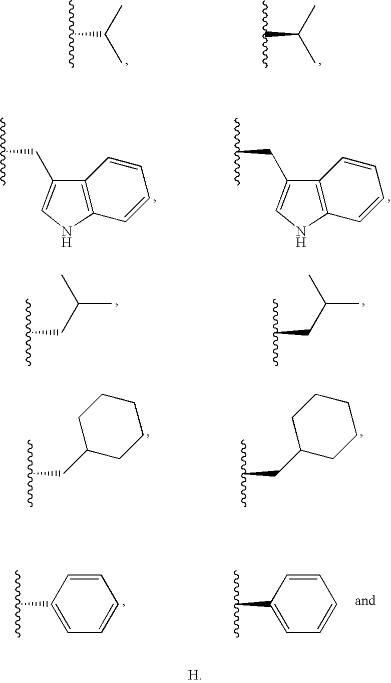

In another preferred embodiment of the first aspect of the present invention, Embodiment TT, there are provided compounds selected from the group consisting of

| Compound No. | Compound |
|---|---|
| 4a | (S)-1,2,3,4-Tetrahydro-N-hydroxy-2-isopropyl-3-oxoquinoxaline-6-carboxamide, |
| 4b | (S)-1,2,3,4-tetrahydro-N-hydroxy-2-(2-(methylthio)ethyl)-3-oxoquinoxaline-6- |
| | carboxamide, |
| 4c | (S)-2-((1H-indol-3-yl)methyl)-1,2,3,4-tetrahydro-N-hydroxy-3-oxoquinoxaline-6- |
| | carboxamide, |
| 4d | (S)-2-sec-butyl-1,2,3,4-tetrahydro-N-hydroxy-3-oxoquinoxaline-6-carboxamide, |
| 4e | (R)-1,2,3,4-tetrahydro-N-hydroxy-2-isopropyl-3-oxoquinoxaline-6-carboxamide, |
| 4f | (R)-2-((1H-indol-3-yl)methyl)-1,2,3,4-tetrahydro-N-hydroxy-3-oxoquinoxaline-6- |
| | carboxamide, |
| 4g | (S)-1,2,3,4-tetrahydro-N-hydroxy-3-oxo-2-((1-trityl-1H-imidazol-4- |
| | yl)methyl)quinoxaline-6-carboxamide, |
| 4h | (S)-2-(4-tert-butoxybenzyl)-1,2,3,4-tetrahydro-N-hydroxy-3-oxoquinoxaline-6- |
| | carboxamide, |
| 4i | (S)-2-Benzyl-1,2,3,4-tetrahydro-N-hydroxy-3-oxoquinoxaline-6-carboxamide, |
| 4k | (S)-1,2,3,4-Tetrahydro-N-hydroxy-2-isobutyl-3-oxoquinoxaline-6-carboxamide, |
| 4l | (S)-1,2,3,4-tetrahydro-N-hydroxy-2-((1-methyl-1-H-indol-3-yl)methyl)-3- |
| | oxoquinoxaline-6-carboxamide, |
| 5c | (S)-2-((1H-indol-3-yl)methyl)-N-(2-aminophenyl)-1,2,3,4-tetrahydro-3- |
| | oxoquinoxaline-6-carboxamide, |
| 6l | (S)-2-(4-hydroxybenzyl)-1,2,3,4-tetrahydro-N-hydroxy-3-oxoquinoxaline-6- |
| | carboxamide, |
| 8 | (S)-1-(4-Methoxybenzyl)-1,2,3,4-tetrahydro-N-hydroxy-2-isopropyl-3 |
| | oxoquinoxaline-6-carboxamide, |
| 11 | (S)—N-(5-(Hydroxycarbamoyl)pentyl)-1,2,3,4-tetrahydro-2-isopropyl-3- |
| | oxoquinoxaline-6-carboxamide, |
| 16a | (S)-1-(4-fluorobenzyl)-N-hydroxy-2-isopropyl-3-oxo-1,2,3,4- |
| | tetrahydroquinoxaline-6-carboxamide, |
| 16b | (S)-1-(4-hydroxybenzyl)-1,2,3,4-tetrahydro-N-hydroxy-2-isopropyl-3- |
| | oxoquinoxaline-6-carboxamide, |
| 16c | (S)-1,2,3,4-tetrahydro-N-hydroxy-2-isopropyl-3-oxo-1-(3- |
| | phenylpropyl)quinoxaline-6-carboxamide, |
| 16d | 4-(((S)-7-(hydroxycarbamoyl)-2,3-dihydro-3-isopropyl-2-oxoquinoxalin-4(1H)- |
| | yl)methyl)benzoic acid, |
| 16e | (S)-1,2,3,4-tetrahydro-N-hydroxy-2-isopropyl-3-oxo-1-((thiophen-2- |
| | yl)methyl)quinoxaline-6-carboxamide, |
| 16f | (S)—N-hydroxy-1-(4-(N-hydroxycarbamimidoyl)benzyl)-2-isopropyl-3-oxo-1,2,3,4- |
| | tetrahydroquinoxaline-6-carboxamide, |
| 16g | (S)-1-benzyl-1,2,3,4-tetrahydro-N-hydroxy-2-isopropyl-3-oxoquinoxaline-6- |
| | carboxamide, |
| 16h | (S)-1,2,3,4-tetrahydro-N-hydroxy-2-isopropyl-1-((naphthalen-3-yl)methyl)-3- |
| | oxoquinoxaline-6-carboxamide, |
| 18a | (S)—N-hydroxy-2-isopropyl-3-oxo-1-(phenylsulfonyl)-1,2,3,4- |
| | tetrahydroquinoxaline-6-carboxamide, |
| 18b | (S)—N-hydroxy-1-(biphenyl-4-sulfonyl)-2-isopropyl-3-oxo-1,2,3,4- |
| | tetrahydroquinoxaline-6-carboxamide, |
| 18c | (S)—N-hydroxy-2-isopropyl-3-oxo-1-(2,4,6-trimethybenzenesulfonyl)-1,2,3,4- |
| | tetrahydroquinoxaline-6-carboxamide, |
| 18e | (S)—N-hydroxy-1-(4-methoxybenzenesulfonyl-2-isopropyl-3-oxo-1,2,3,4- |
| | tetrahydroquinoxaline-6-carboxamide, |
| 18f | (S)—N-hydroxy-1-(1-naphthalenesulfonyl-2-isopropyl-3-oxo-1,2,3,4- |
| | tetrahydroquinoxaline-6-carboxamide, |
| 23a | N1-(2-Aminophenyl)-N8—((S)-1,2,3,4-tetrahydro-2-isopropyl-3-oxoquinoxalin-6- |
| | yl)octanediamide, |
| 24a | N1—((S)-1,2,3,4-Tetrahydro-2-isopropyl-3-oxoquinoxalin-6-yl)-N8- |
| | hydroxyoctanediamide, |
| 24e | N1—((R)-1,2,3,4-Tetrahydro-2-isopropyl-3-oxoquinoxalin-6-yl)-N8- |
| | hydroxyoctanediamide, |
| 24f | N1—((R)-2-((1H-Indol-3-yl)methyl)-1,2,3,4-tetrahydro-3-oxoquinoxalin-6-yl)-N8- |
| | hydroxyoctanediamide, |
| 26e | N1—((R)-1-(4-Methoxybenzyl)-1,2,3,4-tetrahydro-2-isopropyl-3-oxoquinoxalin-6- |
| | yl)-N8-hydroxyoctanediamide, |
| 29a | N—((S)-1,2,3,4-Tetrahydro-2-isopropyl-3-oxoquinoxalin-6-yl)-8-(oxazol-2-yl)-8- |
| | oxooctanamide, |
| 43 | 6-((R)-1-(4-methoxybenzyl)-1,2,3,4-tetrahydro-3-oxoquinoxalin-2-yl)-N-hydroxy- |
| | 4-oxy-hexanamide, |
| 47a | 6-(1-(4-methoxybenzyl)-1,2,3,4-tetrahydro-3-oxoquinoxalin-2-yl)-N- |
| | hydroxyhexanamide, |
| 47b | 6-(1-(4-methoxybenzyl)-1,2,3,4-tetrahydro-3-oxoquinoxalin-2-yl)-N-(2- |
| | aminophenyl)hexanamide, |
| 49 | 6-(1-(4-methoxybenzyl)-1,2,3,4-tetrahydro-3-oxoquinoxalin-2-yl)-N-(2-amino-5- |
| | (thiophen-2-yl)phenyl)hexanamide, |
| 54a₁ | 4-(((S)-2,3-Dihydro-2-oxo-3-((thiophen-2-yl)methyl)quinoxalin-4(1H)-yl)methyl)- |
| | N-hydroxybenzamide, |
| 54a₂ | 4-(((R)-2,3-Dihydro-2-oxo-3-((thiophen-2-yl)methyl)quinoxalin-4(1H)-yl)methyl)- |
| | N-hydroxybenzamide, |
| 54b₁ | N-hydroxy-4-((3-oxo-3,4-dihydroquinoxalin-1(2H)-yl)methyl)-benzamide, |
| 54b₂ | (R)-4-((2-((1H-indol-3-yl)methyl)-3-oxo-3,4-dihydroquinoxalin-1(2H)-yl)methyl)-N- |
| | hydroxybenzamide, |
| 54b₃ | (S)-4-((2-((1H-indol-3-yl)methyl)-3-oxo-3,4-dihydroquinoxalin-1(2H)-yl)methyl)-N- |
| | hydroxybenzamide, |
| 54b₄ | (S)-4-((2-benzyl-3-oxo-3,4-dihydroquinoxalin-1(2H)-yl)methyl)-N- |
| | hydroxybenzamide, |
| 54b₅ | (R)-4-((2-benzyl-3-oxo-3,4-dihydroquinoxalin-1(2H)-yl)methyl)-N- |
| | hydroxybenzamide, |
| 54b₆ | N-hydroxy-4-((3-oxo-3,4-dihydroquinoxalin-1(2H)-yl)methyl)benzamide, |
| 54b₇ | (R)—N-hydroxy-4-((3-oxo-2-phenethyl-3,4-dihydroquinoxalin-1(2H)- |
| | yl)methyl)benzamide, |
| 54b₈ | (R)-4-((2-(cyclohexylmethyl)-3-oxo-3,4-dihydroquinoxalin-1(2H)-yl)methyl)-N- |
| | hydroxybenzamide, |
| 54b₉ | (R)-tert-butyl 4-(1-(4-(hydroxycarbamoyl)benzyl)-3-oxo-1,2,3,4- |
| | tetrahydroquinoxalin-2-yl)butylcarbamate, |
| 54b₁₀ | (R)—N-hydroxy-4-((2-(naphthalen-2-ylmethyl)-3-oxo-3,4-dihydroquinoxalin-1(2H)- |
| | yl)methyl)benzamide, |
| 54b₁₁ | (R)-4-((2-(4-tert-butoxybenzyl)-3-oxo-3,4-dihydroquinoxalin-1(2H)-yl)methyl)-N- |
| | hydroxybenzamide, |
| 54b₁₂ | (R)-tert-butyl 3-(1-(4-(hydroxycarbamoyl)benzyl)-3-oxo-1,2,3,4- |
| | tetrahydroquinoxalin-2-yl)propanoate, |
| 54b₁₃ | (R)-4-((2-(benzo[b]thiophen-3-ylmethyl)-3-oxo-3,4-dihydroquinoxalin-1(2H)- |
| | yl)methyl)-N-hydroxybenzamide, |
| 54b₁₄ | (S)-4-((2-(benzo[b]thiophen-3-ylmethyl)-3-oxo-3,4-dihydroquinoxalin-1(2H)- |
| | yl)methyl)-N-hydroxybenzamide, |
| 54b₁₅ | (R)-4-((2-(4-fluorobenzyl)-3-oxo-3,4-dihydroquinoxalin-1(2H)-yl)methyl)-N- |
| | hydroxybenzamide, |
| 54b₁₆ | (R)-4-((2-(3,4-difluorobenzyl)-3-oxo-3,4-dihydroquinoxalin-1(2H)-yl)methyl)-N- |
| | hydroxybenzamide, |
| 54b₁₇ | (R)-4-((2-(4-trifluoromethylbenzyl)-3-oxo-3,4-dihydroquinoxalin-1(2H)-yl)methyl)- |
| | N-hydroxybenzamide, |
| 54b₁₈ | (R)—N-hydroxy-4-((2-(4-hydroxybenzyl)-3-oxo-3,4-dihydroquinoxalin-1(2H)- |
| | yl)methyl)benzamide, |
| 54b₁₉ | (R)—N-hydroxy-4-((2-(3-(trifluoromethyl)benzyl)-3-oxo-3,4-dihydroquinoxalin- |
| | 1(2H)-yl)methyl)benzamide, |
| 54b₂₀ | (R)-4-((2-(furan-3-ylmethyl)-3-oxo-3,4-dihydroquinoxalin-1(2H)-yl)methyl)-N- |
| | hydroxybenzamide, |
| 54b₂₁ | (R)—N-hydroxy-4-((3-oxo-2-(pyridin-3-ylmethyl)-3,4-dihydroquinoxalin-1(2H)- |
| | yl)methyl)benzamide, |
| 54c | (S)—N-(2-aminophenyl)-4-((3-oxo-2-(thiophen-2-ylmethyl)-3,4-dihydroquinoxalin- |
| | 1(2H)-yl)methyl)benzamide, |
| 56a | 4-(((R)-2,3-Dihydro-2-oxo-3-((thiophen-2-yl)methyl)quinoxalin-4(1H)-yl)methyl)- |
| | N-hydroxybenzamide, |
| 62 | 4-((S)-2-((1H-indol-3-yl)methyl)-1,2,3,4-tetrahydro-3-oxoquinoxalin-6-yl)-N-(2- |
| | amino-5-(thiophen-2-yl)phenyl)benzamide, |
| 66c | (E)-3-((S)-2-((1H-indol-3-yl)methyl)-1,2,3,4-tetrahydro-3-oxoquinoxalin-6-yl)-N- |
| | hydroxyacrylamide, |
| 66f | (E)-3-((R)-2-((1H-indol-3-yl)methyl)-1,2,3,4-tetrahydro-3-oxoquinoxalin-6-yl)-N- |
| | hydroxyacrylamide, |
| 68b₁ | 6-((S)-2,3,4,5-tetrahydro-3-isobutyl-2,5-dioxobenzo[e][1,4]diazepin-1-yl)-N- |
| | hydroxyhexanamide, |
| 68b₃ | 6-((S)-2,3,4,5-tetrahydro-3-neopentyl-2,5-dioxobenzo[e][1,4]diazepin-1-yl)-N- |
| | hydroxyhexanamide, |
| 68b₄ | 6-((R)-2,3,4,5-tetrahydro-2,5-dioxo-3-phenylbenzo[e][1,4]diazepin-1-yl)-N- |
| | hydroxyhexanamide, |
| 68b₆ | 6-((R)-2,3,4,5-tetrahydro-2,5-dioxo-3-((pyridin-4-yl)methyl)benzo[e][1,4]diazepin- |
| | 1-yl)-N-hydroxyhexanamide, |
| 68b₇ | 6-((S)-3-((1H-indol-3-yl)methyl)-2,3,4,5-tetrahydro-2,5- |
| | dioxobenzo[e][1,4]diazepin-1-yl)-N-hydroxyhexanamide, |
| 68b₈ | 6-((R)-3-((1H-indol-3-yl)methyl)-2,3,4,5-tetrahydro-2,5- |
| | dioxobenzo[e][1,4]diazepin-1-yl)-N-hydroxyhexanamide, |
| 68b₉ | 6-((R)-2,3,4,5-tetrahydro-2,5-dioxo-3-((pyridin-4-yl)methyl)benzo[e][1,4]diazepin- |
| | 1-yl)-N-hydroxyhexanamide, |
| 68b₁₁ | 6-((S)-2,3,4,5-tetrahydro-2,5-dioxo-3-phenylbenzo[e][1,4]diazepin-1-yl)-N- |
| | hydroxyhexanamide, |
| 68b₁₂ | 6-((R)-2,3,4,5-tetrahydro-3-isobutyl-2,5-dioxobenzo[e][1,4]diazepin-1-yl)-N- |
| | hydroxyhexanamide, |
| 68b₁₆ | 6-(3,3-spirocyclopentyl-2,5-dioxo-2,3,4,5,tetrahydrobenzo[e][1,4]diazepin-1-yl)- |
| | N-hydroxyhexanamide, |
| 68b₁₇ | 6-((S)-2,3,4,5-tetrahydro-3-neopentyl-2,5-dioxobenzo[e][1,4]diazepin-1-yl)-N- |
| | hydroxyhexanamide, |
| 68b₁₈ | 6-((R)-2,3,4,5-tetrahydro-3-neopentyl-2,5-dioxobenzo[e][1,4]diazepin-1-yl)-N- |
| | hydroxyhexanamide, |
| 68b₁₉ | 6-((S)-3-cyclohexyl-2,3,4,5-tetrahydro-2,5-dioxobenzo[e][1,4]diazepin-1-yl)-N- |
| | hydroxyhexanamide, |
| 68b₂₀ | 6-((S)-3-(cyclohexylmethyl)-2,3,4,5-tetrahydro-2,5-dioxobenzo[e][1,4]diazepin-1- |
| | yl)-N-hydroxyhexanamide, |
| 68b₂₁ | 6-((R)-3-(cyclohexylmethyl)-2,3,4,5-tetrahydro-2,5-dioxobenzo[e][1,4]diazepin-1- |
| | yl)-N-hydroxyhexanamide, |
| 68b₂₂ | 6-((S)-2,3,4,5-tetrahydro-3-methyl-2,5-dioxobenzo[e][1,4]diazepin-1-yl)-N- |
| | hydroxyhexanamide, |
| 68b₂₃ | 6-((R)-2,3,4,5-tetrahydro-3-methyl-2,5-dioxobenzo[e][1,4]diazepin-1-yl)-N- |
| | hydroxyhexanamide, |
| 69 | 3-((S)-2-((1H-indol-3-yl)methyl)-1,2,3,4-tetrahydro-3-oxoquinoxalin-6-yl)-N- |
| | hydroxypropanamide, |
| 71 | N-(2-aminophenyl)-6-((R)-2,3,4,5-tetrahydro-3-isobutyl-2,5- |
| | dioxobenzo[e][1,4]diazepin-1-yl)hexanamide, |
| 72 | N-(4-aminothiophen-3-yl)-6-((R)-2,3,4,5-tetrahydro-3-isobutyl-2,5- |
| | dioxobenzo[e][1,4]diazepin-1-yl)hexanamide, |
| 74 | 6-(2,3,4,5-tetrahydro-2,5-dioxo-1H-benzo[e][1,4]diazepin-3-yl)-N- |
| | hydroxyhexanamide, |
| 79 | 6-(1-(2-(1H-indol-3-yl)ethyl)-2,3,4,5-tetrahydro-2,5-dioxo-1H- |
| | benzo[e][1,4]diazepin-3-yl)-N-hydroxyhexanamide, |
| 80 | 6-(1-benzyl-2,3,4,5-tetrahydro-2,5-dioxo-1H-benzo[e][1,4]diazepin-3-yl)-N- |
| | hydroxyhexanamide, |
| 81 | 6-(1-(3,5-dimethoxybenzyl)-2,3,4,5-tetrahydro-2,5-dioxo-1H- |
| | benzo[e][1,4]diazepin-3-yl)-N-hydroxyhexanamide, |
| 82 | 6-(1-(4-methoxybenzyl)-2,3,4,5-tetrahydro-2,5-dioxo-1H-benzo[e][1,4]diazepin-3- |
| | yl)-N-hydroxyhexanamide, |
| 83 | 6-(2,3,4,5-tetrahydro-2,5-dioxo-1-phenethyl-1H-benzo[e][1,4]diazepin-3-yl)-N- |
| | hydroxyhexanamide, |
| 88a | 6-(2,3,4,5-tetrahydro-2,5-dioxo-7-phenoxy-1H-benzo[e][1,4]diazepin-3-yl)-N- |
| | hydroxyhexanamide, |
| 92a | 6-(7-Benzyloxycarbonylamino-2,3,4,5-tetrahydro-2,5-dioxo-1H- |
| | benzo[e][1,4]diazepin-3-yl)-N-hydroxyhexanamide, |
| 92b | (S)-benzyl 3-(6-(hydroxyamino)-6-oxohexyl)-2,5-dioxo-2,3,4,5-tetrahydro-1H- |
| | benzo[e][1,4]diazepin-7-ylcarbamate, |
| 97a | (R)—N-hydroxy-3-isopropyl-2,5-dioxo-2,3,4,5-tetrahydro-1H- |
| | benzo[e][1,4]diazepine-8-carboxamide, |
| 97b | (S)—N-hydroxy-3-isopropyl-2,5-dioxo-2,3,4,5-tetrahydro-1H- |
| | benzo[e][1,4]diazepine-8-carboxamide, |
| 97c | (S)-3-((1H-indol-3-yl)methyl)-N-hydroxy-2,5-dioxo-2,3,4,5-tetrahydro-1H- |
| | benzo[e][1,4]diazepine-8-carboxamide, |
| 97d | (R)-3-((1H-indol-3-yl)methyl)-N-hydroxy-2,5-dioxo-2,3,4,5-tetrahydro-1H- |
| | benzo[e][1,4]diazepine-8-carboxamide, |
| 97f | (R)—N-hydroxy-3-isobutyl-2,5-dioxo-2,3,4,5-tetrahydro-1H- |
| | benzo[e][1,4]diazepine-8-carboxamide, |
| 97g | (R)-3-(cyclohexylmethyl)-N-hydroxy-2,5-dioxo-2,3,4,5-tetrahydro-1H- |
| | benzo[e][1,4]diazepine-8-carboxamide, |
| 97h | (S)-3-(cyclohexylmethyl)-N-hydroxy-2,5-dioxo-2,3,4,5-tetrahydro-1H- |
| | benzo[e][1,4]diazepine-8-carboxamide, |
| 97i | (S)—N-hydroxy-2,5-dioxo-3-phenyl-2,3,4,5-tetrahydro-1H-benzo[e][1,4]diazepine- |
| | 8-carboxamide, |
| 97j | (R)—N-hydroxy-2,5-dioxo-3-phenyl-2,3,4,5-tetrahydro-1H-benzo[e][1,4]diazepine- |
| | 8-carboxamide and |
| 97k | N-hydroxy-2,5-dioxo-2,3,4,5-tetrahydro-1H-benzo[e][1,4]diazepine-8- |
| | carboxamide. |

In the second aspect of the present invention there is provided compounds of formula (XVI), 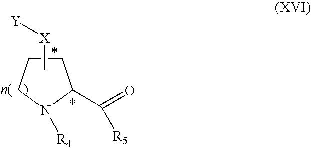
and N-oxides, hydrates, solvates, pharmaceutically acceptable salts, prodrugs and complexes thereof, wherein
n is 1 or 2;
X is selected from the group consisting of —O—, —S—, —N(R¹)— and —CH(R¹)—;
Y is selected from the group consisting of —(C₀-C₇)alkyl-heteroaryl-W, —(C₁-C₇)alkyl-W, —(C₀-C₇)alkyl-aryl-W and —C(O)—(C₁-C₇)alkyl-W;
W is selected from the group consisting of —C(O)—NH—OH, —C(O)—(C₁-C₄)alkyl, —C(O)—N(R¹)₂, —(C₂-C₆)alkyl-N(OH)—C(O)H—, —(C₁-C₆)alkyl-SR¹, —(C₁-C₆)alkyl-SC(O)—(C₁-C₄)alkyl, —C(O)—OR¹, —C(O)—(C₁-C₄)alkylepoxide, —C(O)—(C₁-C₄)alkyl-SH, —C(O)—(C₁-C₄)alkyl-SC(O)R¹, —C(O)—(C₁-C₄)alkyl-5-heteroaryl, —(C₁-C₆)alkyl-NH—C(O)—(C₁-C₆)alkyl-halo, —(C₁-C₆)alkyl-NH—C(O)—(C₁-C₆)alkyl-SH, —(C₁-C₆)alkyl-NH—C(O)—(C₁-C₆)alkyl-SC(O)R¹, —C(O)—NH—(C₂-C₆)alkyl-SH and —C(O)—(C₁-C₆)alkyl,
wherein the alkyl of said —C(O)—(C₁-C₆)alkyl is optionally substituted with one or more substituents selected from the group consisting of mono to per-halogenated —(C₁-C₆)alkyl, —C(O)-heteroaryl, —C(O)—N H-heteroaryl and —C(O)—N H-aryl;
wherein each aryl and heteroaryl is optionally substituted with one or more substituents selected from the group consisting of —NH₂, —OH, SH, —CN, —NO₂, —N(R¹)₂, halo, mono- to per-halogenated-(C₁-C₆)alkyl, aryl, heteroaryl, 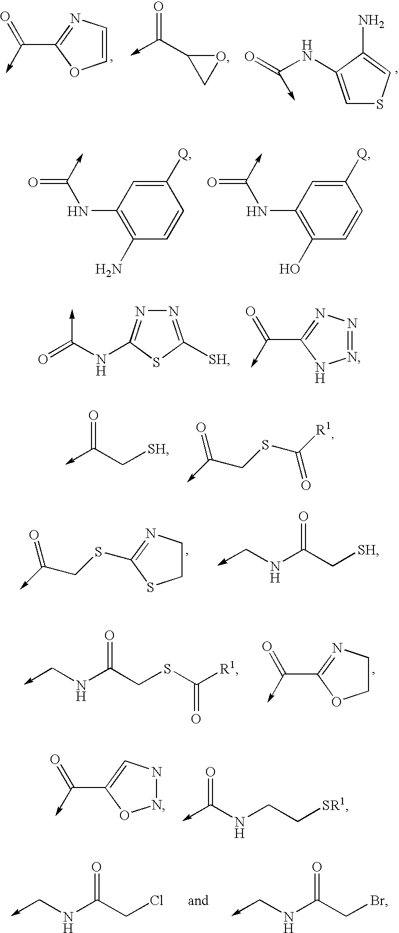
wherein Q is selected from the group consisting of heterocyclic, aryl and heteroaryl;
R¹ is independently selected from the group consisting of —H, —(C₁-C₆)alkyl, —(C₁-C₆)heteroalkyl, —(C₃-C₆)cycloalkyl, -heterocyclyl, —(C₀-C₆)alkyl-aryl and —(C₀-C₆)alkyl-heteroaryl,
wherein each afore-mentioned R¹ aryl, heteroaryl, cycloalkyl and heterocyclyl moiety is optionally substituted with one or more substituents selected from the group consisting of oxo, —OH, —CN, —(C₁-C₆)alkyl, —(C₁-C₆)alkoxy, —NO₂, —N(R¹)₂, halo, —SH, mono- to per-halogenated-(C₁-C₆)alkyl and —(C₂-C₄)alkyl-N(R¹)₂,
wherein optionally the R¹ together with the nitrogen atom to which they are attached form a heterocyclyl group;
R⁴ is selected from the group consisting of —S(O)₂—(C₁-C₆)alkyl, —S(O)₂—(C₁-C₆)heteroalkyl, —S(O)₂—(C₁-C₆)aryl, —S(O)₂—(C₁-C₆)alkylaryl, —S(O)₂—(C₁-C₆)heteroaryl, —S(O)₂—(C₁-C₆)arylalkyl, —S(O)₂—(C₁-C₆)heterocyclic, —C(O)—(C₁-C₆)alkyl, —C(O)—(C₁-C₆)heteroalkyl, —C(O)—(C₁-C₆)aryl, —C(O)—(C₁-C₆)alkylaryl, —C(O)—(C₁-C₆)heteroaryl, —C(O)—(C₁-C₆)arylalkyl, —C(O)—(C₁-C₆)heterocyclic and —C(O)—OR¹;
R⁵ is selected from the group consisting of —OR¹ and —N(R¹)₂; and
the asterisk mark * indicates a chiral carbon atom, with the proviso that when X is N(R¹), Y is —C(O)—(C₁-C₇)alkyl-W or —S(O)₂—(C₁-C₆)alkyl-W.

In a preferred embodiment of Formula (XV) of the present invention, Embodiment UU, Q is selected from the group consisting of thiopheneyl, furanyl, tetrazolyl, imidazolyl, pyridinyl and pyrimidinyl.

In another preferred embodiment of Formula (XV) of the present invention, Embodiment VV,
n is 1;
X is —O—;
Y is selected from the group consisting of —(C₁-C₇)alkyl-W, —(C₀-C₇)alkyl-aryl-W and —C(O)—(C₁-C₇)alkyl-W;
W is —C(O)—NH—OH;
R⁴ is —C(O)—OR¹; and
R⁵ is —N(R¹)₂

A preferred embodiment of Embodiment VV of the present invention, Embodiment WW, provides compounds according to the formula XVII 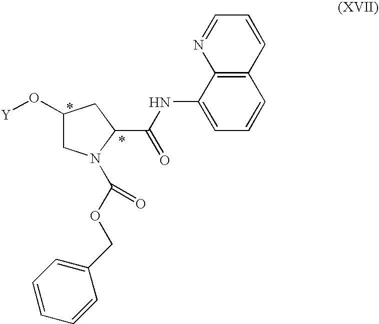
and N-oxides, hydrates, solvates, pharmaceutically acceptable salts, prodrugs and complexes thereof, wherein Y is selected from the group consisting of

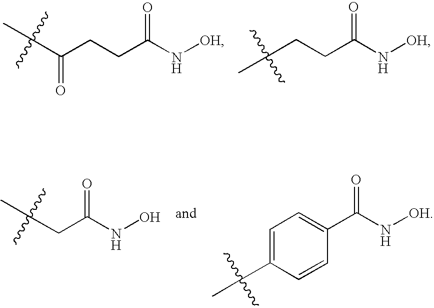

Another preferred embodiment of the second aspect of the present invention, Embodiment XX, provides compounds selected from the group consisting of

| Cpd No. | Compound |
|---|---|
| 109a | (2S,4S)-benzyl 4-(4-(hydroxycarbamoyl)phenoxy)- |
| | 2-(quinolin-8-ylcarbamoyl)pyrrolidine-1-carboxylate, |
| 109b | (2S,4R)-benzyl 4-(4-(hydroxycarbamoyl)phenoxy)- |
| | 2-(quinolin-8-ylcarbamoyl)pyrrolidine-1-carboxylate, |
| 109c | (2R,4S)-benzyl 4-(4-(hydroxycarbamoyl)phenoxy)- |
| | 2-(quinolin-8-ylcarbamoyl)pyrrolidine-1-carboxylate, |
| 112a | (2S,4R)-benzyl 4-(3-(hydroxyamino)-3-oxopropoxy)- |
| | 2-(quinolin-8-ylcarbamoyl)pyrrolidine-1-carboxylate, |
| 112b | (2S,4R)-benzyl 4-(2-(hydroxyamino)-2-oxoethoxy)- |
| | 2-(quinolin-8-ylcarbamoyl)pyrrolidine-1-carboxylate and |
| 112c | (2R,4R)-benzyl 4-(3-(hydroxyamino)-3-oxopropoxy)- |
| | 2-(quinolin-8-ylcarbamoyl)pyrrolidine-1-carboxylate. |

In another preferred embodiment of Formula (I) of the present invention

X¹, X², X³ and X⁴ are absent;

X⁵ is a covalent bond;

X⁶ is CH₂;

n is 1;

B is —(C₀-C₇)alkyl-aryl-(C₀-C₄)alkyl-W;

W is —C(O)NHOH;

A is H; and

E and D are independently selected from a group consisting of —H, —(C₀-C₆)alkyl-aryl- and —(C₀-C₆)alkyl-heteroaryl-, wherein each aryl and heteroaryl moiety is optionally substituted with one or more R². Preferably, E and D are independently selected from the group consisting of —H, —(C₁-C₆)alkyl-aryl- and —(C₁-C₆)alkyl-heteroaryl-.

Another preferred embodiment of Formula (I) of the present invention provides compounds according to Formula (XVIII) 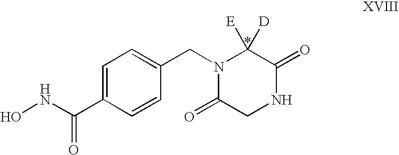
and N-oxides, hydrates, solvates, pharmaceutically acceptable salts, prodrugs and complexes thereof. In a preferred embodiment, one of D and E is H and the other is selected from the group consisting of 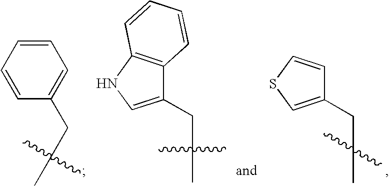
wherein each aryl and heteroaryl moeity is optionally substituted with one or more groups selected from R².

In another preferred embodiment of Formula (I), only one of Z, A, B. D and E end in with the moiety W.

In the third aspect of the present invention, the invention provides a composition comprising a compound according to the first aspect or second aspects or Embodiments A to XX, and a pharmaceutically acceptable carrier, diluent or excipient. In one embodiment, the composition comprises a compound according to the first aspect or second aspects or Embodiments A to XX, together with an additional HDAC inhibitor known in the art or which will be discovered, and a pharmaceutically acceptable carrier, diluent or excipient. In a preferred embodiment, the additional HDAC inhibitor is a small molecule or a nucleic acid level inhibitor of histone deacetylase.

In the fourth aspect, the invention provides a method of inhibiting histone deacetylase. In one embodiment, the method comprises contacting the histone deacetylase with an inhibiting effective amount of a compound according to the first aspect or second aspect or Embodiments A to XX. In a further embodiment of the fourth aspect, the method comprises contacting the histone deacetylase with an inhibiting effective amount of a composition according to the third aspect. In still another embodiment, the method of inhibiting histone deacetylase further comprises contacting the histone deacetylase with an additional HDAC inhibitor known in the art or which will be discovered in an amount sufficient to inhibit histone decetylase. In a preferred embodiment, the HDAC inhibitors act synergistically to inhibit histone deacetylase. In yet another embodiment, the invention provides a method of inhibiting histone deacetylase in a cell, the method comprising contacting the cell with an inhibiting effective amount of compound according to the first aspect or the second aspect or Embodiments A to XX. In still another embodiment, the method of inhibiting histone deacetylase in a cell comprises contacting the cell with an inhibiting effective amount of a composition according to the third aspect. In still another embodiment, the method of inhibiting histone deacetylase in a cell further comprises contacting the cell with an additional HDAC inhibitor known in the art or which will be discovered and/or a nucleic acid level inhibitor of histone deacetylase in an amount sufficient to inhibit histone decetylase. In a preferred embodiment, the HDAC inhibitors act synergistically to inhibit histone deacetylase activity.

For purposes of the present invention, the following definitions will be used (unless expressly stated otherwise):

As used herein, the terms “histone deacetylase” and “HDAC” are intended to refer to any one of a family of enzymes that remove acetyl groups from the ε-amino groups of lysine residues at the N-terminus of a histone. Unless otherwise indicated by context, the term “histone” is meant to refer to any histone protein, including H1, H2A, H₂B, H3, H4, and H5, from any species. Preferred histone deacetylases include class I and class II enzymes. Other preferred histone deacetylases include class III enzymes. Preferably the histone deacetylase is a human HDAC, including, but not limited to, HDAC-1, HDAC-2, HDAC-3, HDAC-4, HDAC-5, HDAC-6, HDAC-7, HDAC-8, HDAC-9, HDAC-10, HDAC-11, SirT1, SirT2, SirT3, SirT4, SirT5, SirT6 and SirT7. In some other preferred embodiments, the histone deacetylase is derived from a plant, protozoal or fungal source.

The terms “histone deacetylase inhibitor” and “inhibitor of histone deacetylase” are intended to mean a compound having a structure as defined herein, which is capable of interacting with a histone deacetylase and inhibiting its enzymatic activity.

The term “inhibiting histone deacetylase enzymatic activity” is intended to mean reducing the ability of a histone deacetylase to remove an acetyl group from a histone. The concentration of inhibitor which reduces the activity of a histone deacetylase to 50% of that of the uninhibited enzyme is determined as the IC₅₀ value.

The term “inhibiting effective amount” is meant to denote a dosage sufficient to cause inhibition of histone deacetylase activity. The histone deacetylase can be in a cell, which cell can be in a multicellular organism. The multicellular organism can be, for example, a plant, a fungus or an animal, preferably a mammal and more preferably a human. If in a multicellular organism, the method according to this aspect of the invention comprises administering to the organism a compound or composition according to the present invention. Administration may be by any route, including, without limitation, parenteral, oral, sublingual, transdermal, topical, intranasal, intratracheal, or intrarectal. In certain particularly preferred embodiments, compounds of the invention are administered intravenously in a hospital setting. In certain other preferred embodiments, administration may preferably be by the oral route.

Preferably, such inhibition is specific, i.e., the histone deacetylase inhibitor reduces the ability of a histone deacetylase to remove an acetyl group from a histone at a concentration that is lower than the concentration of the inhibitor that is required to produce another, unrelated biological effect. Preferably, the concentration of the inhibitor required for histone deacetylase inhibitory activity is at least 2-fold lower, more preferably at least 5-fold lower, even more preferably at least 10-fold lower, and most preferably at least 20-fold lower than the concentration required to produce an unrelated biological effect.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for 0, and 2, 4, or 6 for S, depending on the oxidation state of the S). On occasion a moiety may be defined, for example, as (A)ₐ-B-, wherein a is 0 or 1. In such instances, when a is 0 the moiety is B— and when a is 1 the moiety is A-B—.

For simplicity, reference to a “Cₙ—Cₘ” heterocyclyl or “Cₙ—Cₘ” heteroaryl means a heterocyclyl or heteroaryl having from “n” to “m” annular atoms, where “n” and “m” are integers. Thus, for example, a C₅-C₆-heterocyclyl is a 5- or 6-membered ring having at least one heteroatom, and includes pyrrolidinyl (C₅) and piperidinyl (C₆); C₆-heteroaryl includes, for example, pyridyl and pyrimidyl.

The term “hydrocarbyl” refers to a straight, branched, or cyclic alkyl, alkenyl, or alkynyl, each as defined herein. A “C₀” hydrocarbyl is used to refer to a covalent bond. Thus, “C₀-C₃-hydrocarbyl” includes a covalent bond, methyl, ethyl, ethenyl, ethynyl, propyl, propenyl, propynyl, and cyclopropyl.

The term “alkyl” is intended to mean a straight or branched chain aliphatic group having from 1 to 12 carbon atoms, preferably 1-8 carbon atoms, and more preferably 1-6 carbon atoms, which is optionally substituted with one, two or three substituents. Other preferred alkyl groups have from 2 to 12 carbon atoms, preferably 2-8 carbon atoms and more preferably 2-6 carbon atoms. Preferred alkyl groups include, without limitation, methyl, ethyl, propyl, isopropyl, butyl, isobutyl, sec-butyl, tert-butyl, pentyl, and hexyl. A “C₀” alkyl (as in “C₀-C₃-alkyl”) is a covalent bond.

The term “alkenyl” is intended to mean an unsaturated straight or branched chain aliphatic group with one or more carbon-carbon double bonds, having from 2 to 12 carbon atoms, preferably 2-8 carbon atoms, and more preferably 2-6 carbon atoms, which is optionally substituted with one, two or three substituents. Preferred alkenyl groups include, without limitation, ethenyl, propenyl, butenyl, pentenyl, and hexenyl.

The term “alkynyl” is intended to mean an unsaturated straight or branched chain aliphatic group with one or more carbon-carbon triple bonds, having from 2 to 12 carbon atoms, preferably 2-8 carbon atoms, and more preferably 2-6 carbon atoms, which is optionally substituted with one, two or three substituents. Preferred alkynyl groups include, without limitation, ethynyl, propynyl, butynyl, pentynyl, and hexynyl.

The terms “alkylene,” “alkenylene,” or “alkynylene” as used herein are intended to mean an alkyl, alkenyl, or alkynyl group, respectively, as defined hereinabove, that is positioned between and serves to connect two other chemical groups. Preferred alkylene groups include, without limitation, methylene, ethylene, propylene, and butylene. Preferred alkenylene groups include, without limitation, ethenylene, propenylene, and butenylene. Preferred alkynylene groups include, without limitation, ethynylene, propynylene, and butynylene.

The term “cycloalkyl” is intended to mean a saturated or unsaturated cyclic hydrocarbon group having 3 to 12 carbons, preferably 3 to 8 carbons, and more preferably 3 to 6 carbons, wherein the cycloalkyl group additionally is optionally substituted. Preferred cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” is intended to mean a saturated or unsaturated, straight or branched chain aliphatic group, wherein one or more carbon atoms in the chain are independently replaced by a heteroatom selected from the group consisting of O, S, and N.

The term “aryl” is intended to mean a C₆-C₁₄ aromatic moiety comprising one to three aromatic rings, which is optionally substituted. Preferably, the aryl group is a C₆-C₁₀ aryl group, more preferably a C₆ aryl group. Preferred aryl groups include, without limitation, phenyl, naphthyl, anthracenyl, and fluorenyl.

The terms “aralkyl” or “arylalkyl” is intended to mean a group comprising an aryl group covalently linked to an alkyl group, either of which may independently be optionally substituted or unsubstituted. Preferably, the aralkyl group is (C₁-C₆)alk(C₆-C₁₀)aryl, including, without limitation, benzyl, phenethyl, and naphthylmethyl. For simplicity, when written as “arylalkyl” this term, and terms related thereto, is intended to indicate the order of groups in a compound as “aryl-alkyl”. Similarly, “alkyl-aryl” is intended to indicate the order of the groups in a compound as “alkyl-aryl”.

The terms “heterocyclyl”, “heterocyclic” or “heterocycle” are intended to mean a group which is an optionally substituted aromatic or, preferably, non-aromatic mono-, bi-, or tricyclic structure having from about 3 to about 14 atoms, wherein one or more atoms are independently selected from the group consisting of N, O, and S. One ring of a bicyclic heterocycle or one or two rings of a tricyclic heterocycle may be aromatic, as in indan and 9,10-dihydro anthracene. The heterocyclic group is optionally substituted on carbon with, for example, oxo or with one of the substituents listed above. The heterocyclic group may also independently be substituted on nitrogen with, for example, alkyl, aryl, aralkyl, alkylcarbonyl, alkylsulfonyl, arylcarbonyl, arylsulfonyl, alkoxycarbonyl, aralkoxycarbonyl, or on sulfur with oxo or lower alkyl. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain preferred embodiments, the heterocyclic group is fused to an aryl, heteroaryl, or cycloalkyl group. Examples of such fused heterocycles include, without limitation, tetrahydroquinoline and dihydrobenzofuran. Specifically excluded from the scope of this term are compounds where an annular O or S atom is adjacent to another O or S atom.

In certain preferred embodiments, the heterocyclic group is a heteroaryl group. As used herein, the term “heteroaryl” is intended to mean an optionally substituted group having 5 to 14 ring atoms, preferably 5, 6, 9, or 10 ring atoms; having 6, 10, or 14 pi electrons shared in a cyclic array; and having, in addition to carbon atoms, between one or more heteroatoms independently selected from the group consisting of N, O, and S. For example, a heteroaryl group may be pyrimidinyl, pyridinyl, benzimidazolyl, thienyl, benzothiazolyl, benzofuranyl and indolinyl. Preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

The terms “arylene,” “heteroarylene,” or “heterocyclylene” are intended to mean an aryl, heteroaryl, or heterocyclyl group, respectively, as defined hereinabove, that is positioned between and serves to connect two other chemical groups.

Preferred heterocyclyls and heteroaryls include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

As employed herein, when a moiety (e.g., alkyl, heteroalkyl, cycloalkyl, aryl, heteroaryl, heterocyclyl, etc.) is described as “optionally substituted” it is meant that the group optionally has from one to four, preferably from one to three, more preferably one or two, non-hydrogen substituents. Suitable substituents include, without limitation, halo, hydroxy, oxo (e.g., an annular —CH— substituted with oxo is —C(O)—) nitro, halohydrocarbyl, hydrocarbyl, aryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise) are:
(a) halo, cyano, oxo, carboxy, formyl, nitro, amino, amidino, guanidino,
(b) C₁-C₅ alkyl or alkenyl or arylalkyl imino, carbamoyl, azido, carboxamido, mercapto, hydroxy, hydroxyalkyl, alkylaryl, arylalkyl, C₁-C₈ alkyl, C₁-C₈ alkenyl, C₁-C₈ alkoxy, C₁-C₈ alkoxycarbonyl, aryloxycarbonyl, C₂-C₈ acyl, C₂-C₈ acylamino, C₁-C₈ alkylthio, arylalkylthio, arylthio, C₁-C₈ alkylsulfinyl, arylalkylsulfinyl, arylsulfinyl, C₁-C₈ alkylsulfonyl, arylalkylsulfonyl, arylsulfonyl, C₀-C₆ N-alkyl carbamoyl, C₂-C₁₅ N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇ heterocycle, C₅-C₁₅ heteroaryl or any of these rings fused or spiro-fused to a cycloalkyl, heterocyclyl, or aryl, wherein each of the foregoing is further optionally substituted with one more moieties listed in (a), above; and
(c) —(CH₂), —NR₃₀R₃₁, wherein n is from 0 (in which case the nitrogen is directly bonded to the moiety that is substituted) to 6, and R₃₀ and R₃₁ are each independently hydrogen, cyano, oxo, carboxamido, amidino, C₁-C₈ hydroxyalkyl, C₁-C₃ alkylaryl, aryl-C₁-C₃ alkyl, C₁-C₈ alkyl, C₁-C₈ alkenyl, C₁-C₈ alkoxy, C₁-C₈ alkoxycarbonyl, aryloxycarbonyl, aryl-C₁-C₃ alkoxycarbonyl, C₂-C₈ acyl, C₁-C₈ alkylsulfonyl, arylalkylsulfonyl, arylsulfonyl, aroyl, aryl, cycloalkyl, heterocyclyl, or heteroaryl, wherein each of the foregoing is further optionally substituted with one more moieties listed in (a), above; or
   R³⁰ and R³¹ taken together with the N to which they are attached form a heterocyclyl or heteroaryl, each of which is optionally substituted with from 1 to 3 substituents from (a), above.

A “halohydrocarbyl” is a hydrocarbyl moiety in which from one to all hydrogens have been replaced with one or more halo.

The term “halogen” or “halo” is intended to mean chlorine, bromine, fluorine, or iodine. As herein employed, the term “acyl” refers to an alkylcarbonyl or arylcarbonyl moiety. The term “acylamino” refers to an amide group attached at the nitrogen atom (i.e., R—CO—NH—). The term “carbamoyl” refers to an amide group attached at the carbonyl carbon atom (i.e., NH₂—CO—). The nitrogen atom of an acylamino or carbamoyl substituent is additionally optionally substituted. The term “sulfonamido” refers to a sulfonamide substituent attached by either the sulfur or the nitrogen atom. The term “amino” is meant to include NH₂, alkylamino, arylamino, and cyclic amino groups. The term “ureido” as employed herein refers to a substituted or unsubstituted urea moiety.

The term “radical” is intended to mean a chemical moiety comprising one or more unpaired electrons.

A moiety that is substituted is one in which one or more hydrogens have been independently replaced with another chemical substituent. As a non-limiting example, substituted phenyls include 2-flurophenyl, 3,4-dichlorophenyl, 3-chloro-4-fluoro-phenyl, 2-fluoro-3-propylphenyl. As another non-limiting example, substituted N-octyls include 2,4-dimethyl-5-ethyl-octyl and 3-cyclopentyl-octyl. Included within this definition are methylenes (—CH₂—) substituted with oxygen to form carbonyl —CO—).

Where optional substituents are chosen from “one or more” groups it is to be understood that this definition includes all substituents being chosen from one of the specified groups or the substituents being chosen from two or more of the specified groups.

In addition, substituents on cyclic moieties (i.e., cycloalkyl, heterocyclyl, aryl, heteroaryl) include 5-6 membered mono- and 9-14 membered bi-cyclic moieties fused to the parent cyclic moiety to form a bi- or tri-cyclic fused ring system. Substituents on cyclic moieties also include 5-6 membered mono- and 9-14 membered bi-cyclic moieties attached to the parent cyclic moiety by a covalent bond to form a bi- or tri-cyclic bi-ring system. For example, an optionally substituted phenyl includes, but is not limited to, the following:

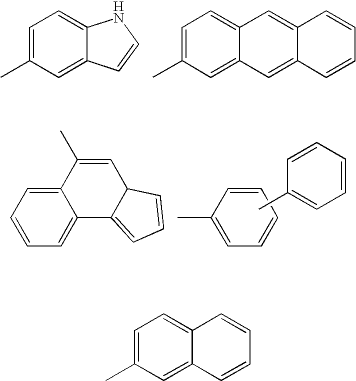

As will be understood by those skilled in the art, when X⁵ and X⁶ together are —C═C—, different, but equivalent resonance structures may be drawn, as in, for example:

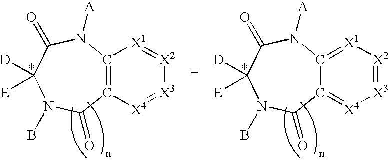

An “unsubstituted” moiety as defined above (e.g., unsubstituted cycloalkyl, unsubstituted heteroaryl, etc.) means that moiety as defined above that does not have any of the optional substituents for which the definition of the moiety (above) otherwise provides. Thus, for example, while an “aryl” includes phenyl and phenyl substituted with a halo, “unsubstituted aryl” does not include phenyl substituted with a halo.

Some compounds of the invention may have chiral centers and/or geometric isomeric centers (E- and Z-isomers), and it is to be understood that the invention encompasses all such optical, diastereoisomers and geometric isomers. The invention also comprises all tautomeric forms of the compounds disclosed herein.

The present invention also includes prodrugs of compounds of the invention. The term “prodrug” is intended to represent covalently bonded carriers, which are capable of releasing the active ingredient of the prodrug when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of the present invention include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of the invention, amides (e.g., trifluoroacetylamino, acetylamino, and the like), and the like.

The compounds of the invention may be administered as is or in the form of an in vivo hydrolyzable ester or in vivo hydrolyzable amide. An in vivo hydrolyzable ester of a compound of the invention containing carboxy or hydroxy group is, for example, a pharmaceutically acceptable ester which is hydrolyzed in the human or animal body to produce the parent acid or alcohol. Suitable pharmaceutically acceptable esters for carboxy include C₁₋₆-alkoxymethyl esters (e.g., methoxymethyl), C₁₋₆-alkanoyloxymethyl esters (e.g., for example pivaloyloxymethyl), phthalidyl esters, C₃₋₈-cycloalkoxycarbonyloxyC₁₋₆-alkyl esters (e.g., 1-cyclohexylcarbonyloxyethyl); 1,3-dioxolen-2-onylmethyl esters (e.g., 5-methyl-1,3-dioxolen-2-onylmethyl; and C₁₋₆-alkoxycarbonyloxyethyl esters (e.g., 1-methoxycarbonyloxyethyl) and may be formed at any carboxy group in the compounds of this invention.

An in vivo hydrolyzable ester of a compound of the invention containing a hydroxy group includes inorganic esters such as phosphate esters and a-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolyzable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(N,N-dialkylaminoethyl)-N-alkylcarbamoyl (to give carbamates), N,N-dialkylaminoacetyl and carboxyacetyl. Examples of substituents on benzoyl include morpholino and piperazino linked from a ring nitrogen atom via a methylene group to the 3- or 4-position of the benzoyl ring. A suitable value for an in vivo hydrolyzable amide of a compound of the invention containing a carboxy group is, for example, a N-C₁₋₆-alkyl or N,N-di-C₁₋₆-alkyl amide such as N-methyl, N-ethyl, N-propyl, N,N-dimethyl, N-ethyl-N-methyl or N,N-diethyl amide.

The foregoing merely summarizes the various aspects and preferred embodiments thereof, of the invention and is not intended to be limiting in nature. These aspects and embodiments are described more fully below.

Compounds

The data presented herein demonstrate the histone deacetylase inhibitory effects of the compounds of the invention. These data lead one to reasonably expect that the compounds of the invention are useful for inhibition of histone deacetylase.

Preferred compounds according to the invention include those in the Table 1, which were prepared essentially using the methods described herein and illustrated below in the Schemes. These examples merely serve to exemplify some of the compounds of the first and second aspects of the invention and do not limit the scope of the invention. All of the compounds in this application were named using Chemdraw Ultra version 10.0, which is available through Cambridgesoft.co, 100 Cambridge Park Drive, Cambridge, Mass. 02140, Namepro version 5.09, which is available from ACD labs, 90 Adelaide Street West, Toronto, Ontario, M5H, 3V9, Canada, or were derived therefrom.

TABLE 1

| Cpd No. | Compound |
|---|---|
| 4a | (S)-1,2,3,4-Tetrahydro-N-hydroxy-2-isopropyl-3-oxoquinoxaline-6-carboxamide |
| 4b | (S)-1,2,3,4-tetrahydro-N-hydroxy-2-(2-(methylthio)ethyl)-3-oxoquinoxaline-6- |
| | carboxamide |
| 4c | (S)-2-((1H-indol-3-yl)methyl)-1,2,3,4-tetrahydro-N-hydroxy-3-oxoquinoxaline-6- |
| | carboxamide |
| 4d | (S)-2-sec-butyl-1,2,3,4-tetrahydro-N-hydroxy-3-oxoquinoxaline-6-carboxamide |
| 4e | (R)-1,2,3,4-tetrahydro-N-hydroxy-2-isopropyl-3-oxoquinoxaline-6-carboxamide |
| 4f | (R)-2-((1H-indol-3-yl)methyl)-1,2,3,4-tetrahydro-N-hydroxy-3-oxoquinoxaline-6- |
| | carboxamide |
| 4g | (S)-1,2,3,4-tetrahydro-N-hydroxy-3-oxo-2-((1-trityl-1H-imidazol-4- |
| | yl)methyl)quinoxaline-6-carboxamide |
| 4h | (S)-2-(4-tert-butoxybenzyl)-1,2,3,4-tetrahydro-N-hydroxy-3-oxoquinoxaline-6- |
| | carboxamide |
| 4i | (S)-2-Benzyl-1,2,3,4-tetrahydro-N-hydroxy-3-oxoquinoxaline-6-carboxamide |
| 4k | (S)-1,2,3,4-Tetrahydro-N-hydroxy-2-isobutyl-3-oxoquinoxaline-6-carboxamide |
| 4l | (S)-1,2,3,4-tetrahydro-N-hydroxy-2-((1-methyl-1-H-indol-3-yl)methyl)-3- |
| | oxoquinoxaline-6-carboxamide |
| 5c | (S)-2-((1H-indol-3-yl)methyl)-N-(2-aminophenyl)-1,2,3,4-tetrahydro-3-oxoquinoxaline- |
| | 6-carboxamide |
| 6l | (S)-2-(4-hydroxybenzyl)-1,2,3,4-tetrahydro-N-hydroxy-3-oxoquinoxaline-6- |
| | carboxamide |
| 8 | (S)-1-(4-Methoxybenzyl)-1,2,3,4-tetrahydro-N-hydroxy-2-isopropyl-3 oxoquinoxaline- |
| | 6-carboxamide |
| 11 | (S)—N-(5-(Hydroxycarbamoyl)pentyl)-1,2,3,4-tetrahydro-2-isopropyl-3-oxoquinoxaline- |
| | 6-carboxamide |
| 16a | (S)-1-(4-fluorobenzyl)-N-hydroxy-2-isopropyl-3-oxo-1,2,3,4-tetrahydroquinoxaline-6- |
| | carboxamide |
| 16b | (S)-1-(4-hydroxybenzyl)-1,2,3,4-tetrahydro-N-hydroxy-2-isopropyl-3-oxoquinoxaline-6- |
| | carboxamide |
| 16c | (S)-1,2,3,4-tetrahydro-N-hydroxy-2-isopropyl-3-oxo-1-(3-phenylpropyl)quinoxaline-6- |
| | carboxamide |
| 16d | 4-(((S)-7-(hydroxycarbamoyl)-2,3-dihydro-3-isopropyl-2-oxoquinoxalin-4(1H)- |
| | yl)methyl)benzoic acid |
| 16e | (S)-1,2,3,4-tetrahydro-N-hydroxy-2-isopropyl-3-oxo-1-((thiophen-2- |
| | yl)methyl)quinoxaline-6-carboxamide |
| 16f | (S)—N-hydroxy-1-(4-(N-hydroxycarbamimidoyl)benzyl)-2-isopropyl-3-oxo-1,2,3,4- |
| | tetrahydroquinoxaline-6-carboxamide |
| 16g | (S)-1-benzyl-1,2,3,4-tetrahydro-N-hydroxy-2-isopropyl-3-oxoquinoxaline-6- |
| | carboxamide |
| 16h | (S)-1,2,3,4-tetrahydro-N-hydroxy-2-isopropyl-1-((naphthalen-3-yl)methyl)-3- |
| | oxoquinoxaline-6-carboxamide |
| 18a | (S)—N-hydroxy-2-isopropyl-3-oxo-1-(phenylsulfonyl)-1,2,3,4-tetrahydroquinoxaline-6- |
| | carboxamide |
| 18b | (S)—N-hydroxy-1-(biphenyl-4-sulfonyl)-2-isopropyl-3-oxo-1,2,3,4- |
| | tetrahydroquinoxaline-6-carboxamide |
| 18c | (S)—N-hydroxy-2-isopropyl-3-oxo-1-(2,4,6-trimethybenzenesulfonyl)-1,2,3,4- |
| | tetrahydroquinoxaline-6-carboxamide |
| 18e | (S)—N-hydroxy-1-(4-methoxybenzenesulfonyl-2-isopropyl-3-oxo-1,2,3,4- |
| | tetrahydroquinoxaline-6-carboxamide |
| 18f | (S)—N-hydroxy-1-(1-naphthalenesulfonyl-2-isopropyl-3-oxo-1,2,3,4- |
| | tetrahydroquinoxaline-6-carboxamide |
| 23a | N1-(2-Aminophenyl)-N8—((S)-1,2,3,4-tetrahydro-2-isopropyl-3-oxoquinoxalin-6- |
| | yl)octanediamide |
| 24a | N1—((S)-1,2,3,4-Tetrahydro-2-isopropyl-3-oxoquinoxalin-6-yl)-N8- |
| | hydroxyoctanediamide |
| 24e | N1—((R)-1,2,3,4-Tetrahydro-2-isopropyl-3-oxoquinoxalin-6-yl)-N8- |
| | hydroxyoctanediamide |
| 24f | N1—((R)-2-((1H-Indol-3-yl)methyl)-1,2,3,4-tetrahydro-3-oxoquinoxalin-6-yl)-N8- |
| | hydroxyoctanediamide |
| 26e | N1—((R)-1-(4-Methoxybenzyl)-1,2,3,4-tetrahydro-2-isopropyl-3-oxoquinoxalin-6-yl)-N8- |
| | hydroxyoctanediamide |
| 29a | N—((S)-1,2,3,4-Tetrahydro-2-isopropyl-3-oxoquinoxalin-6-yl)-8-(oxazol-2-yl)-8- |
| | oxooctanamide |
| 43 | 6-((R)-1-(4-methoxybenzyl)-1,2,3,4-tetrahydro-3-oxoquinoxalin-2-yl)-N-hydroxy-4-oxy- |
| | hexanamide |
| 47a | 6-(1-(4-methoxybenzyl)-1,2,3,4-tetrahydro-3-oxoquinoxalin-2-yl)-N- |
| | hydroxyhexanamide |
| 47b | 6-(1-(4-methoxybenzyl)-1,2,3,4-tetrahydro-3-oxoquinoxalin-2-yl)-N-(2- |
| | aminophenyl)hexanamide |
| 49 | 6-(1-(4-methoxybenzyl)-1,2,3,4-tetrahydro-3-oxoquinoxalin-2-yl)-N-(2-amino-5- |
| | (thiophen-2-yl)phenyl)hexanamide |
| 54a₁ | 4-(((S)-2,3-Dihydro-2-oxo-3-((thiophen-2-yl)methyl)quinoxalin-4(1H)-yl)methyl)-N- |
| | hydroxybenzamide |
| 54a₂ | 4-(((R)-2,3-Dihydro-2-oxo-3-((thiophen-2-yl)methyl)quinoxalin-4(1H)-yl)methyl)-N- |
| | hydroxybenzamide |
| 54b₁ | N-hydroxy-4-((3-oxo-3,4-dihydroquinoxalin-1(2H)-yl)methyl)-benzamide |
| 54b₂ | (R)-4-((2-((1H-indol-3-yl)methyl)-3-oxo-3,4-dihydroquinoxalin-1(2H)-yl)methyl)-N- |
| | hydroxybenzamide |
| 54b₃ | (S)-4-((2-((1H-indol-3-yl)methyl)-3-oxo-3,4-dihydroquinoxalin-1(2H)-yl)methyl)-N- |
| | hydroxybenzamide |
| 54b₄ | (S)-4-((2-benzyl-3-oxo-3,4-dihydroquinoxalin-1(2H)-yl)methyl)-N-hydroxybenzamide |
| 54b₅ | (R)-4-((2-benzyl-3-oxo-3,4-dihydroquinoxalin-1(2H)-yl)methyl)-N-hydroxybenzamide |
| 54b₆ | N-hydroxy-4-((3-oxo-3,4-dihydroquinoxalin-1(2H)-yl)methyl)benzamide |
| 54b₇ | (R)—N-hydroxy-4-((3-oxo-2-phenethyl-3,4-dihydroquinoxalin-1(2H)- |
| | yl)methyl)benzamide |
| 54b₈ | (R)-4-((2-(cyclohexylmethyl)-3-oxo-3,4-dihydroquinoxalin-1(2H)-yl)methyl)-N- |
| | hydroxybenzamide |
| 54b₉ | (R)-tert-butyl 4-(1-(4-(hydroxycarbamoyl)benzyl)-3-oxo-1,2,3,4-tetrahydroquinoxalin- |
| | 2-yl)butylcarbamate |
| 54b₁₀ | (R)—N-hydroxy-4-((2-(naphthalen-2-ylmethyl)-3-oxo-3,4-dihydroquinoxalin-1(2H)- |
| | yl)methyl)benzamide |
| 54b₁₁ | (R)-4-((2-(4-tert-butoxybenzyl)-3-oxo-3,4-dihydroquinoxalin-1(2H)-yl)methyl)-N- |
| | hydroxybenzamide |
| 54b₁₂ | (R)-tert-butyl 3-(1-(4-(hydroxycarbamoyl)benzyl)-3-oxo-1,2,3,4-tetrahydroquinoxalin- |
| | 2-yl)propanoate |
| 54b₁₃ | (R)-4-((2-(benzo[b]thiophen-3-ylmethyl)-3-oxo-3,4-dihydroquinoxalin-1(2H)- |
| | yl)methyl)-N-hydroxybenzamide |
| 54b₁₄ | (S)-4-((2-(benzo[b]thiophen-3-ylmethyl)-3-oxo-3,4-dihydroquinoxalin-1(2H)-yl)methyl)- |
| | N-hydroxybenzamide |
| 54b₁₅ | (R)-4-((2-(4-fluorobenzyl)-3-oxo-3,4-dihydroquinoxalin-1(2H)-yl)methyl)-N- |
| | hydroxybenzamide |
| 54b₁₆ | (R)-4-((2-(3,4-difluorobenzyl)-3-oxo-3,4-dihydroquinoxalin-1(2H)-yl)methyl)-N- |
| | hydroxybenzamide |
| 54b₁₇ | (R)-4-((2-(4-trifluoromethylbenzyl)-3-oxo-3,4-dihydroquinoxalin-1(2H)-yl)methyl)-N- |
| | hydroxybenzamide |
| 54b₁₈ | (R)—N-hydroxy-4-((2-(4-hydroxybenzyl)-3-oxo-3,4-dihydroquinoxalin-1(2H)- |
| | yl)methyl)benzamide |
| 54b₁₉ | (R)—N-hydroxy-4-((2-(3-(trifluoromethyl)benzyl)-3-oxo-3,4-dihydroquinoxalin-1(2H)- |
| | yl)methyl)benzamide |
| 54b₂₀ | (R)-4-((2-(furan-3-ylmethyl)-3-oxo-3,4-dihydroquinoxalin-1(2H)-yl)methyl)-N- |
| | hydroxybenzamide |
| 54b₂₁ | (R)—N-hydroxy-4-((3-oxo-2-(pyridin-3-ylmethyl)-3,4-dihydroquinoxalin-1(2H)- |
| | yl)methyl)benzamide |
| 54c | (S)—N-(2-aminophenyl)-4-((3-oxo-2-(thiophen-2-ylmethyl)-3,4-dihydroquinoxalin-1(2H)- |
| | yl)methyl)benzamide |
| 56a | 4-(((R)-2,3-Dihydro-2-oxo-3-((thiophen-2-yl)methyl)quinoxalin-4(1H)-yl)methyl)-N- |
| | hydroxybenzamide |
| 62 | 4-((S)-2-((1H-indol-3-yl)methyl)-1,2,3,4-tetrahydro-3-oxoquinoxalin-6-yl)-N-(2-amino- |
| | 5-(thiophen-2-yl)phenyl)benzamide |
| 66c | (E)-3-((S)-2-((1H-indol-3-yl)methyl)-1,2,3,4-tetrahydro-3-oxoquinoxalin-6-yl)-N- |
| | hydroxyacrylamide |
| 66f | (E)-3-((R)-2-((1H-indol-3-yl)methyl)-1,2,3,4-tetrahydro-3-oxoquinoxalin-6-yl)-N- |
| | hydroxyacrylamide |
| 68b₁ | 6-((S)-2,3,4,5-tetrahydro-3-isobutyl-2,5-dioxobenzo[e][1,4]diazepin-1-yl)-N- |
| | hydroxyhexanamide |
| 68b₃ | 6-((S)-2,3,4,5-tetrahydro-3-neopentyl-2,5-dioxobenzo[e][1,4]diazepin-1-yl)-N- |
| | hydroxyhexanamide |
| 68b₄ | 6-((R)-2,3,4,5-tetrahydro-2,5-dioxo-3-phenylbenzo[e][1,4]diazepin-1-yl)-N- |
| | hydroxyhexanamide |
| 68b₆ | 6-((R)-2,3,4,5-tetrahydro-2,5-dioxo-3-((pyridin-4-yl)methyl)benzo[e][1,4]diazepin-1-yl)- |
| | N-hydroxyhexanamide |
| 68b₇ | 6-((S)-3-((1H-indol-3-yl)methyl)-2,3,4,5-tetrahydro-2,5-dioxobenzo[e][1,4]diazepin-1- |
| | yl)-N-hydroxyhexanamide |
| 68b₈ | 6-((R)-3-((1H-indol-3-yl)methyl)-2,3,4,5-tetrahydro-2,5-dioxobenzo[e][1,4]diazepin-1- |
| | yl)-N-hydroxyhexanamide |
| 68b₉ | 6-((R)-2,3,4,5-tetrahydro-2,5-dioxo-3-((pyridin-4-yl)methyl)benzo[e][1,4]diazepin-1-yl)- |
| | N-hydroxyhexanamide |
| 68b₁₁ | 6-((S)-2,3,4,5-tetrahydro-2,5-dioxo-3-phenylbenzo[e][1,4]diazepin-1-yl)-N- |
| | hydroxyhexanamide |
| 68b₁₂ | 6-((R)-2,3,4,5-tetrahydro-3-isobutyl-2,5-dioxobenzo[e][1,4]diazepin-1-yl)-N- |
| | hydroxyhexanamide |
| 68b₁₆ | 6-(3,3-spirocyclopentyl-2,5-dioxo-2,3,4,5,tetrahydrobenzo[e][1,4]diazepin-1-yl)-N- |
| | hydroxyhexanamide |
| 68b₁₇ | 6-((S)-2,3,4,5-tetrahydro-3-neopentyl-2,5-dioxobenzo[e][1,4]diazepin-1-yl)-N- |
| | hydroxyhexanamide |
| 68b₁₈ | 6-((R)-2,3,4,5-tetrahydro-3-neopentyl-2,5-dioxobenzo[e][1,4]diazepin-1-yl)-N- |
| | hydroxyhexanamide |
| 68b₁₉ | 6-((S)-3-cyclohexyl-2,3,4,5-tetrahydro-2,5-dioxobenzo[e][1,4]diazepin-1-yl)-N- |
| | hydroxyhexanamide |
| 68b₂₀ | 6-((S)-3-(cyclohexylmethyl)-2,3,4,5-tetrahydro-2,5-dioxobenzo[e][1,4]diazepin-1-yl)-N- |
| | hydroxyhexanamide |
| 68b₂₁ | 6-((R)-3-(cyclohexylmethyl)-2,3,4,5-tetrahydro-2,5-dioxobenzo[e][1,4]diazepin-1-yl)-N- |
| | hydroxyhexanamide |
| 68b₂₂ | 6-((S)-2,3,4,5-tetrahydro-3-methyl-2,5-dioxobenzo[e][1,4]diazepin-1-yl)-N- |
| | hydroxyhexanamide |
| 68b₂₃ | 6-((R)-2,3,4,5-tetrahydro-3-methyl-2,5-dioxobenzo[e][1,4]diazepin-1-yl)-N- |
| | hydroxyhexanamide |
| 69 | 3-((S)-2-((1H-indol-3-yl)methyl)-1,2,3,4-tetrahydro-3-oxoquinoxalin-6-yl)-N- |
| | hydroxypropanamide |
| 71 | N-(2-aminophenyl)-6-((R)-2,3,4,5-tetrahydro-3-isobutyl-2,5- |
| | dioxobenzo[e][1,4]diazepin-1-yl)hexanamide |
| 72 | N-(4-aminothiophen-3-yl)-6-((R)-2,3,4,5-tetrahydro-3-isobutyl-2,5- |
| | dioxobenzo[e][1,4]diazepin-1-yl)hexanamide |
| 74 | 6-(2,3,4,5-tetrahydro-2,5-dioxo-1H-benzo[e][1,4]diazepin-3-yl)-N-hydroxyhexanamide |
| 79 | 6-(1-(2-(1H-indol-3-yl)ethyl)-2,3,4,5-tetrahydro-2,5-dioxo-1H-benzo[e][1,4]diazepin-3- |
| | yl)-N-hydroxyhexanamide |
| 80 | 6-(1-benzyl-2,3,4,5-tetrahydro-2,5-dioxo-1H-benzo[e][1,4]diazepin-3-yl)-N- |
| | hydroxyhexanamide |
| 81 | 6-(1-(3,5-dimethoxybenzyl)-2,3,4,5-tetrahydro-2,5-dioxo-1H-benzo[e][1,4]diazepin-3- |
| | yl)-N-hydroxyhexanamide |
| 82 | 6-(1-(4-methoxybenzyl)-2,3,4,5-tetrahydro-2,5-dioxo-1H-benzo[e][1,4]diazepin-3-yl)- |
| | N-hydroxyhexanamide |
| 83 | 6-(2,3,4,5-tetrahydro-2,5-dioxo-1-phenethyl-1H-benzo[e][1,4]diazepin-3-yl)-N- |
| | hydroxyhexanamide |
| 88a | 6-(2,3,4,5-tetrahydro-2,5-dioxo-7-phenoxy-1H-benzo[e][1,4]diazepin-3-yl)-N- |
| | hydroxyhexanamide |
| 92a | 6-(7-Benzyloxycarbonylamino-2,3,4,5-tetrahydro-2,5-dioxo-1H-benzo[e][1,4]diazepin- |
| | 3-yl)-N-hydroxyhexanamide |
| 92b | (S)-benzyl 3-(6-(hydroxyamino)-6-oxohexyl)-2,5-dioxo-2,3,4,5-tetrahydro-1H- |
| | benzo[e][1,4]diazepin-7-ylcarbamate |
| 97a | (R)—N-hydroxy-3-isopropyl-2,5-dioxo-2,3,4,5-tetrahydro-1H-benzo[e][1,4]diazepine-8- |
| | carboxamide |
| 97b | (S)—N-hydroxy-3-isopropyl-2,5-dioxo-2,3,4,5-tetrahydro-1H-benzo[e][1,4]diazepine-8- |
| | carboxamide |
| 97c | (S)-3-((1H-indol-3-yl)methyl)-N-hydroxy-2,5-dioxo-2,3,4,5-tetrahydro-1H- |
| | benzo[e][1,4]diazepine-8-carboxamide |
| 97d | (R)-3-((1H-indol-3-yl)methyl)-N-hydroxy-2,5-dioxo-2,3,4,5-tetrahydro-1H- |
| | benzo[e][1,4]diazepine-8-carboxamide |
| 97f | (R)—N-hydroxy-3-isobutyl-2,5-dioxo-2,3,4,5-tetrahydro-1H-benzo[e][1,4]diazepine-8- |
| | carboxamide |
| 97g | (R)-3-(cyclohexylmethyl)-N-hydroxy-2,5-dioxo-2,3,4,5-tetrahydro-1H- |
| | benzo[e][1,4]diazepine-8-carboxamide |
| 97h | (S)-3-(cyclohexylmethyl)-N-hydroxy-2,5-dioxo-2,3,4,5-tetrahydro-1H- |
| | benzo[e][1,4]diazepine-8-carboxamide |
| 97i | (S)—N-hydroxy-2,5-dioxo-3-phenyl-2,3,4,5-tetrahydro-1H-benzo[e][1,4]diazepine-8- |
| | carboxamide |
| 97j | (R)—N-hydroxy-2,5-dioxo-3-phenyl-2,3,4,5-tetrahydro-1H-benzo[e][1,4]diazepine-8- |
| | carboxamide |
| 97k | N-hydroxy-2,5-dioxo-2,3,4,5-tetrahydro-1H-benzo[e][1,4]diazepine-8-carboxamide |
| 109a | (2S,4S)-benzyl 4-(4-(hydroxycarbamoyl)phenoxy)-2-(quinolin-8- |
| | ylcarbamoyl)pyrrolidine-1-carboxylate |
| 109b | (2S,4R)-benzyl 4-(4-(hydroxycarbamoyl)phenoxy)-2-(quinolin-8- |
| | ylcarbamoyl)pyrrolidine-1-carboxylate |
| 109c | (2R,4S)-benzyl 4-(4-(hydroxycarbamoyl)phenoxy)-2-(quinolin-8- |
| | ylcarbamoyl)pyrrolidine-1-carboxylate |
| 112a | (2S,4R)-benzyl 4-(3-(hydroxyamino)-3-oxopropoxy)-2-(quinolin-8- |
| | ylcarbamoyl)pyrrolidine-1-carboxylate |
| 112b | (2S,4R)-benzyl 4-(2-(hydroxyamino)-2-oxoethoxy)-2-(quinolin-8- |
| | ylcarbamoyl)pyrrolidine-1-carboxylate |
| 112c | (2R,4R)-benzyl 4-(3-(hydroxyamino)-3-oxopropoxy)-2-(quinolin-8- |
| | ylcarbamoyl)pyrrolidine-1-carboxylate |

Synthetic Schemes and Experimental Procedures

The compounds of the invention can be prepared according to the reaction schemes for the examples illustrated below utilizing methods known to one of ordinary skill in the art. These Schemes serve to exemplify some procedures that can be used to make the compounds of the invention. One skilled in the art will recognize that other general synthetic procedures may be used. The compounds of the invention can be prepared from starting components that are commercially available. Any kind of substitutions can be made to the starting components to obtain the compounds of the invention according to procedures that are well known to those skilled in the art.

The present invention will be more readily understood by referring to the following examples, which are given to illustrate the invention rather than to limit its scope.

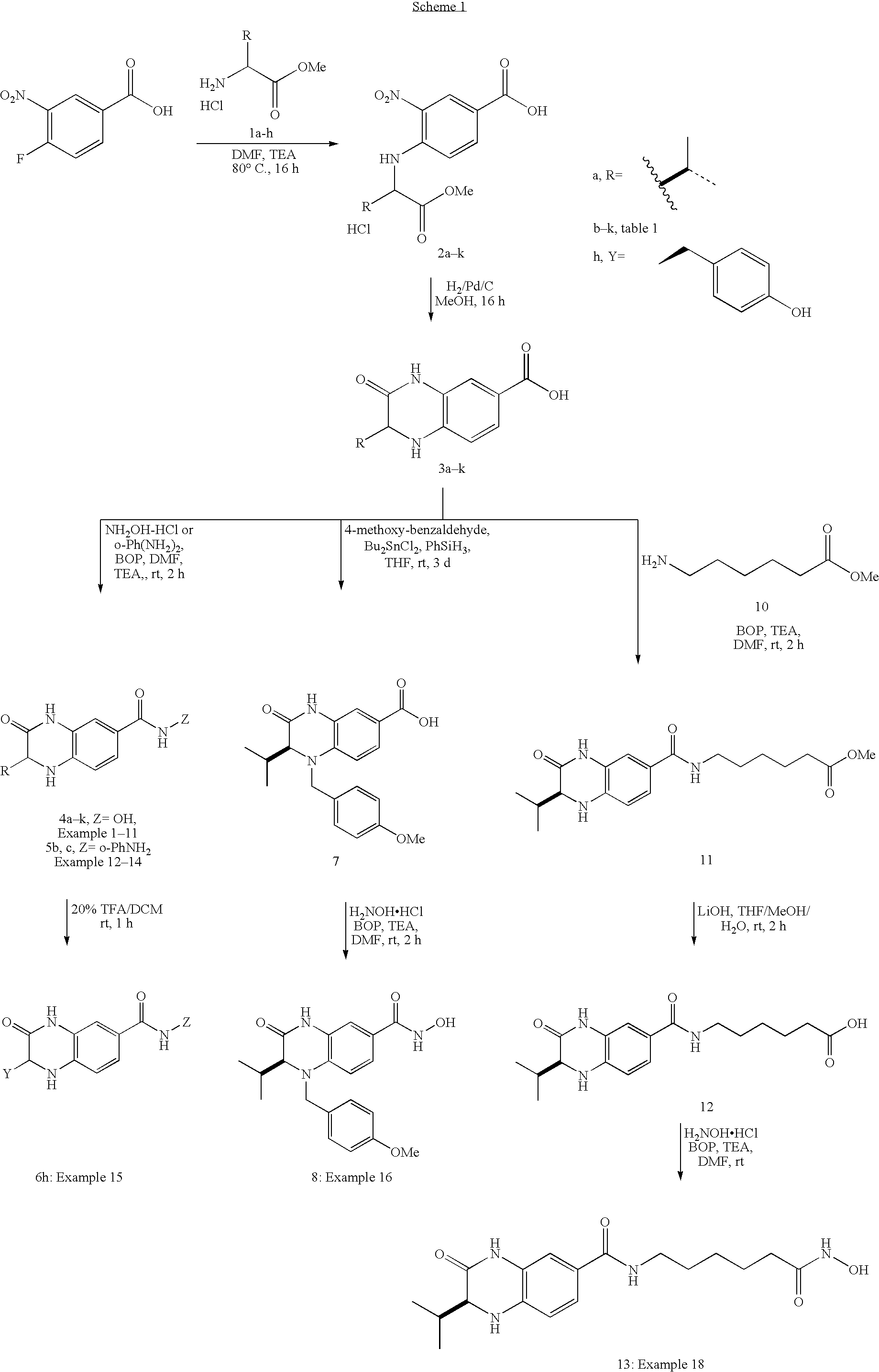

### EXAMPLE 1

### (S)-1,2,3,4-Tetrahydro-N-hydroxy-2-isopropyl-3-oxoquinoxaline-6-carboxamide (Compound 4a)

### Step 1: 4-((S)-1-(Methoxycarbonyl)-2-methylpropylamino)-3-nitrobenzoic acid (Compound 2a)

Both (L)-valine methyl ester hydrochloride (1.54 g, 9.19 mmol) (1a) and 4-fluoro-3-nitrobenzoic acid (1.70 g, 9.19 mmol) were dissolved in DMF (10 mL) at room temperature. Triethylamine (3.84 mL, 27.6 mmol) was then added, and the resulting solution heated to 80° C. for 16 h. After cooling, the solution was filtered, and the solvent removed. The residue, acid 2a, was obtained in near quantitative yield, and used in the subsequent reaction without further purification. LRMS (ESI): (calc.) 296.3; (found) 297.1 (MH)⁺.

### Step 2: (S)-1,2,3,4-Tetrahydro-2-isopropyl-3-oxoquinoxaline-6-carboxylic acid (Compound 3a)

Acid 2a (2.72 g, 9.19 mmol) was dissolved in MeOH (50 mL), and 10% Pd/C (982 mg, 0.919 mmol) was added to the resulting solution. After stirring for 16 h under a hydrogen atmosphere, the solution was filtered through a pad of celite, and the filtrate concentrated. After purification of the residue by flash chromatography (eluent 0-80% EtOAc in hexanes), 1.83 g (85%) of Compound 3a was obtained as a light yellow crystalline solid. LRMS (ESI): (calc.) 234.3; (found) 235.4 (MH)⁺.

### Step 3: (S)-1,2,3,4-Tetrahydro-N-hydroxy-2-isopropyl-3-oxoquinoxaline-6-carboxamide (Compound 4a)

Acid 3a (237 mg, 1.01 mmol) was dissolved in DMF (4 mL), and BOP (535 mg, 1.21 mmol) was added in one portion. After stirring for 5 min, hydroxylamine hydrochloride (84 mg, 1.21 mmol) was added, followed by the addition of triethylamine (0.56 mL, 4.04 mmol). The resulting solution was stirred for 2 h prior to the removal of all solvents. After purification of the residue by flash chromatography (eluent 0-20% MeOH in EtOAc), 86 mg (34%) of Compound 4a was obtained as a light pink crystalline solid. ¹H NMR: (DMSO-d₆) δ (ppm): 10.37 (s, 1H), 8.79 (br s, 1H), 7.18 (s,1H), 7.15 (s, 1H), 6.69 (d, J=8.0 Hz, 1H), 6.51 (br s,1H), 3.68 (d, J=3.1 Hz, 1H), 3.38 (br s, 1H), 2.00-2.10 (m, 1H), 0.98 (d, J=6.8 Hz, 3H), 0.87 (d, J=6.7 Hz, 3H). LRMS (ESI): (calc.) 249.3; (found) 250.1 (MH)⁺.

### EXAMPLES 2-14

Examples 2-13 describe the preparation of Compounds 4b-k and 5b-c using the same procedures as described for Compound 4a in Example 1. Characterization data are presented in Table 2.

TABLE 2

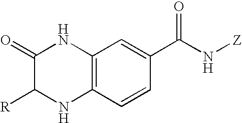

| Ex. | Cpd. | R | Z | Name | Characterization | Scheme |
|---|---|---|---|---|---|---|
| 2 | 4b | 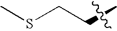 | —OH | (S)-1,2,3,4- Tetrahydro-N- hyroxy-2-(2- (methylthio)ethyl) -3- oxoquinoxaline- 6-carboxamide | ¹H NMR: (MeOD-d₄) δ (ppm): 7.97 (s, 1 H), 7.27 (dd, J=8.2, 2.0Hz, 1 H), 7.19 (d, J=1.7Hz, 1 H), 6.75 (d, J =8.4Hz, 1 H), 4.12-4.09 ( H), 3.33 (t, J=1.6Hz, 2 H), 2.88 (s, 2 H), 2.09 (s, | |
| # 3 H). LRMS (ESI): (calc.) 281.1; (found) 282.1 (MH)⁺. | 1 | | | | | |
| 3 | 4c | 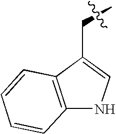 | —OH | (S)-2-((1H-Indol- 3-yl)methyl)- 1,2,3,4- tetrahydro-N- hydroxy-3- oxoquinoxal ine- 6-carboxamide | ¹H NMR: (MeOD-d₄) δ (ppm): 7.52 (d, J=7.8Hz, 1H), 7.29 (d, J=8.0Hz, 1H), 7.18 (dd, J=8.2, 2.0Hz, 1H), 7.09-6.96 (m, 4H), 6.57 (d, J=8.2Hz, 1H), 4.22 (dd, J=8.2, 3.9Hz, 1H), | |
| # 3.31-3.17 (m, 2H). LRMS (ESI): 336.2; (found) 337.2 (MH)⁺. | 1 | | | | | |
| 4 | 4d | 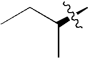 | —OH | (S)-2-sec-Butyl- 1,2,3,4- tetrahydro-N- hydroxy-3- oxoquinoxaline- 6-carboxamide | ¹H NMR: (DMSO-d₆) δ (ppm): 10.75 (br s, 1H), 10.32 (s, 1H), 8.93 (br s, 1H), 7.12-7.11 (m, 2H), 6.62 (d, J =8.0Hz, 1H), 6.50 (d, J=1.6Hz, 1H), 3.73-3.72 (m, 1H), 1.78-1.74 (m, 1H), 1.46-1.40 (m, 1H), 1.15-1.08 (m, | |
| # 1H), 0.90 (d, J=7.0Hz, 3H), 0.81 (t, J =7.4Hz, 3H). LRMS (ESI): (calc.) 358.2; (found) 359.2 (MH)⁼. | 1 | | | | | |
| 5 | 4e | 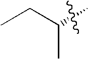 | —OH | (R)-1,2,3,4- Tetrahydro-N- hydroxy-2- isopropyl-3-(S)-2-sec-Butyl- 1,2,3,4- tetrahydro-N- hydroxy-3- oxoquinoxaline- 6-carboxamide | ¹H NMR: (MeOD-d₄) δ (ppm): 7.23 (dd, J=8.0Hz, J=1.6Hz, 1H), 7.14 (d, J=2.0Hz, 1H), 6.70 (d, J=8.4 Hz, 1H), 3.77 (d, J=4.8Hz, 1H), | |
| # 2.16-2.06 (m, 1H), 1.02 (d, J=6.8Hz, 3H), 0.93 (d, J=6.8Hz, 3H). LRMS (ESI): (calc.) 249.1; (found) 250.1 (MH)⁺. | 1 | | | | | |
| 6 | 4f | 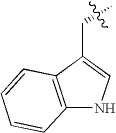 | —OH | (R)-2-((1H- Indol-3- yl)methyl)- 1,2,3,4- tetrahydro-N- oxoquinoxaline- 6-carboxamide | ¹H NMR: (DMSO-d₆) δ (ppm): 10.82 (br s, 1H), 10.77 (br s, 1H), 1039 (br s, 1H), 8.70 (br s, 1H), 7.48 (d, J=8.8Hz, 1H), 7.29 (d, J=8.0Hz, 1H), 7.13-7.11 (m, 2H), 7.07 (d, J=2.4Hz, 1H), 7.02 (t, J = | |
| # 7.9Hz, 1H), 6.58 (d, J =8.8Hz, 1H), 6.33 (s, 1H), 4.14-4.11 (m, 1H), 3.09-2.95 (m, 2H). LRMS (ESI): (calc.) 336.1; (found) 337.1 (MH)⁺. | 1 | | | | | |
| 7 | 4g | 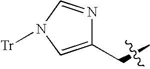 | —OH | (S)-1,2,3,4- Tetrahydro-N- hydroxy-3-oxo- 2-((1-trityl-1H- imidazol-4- yl)methyl) quinoxaline-6- carboxamide | ¹H NMR: (DMSO-d₆) δ(ppm): 10.78 (br s, 1H), 10.31 (s, 1H), 8.71 (br s, 1H), 7.37-7.30 (m, 9H), 7.18 (d, J =1.4Hz, 1H), 7.13-7.10 (m, 2H), 7.03-7.00 (m, 6H), 6.56 (d, J=1.4Hz, 1H), | |
| # 6.48 (d, J=8.4Hz, 1H), 6.34 (s, 1H), 4.08-4.07 (m, 1H), 2.78-2.72 (m, 2H). LRMS (ESI): (calc.) 529.1; (found) 530.1 (MH)⁺. | 1 | | | | | |
| 8 | 4h | 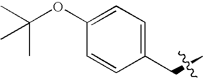 | —OH | (S)-2-(4-tert- Butoxybenzyl)- 1,2,3,4- tetrahydro-N- hydroxy-3- oxoquinoxaline- 6-carboxamide | ¹H NMR: (DMSO-d₆) δ (ppm): 10.77 (s, 1H), 10.32 (s, 1H), 8.68 (br s, 1H), 7.14-7.04 (m, 4H), 6.78 (d, J=7.5Hz, 2H), 6.58 (d, J=8.5Hz, 1H), 6.43 (s, 1H), 4.11-4.13 (m, 1H), 2.88-2.85 (m, 2H), 1.20 (s, | |
| # 9H). 1 | | | | | | |
| 9 | 4i | 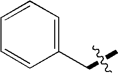 | —OH | (S)-1,2,3,4- Tetrahydro-N- hydroxy-2 isobutyl-3-oxoquinoxaline- 6-carboxamide | ¹H NMR: (DMSO-d₆) δ (ppm): 10.84 (s, 1H), 10.34 (s, 1H), 7.19 (d, J=1.8Hz, 2H), 6.70 (d, J=8.6Hz, 1H), 6.50 (br s, 1H), 3.83 (t, J=5.7Hz, 1H), 1.80-1.90 (m, 1H), 1.42-1.52 (M, 2H), 0.92 (d, J=6.5Hz, 6H) LRMS (ESI): | |
| # (calc.) 263.3; (found) 264.1 (MH)⁺. | 1 | | | | | |
| 11 | 4k | 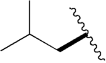 | —OH | (S)-1,2,3,4- Tetrahydro-N- hydroxy-2 isobutyl-3- oxoquinoxaline- 6-carboxamide | ¹H NMR: (DMSO-d₆) δ (ppm): 10.84 (s, 1H), 10.34 (s, 1H), 7.19 (d, J=1.8Hz, 2H), 6.70 (d, J=8.6Hz, 1H), 6.50 (br s, 1H), 3.83 (t, J=5.7Hz, 1H), 1.80-1.90 (m, 1H), 1.42-1.52 (m, 2H), 0.92 (d, J=6.5Hz, 6H) | |
| # LRMS (ESI): (calc.) 263.3; (found) 264.1 (MH)⁺. | 1 | | | | | |
| 12 | 4l | 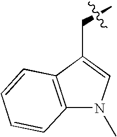 | —OH | (S)-1,2,3,4- Tetrahydro-N- hydroxy-2((1- methyl-1-H- indol-3- yl)methyl)-3- oxoquinoxaline- 6-carboxamide | ¹H NMR (DMSO-d₆) δppm: 10.88 (bs, 1H), 20.42 (s, 1H), 8.81 (s, 1H), 7.61 (d, J=7.4Hz, 1H), 7.44 (d, J=8.2Hz, 1H), 7.24-7.16 (m, 4H), 7.08 (t, J =7.6Hz, 1H), 6.70 (d, J=8.0Hz, | |
| # 1H), 6.46 (s, 1H), 4.21 (s, 1H), 3.81 (s, 3H), 3.17 (dd, J=4.1, 10.4Hz, 1H), 3.07 (dd, J=7.4, 7.0Hz, 1H). LRMS (ESI): (calc.) 350.37; (found) 351 (MH)⁺. | 1 | Steps 1-2 (ex. 1), steps 1 and 3 (Ex. 19) | | | | |
| 14 | 5c | 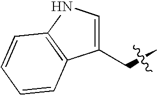 | 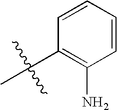 | (S)-2-((1H-Indol- 3-yl)methyl)-N- (2- aminophenyl)- 1,2,3,4- tetrahydro-3- oxoquinoxaline- 6-carboxamide | ¹H NMR: (DMSO-d₆) δ(ppm): 10.84 (s, 1H), 10.35 (s, 1H), 9.30 (s, 1H), 7.50 (d, J=7.8Hz, 1H), 7.43 (d, J =10.0Hz, 1H), 7.30 (d, J=8.0Hz, 1H), 7.27 (s, 1H), 7.11-7.09 (m, 2H), | |
| # 7.04 (t, J =6.9Hz, 1H), 6.97-6.89 (m, 2H), 6.73 (d, J=8.2Hz, 1H), 6.65 (d, J =8.2Hz, 1H), 6.56 (t, J=7.2Hz, 1H), 6.44 (s, 1H), 4.80 (br s, 2H), 4.17-4.15 (m, 1H), 3.07-2.98 (m, 2H). LRMS (ESI): (calc.) 411.2; (found) 4.12.4 (MH)⁺. | 1 | | | | | |

### EXAMPLE 15

### (S)-2-(4-Hydroxybenzyl)-1,2,3,4-tetrahydro-N-hydroxy-3-oxoquinoxaline-6-carboxamide (Compound 6h)

### Step1-3: (S)-2-(4-tert-Butoxybenzyl)-N-hydroxy-3-oxo-1,2,3,4-tetrahydroquinoxaline-6-carboxamide (Compound 4h)

Following the same procedure Example 1, step 1-3, Compound 4a, but substituting 3h for 3a, the title Compound 4h was obtained in 36% yield. ¹H NMR: (DMSO-d₆) δ (ppm): 10.77 (s, 1H), 10.32 (s, 1H), 8.68 (br s, 1H), 7.14-7.04 (m, 4H), 6.78 (d, J=7.5 Hz, 2H), 6.58 (d, J=8.5 Hz, 1H), 6.43 (s, 1H), 4.11-4.13 (m, 1H), 2.88-2.85 (m, 2H), 1.20 (s, 9H). LRMS (ESI): (calc.) 369.1; (found) 314.0 (M-tBu)⁺.

### Step 4: (S)-2-(4-Hydroxybenzyl)-1,2,3,4-tetrahydro-N-hydroxy-3-oxoquinoxaline-6-carboxamide (Compound 6h)

To a stirred solution of 4h (75 mg, 0.2 mmol) in DCM (15 mL) was added concentrated trifluroacetic acid (3 mL). The solution was stirred for 1 h and then diluted with water (10 mL). Aqueous extraction was performed with DCM (2×10 mL) and EtOAc (10 mL). The organic layers were concentrated and the residue was purified by preparative reverse phase HPLC (aquasil C-18, 100×4.6, 5 uM) with MeOH (15-95%) in H₂O to afford the title Compound 6h as a yellow solid (15 mg, 24%). ¹H NMR: (DMSO-d₆) δ (ppm): 10.80 (s, 1H), 10.31 (s, 1H), 9.18 (s, 1H), 8.71 (s, 1H), 7.13-7.11 (m, 2H), 6.94 (d, J=7.5 Hz, 2H), 6.61-6.60 (m, 3H), 6.31 (s, 1H), 4.02-3.99 (m, 1H), 2.81-2.71 (m, 2H). LRMS (ESI): (calc.) 313.0; (found) 314.0 (MH)⁺.

### EXAMPLE 16

### (S)-1-(4-Methoxybenzyl)-1,2,3,4-tetrahydro-N-hydroxy-2-isopropyl-3 oxoquinoxaline-6-carboxamide (Compound 8)

### Step 3: (S)-1-(4-Methoxybenzyl)-1,2,3,4-tetrahydro-2-isopropyl-3-oxoquinoxaline-6-carboxylic acid (Compound 7)

Acid 3a (see Example 1, Compound 4a, steps 1-2, Scheme 1 for preparation) (130 mg, 0.555 mmol), 4-methoxy-benzaldehyde (0.070 mL, 0.555 mmol), dibutyltin dichloride (17 mg, 0.056 mmol) and phenyl silane (0.08 mL, 0.610 mmol) were all dissolved in THF/DMF (2:1, 3 mL), and the resulting solution stirred for 3 days. Following removal of the solvent and purification of the residue by flash chromatography (eluent 0-100% EtOAc in hexanes), 114 mg (58%) of Compound 7 was obtained as a white crystalline solid. ¹H NMR: (DMSO-d₆) δ (ppm): 12.28 (br s,1H), 10.62 (br s,1H), 7.40 (d, J=8.6 Hz, 1H), 7.36 (s,1H), 7.19 (d, J=8.4 Hz, 2H), 6.89 (d, J=8.3 Hz, 2H), 6.77 (d, J=8.6 Hz, 1H), 4.83 (d, J=15.5 Hz, 1H), 4.44 (d, J=15.3 Hz, 1H), 3.82 (d, J=5.9 Hz, 1H), 3.74 (s, 3H), 1.92-2.02 (m, 1H), 0.94 (d, J=6.8 Hz, 3H), 0.86 (d, J=6.7 Hz, 3H). LRMS (ESI): (calc.) 354.4; (found) 355.1 (MH)⁺.

### Step 4: (S)-1-(4-Methoxybenzyl)-1,2,3,4-tetrahydro-N-hydroxy-2-isopropyl-3-oxoquinoxaline-6-carboxamide (Compound 8)

Following the procedure described in Example 4a, step 3 (Scheme 1) but substituting acid 7 for acid 3a, the title Compound 8 was obtained in 39% yield as a white crystalline solid. ¹H NMR: (DMSO-d₆) δ (ppm): 10.85 (br s,1H), 10.57 (br s,1H), 8.79 (br s,1H), 7.18 (m, 4H), 6.88 (d, J=8.0 Hz, 1H), 6.71 (d, J=7.7 Hz, 1H), 4.82 (d, J=15.7 Hz, 1H), 4.42 (d, J=15.7 Hz, 1H), 3.78 (d, J=6.1 Hz, 1H), 3.73 (s, 3H), 1.90-2.00 (m, 1H), 0.93 (d, J=6.5 Hz, 3H), 0.86 (d, J=6.3 Hz, 3H). LRMS (ESI): (calc.) 369.4; (found) 370.2 (MH)⁺.

### EXAMPLE 18

### (S)—N-(5-(Hydroxycarbamoyl)pentyl)-1,2,3,4-tetrahydro-2-isopropyl-3-oxoquinoxaline-6-carboxamide (Compound 13)

### Step 3: (S)-Methyl 6-(2-isopropyl-3-oxo-1,2,3,4-tetrahydroquinoxaline-6-carboxamido)hexanoate (Compound 11)

Acid 3a (see Example 1 (Compound 4a), steps 1-2, Scheme 1 for preparation) (356 mg, 1.52 mmol) was dissolved in DMF (5 mL), and BOP (805 mg, 1.82 mmol) was subsequently added in one portion. After stirring at room temperature for 5 min, 6-amino-hexanoic acid methyl ester 10 (331 mg, 1.82 mmol) was added, followed by the addition of triethylamine (1.06 mL, 7.60 mmol). The resulting solution was stirred for 2 h prior to the removal of the solvent. After purification of the residue by flash chromatography (eluent 0-100% EtOAc in hexanes), Compound 11 was obtained as a light yellow crystalline solid (547 mg, 99%). LRMS (ESI): (calc.) 361.4; (found) 362.1 (MH)⁺.

### Step 4: (S)-6-(2-Isopropyl-3-oxo-1,2,3,4-tetrahydroquinoxaline-6-carboxamido)-hexanoic acid (Compound 12)

Methyl ester 11 (547 mg, 1.50 mmol) was dissolved in THF/MeOH/H₂O (1:2:1, 4 mL), and lithium hydroxide monohydrate (319 mg, 7.20 mmol) was added to the resulting solution. After stirring at room temperature for 2 h, the solution was acidified to pH=6 with HCl, and extracted from brine with EtOAc. Following purification of the residue by flash chromatography (eluent 0-100% EtOAc in hexanes), 428 mg (81%) of Compound 12 was obtained as a light yellow crystalline solid. LRMS (ESI): (calc.) 347.4; (found) 348.6 (MH)⁺.

### Step 5: (S)—N-(5-(Hydroxycarbamoyl)pentyl)-1,2,3,4-tetrahydro-2-isopropyl-3-oxoquinoxaline-6-carboxamide (Compound 13)

Following the procedure described in Example 1, Compound 4a, step 3 (Scheme 1) but substituting acid 12 for acid 3a, the title Compound was obtained in 44% yield as a light yellow crystalline solid. ¹H NMR: (DMSO-d₆) δ (ppm): 10.34 (br s, 1H), 8.67 (br s, 1H), 8.03 (br s, 1H), 7.27 (d, J=8.2 Hz, 1H), 7.21 (s, 1H), 6.69 (d, J=8.2 Hz, 1H), 6.52 (s, 1H), 3.69 (d, J=2.7 Hz, 1H), 3.35 (s, 1H), 3.14-3.24 (m, 2H), 2.00-2.10 (m, 1H), 1.97 (t, J=7.3 Hz, 2H), 1.42-1.60 (m, 4H), 1.24-1.34 (m, 2H), 0.97 (d, J=6.8 Hz, 3H), 0.86 (d, J=6.7 Hz, 3H). LRMS (ESI): (calc.) 362.4; (found) 363.1 (MH)⁺.

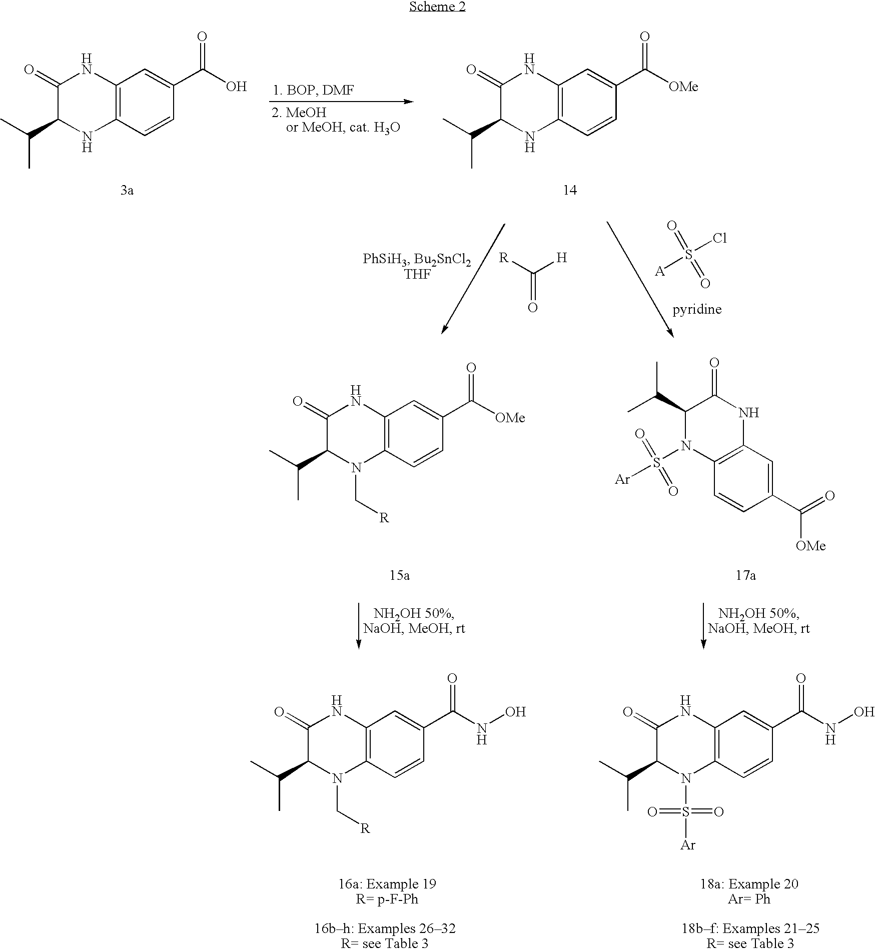

### EXAMPLE 19

### (S)-1-(4-Fluorobenzyl)-N-hydroxy-2-isopropyl-3-oxo-1,2,3,4-tetrahydroquinoxaline-6-carboxamide (Compound 16a)

### Step 1: (S)-Methyl-1,2,3,4-tetrahydro-2-isopropyl-3-oxoquinoxaline-6-carboxylate (Compound 14)

Acid 3a (see Example 1 (Compound 4a), steps 1-2, Scheme 1 for preparation) (1.5 g, 6.40 mmol) and BOP (1.813 g, 4.098 mmol) were dissolved in DMF (4 mL). Reaction was stirred for 3 h. The reaction was quenched with MeOH (4 mL) and stirred for 16 h. EtOAc (10 mL) was added, and the organic phase was washed twice with NaHCO₃, and once with brine. The organic layer was dried over MgSO₄, filtered and concentrated. The title Compound 14 was obtained as white solid by crystallization from MeOH (1.34 g, 84%). ¹H NMR: (DMSO-d₆) δ (ppm): 10.46 (s, 1H), 7.43 (dd, J=2, 6.5 Hz, 1H), 7.33 (d, J=2 Hz, 1H), 6.91 (s, 1H), 6.78 (d, J=8.4 Hz, 1H), 3.83 (dd, J=2.2, 1 Hz), 3.79 (s, 3H), 2.15-2.09 (m, 1H), 1.01 (d, J=7.0 Hz, 3H), 0.89 (d, J=6.7 Hz, 3H).

### Step 2: (S)-Methyl1-(4-fluorobenzyl)-2-isopropyl-3-oxo-1,2,3,4-tetrahydro-quinoxaline-6-carboxylate (Compound 15a)

Following the procedure described in Example 16, step 3 (Scheme 1) but substituting 14 for acid 3a, the title Compound 15 was obtained and used in the following step without further purification.

### Step 3: (S)-1-(4-Fluorobenzyl)-N-hydroxy-2-isopropyl-3-oxo-1,2,3,4-tetrahydroquinoxaline-6-carboxamide (Compound 16a)

To the reaction mixture of 15a, NH₂OH 50% wt solution (1 mL) and 1N NaOH (5 eq.) was added and stirred for 2 h. The reaction mixture was concentrated and the residue was purified by preparative reverse phase HPLC (aquasil C-18, 100×4.6, 5 uM) with MeOH (10-95%) in H₂O to afford the title Compound 16a.

The title Compound 16a was obtained as white solid (7 mg, 10%). ¹H NMR (CD₃OD) δ ppm: 7.28-7.25 (m, 2H), 7.22 (bs, 1H), 7.02 (t, J=8.8 Hz, 2H), 6.75 (d, J=7.8 Hz, 1H), 4.47 (d, J=15.7 Hz, 1H), 3.75 (d, 6.5 Hz, 1H), 2.05-2.0 (m, 1H), 0.99 (d, J=6.8 Hz, 3H), 0.92 (d, J=6.8 Hz, 3H). LRMS (ESI): (calc.) 357.38; (found) 358 (MH)⁺.

### EXAMPLE 20

### (S)—N-Hydroxy-2-isopropyl-3-oxo-1-(phenylsulfonyl)-1,2,3,4-tetrahydroquinoxaline-6-carboxamide (Compound 18a)

### Step 2: (S)-Methyl 2-isopropyl-3-oxo-1-(phenylsulfonyl)-1,2,3,4-tetrahydro-quinoxaline-6-carboxylate (Compound 17a)

General Procedure for the Preparation of sulfonamides: Methyl ester 14 (500 mg, 2.0 mmol) and benzenesulfonyl chloride (1.13 g, 6.4 mmol) were stirred in pyridine (2 mL) at room temperature and the progress of the reaction is followed by MS and TLC. After 16 h, pyridine was removed under reduced pressure and the crude product was purified by column chromatography eluting with 40% EtOAc in hexanes to give 17a in 81% yield. LRMS (ESI): (calc.) 388.4; (found) 389.1 (MH)⁺.

### Step 3: (S)—N-Hydroxy-1-benzenesulfonyl-2-isopropyl-3-oxo-1,2,3,4-tetrahydroquinoxaline-6-carboxamide (Compound 18a)

Following the procedure described in Example 19, step 3 (Scheme 2) but substituting methyl ester 17a for 15a, the title Compound 18a was obtained as a pink solid (20 mg, 30%). ¹H NMR: (DMSO-d₆) δ (ppm): 10.39 (s, 1H), 7.59 (m, 2H), 7.41 (t, J=7.6 Hz, 2H), 7.35 (d, J=8.2 Hz, 1H), 7.29 (d, J=8.2 Hz, 2H), 7.1 (s, 1H), 3.98 (d, J=9.6 Hz, 1H), 1.45-1.38 (m, 1H), 0.85 (d, J=6.7 Hz, 3H), 0.82 (d, J=6.7 Hz, 3H).

### EXAMPLES 21-32

Examples 21-32 describe the preparation of Compound 16 (b-h) and 18 (b-f), using the same procedures as described for Compound 16a in Example 19 or Compound 18a in Example 20. Characterization data are presented in Table 3.

TABLE 3

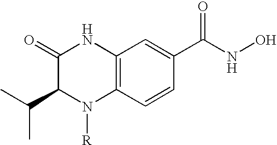

| Ex | Cpd | R | Name | Characterization | Scheme |
|---|---|---|---|---|---|
| 21 | 18b | 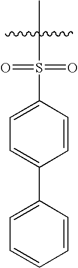 | (S)-N- Hydroxy-1- (biphenyl-4- sulfonyl)-2- isopropyl-3- oxo-1,2,3,4- tetrahydroquin oxaline-6- carboxamide | ¹H NMR: (MeOD-d₄) δ (ppm): 10.53 (b, 1H), 7.82 (d, J=8.2Hz, 2H), 7.7 (d, J=7.0Hz, 2H), 7.65 (d, J=8.2Hz, 1H), 7.48-7.41 (m, 6H), 7.19 (d, J=2Hz, 1H), 4.09 (d, J = | |
| # 9.6Hz, 1H), 1.54-1.47 (m, 1H), 0.93 (d, J=6.7,Hz, 3H), 0.91 (d J=6.7,Hz, 3H). LRMS (ESI): (calc.) 465.5; (found) 466 (MH)⁺. | 2 | | | | |
| 22 | 18c | 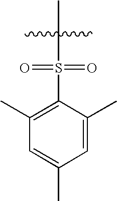 | (S)-N- Hydroxy-2- isopropyl-3- oxo-1(2,4,6- trimethylbenzene- sulfonyl)- 1,2,3,4- tetrahydroquin oxaline-6- carboxamide | ¹H NMR: (MeOD-d₄) δ (ppm): 10.67 (b, 1H), 7.29 (dd, J=8.4, 15.0Hz, 2H), 7.21 (s, 1H), 6.97 (s, 2H), 3.75 (d, J =9.6Hz, 1H), 2.30 (s, 6H), 2.18 (s, 3H), 1.46-1.41 (m, | |
| # 1H), 0.81 (d, J=6.7Hz, 3H), 0.75 (d, J=6.5Hz, 3H). LRMS (ESI): (calc.) 431.5; (found) 432 (MH)⁺. | 2 | | | | |
| 24 | 18e | 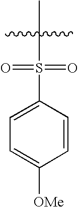 | (S)-N- Hydroxy-1-(4- methoxybenzene- sulfonyl-2- isopropyl-3- oxo-1,2,3,4- tetrahydroquin oxaline-6- carboxamide | ¹H NMR: (MeOD-d₄) δ (ppm): 10.50 (b, 1H), 7.60 (d, J=8.4Hz, 1H), 7.4 (dd, J=1.8, 6.5Hz, 1H), 7.28 (d, J=8.8Hz, 2H), 7.18 (d, J=1.8Hz, 1H), 6.99 (d, J=8.8Hz, 2H), | |
| # 4.03 (d, J=9.6Hz, 1H), 3.78 (s, 3H), 1.51-1.44 (m, 1H), 0.92 (d, J =6.7,Hz, 3H), 0.89 (d, J=6.5Hz, 3H). LRMS (ESI): (calc.) 407.42; (found) 408 (MH)⁺. | 2 | | | | |
| 25 | 18f | 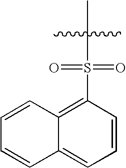 | (S)-N- Hydroxy-1- naphthalene- sulfonyl-2- isopropyl-3- oxo-1,2,3,4- tetrahydroquin oxaline-6- carboxamide | ¹H NMR: (MeOD-d₄) δ (ppm): 10.21 (s, 1H), 8.22 (d, J=8.4Hz, 1H), 8.14 (dd, J=1, 6.5Hz, 1H), 8.0 (d, J=8.2Hz, 1H), 7.79 (d, J=8.8Hz, 1H), 7.69 (d, J=8.2Hz, 1H), 7.60 (t, J = | |
| # 7.8Hz, 1H), 7.52 (t, J=6.8Hz, 1H), 7.44 (dd, J=2, 6.5Hz, 1H), 7.26-7.21 (m, 1H), 6.95 (d, J =2Hz, 1H), 4.06 (d, J=9.6Hz, 1H), 1.53-1.47 (m, 1H), 0.91 (d, J=6.5,Hz, 3H), 0.90 (d, J=6.7,Hz, 3H). LRMS (ESI): (calc.) 439.5; (found) 440 (MH)⁺. | 2 | | | | |
| 26 | 16b | 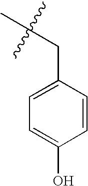 | (S)-1-(4- Hydroxybenzyl)- 1,2,3,4- tetrahydro-N- hydroxy-2- isopropyl-3- oxaquinoxaline-6- carboxamide | ¹H NMR: (MeOD-d₄) δ (ppm): 7.17 (d, J=8.4Hz, 1H), 7.09 (d, J=1.6Hz, 1H), 6.96 (d, J=8.4Hz, 2H), 6.68 (d, J=8.6Hz, 1H), 6.59 (d, J=8.6Hz, 2H), 4.68 (d, J=14.9Hz, 1H), 4.24 (d, | |
| # J=15Hz, 1H), 3.59 (d, J=6.5Hz, 1H), 1.85-1.93 (m, 1H), 0.92 (d, J=6.8,Hz, 3H), 0.88 (d, J=6.8,Hz, 3H). LRMS (ESI): (calc.) 355.4; (found) 356 (MH)⁺. | 2 | | | | |
| 27 | 16c | 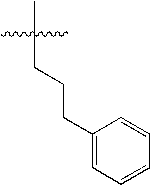 | (S)-1,2,3,4- Tetrahydro-N- hydroxy-2- isopropyl-3- oxo-1-(3- phenylpropyl) quinoxaline-6- carboxamide | ¹H NMR: (MeOD-d₄) δ (ppm): 7.33 (dd, J=8.4, 1.4Hz, 1H), 7.25-7.12 (m, 8H), 6.71 (d, J=8.6Hz, 1H), 3.83-3.77 (m, 1H), 3.63 (d, J=6.7Hz, 2H), 3.20-3.14 (m, 1H), 2.64-2.6 (m, 1H), 2.0-1.88 | |
| # (m, 1H), 0.85 (d, J=6.8,Hz, 3H), 0.78 (d, J=6.8,Hz, 3H). LRMS (ESI): (calc.) 367.4; (found) 368 (MH)⁺. | 2 | | | | |
| 28 | 16d | 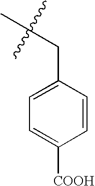 | 4-(((S)-7- (Hydroxy- carbamoyl)-2,3- dihydro-3- isopropyl-2- oxoquinoxalin- 4(1H)- yl)methyl) benzoic acid | ¹H NMR: (MeOD-d₄) δ (ppm): 7.91 (d, J=8.0Hz, 2H), 7.32 (d, J=8.0Hz, 2H), 7.24 (d, J=7.6Hz, 2H), 6.72 (d, J=8.6Hz, 2H), 4.95 (d, J=9Hz, 1H), 4.57 (d, J=0.2Hz, | |
| # 1H), 3.81 (d, J=6.3Hz, 1H), 2.08-2.03 (m, 1H), 1.01 (d, J =6.8,Hz, 3H), 0.94 (d, J=6.8,Hz, 3H). LRMS (ESI): (calc.) 383.3; (found) 384 (MH)⁺. | 2 | | | | |
| 29 | 16e | 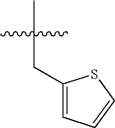 | (S)-1,2,3,4- Tetrahydro-N- hydroxy-2- isopropyl-3- oxo-1-(3- ((thiophen-2- yl)methyl) quinoxaline-6- carboxamide | ¹H NMR: (MeOD-d₄) δ (ppm): 7.32 (d, J=9Hz, 1H), 7.26 (d, J=4.1Hz, 1H), 7.22 (s, 1H), 7.02 (d, J=3.5Hz, 1H), 6.94-6.91 (m, 2H), 5.11 (d, J=16.0Hz, 1H), 4.63 (d, J = | |
| # 15.7Hz, 1H), 3.77 (d, J =6.5Hz, 1H), 1.99-1.98 (m, 1H), 0.98 (d, J =6.8,Hz, 3H), 0.91 (d, J=6.7,Hz, 3H). LRMS (ESI): (calc.) 345.4; (found) 346 (MH)⁺. | 2 | | | | |
| 30 | 16f | 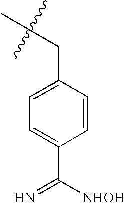 | (S)-N-hydroxy- 1-(4-(N- hydroxy- carbamimidoyl) benzyl)-2- benzyl)-2- isopropyl-3- oxo-1,2,3,4- tetrahydro- quinoxaline-6- carboxamide | ¹H NMR: (MeOD-d₄) δ (ppm): 7.56 (d, J = 8.2Hz, 2H), 7.29 (d, J=8.2Hz, 2H), 7.24-7.22 (m, 2H), 6.73 (d, J =8.2Hz, 1H), 5.0 (d, J=16.4Hz, 1H, | |
| # overlapped with water signal), 4.54 (d, J =16.2Hz, 1H), 3.8 (d, J=6.3Hz, 1H), 2.07-2.0 (m, 1H), 1.00 (d, J=6.8,Hz, 3H), 0.94 (d, J=6.8,Hz, 3H). LRMS (ESI): (calc.) 397.4; (found) 398 (MH)⁺. | 2 | | | | |
| 31 | 16g | 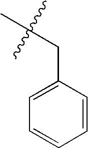 | (S)-1-Benzyl 1,2,3,4- tetrahydro-N- hydroxy-2- isopropyl-3- oxoquinoxaline-6- carboxamide | ¹H NMR: (MeOD-D₄) δ (ppm): 7.19-7.08 (m, 7H), 6.65 (d, J=8.4Hz, 1H), 4.37 (d, J=15.8Hz, 1H), 3.64 (d, J =6.3Hz, 1H), 1.96-1.88 (m, 1H), 0.90 (d, J =6.7,Hz, 3H), 0.83 (d, J=6.8,Hz, 3H). LRMS (ESI): | |
| # (calc.) 339.4; (found) 340 (MH)⁺. | 2 | | | | |
| 32 | 16h | 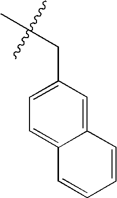 | (S)-1,2,3,4- Tetrahydro-N- hydroxy-2- isopropyl-1- ((naphthalen- 3-yl)methyl)-3- oxoquinoxaline-6- carboxamide | ¹H NMR: (MeOD-d₄) δ (ppm): 10.78 (bs, 1H), 10.58 (s, 1H), 8.76 (bs, 1H), 7.84-7.77 (m, 4H), 7.48-7.43 (m, 2H), 7.36 (d, J=8.4Hz, 1H), 7.20 (s, 1H), 7.12 (d, J=6.7Hz, 1H), 5.01 (d, J=15.8Hz, | |
| # 1H), 4.65 (d, 16.2Hz, 1H), 3.84 (d, 6.5Hz, 1H), 1.99-1.93 (m, 1H), 0.94 (d, J=7.0, Hz, 3H), 0.86 (d, J=6.8,Hz, 3H). LRMS (ESI): (calc.) 389.5; (found) 390 (MH)⁺. | 2 | | | | |

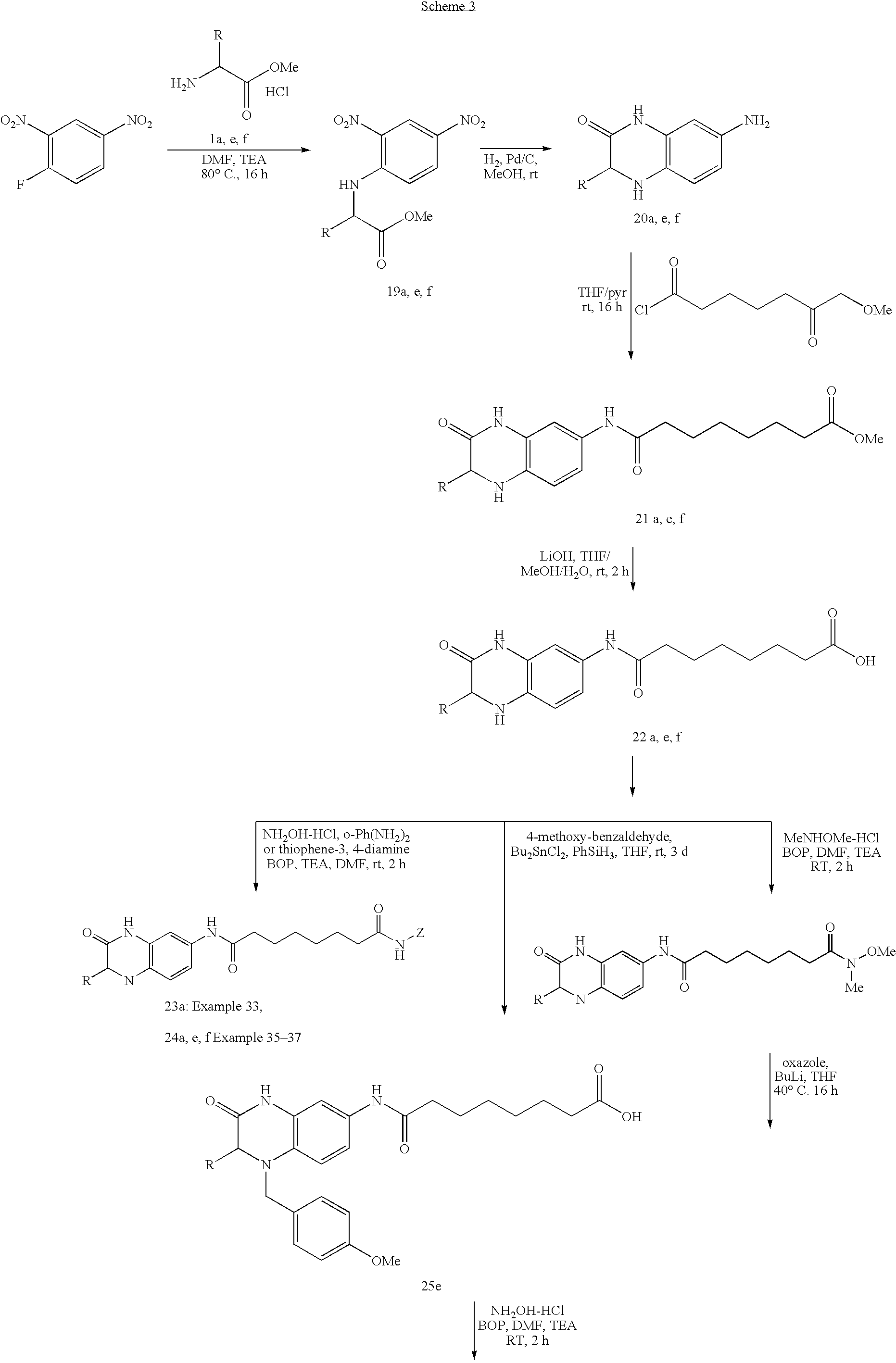

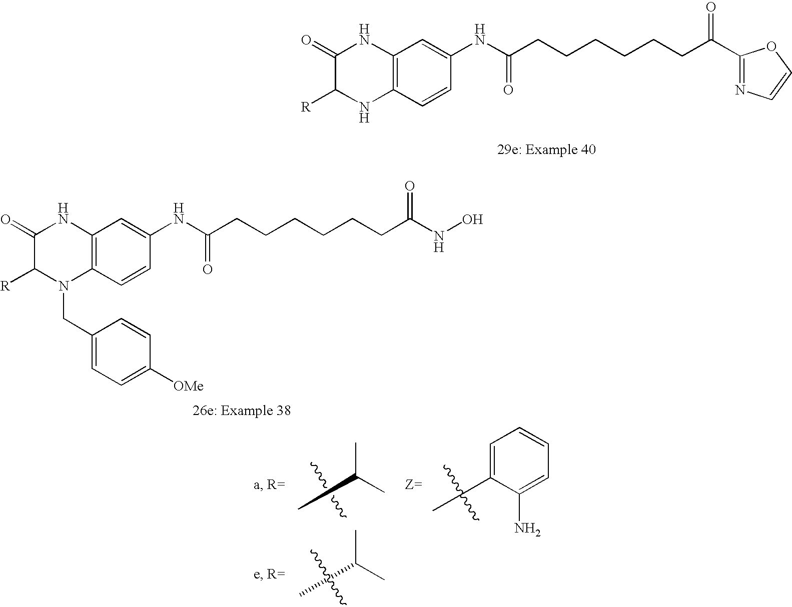

### EXAMPLE 33

### N1-(2-Aminophenyl)-N-8-((S)-1,2,3,4-tetrahydro-2-isopropyl-3-oxoquinoxalin-6-yl)octanediamide (Compound 23a)

### Step 1: (S)-Methyl 2-(2,4-dinitrophenylamino)-3-methylbutanoate (Compound 19a)

Following the procedure described in Example 1, Compound 4a, step 1 (Scheme 1) but substituting 1-fluoro-2,4-dinitrobenzene for 4-fluoro-3-nitrobenzoic acid, the title Compound 19a was obtained in near quantitative yield, and used in the subsequent reaction without further purification. LRMS (ESI): (calc.) 297.3; (found) 298.1 (MH)⁺.

### Step 2: (S)-7-Amino-3,4-dihydro-3-isopropylquinoxalin-2(1H)-one (Compound 20a)

Following the procedure described in Example 1, 4a, step 2 (Scheme 1) but substituting Compound 19a for 2a, the title Compound 20a was obtained in 88% yield as a light brown crystalline solid. LRMS (ESI): (calc.) 205.3; (found) 206.2 (MH)⁺.

### Step 3: Methyl 7-((S)-1,2,3,4-Tetrahydro-2-isopropyl-3-oxoquinoxalin-6-ylcarbamoyl)-heptanoate (Compound 21a)

Aniline 20a (508 mg, 2.48 mmol) was dissolved in THF/pyridine (2:1, 6 mL), followed by the addition of methyl 7-chlorocarbonyl-heptanate (0.387 mL, 2.72 mmol). After stirring for 16 h at room temperature, the solvent was removed. The residue was purified by flash chromatography (eluent 0-80% EtOAc in hexanes) to afford Compound 21a as a light pink crystalline solid (247 mg, 27%). LRMS (ESI): (calc.) 375.5; (found) 376.1 (MH)⁺.

### Step 4: 7-((S)-1,2,3,4-Tetrahydro-2-isopropyl-3-oxoquinoxalin-6-ylcarbamoyl)-heptanoic acid (Compound 22a)

Following the procedure described in Example 18, step 4 (Scheme 1) but substituting methyl ester 21 a for 11, the title Compound 22a was obtained in 76% yield as a light yellow crystalline solid. LRMS (ESI): (calc.) 361.4; (found) 362.3 (MH)⁺.

### Step 5: N1-(2-Aminophenyl)-N-8-((S)-1,2,3,4-tetrahydro-2-isopropyl-3-oxoquinoxalin-6-yl)octanediamide (Compound 23a)

Following the procedure described in Example 1, Compound 4a, step 3 (Scheme 1) but substituting acid 22a for acid 3a, and benzene-1,2-diamine for hydroxylamine hydrochloride, the title Compound 23a was isolated in 71% yield as a light yellow crystalline solid. ¹H NMR: (DMSO-d₆) δ (ppm): 12.25 (br s, H), 10.21 (br s, 1H), 9.11 (br s, 1H), 7.96 (br s,2H), 7.86 (s, 1H), 7.63 (d, J=8.6 Hz, 1H), 7.35 (d, J=8.8 Hz, 1H), 7.15 (d, J=7.6 Hz, 1H), 6.88 (t, J=7.6 Hz, 1H), 6.72 (d, J=7.6 Hz, 1H), 6.54 (t, J=7.2 Hz, 1H), 4.84 (s, 1H), 2.55 (d, J=9.4 Hz, 1H), 3.44 (m, 1H), 2.34 (m, 4H), 1.64 (m, 4H), 1.38 (m, 4H), 1.23 (d, J=6.7 Hz, 6H). LRMS (ESI): (calc.) 451.6; (found) 450.5 (MH)⁺.

### EXAMPLES 34-37

Examples 34-37 describe the preparation of Compound 23a2, 24a, 24e and 24f using the same procedures as described for Compound 23a1 in Example 33. Characterization data are presented in Table 4.

TABLE 4

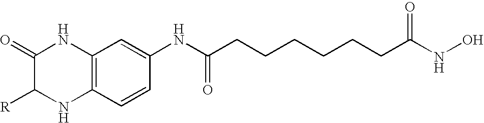

| Ex. | Cpd. | R | Name | Characterization | Scheme |
|---|---|---|---|---|---|
| 35 | 24a | 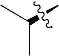 | N1-((S)-1,2,3,4- Tetrahydro-2- isopropyl-3- oxoquinoxalin- 6-yl)-N3- hydroxyoctane- diamide | ¹H NMR: (MeOD-d₄) δ (ppm): 10.43 (br s, 1H), 8.73 (br s, 1H), 7.89 (s, 1H), 7.62 (d, J=8.6Hz, 1H), 7.39 (d, J =8.8Hz, 1H), 2.39 (t, J=7.4Hz, 2H), 3.44 (m, 1H), 1.94-2.01 (m, 2H), 1.58-1.66 (m, 2H), 1.45-1.56 | |
| # (m, 2H), 1.28-1.36 (m, 4H), 1.23 (d, J=6.8Hz, 6H). LRMS (ESI): (calc.) 376.5; (found) 375.4 (MH)⁺. | 3 | | | | |
| 36 | 24e | 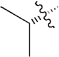 | N1-((R)-1,2,3,4- Tetrahydro-2- isopropyl-3- oxoquinoxalin- 6-yl)-N3- hydroxyoctane- diamide | ¹H NMR: (MeOD-d₄) δ (ppm): 12.25 (br s, 1H), 10.35 (s, 1H), 8.68 (d, J =1.6Hz, 1H), 10.20 (s, 1H), 7.86 (d, J =2.2Hz, 1H), 7.63 (d, J=8.8Hz, 1H), 7.35 (dd, J=8.8, 2.3Hz, 1H), 3.40-3.50 (m, 1H), 2.37 (t, J = | |
| # 7.4Hz, 2H), 1.98 (t, J=7.4Hz, 2H), 1.58-1.68 (m, 2H), 1.49-1.58 (m, 2H), 1.28-1.39 (m, 4H), 1.23 (d, J=6.8Hz, 6H). LRMS (ESI): (calc.) 376.5; (found) 375.2 (M H)⁺. | 3 | | | | |
| 37 | 24f | 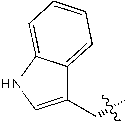 | N1-((R)-2-((1H- Indol-3-yl)- methyl)-1,2,3,4- tetrahydro-3- oxoquinoxalin- 6-yl)-N3- hydroxyoctane- diamide | ¹H NMR: (MeOD-d₄) δ (ppm): 10.34 (br s, 1H), 8.68 (br s, 1H), 7.84 (br s, 1H), 7.63 (t, J=8.4Hz, 1H), 7.33 (d, J=7.0Hz, 1H), 6.80-7.45 (m, 3H), 4.19 (br s, 1H), 3.37 (br s, 2H), 2.20-2.40 | |
| # (m, 2H), 1.90-2.00 (m, 2H), 1.40-1.70 (m, 4H), 1.31 (m, 4H). LRMS (ESI): (calc.) 463.5; (found) 462.3 (M-H)⁺. | 3 | | | | |

### EXAMPLE 38

### N1-((R)-1-(4-Methoxybenzyl)-1,2,3,4-tetrahydro-2-isopropyl-3-oxoquinoxalin-6-yl)-N-8-hydroxyoctanediamide (Compound 26e)

### Step 5: 7-((R)-1-(4-Methoxybenzyl)-1,2,3,4-tetrahydro-2-isopropyl-3-oxoquinoxalin-6-ylcarbamoyl)heptanoic acid (Compound 25e)

Following the procedure described in steps 1-4 (Scheme 3) for preparation of the acid 22a to obtain 22e. Then, following the procedure described in Example 16, step 3 (Scheme 1) but substituting acid 22e for acid 3a, the title Compound 25e was obtained in 44% yield as a white crystalline solid. ¹H NMR: (DMSO-d₆) δ (ppm): 11.99 (br s,1H), 10.38 (s, 1H), 9.60 (s, 1H), 7.18 (m, 3H), 6.93 (d, J=6.0 Hz, 1H), 6.87 (d, J=8.5 Hz, 2H), 6.66 (d, J=8.6 Hz, 1H), 4.65 (d, J=15.1 Hz, 1H), 4.29 (d, J=15.1 Hz, 1H), 3.74 (s, 3H), 3.52 (d, J=7.0 Hz, 1H), 2.18-2.30 (m, 4H), 1.85 (m, 1H), 1.48-1.62 (m, 4H), 1.25-1.38 (m, 4H), 0.90 (d, J=6.7 Hz, 3H), 0.84 (d, J=6.8 Hz, 3H). LRMS (ESI): (calc.) 481.6; (found) 482.2 (MH)⁺.

### Step 6: N1-((R)-1-(4-Methoxybenzyl)-1,2,3,4-tetrahydro-2-isopropyl-3-oxoquinoxalin-6-yl)-N8-hydroxyoctanediamide (Compound 26e)

Following the procedure described in Example 1, 4a, step 3 (Scheme 1) but substituting acid 25e for acid 3a, the title Compound 26e was obtained in 27% yield as a white crystalline solid. ¹H NMR: (DMSO-d₆) δ (ppm): 10.39 (br s, 2H), 9.62 (br s, 1H), 7.18 (s, 1H), 7.14 (d, J=8.4 Hz, 2H), 6.93 (d, J=8.4 Hz, 1H), 6.84 (d, J=8.4 Hz, 2H), 6.64 (d, J=8.6 Hz, 1H), 4.63 (d, J=15.1 Hz, 1H), 4.26 (d, J=15.3 Hz, 1H), 3.50 (d, J=7.0 Hz, 1H), 3.71 (s, 3H), 2.23 (t, J=7.2 Hz, 2H), 1.96 (t, J=7.2 Hz, 2H), 1.78-1.88 (m, 1H), 1.44-1.62 (m, 4H), 1.19 (m, 4H), 0.85 (d, J=6.7 Hz, 3H), 0.82 (d, J=6.7 Hz, 3H). LRMS (ESI): (calc.) 496.6; (found) 497.4 (MH)⁺.

### EXAMPLE 40

### N-((S)-1,2,3,4-Tetrahydro-2-isopropyl-3-oxoquinoxalin-6-yl)-8-(oxazol-2-yl)-8-oxooctanamide (Compound 29a)

### Step 6: N-((S)-1,2,3,4-Tetrahydro-2-isopropyl-3-oxoquinoxalin-6-yl)-8-(oxazol-2-yl)-8-oxooctanamide (Compound 29a)

Following the procedure described in Example 1, Compound 4a, step 3 (Scheme 1) and Example 39, step 5 (Scheme 3) for preparation of amide 27a. Oxazole (0.31 mL, 4.72 mmol) was dissolved in THF (5 mL), and the resulting solution cooled to −78° C. Butyllithium (2.95 mL, 4.72 mmol, 1.6 M solution in hexanes) was subsequently added drop wise over 15 min, followed by the addition of amide 27a (159 mg, 0.393 mmol). The resulting solution was warmed to room temperature, and then heated to 40° C. for 16 h. After cooling, the solution was diluted with aqueous ammonium chloride, and extracted with EtOAc. The organic layer was dried with Na₂SO₄, filtered, and concentrated. After purification of the residue by flash chromatography (eluent 0-100% EtOAc in hexanes), 22 mg (23%) of Compound 29a was obtained as a light yellow crystalline solid. ¹H NMR: (DMSO-d₆) δ (ppm):10.15 (br s,1H), 7.80 (d, J=2.2 Hz, 1H), 8.36 (s, 1H), 7.58 (d, J=8.5 Hz, 1H), 7.50 (d, J=1.6 Hz, 1H), 7.30 (dd, J=8.8, 2.2 Hz, 1H), 3.63 (br s, 1H), 3.36-3.44 (m, 1H), 3.06 (br s, 1H), 3.02 (m, 2H), 2.33 (t, J=7.4 Hz, 2H), 1.54-1.68 (m, 4H), 1.30-1.38 (m, 4H), 1.18 (d, J=6.7 Hz, 6H). LRMS (ESI): (calc.) 412.5; (found) 411.1 (M-H)⁻.

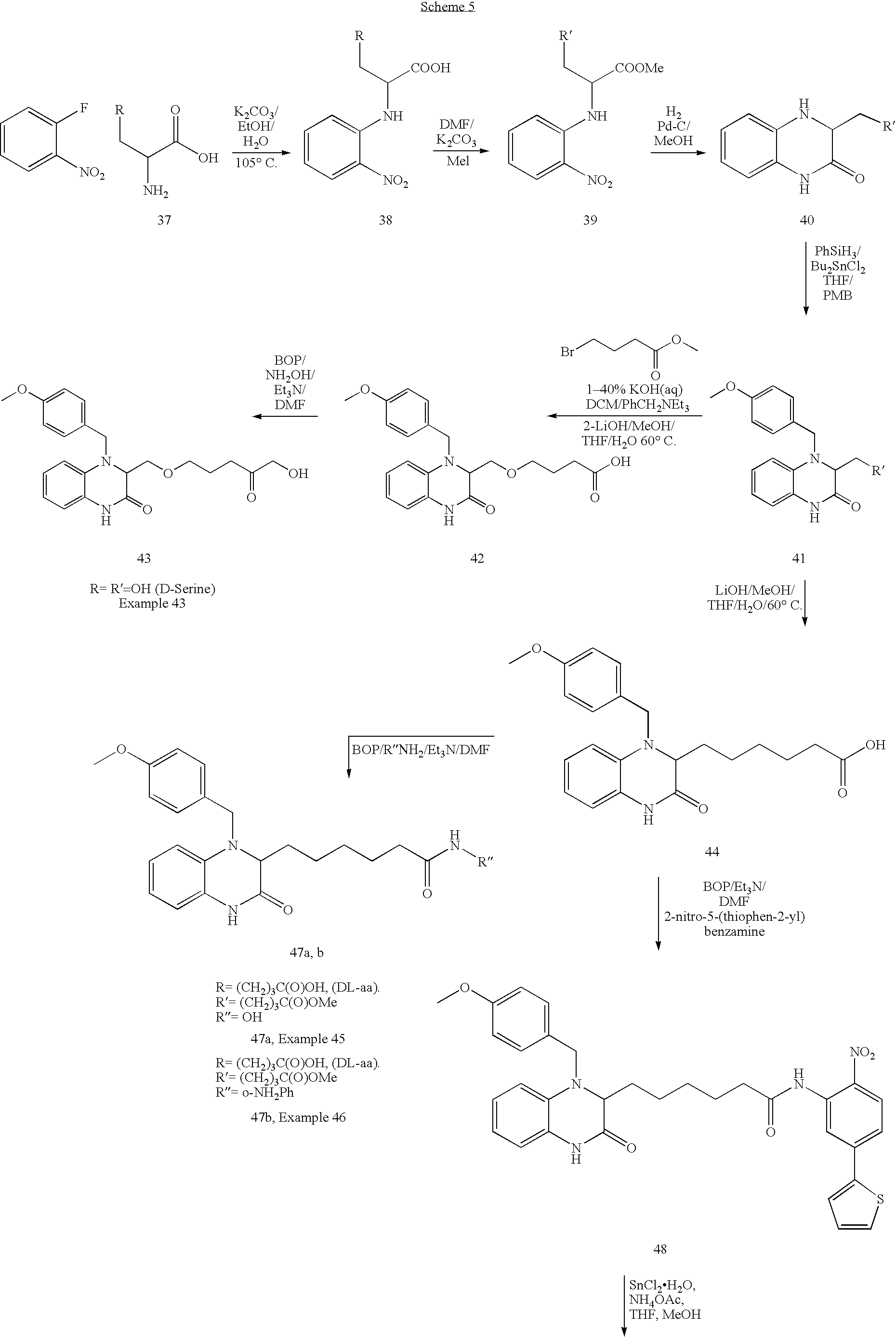

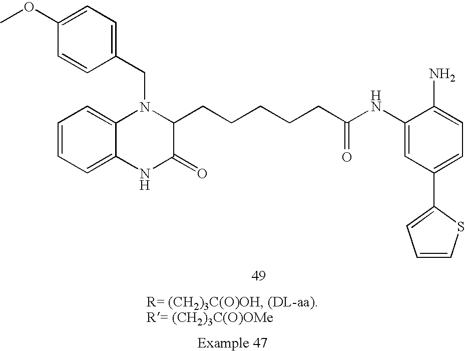

### EXAMPLE 43

### 6-((R)-1-(4-methoxybenzyl)-1,2,3,4-tetrahydro-3-oxoquinoxalin-2-yl)-N-hydroxy-4-oxy-hexanamide (Compound 43)

### Step 1: R-2-(2-Nitrophenylamino)-3-hydroxyrropanoic acid (Compound 38)

(D)-serine 37 (2.33 g, 22.2 mmol) and 1-fluoro-2-nitrobenzene (2.34 mL, 22.2 mmol) were dissolved in EtOH:H₂O (2:1, 15 mL) at room temperature. Then K₂CO₃ (6.10 g, 44.0 mmol) was added and the solution was heated at 105° C. for 16 h. After cooling the reaction, the suspension was filtered and washed with EtOH:H₂O to yield 1.16 g of orange solid (23%). LRMS (ESI): (calc.) 226; (found) 227 (MH)⁺.

### Step 2: R-Methyl 2-(2-nitrorphenylamino)-3-hydroxyrropanoate (Compound 39)

Compound 38 (1.16 g) was dissolved in DMF (10 mL). Then K₂CO₃ (829 mg, 1.2 mmol) and Mel (968 uL, 15 mmol) were added to the previous solution at room temperature. The reaction mixture was stirring for 16 h, K₂CO₃ was filtered and DMF was removed. The residue was dissolved in DCM-CHCl₃ and was washed with brine, dried over Na₂SO₄, filtered and concentrated. Yellow solid 39 was obtained in 84% (1.01 g). LRMS (ESI): (calc.) 240.2; (found) 241.2 (MH)⁺.

### Step 3: R-3,4-Dihydro-3-(hydroxymethyl)quinoxalin-2(1H)-one (Compound 40)

Following the procedure described in Scheme 1, step 3, Example 1, Compound 4a but substituting Compound 2a for Compound 39, the title Compound 40 was obtained in 52% (389 mg). LRMS (ESI): (calc.) 178.2; (found) 179.3 (MH)⁺.

### Step 4: R-4-(4-Methoxybenzyl)-3,4-dihydro-3-(hydroxymethyl)quinoxalin-2(1H)-one (Compound 41)

Following the procedure described in Scheme 1, step 3, and Example 16 but substituting Compound 3a for Compound 40, the title Compound 41 was obtained as a white solid (95%, 460 mg). ¹H NMR: (DMSO-d₆) δ (ppm): 10.37 (s, 1H), 7.20 (d, J=8.4 Hz, 2H), 6.84 (d, J=8.4 Hz, 2H), 6.73-6.68 (m, 2H), 6.58-6.53 (m, 2H), 4.60 (d, J=15.6 Hz, 1H), 4.32 (d, J=15.6 Hz, 1H), 3.84 (t, J=4.4 Hz, 1H), 3.70 (s, 3H), 3.54-3.51 (m, 2H). LRMS (ESI): (calc.) 298; (found) 299 (MH)⁺.

### Step 5: 6-((R)-1-(4-Methoxybenzyl)-1,2,3,4-tetrahydro-3-oxoquinoxalin-2-yl)-4-oxy-hexanoic acid (Compound 42)

A mixture of Compound 41 (460 mg, 1.54 mmol), benzyltriethylammonium chloride (626 mg, 2.77 mmol), methyl 4-bromobutanoate (7.2 mL, 61.4 mmol), and DCM (2 mL) was stirred at room temperature for 3 days in the presence of 40% KOH (5 mL). Then water and DCM were added. The organic phase was separated, washed with brine, dried over Na₂SO₄ and evaporated. The residue was chromatographed on silica gel (AcOEt:Hexanes:1:2 to AcOEt) to give the product 42 (145 mg, 24%). ¹H NMR: (DMSO-d₆) δ (ppm): 7.30 (bs, 1H), 7.21 (d, J=8 Hz, 2H), 6.83 ((d, J=8.0 Hz, 2H), 6.82-6.80 (m, 1H), 6.73-6.65 (m, 2H), 4.60 (d, 15.2 Hz, 1H), 4.36 (d, J=15.6, 1H), 3.92 (bs, 1H), 3.91-3.89 (m, 2H), 3.72 (s, 3H), 3.42 (bs, 2H), 2.05-1.96 (m, 2H), 1.70-1.65 (m, 2H). LRMS (ESI): (calc.) 384; (found) 383 (MH)⁺.

### Step 6: R-6-((R)-1-(4-Methoxybenzyl)-1,2,3,4-tetrahydro-3-oxoquinoxalin-2-yl)-N-hydroxy-4-oxy-hexanamide (Compound 43)

Following the procedure described in Scheme 1, step 3, Example 1 but substituting Compound 3a for Compound 42, the title Compound 43 was obtained as a beige solid (49 mg, 68%). ¹H NMR: (DMSO-d₆) δ (ppm): 7.25 (d, J=8 Hz, 2H), 7.07 (d, J=8 Hz, 1H), 6.85 (d, J=8.0 Hz, 2H), 6.87-6.84 (m, 1H), 6.75-6.69 (m, 2H), 4.63 (d, 15.2 Hz, 1H), 4.37 (d, J=15.6 Hz, 1H), 3.95 (bs, 1H), 3.86-3.80 (m, 2H), 3.72 (s, 3H), 3.48 (bs, 2H), 2.0-1.98 (m, 2H), 1.76-1.72 (m, 2H). LRMS (ESI): (calc.) 399; (found) 400 (MH)⁺.

### EXAMPLE 45

### 6-(1-(4-Methoxybenzyl)-1,2,3,4-tetrahydro-3-oxoquinoxalin-2-yl)-N-hydroxyhexanamide (Compound 47a)

### Step 5: 6-(1-(4-Methoxybenzyl)-1,2,3,4-tetrahydro-3-oxoquinoxalin-2-yl)hexanoic acid (Compound 44)

Following the procedure described in Scheme 1, step 4, Example 18 but substituting Compound 3a (see Example 28, step1-4, Scheme 4 for preparation) for Compound 41, the title Compound 44 was obtained as solid (74%, 298 mg). ¹H NMR: (DMSO-d₆) δ (ppm): 11.93 (s, 1H), 10.34 (s, 1H), 7.21 (d, J=8.0 Hz, 2H), 6.86 (d, J=8.0 Hz, 2H), 6.74-6.79 (m, 2H), 6.61-6.67 (m, 2H), 4.53 (d, J=7.2 Hz, 1H), 4.19 (d, J=7.2 Hz, 1H), 3.71 (s, 3H), 3.68 (t, J=2.0 Hz, 1H), 2.1 (t, J=7.2 Hz, 2H), 1.33-1.50 (m, 4H), 1.15-1.23 (m, 4H). LRMS (ESI): (calc.) 382.45; (found) 383.4 (MH)⁺.

### Step 6: 6-(1-(4-Methoxybenzyl)-1,2,3,4-tetrahydro-3-oxoquinoxalin-2-yl)-N-hydroxyhexanamide (Compound 47a)

Following the procedure described in Scheme 1, step 3, Example 1, Compound 4a but substituting Compound 3a for Compound 44 (see Example 44, step1-5, Scheme 5, for preparation), the title Compound 47a was obtained in 22% (23 mg) as a yellow solid. ¹H NMR: (DMSO-d₆) δ (ppm): 10.33 (s, 1H), 10.27 (s, 1H), 8.61 (s, 1H), 7.20 (d, J=8.8 Hz, 2H), 6.86 (d, 8.0 Hz, 2H), 6.79-6.74 (m, 2H), 6.66-6.61 (m, 2H), 4.54 (d, J=14.8 Hz, 1H), 4.22 (d, J=14.8 Hz, 1H), 3.71 (bs, 4H), 1.86 (dd, J=7.6, 8.0 Hz, 2H), 1.40-1.39 (m, 4H), 1.19-1.14 (m 4H). LRMS (ESI): (calc.) 397.4; (found) 398.4 (MH)⁺.

### EXAMPLE 46

### 6-(1-(4-Methoxybenzyl)-1,2,3,4-tetrahydro-3-oxoquinoxalin-2-yl)-N-(2-aminophenyl)hexanamide (Compound 47b)

### Step 6: 6-(1-(4-Methoxybenzyl)-1,2,3,4-tetrahydro-3-oxoquinoxalin-2-yl)-N-(2-aminophenyl)hexanamide (Compound 47b)

Following the procedure described in Scheme 1, step 3, Example 1, Compound 4a but substituting Compound 3a for Compound 44 (see Example 44, step1-5, Scheme 5, for preparation) and substituting hydroxylamine for benzene-1,2-diamine, the title Compound 47b was obtained as a yellow solid (116 mg, 94%). ¹H NMR: (DMSO-d₆) δ (ppm): 10.34 (s, 1H), 9.03 (s, 1H), 7.20 (d, J=8.4 Hz, 2H), 7.10 (d, J=7.6 Hz, 1H), 6.86-6.83 (m, 3H), 6.77-6.74 (m, 2H), 6.68-6.63 (m, 3H), 6.48 (dd, J=7.6, 7.2 Hz, 1H), 4.78 (bs, 2H), 4.54 (d, J=14.4 Hz, 1H), 4.20 (d, J=14.4 Hz, 1H), 3.70 (bs, 4H), 2.23 (dd, J=7.6, 7.2 Hz, 2H), 1.52-1.49 (m, 4H), 1.23 (bs, 4H). LRMS (ESI): (calc.) 472; (found) 473.5 (MH)⁺.

### EXAMPLE 47

### 6-(1-(4-methoxybenzyl)-1,2,3,4-tetrahydro-3-oxoquinoxalin-2-yl)-N-(2-amino-5-(thiophen-2-yl)phenyl)hexanamide (Compound 49)

### Step 6: 6-(1-(4-methoxybenzyl)-1,2,3,4-tetrahydro-3-oxoquinoxalin-2-yl)-N-(2-nitro-5-(thiophen-2-yl)phenyl)hexanamide (Compound 48)

Following the procedure described in Scheme 8, step 2, Example 62 but substituting Compound 61 for Compound 44, and oxalyl dichloride/DCM for BOP/DMF, the title Compound was obtained in 16% (17 mg) as a solid. LRMS (ESI): (calc.) 585.1; (found) 586.2 (MH)⁺.

### Step 7: 6-(1-(4-Methoxybenzyl)-1,2,3,4-tetrahydro-3-oxoquinoxalin-2-yl)-N-(2-amino-5-(thiophen-2-yl)phenyl)hexanamide (Compound 49)

Following the procedure described in Scheme 8, step 4, Example 62 but substituting Compound 62 for Compound 48, the title Compound 49 was obtained in 99% (16 mg) as a solid. ¹H NMR: (DMSO-d₆) δ (ppm): 10.42 (s, 1H), 9.16 (s, 1H), 7.53 (s, 1H), 7.38 (d, J=4.4 Hz, 1H), 7.28-7.24 (m, 4H), 7.08 (bs, 1H), 6.92 (d, J=7.6 Hz, 2H), 6.84 (d, J=8.0 Hz, 2H), 6.79-6.70 (m, 3H), 5.12 (bs, 1H), 4.61 (d, J=14.8 Hz, 1H), 4.27 (d, J=15.6 Hz, 1H), 3.70 (bs, 4H), 2.37-2.33 (m, 2H), 1.61-1.58 (m, 4H), 1.26-1.22 (m, 4H). LRMS (ESI): (calc.) 554.7; (found) 555 (MH)⁺.

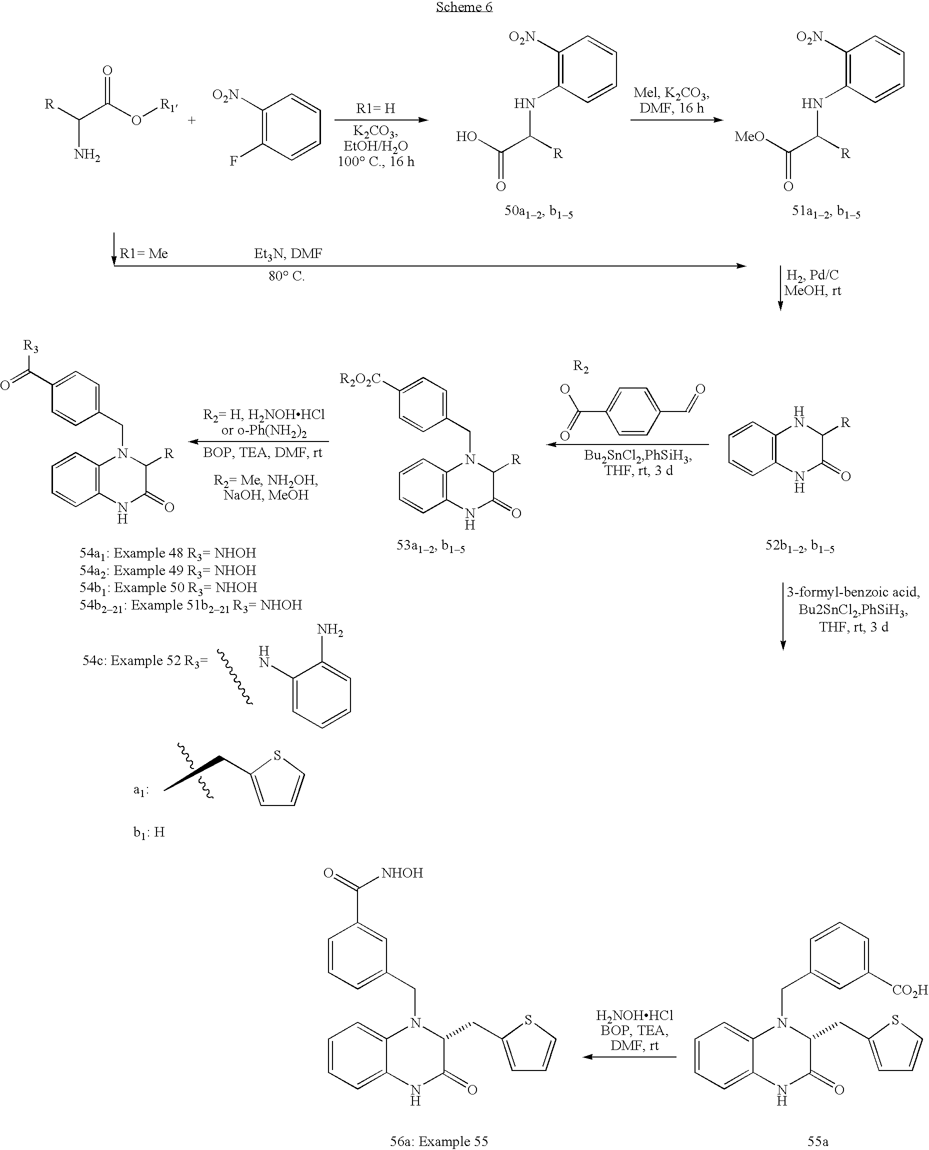

### EXAMPLE 48

### 4-(((S)-2,3-Dihydro-2-oxo-3-((thiophen-2-yl)methyl)quinoxalin-4(1H)-yl)methyl)-N-hydroxybenzamide (Compound 54a₁)

### Step 1: (S)-2-(2-nitrophenylamino)-3-(thiophen-2-yl)propanoic acid (Compound 50a₁)

Both (S)-2-amino-3-(thiophen-2-yl)propanoic acid (2.51 g, 14.66 mmol) and 1-fluoro-2-nitrobenzene (1.53 mL, 14.66 mmol) were dissolved in EtOH/H₂O (5:1, 24 mL) at room temperature. Potassium carbonate (1.56 g, 11.28 mmol) was then added, and the resulting solution heated to 100° C. for 16 h. After cooling, the solution was filtered, and the solvents removed. The residue, aniline 50a₁, was obtained in near quantitative yield, and used in the subsequent reaction without further purification. LRMS (ESI): (calc.) 292.3; (found) 293.1 (MH)⁺.

### Step 2: (S)-Methyl 2-(2-nitrophenylamino)-3-(thiophen-2-yl)propanoate (Compound 51a₁)

Aniline 50a₁ (4.29 g, 14.66 mmol) was dissolved in DMF (20 mL) at room temperature. Potassium carbonate (8.10 g, 58.64 mmol) and methyl iodide (2.74 mL, 43.98 mmol) were then added, and the resulting solution stirred at room temperature for 16 h. Following extraction from brine with EtOAc, the organic layer was concentrated, and the residue, aniline 51a₁, was obtained in near quatitative yield. This material was used in the subsequent reaction without further purification. LRMS (ESI): (calc.) 306.3; (found) 307.2 (MH)⁺.

### Step 3: (S)-3-(Thiophen-2-ylmethyl)-3,4-dihydroquinoxalin-2(1H)-one (Compound 52a₁)

Following the same procedure described in Example 1, Compound 4a, step 2, Scheme 1, but substituting ester 51a₁ for acid 2a, the title Compound was isolated in 76% yield as a light orange crystalline solid. LRMS (ESI): (calc.) 244.3; (found) 245.1 (MH)⁺.

### Step 4: 4-(((S)-2,3-Dihydro-2-oxo-3-((thiophen-2-yl)methyl)quinoxalin-4(1H)-yl)methyl)benzoic acid (Compound 53a₁)

Following the same procedure described in Example 16, step 3, Scheme 1, but substituting Compound 52a₁ for acid 3a, the title Compound was isolated in 81% yield as light yellow foam. LRMS (ESI): (calc.) 378.4; (found) 379.1 (MH)⁺.

### Step 5: 4-(((S)-2,3-Dihydro-2-oxo-3-((thiophen-2-yl)methyl)quinoxalin-4(1H)-yl)methyl)-N-hydroxybenzamide (Compound 54a₁)

Following the procedure described in Example 1, Compound 4a, step 3 (Scheme 1) but substituting acid 53a₁ for acid 3a, the title Compound was obtained in 18% yield as a light beige crystalline solid. ¹H NMR: (DMSO-d₆) δ (ppm): 11.16 (s,1H), 10.52 (s,1H), 9.02 (s, 1H), 7.67 (d, J=8.2 Hz, 2H), 7.33 (d, J=5.1 Hz, 1H), 7.30 (d, J=8.2 Hz, 2H), 6.92 (m, 2H), 6.75-6.84 (m, 4H), 6.66 (t, J=7.8 Hz, 2H), 4.64 (d, J=15.8 Hz, 1H), 4.22 (d, J=15.8 Hz, 1H), 4.17 (t, J=6.3 Hz, 1H), 3.08 (t, J=5.7 Hz, 2H). LRMS (ESI): (calc.) 393.5; (found) 394.1 (MH)⁺.

### EXAMPLE 50

N-hydroxy-4-((3-oxo-3,4-dihydroquinoxalin-1(2H)-yl)methyl)-benzamide (Compound 54b₁)

### Step 1: Methyl 2-(2-nitrophenylamino)acetate (Compound 51b₁)

Glycine methyl ester hydrochloride (0.7534 g, 6 mmol) and 1-fluoro-3-nitrobenzene (0.8748 g, 6.2 mmol) were dissolved in DMF (6 mL). Triethylamine (2.1 mL, 15 mmol) was added to the mixture, and the reaction was heated under N₂ atmosphere, at 80° C. for 16 h. The solvent was evaporated, EtOAc was added (30 mL), the organic phase was washed with water, dried over MgSO₄, filtered and concentrated to give crude methyl 2-(2-nitrophenylamino)acetate as an orange gum.

### Step 2: 3,4-Dihydroquinoxalin-2(1H)-one (Compound 52b₁)

Crude Compound 51b₁ (0.932 g, 4.42 mmol) and 5% Pd/C in MeOH (25 mL) were placed under a hydrogen atmosphere (40 psi). After 1 h, the catalyst was filtered, methanol was removed and the residue was purified by flash chromatography eluting with 1:1 EtOAc/Hexanes. The title Compound 52b₁ was obtained as a brown solid (0.325 g, 50%). ¹H NMR: (CD₃OD) δ (ppm): 8.04 (s, 1H), 6.76 (dt, J=2.0, 7.2 Hz, 1H), 6.63 (dt, J=1.2, 7.6 Hz, 1H), 6.61-6.57 (m, 2H), 3.87 (s, 2H), 3.75 (s, 2H). LRMS (ESI): (calc.) 148.1; (found) 149.1 (MH)⁺.

### Step 3: Methyl 4-((3-oxo-3,4-dihydroquinoxalin-1(2H)-yl)methyl)benzoate (Compound 53b₁)

Following the procedure described in Scheme 1, step 3, and Example 16 but substituting Compound 3a for Compound 52b₁, the title Compound 53b₁ was obtained as white fluffy solid (77%). ¹H NMR: (DMSO-d₆) δ (ppm): 10.43 (s, 1H), 7.90 (d, J=8.4 Hz, 2H), 7.44 (d, J=8.0 Hz, 2H), 6.80-6.74 (m, 2H), 6.67-6.63 (m, 1H), 6.60-6.58 (m, 1H), 4.50 (s, 2H), 3.83 (s, 3H), 3.77 (s, 2H). LRMS (ESI): (calc.) 296.2; (found) 297.2 (MH)⁺.

### Step 4: N-Hydroxy-4-((3-oxo-3,4-dihydroquinoxalin-1(2H)-yl)methyl)-benzamide, (Compound 54b₁)

To a solution of 53b₁ (41 mg, 0.139 mmol) in 1:1 THF/methanol (0.83 mL) was added a 50% wt solution of hydroxylamine in water (0.87 mL). Sodium hydroxide powder (44 mg, 1.112 mmol) was then added to the mixture. After stirring at room temperature for 1.5 h the reaction was quenched with glacial acetic acid (0.15 mL) and then concentrated under vacuum. The product was then suspended in methanol/water (2:1) and filtered. The residue was further washed with methanol and then dried under vacuum to give the title Compound 54b₁ as a white solid (30 mg, 73%). ¹H NMR: (DMSO-d₆) δ (ppm): 11.1 (s, 1H), 10.4 (s, 1H), 8.99 (s, 1H), 7.68 (d, J=8.2 Hz, 2H), 7.36 (d, J=8.4 Hz, 2H), 6.89-6.75 (m, 2H), 6.67-6.62 (m, 2H), 4.46 (s, 2H), 3.75 (s, 2H). LRMS (ESI): (calc.) 297.3; (found) 298.3 (MH)⁺.

### EXAMPLES 49 AND 51-54

Examples 49 and 51-54 describe the preparation of Compound 54a₂ and 54b₂₋₂₁ using the same procedures as described for Compound 54a₁ in Example 48 and for Compound 54b₁ in Example 50. Characterization data are presented in Table 5.

TABLE 5

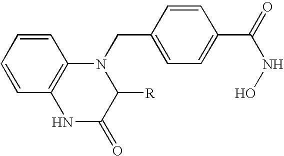

| Ex | Cpd | R | Name | Characterization | Scheme |
|---|---|---|---|---|---|
| 49 | 54a₂ | 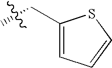 | 4-(((R)-2,3- Dihydro-2- oxo-3- ((thiophen-2- yl)methyl) quinoxalin-4(1H)- yl)methyl)-N- hydroxy- benzamide | ¹H NMR: (DMSO-d₆) δ (ppm): 11.16 (s, 1H), 10.52 (s, 1H), 9.02 (s, 1H), 7.67 (d, J=8.2Hz, 2H), 7.33 (d, J=5.1Hz, 1H), 7.30 (d, J=8.2Hz, 2H), 6.92 (m, 2H), 6.75-6.84 (m, 4H), 6.66 (t, | |
| # J=7.8Hz, 2H), 4.64 (d, J=15.8Hz, 1H), 4.22 (d, J=15.8Hz, 1H), 4.17 (t, J=6.3Hz, 1H), 3.08 (t, J=5.7Hz, 2H). LRMS (ESI): (calc) 393.5; (found) 394.1 (MH)⁺. | 6 | | | | |
| 51b₂ | 54b₂ | 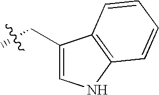 | (R)-4-((2- ((1H-indol-3- yl)methyl)-3- oxo-3,4- dihydroquinoxalin- 1(2H)- yl)methyl)-N- hydroxybenzamide | ¹H NMR: (DMSO-d₆) δ (ppm): 11.1 (br s, 1H), 10.8 (s, 1H), 10.4 (s, 1H), 8.97 (br s, 1H), 7.60 (d, J=8.4Hz, 2H), 7.30 (d, J=7.6Hz, 1H), 7.29 (d, J=7.6Hz, 1H), 7.17 (d, J=8.4Hz, 2H), 7.03 | |
| # (t, J =6.8Hz, 1H), 6.79-6.76 (m, 2H), 6.65 (t, J=7.2Hz, 1H), 6.58 (d, J=7.6Hz, 1H), 4.53 (d, J=16.4Hz, 1H), 4.14-4.06 (m, 2H), 2.95 (dd, J=6.4, 14.0Hz, 1H), 2.83 (dd, J=6.4, 14.0Hz, 1H). LRMS (ESI): (calc) 426.5; (found) 427.4 (MH)⁺, 449.2 (MNa+). | 6 | | | | |
| 51b₃ | 54b₃ | 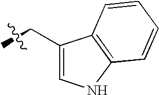 | (S)-2-((2- ((1H-indol-3- yl)methyl)-3- oxo-3,4- dihydroquinoxalin- 1(2H)- yl)methyl)-N- hydroxybenzamide | ¹H NMR: (DMSO-d₆) δ (ppm): 11.1 (br s, 1H), 10.8 (s, 1H), 10.4 (s, 1H), 8.99 (br s, 1H), 7.60 (d, J=8.4Hz, 2H), 7.30 (d, J=7.6Hz, 1H), 7.29 (d, J=7.6Hz, 1H), 7.16 (d, J=8.4Hz, 2H), 7.03 | |
| # (t, J =6.8Hz, 1H), 6.99 (m, 1H), 6.91 (t, J =6.8Hz, 1H), 6.79-6.76 (m, 2H), 6.65 (t, J=7.2Hz, 1H), 6.58 (d, J=7.6Hz, 1H), 4.53 (d, J=16.4Hz, 1H), 4.14-4.06 (m, 2H), 2.95 (dd, J=6.4, 14.0Hz, 1H), 2.83 (dd, J=6.4, 14.0Hz, 1H). LRMS (ESI): (calc) 426.5; (found) 427.2 (MH)⁺, 449.2 (MNa⁺). | 6 | | | | |
| 51b₄ | 54b₄ | 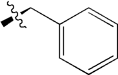 | (S)-4-((2- benzyl-3-oxo 3,4- dihydroquinoxalin- 1(2H)- yl)methyl)-N- hydroxybenzamide | ¹H NMR: (CD₃OD) δ (ppm): 7.61 (d, J =8.0Hz, 2H), 7.24-7.16 (m, 5H), 7.08 (d, J=7.6Hz, 2H), 6.88 (t, J=8.0Hz, 1H), 6.80 (d, J=7.6Hz, 1H), 6.73 (d, J =7.2Hz, 1H), 6.65 (d, J=8.0Hz, 1H), | |
| # 4.49 (d, J=15.6Hz, 1H), 4.07 (t, J =6.4Hz, 1H), 3.95 (d, J=15.6Hz, 1H), 2.84 (d, J=6.8Hz, 2H). LRMS (ESI): (calc) 387.4; (found) 388.3 (MH)⁺, 410.2 (MNa⁺). | 6 | | | | |
| 51b₅ | 54b₅ | 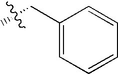 | (R)-4-((2- benzyl-3-oxo 3,4- dihydroquinoxalin- 1(2H)- yl)methyl)-N- hydroxybenzamide | ¹H NMR: (CD₃OD) δ (ppm): 7.60 (d, J =8.4Hz, 2H), 7.20-7.16 (m, 5H), 7.08 (d, J=8Hz, 2H), 6.89 (dt, J=1.2, 8.0Hz, 1H), 6.80 (dd, J=1.2, 7.6Hz, 1H), 6.73 (dt, J=1.2, 7.6Hz, 1H), 6.67 (d, J=8Hz, | |
| # 1H), 4.49 (d, J=15.6Hz, 1H), 4.07 (t, J=6.4Hz, 1H), 3.96 (d, J = 15.6Hz, 1H), 2.83 (d, J=6.8Hz, 2H). LRMS (ESI): (calc) 387.1; (found) 388.3 (MH)⁺. | 6 | | | | |
| 51b₆ | 54b₆ | H | N-Hydroxy-4-((2- ((3-oxo-3,4- dihydroquinoxalin- 1(2H)- yl)methyl) benzamide | ¹H NMR: (DMSO-d6) δ (ppm): 11.1 (s, 1H), 10.4 (s, 1H), 8.99 (s, 1H), 7.68 (d, J=8.2Hz, 2H), 7.36 (d, J=8.4Hz, 2H), 6.89-6.75 (m, 2H), 6.67-6.62 (m, 2H), 4.46 (s, 2H), 3.75 (s, 2H). LRMS (ESI): (calc) 297.3; (found) 298.3 (MH)⁺. | 6 |
| 51b₇ | 54b₇ | 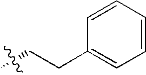 | (R)-N- Hydroxy-4- ((3-oxo-2- phenethyl- 3,4- dihydroquinoxalin- 1(2H)- yl)methyl) benzamide | ¹H NMR: (DMSO-d₆) δ (ppm): 11.1 (s, 1H), 10.5 (s, 1H), 9.00 (s, 1H), 7.64 (d, J=8.4 HZ, 2H), 7.30 (d, J=8.0Hz, 2H), 7.21 (t, J=6.8Hz, 2H), 7.14 (d, J=7.2Hz, 1H), 7.10 (d, J=8.0Hz, 2H), | |
| # 6.81-6.75 (m, 2H), 6.65 (t, J=7.6Hz, 1H), 6.61 (d, J=8.0Hz, 1H), 4.69 (d, J=16.0Hz, 1H), 4.34 (d, J=15.6Hz, 1H), 3.82 (dd, J=4.8, 8.0Hz, 1H), 2.6-2.50 (m, 2H), 1.88-1.82 (m, 1H), 1.71-1.67 (m, 1H). LRMS (ESI): (calc) 401.2; (found) 402.2 (MH)⁺424.2 (MNa). | 6 | | | | |
| 51b₈ | 54b₈ | 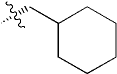 | (R)-4-((2- (Cyclohexylm ethyl)-3-oxo- 3,4- dihydroquinoxalin- 1(2H)- yl)methyl)-N- hydroxybenzamide | ¹H NMR: (DMSO-d₆) δ (ppm): 11.1 (s, 1H), 10.4 (s, 1H), 9.00 (s, 1H), 7.67 (d, J=8.4Hz, 2H), 7.36 (d, J=8.0Hz, 2H), 6.79-6.75 (m, 2H), 6.66 (t, J=8.4Hz, 1H), 6.62 (d, J=8.0Hz, 1H), 4.64 (d, | |
| # J=15.2Hz, 1H), 4.29 (d, J=15.6Hz, 1H), 3.78 (dd, J=5.2, 8.8Hz, 1H), 1.71 (m, 1H), 1.66-1.50 (m, 4H), 1.40-1.00 (m, 6H), 0.85-0.76 (m, 2H). LRMS (ESI): (calc) 393.2; (found) 394.4 (MH)⁺. | 6 | | | | |
| 51b₉ | 54b₉ | 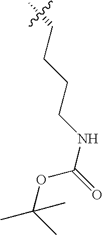 | (R)-tert-Butyl 4-(1-(4- (hydroxycarba- moyl)benzyl)- 3-oxo- 1,2,3,4- tetrahydro- quinoxalin-2- yl)butylcarba mate | ¹H NMR: (DMSO-d₆) δ (ppm): 11.1 (s, 1H), 10.4 (s, 1H), 8.99 (s, 1H), 7.66 (d, J=8.0Hz, 2H), 7.34 (d, J=8.4Hz, 2H), 6.77-6.73 (m, 3H), 6.63 (t, J=7.2Hz, 1H), 6.57 (d, J= | |
| # 7.6Hz, 1H), 4.66 (d, J=16.0Hz, 1H), 4.37 (d, J=15.6Hz, 1H), 3.78 (m, 1H), 2.82 (m, 2H), 1.60-1.50 (m, 1H), 1.55-1.30 (m, 1H), 1.36 (s, 9H), 1.31-1.24 (m, 4H). LRMS (ESI): (calc) 468.6; (found) 491.4 (MNa⁺). | 6 | | | | |
| 51b₁₀ | 54b₁₀ | 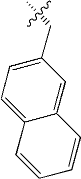 | (R)-N- hydroxy-4- ((2- (naphthalen- 2-ylmethyl)-3- oxo-3,4- dihydroquinoxalin- 1(2H)- yl)methyl) benzamide | ¹H NMR: (DMSO-d₆) δ (ppm): 11.1 (s, 1H), 10.5 (s, 1H), 8.98 (s, 1H), 7.91 (d, J=7.2Hz, 1H), 7.84 (d, J=8.4Hz, 1H), 7.79 (d, J=8.0Hz, 1H), 7.57 (d, J=8.0Hz, 2H), 7.51-7.43 | |
| # (m, 2H), 7.37 (t, J=7.2Hz, 1H), 7.13 (d, J=7.2Hz, 1H), 7.09 (d, J=8.0Hz, 2H), 6.88-6.85 (m, 2H), 6.75 (t, J=8.0Hz, 1H), 6.71 (d, J=7.6Hz, 1H), 4.51 (d, J=15.2Hz, 1H), 4.12 (t, J=8.4Hz, 1H), 3.96 (d, J=15.2Hz, 1H), 3.28 (dd, J=7.2, 13.6Hz, 1H), 3.09 (dd, J=6.8, 13.6Hz, 1H). LRMS (ESI): (calc) 437.2; (found) 438.3 (MH)⁺. | 6 | | | | |
| 51b₁₁ | 54b₁₁ | 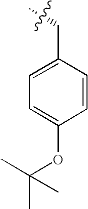 | (R)-4-((2-(4- tert- butoxybenzyl)- 3-oxo-3,4- dihydroquinoxalin- 1(2H)- yl)methyl)-N- hydroxybenzamide | ¹H NMR: (DMSO-d₆) δ (ppm): 11.1 (s, 1H), 10.4 (s, 1H), 8.98 (s, 1H), 7.63 (d, J=8.4Hz, 2H), 7.23 (d, J=8.4Hz, 2H), 6.93 (d=8.4, 2H), 6.74 (d, J=8.4Hz, 2H), 6.73-6.69 (m, 1H), 6.64 (dd, | |
| # J=7.6, 1.6Hz, 1H), 6.55 (t, J=8.4Hz, 1H), 6.51 (d, J=8.0Hz, 1H), 4.42 (d, J=15.6Hz, 1H), 4.17 (d, J=16.0Hz, 1H), 4.10 (t, J=6.0Hz, 1H), 2.77 (d, J=6.0Hz, 1H), 1.22 (s, 9H). LRMS (ESI): (calc) 459.2; (found) 460.2 (MH)⁺. | 6 | | | | |
| 51b₁₂ | 54b₁₂ | 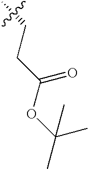 | (R)-tert-Butyl 3-(1-(4- (hydroxycarba- moyl)benzyl)- 3-oxo- 1,2,3,4- tetrahydro- quinoxalin-2- yl)propanoate | ¹H NMR: (DMSO-d₆) δ (ppm): 11.1 (s, 1H), 10.5 (s, 1H), 8.99 (s, 1H), 7.66 (d, J=8.0Hz, 2H), 7.34 (d, J=8.0Hz, 2H), 6.78-6.75 (m, 2H), 6.66 (m, 1H), 6.58 (m, 1H), 4.70 (d, J=15.6Hz, | |
| # 1H), 4.37 (d, J=16.4Hz, 1H), 3.85 (m, 1H), 2.22 (m, 2H), 1.78 (m, 1H), 1.63 (m, 1H). LRMS (ESI): (calc) 425.2; (found) 426.2 (MH)⁺. | 6 | | | | |
| 51b₁₃ | 54b₁₃ | 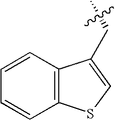 | (R)-4-((2- (Benzo[b]thiophen-3- ylmethyl)-3- oxo-3,4- dihydroquinoxalin- 1(2H)- yl)methyl)-N- hydroxybenzamide | ¹H NMR: (DMSO-d₆) δ (ppm): 11.1 (s, 1H), 10.5 (s, 1H), 9.00 (s, 1H), 7.94 (m, 1H), 7.62-7.61 (m, 3H), 7.34 (m, 3H), 7.21 (m, 2H), 6.80-6.78 (m, 2H), 6.68-6.64 (m, 2H), 4.59 (d, J=14.8Hz, | |
| # 1H), 4.21-4.17 (m, 2H), 3.20-3.07 (m, 1H), 3.03-2.91 (m, 1H). LRMS (ESI): (calc) 443.1; (found) 444.2 (MH)⁺. | 6 | | | | |
| 54b₁₄ | 54b₁₄ | 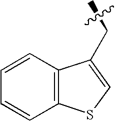 | (S)-4-((2- (nenzo[b]thiophen-3- ylmethyl)-3- oxo-3,4- dihydroquinoxalin- 1(2H)- yl)methyl)-N- hydroxybenzamide | ¹H NMR: (DMSO-d₆) δ (ppm): 11.1 (br s, 1H), 10.5 (s, 1H), 9.00 (brs, 1H), 7.93 (d, J=8.4Hz, 2H), 7.61 (d, J=8.4Hz, 2H), 7.59-7.57 (m, 1H), 7.34-7.29 (m, 3H), 7.20 (d, J=8.0Hz, 2H), | |
| # 7.6Hz, 2H), 6.88 (t, J=7.6Hz, 1H), 6.62 (d, J=8.0Hz, 1H), 4.59 (d, J=15.6Hz, 1H), 4.21-4.15 (m, 2H), 3.11 (dd, J=6.4, 14.0Hz, 1H), 2.96 (dd, J=7.2, 14.0Hz, 1H). LRMS (ESI): (calc) 443.1; (found) 444.2 (MH)⁺. | 6 | | | | |
| 51b₁₅ | 54b₁₅ | 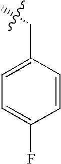 | (R)-4-((2-(4- Fluorobenzyl)- 3-oxo-3,4- dihydroquinoxalin- 1(2H)- yl)methyl)-N- hydroxybenzamide | ¹H NMR: (DMSO-d₆) δ (ppm): 11.1 (br s, 1H), 10.4 (s, 1H), 8.98 (brs, 1H), 7.63 (d, J=8.0Hz, 2H), 7.26 (d, J=8.4Hz, 2H), 7.08 (dd, J=8.4, 5.6Hz, 2H), 7.00 (t, J=8.8Hz, 2H), 6.78-6.70 (m, 2H), 6.62 | |
| # (dt, J=1.2, 7.2Hz, 1H), 6.56 (d, J=8.0Hz, 1H), 4.54 (d, J=16.0Hz, 1H), 4.22 (d, J=15.6Hz, 1H), 4.10 (t, J=6.4Hz, 1H), 2.81 (dd, J=6.4, 13.6Hz, 1H), 2.75 (dd, J=6.8, 13.6Hz, 1H). LRMS (ESI): (calc) 405.15; (found) 406.2 (MH)⁺. | 6 | | | | |
| 54b₁₆ | 54b₁₆ | 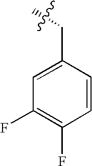 | (R)-4-((2- (3,4- Difluorobenzyl)- 3-oxo-3,4- dihydroquinoxalin- 1(2H)- yl)methyl)-N- hydroxybenzamide | ¹H NMR: (DMSO-d₆) δ (ppm): 11.1 (s, 1H), 10.5 (s, 1H), 9.00 (brs, 1H), 7.65 (d, J=8.0Hz, 2H), 7.30 (d, J=8.4Hz, 2H), 7.22-7.13 (m, 2H), 6.89 (m, 1H), 6.74-6.70 (m, 2H), 6.62-6.55 (m, 2H), | |
| # 4.60 (d, J=15.6Hz, 1H), 4.33 (d, J=15.6Hz, 1H), 4.16 (m, 1H), 2.83-2.80 (m, 2H). LRMS (ESI): (calc) 423.14; (found) 424.2 (MH)⁺. | 6 | | | | |
| 51b₁₇ | 54b₁₇ | 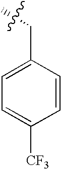 | (R)-4-((2-4- Trifluoromethyl- benzyl)-3- oxo-3,4- dihydroquinoxalin- 1(2H)- yl)methyl)-N- hydroxybenzamide | ¹H NMR: (DMSO-d6) δ (ppm): 11.1 (s, 1H), 10.5 (s, 1H), 8.99 (brs, 1H), 7.65 (d, J=8.4Hz, 2H), 7.52 (d, J=8.0Hz, 2H), 7.29 (d, J=8.0Hz, 4H), 6.76 (dt, J=1.6, 8.4Hz, 1H), 6.72 (dd, J=1.6, | |
| # 7.6Hz, 1H), 6.63-6.58 (m, 2H), 4.59 (d, J=15.6Hz, 1H), 4.21 (d, J=15.6Hz, 1H), 4.16 (t, J=6.0Hz, 1H), 2.94 (dd, J=6.0, 13.2Hz, 1H), 2.82 (dd, J=7.2, 13.2Hz, 1H). LRMS (ESI): (calc) 455.2; (found) 456.2 (MH)⁺. | 6 | | | | |
| 51b₁₈ | 54b₁₈ | 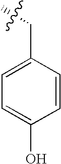 | (R)-N- Hydroxy-4- ((2-(4- hydroxybenzyl)- 3-oxo-3,4- dihydroquinoxalin- 1(2H)- yl)methyl) benzamide | ¹H NMR: (CD₃OD) δ (ppm): 7.51 (m, 2H), 7.09 (m, 2H), 6.79-6.77 (m, 3H), 6.68 (m, 1H), 6.63-6.62 (m, 1H), 6.56-6.54 (m, 3H), 4.39 (d, J=15.6Hz, 1H), 3.89-3.84 (m, 2H), 2.65 (m, 2H). | |
| # LRMS (ESI): (calc) 403.4; (found) 404.2 (MH)⁺. | 6 | | | | |
| 51b₁₉ | 54b₁₉ | 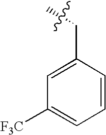 | (R)-N- Hydroxy-4- ((2-(3- (trifluoromethyl) benzyl)-3- oxo-3,4- dihydroquinoxalin- 1(2H)- yl)methyl) benzamide | ¹H NMR: (DMSO-d₆) δ (ppm): 11.1 (s, 1H), 10.5 (s, 1H), 8.99 (s, 1H), 7.65 (d, J=8.4Hz, 2H), 7.47 (d, J=7.6Hz, 1H), 7.40-7.35 (m, 3H), 7.29 (d, J=8.0Hz, 2H), 6.74 (dt, J= | |
| # 1.6, 8.0Hz, 1H), 6.68 (dd, J=1.2, 7.2Hz, 1H), 6.60 (dt, J=1.2, 7.6Hz, 1H), 6.55 (d, J=8.0Hz, 1H), 4.59 (d, J=16.0Hz, 1H), 4.34 (d, J=16.0Hz, 1H), 4.18 (t, J=6.4Hz, 1H), 2.95 (dd, J=6.0, 13.6Hz, 1H), 2.88 (dd, J=7.6, 14.0Hz, 1H). LRMS (ESI): (calc) 455.2; (found) 456.2 (MH)⁺. | 6 | | | | |
| 51b₂₀ | 54b₂₀ | 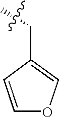 | (R)-4-((2- (Furan-3- ylmethyl)-3- oxo-3,4- dihydroquinoxalin- 1(2H)- yl)methyl)-N0 hydroxybenzamide | ¹H NMR: (DMSO-d₆) δ (ppm): 11.1 (s, 1H), 10.5 (s, 1H), 8.99 (s, 1H), 7.64 (d, J=8.4Hz, 2H), 7.44 (s, 1H), 7.26 (d, J=8.0Hz, 2H), 6.77-6.73 (m, 2H), 6.63 (t, J=8.0Hz, 1H), 6.55 (d, J=8.0Hz, | |
| # (t, J=8.0Hz, 1H), 6.55 (d, J=8.0Hz, 1H), 6.28 (m, 1H), 6.05 (m, 1H), 4.52 (d, J=15.6Hz, 1H), 4.14-4.11 (m, 2H), 2.84 (dd, J=6.4, 14.8Hz, 1H), 2.80 (dd, J=6.0, 14.8Hz, 1H). LRMS (ESI): (calc) 377.1; (found) 378.3 (MH)⁺. | 6 | | | | |
| 51b₂₁ | 54b₂₁ | 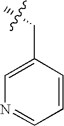 | (R)-N- Hydroxy-4- ((3-oxo-2- (pyridin-3- ylmethyl)-3,4- dihydroquinoxalin- 1(2H)- yl)methyl) benzamide | ¹H NMR: (DMSO-d₆) δ (ppm): 11.0 (s, 1H), 10.4 (s, 1H), 8.25 (d, J=3.6Hz, 1H), 8.16 (s, 1H), 7.57 (d, J=8.4Hz, 2H), 7.40 (dd, J=6.0, 2.0Hz, 1H), 7.22 (d, J=8.4Hz, 2H), 7.11 (dd, J=4.8, | |
| # 6.8Hz, 1H), 6.68 (dt, J=1.2, 7.6Hz, 1H), 6.62 (dd, J=1.2, 7.6Hz, 1H), 6.55-6.50 (m, 2H), 4.52 (d, J=15.6Hz, 1H), 4.24 (d, J=16.0Hz, 1H), 4.08 (t, J=5.6Hz, 1H), 2.79 (dd, J=6.0, 13.6Hz, 1H), 2.72 (dd, J=7.2, 14.0Hz, 1H). LRMS (ESI): (calc) 388.2; (found) 389.2 (MH)⁺. | 6 | | | | |

### EXAMPLE 52

Example 52 describes the preparation of Compound 54c using the same procedures as described for Compound 54b₁ in Example 50. Characterization data are presented in Table 6.

TABLE 6

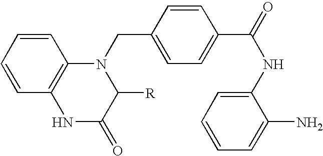

| Ex | Cpd | R | Name | Characterization | Scheme |
|---|---|---|---|---|---|
| 52 | 54c | 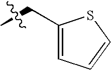 | (S)-N-(2- Aminophenyl)- 4-((3-oxo-2- (thiophen-2- ylmethyl)-3,4- dihydroquinoxalin- 1(2H)- yl)methyl)benzamide | ¹H NMR: (DMSO-d₆) δ (ppm): 10.53 (s, 1H), 9.6 (s, 1H), 7.91 (d, J=8.2Hz, 2H), 7.40-7.33 (m, 3H), 7.15 (d, J=7.8Hz, 1H), 7.10-6.95 (m, 1H), 6.95-6.90 (m, 1H), 6.85-6.76 (m, 4H), | |
| # 6.71-6.65 (m, 2H), 6.64-6.57 (m, 1H), 4.92 (s, 2H), 4.68 (d, J=15.8Hz, 1H), 4.32 (d, J=15.6Hz, 1H), 4.18 (t, J=6.3Hz, 1H), 3.15-3.04 (m, 2H) LRMS (ESI): (calc.) 468.6; (found) 469.2 (MH)^{+.} | 6 | | | | |

### EXAMPLE 55

### 4-(((R)-2,3-Dihydro-2-oxo-3-((thiophen-2-yl)methyl)quinoxalin-4(1H)-yl)methyl)-N-hydroxybenzamide (Compound 56a)

### Step 4: 4-(((R)-2,3-Dihydro-2-oxo-3-((thiophen-2-yl)methyl)quinoxalin-4(1H)-yl)methyl)-N-hydroxybenzamide (Compound 56a)

Following the procedure described in Example 48, Compound 54a₂, steps 1-5 (Scheme 6) but substituting (L)-2-amino-3-thiophen-2-yl-propionic acid for (D)-2-amino-3-thiophen-2-yl-propionic acid in step 1 and 3-formyl-benzoic acid for 4-formyl-benzoic acid in step 4, the title Compound was obtained in 12% yield as a light pink crystalline solid. ¹H NMR: (DMSO-d₆) δ (ppm): ¹H NMR: (DMSO-d₆) δ (ppm): 11.25 (br s, 1H), 10.52 (s, 1H), 7.97 (s, 2H), 7.60-7.70 (m, 2H), 7.28-7.42 (m, 3H), 6.91 (s, 1H), 6.81 (m, 3H), 6.67 (d, J=6.1 Hz, 2H), 4.63 (d, J=15.3 Hz, 1H), 4.27 (d, J=15.7 Hz, 1H), 4.19 (s, 1H), 3.09 (br s, 2H).

LRMS (ESI): (calc.) 393.5; (found) 393.9 (M-H)⁻.

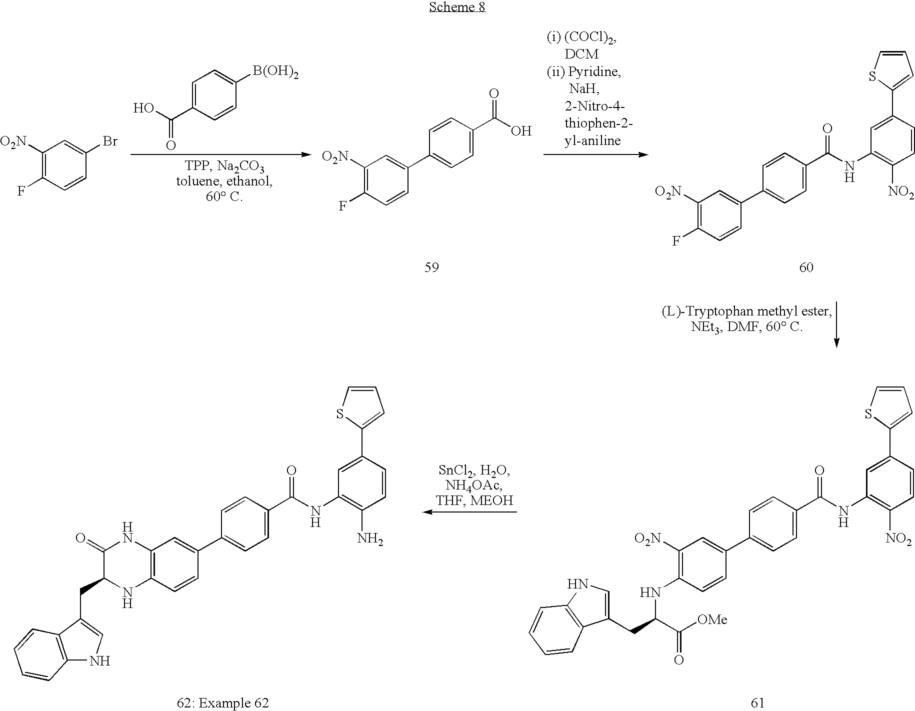

### EXAMPLE 62

### 4-((S)-2-((1H-Indol-3-yl)methyl)-1,2,3,4-tetrahydro-3-oxoquinoxalin-6-yl)-N-(2-amino-5-(thiophen-2-yl)phenyl)benzamide (Compound 62)

### Step 1: p-(4-Fluro-3-nitrobenzene)-benzoic acid (Compound 59)

To a stirred solution of 4-bromo-1-fluoro-2-nitrobenzene (2.10 g, 9.5 mmol) and 4-carboxybenzeneboronic acid (1.74 g, 10.5 mmol) in a 1:1 mixture of toluene:ethanol (40 mL), was added Pd(PPh₃)₄ (329 mg, 0.29 mmol), and sodium carbonate (2 M, 9.2 mL). The solution was degassed with N₂ for 5 min and then heated to 60° C. and left to stir for 3 h. Water (50 mL) was added and aqueous extraction performed with EtOAc (2×40 mL). The organic layer was separated, dried with sodium sulfate and evaporated under reduced pressure. Purification was achieved via silica gel chromatography, employing a 1:1 AcOEt:hexanes moving to pure AcOEt solvent system. This afforded 59 as a white solid (850 mg, 35%). ¹H NMR: (DMSO-d₆) δ (ppm): 8.45 (dd, J=7.2, 2.6 Hz, 1H), 8.21-8.18 (m, 1H), 8.04 (d, J=8.2 Hz, 2H), 7.89 (d, J=8.2 Hz, 2H), 7.74 (dd, J=10.0, 8.8 Hz, 1H).

### Step 2 N-(2-Nitro-5-(thiophen-2-yl)4-[4-fluro-3-nitro]biphenyl)benzamide (Compound 60)

To a stirred solution of 59 (150 mg, 0.574 mmol) in DCM (10 mL) was added oxalyl chloride (2 M, 431 mL, 0.862 mmol) and DMF (1 drop). The resulting solution was stirred for 20 min. DCM was removed via rotary evaporation and pyridine was added (10 mL), followed by 2-Nitro-4-thiophen-2-yl-aniline (126 mg, 0.574 mmol), and NaH (91 mg, 2.29 mmol). The reaction was stirred for 1 h before quenching with acetic acid (1 mL). The pyridine was removed under reduced pressure and purification was achieved through silica gel chromatography employing 4:1 AcOEt:hexanes solvent system. This afforded 60 as a yellow solid (160 mg, 60%). ¹H NMR: (DMSO-d₆) δ (ppm): 11.23 (s, 1H), 8.52 (dd, J=7.0, 2.6 Hz, 1H), 8.29-8.25 (m, 1H), 8.22-8.17 (m, 3H), 8.08 (d, J=8.6 Hz, 1H), 8.00 (d, J=8.6 Hz, 2H), 7.80-7.72 (m, 4H), 7.25 (dd, J=5.1, 4.7 Hz, 1H).

### Step 3: N-(2-Nitro-5-(thiophen-2-yl)4-[4-(S)-methyl 2-(2-nitrophenylamino)-3-(1H-indol-3-yl)propanoate-3-nitro]biphenyl)benzamide (Compound 61)

To a stirred solution of 60 (145 mg, 0.31 mmol) in DMF (3 mL) was added L-tryptophan methyl ester hydrochloride (79 mg, 0.31 mmol) and triethylamine (0.11 mL, 0.78 mmol). The solution was heated to 60° C. and stirred for 15 h. Water (50 mL) was added and aqueous extraction performed with EtOAc (2×40 mL). The organic layer was separated, dried with sodium sulfate and evaporated under reduced pressure. Purification was achieved via silica gel chromatography, employing a 1:2 AcOEt:hexanes solvent system. This afforded 61 as an orange solid (90 mg, 43%). ¹H NMR: (DMSO-d₆) δ (ppm): 11.01 (s, 1H), 10.92 (s, 1H), 8.47 (d, J=2.4 Hz, 1H), 8.37 (d, J=7.8 Hz, 1H), 8.20 (d, J=2.0 Hz, 1H), 8.11-8.02 (m, 4H), 7.92 (d, J=8.4 Hz, 1H), 7.78-7.71 (m, 3H), 7.40-7.34 (m, 2H), 7.27-7.21 (m, 3H), 7.08 (t, J=7.2 Hz, 1H), 6.96 (t, J=7.2 Hz, 1H), 5.07-5.05 (m, 1H), 3.71 (s, 3H), 3.45 (t, J=4.7 Hz, 2H).

### Step 4: 4-((S)-2-((1H-Indol-3-yl)methyl)-1,2,3,4-tetrahydro-3-oxoquinoxalin-6-yl)-N-(2-amino-5-(thiophen-2-yl)phenyl)benzamide (Compound 62)

A suspension of Compound 61 (70 mg, 0.106 mmol) and tin(II) chloride dihydrate (143 mg, 0.635 mmol) in a 2:3 mixture MeOH/THF (5 mL) was stirred at 75° C. in a sealed tube for 1 h, diluted with EtOAc and washed with saturated aqueous solution of NaHCO₃, dried over Na₂SO₄ and purified by flash chromatography, eluent 20% EtOAc in DCM, to afford Compound 62 as an orange solid (25 mg, 42%). ¹H NMR: (DMSO-d₆) δ (ppm): 10.85 (s, 1H), 10.31 (s, 1H), 9.70 (s, 1H), 8.00 (d, J=8.4 Hz, 2H), 7.60 (d, J=8.6 Hz, 2H), 7.51 (d, J=7.8 Hz, 1H), 7.46 (d, J=2.0 Hz, 1H), 7.33 (dd, J=5.1, 1.2 Hz, 1H), 7.30 (d, J=8.0 Hz, 1H), 7.28 (dd, J=8.2, 2.2 Hz, 1H), 7.23 (dd, J=3.5, 1.2 Hz, 1H), 7.17 (dd, J=8.2, 2.2 Hz, 1H), 7.12 (d, J=2.1 Hz, 1H), 7.07-7.02 (m, 3H), 6.95 (td, J=8.0, 1.0 Hz, 1H), 6.79 (d, J=8.2 Hz, 1H), 6.76 (d, J=8.0 Hz, 1H), 6.14 (s, 1H), 5.15 (s, 2H), 4.13-4.10 (m, 1H), 3.12-2.97 (m, 2H).

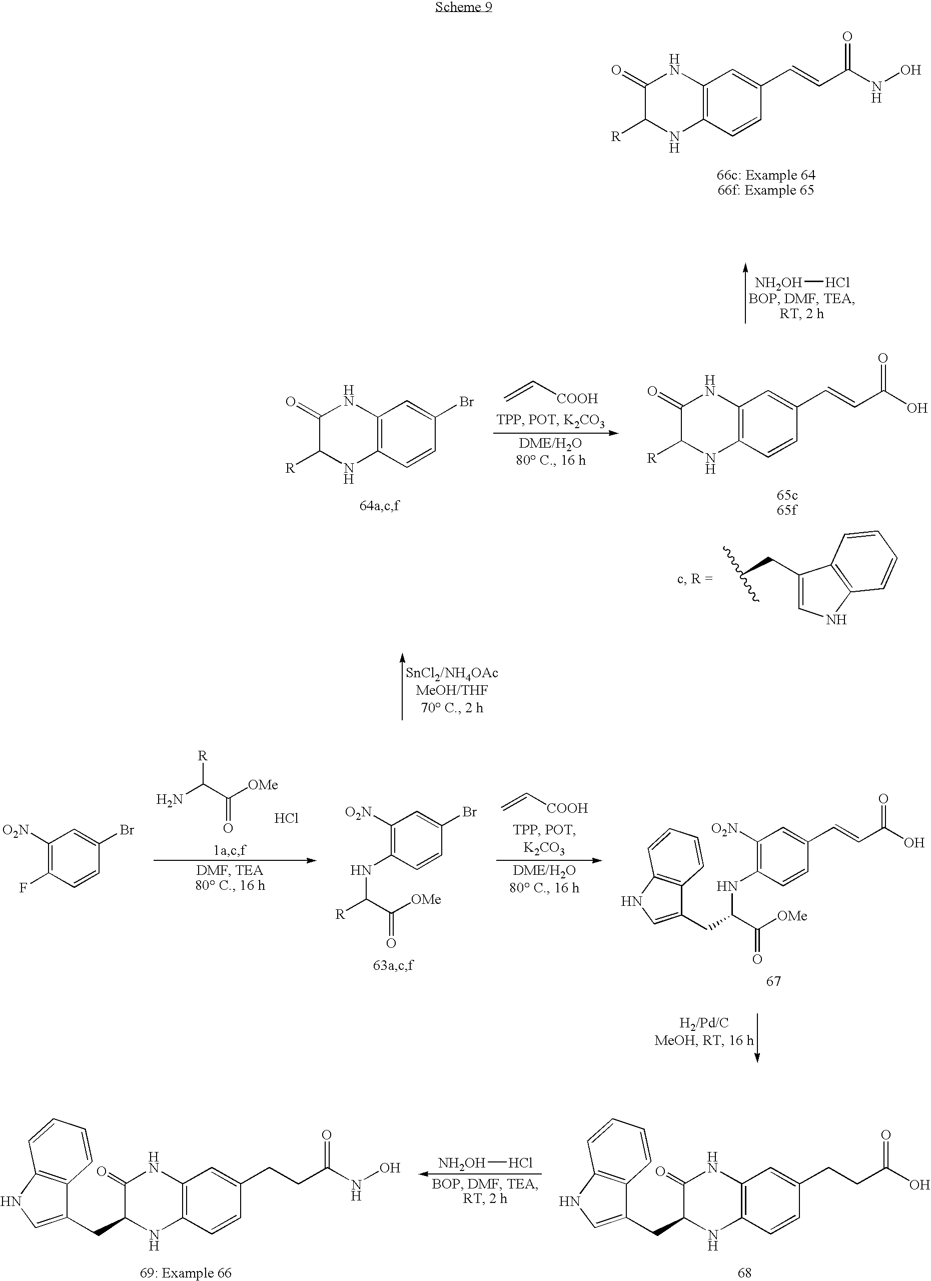

### EXAMPLE 64

(E)-3-((S)-2-((1H-Indol-3-yl)methyl)-1,2,3,4-tetrahydro-3-oxoquinoxalin-6-yl)-N-hydroxyacrylamide (Compound 66c)

### Step 1: Step 1: (S)-Methyl 2-(4-bromo-2-nitrophenylamino)-3-(1H-indol-3-yl)propanoate (Compound 63c)

Following the procedure described in Example 1, Compound 4a, step 1, Scheme 1, but substituting (L)-valine methyl ester hydrochloride with (L)-tryptophan methyl ester hydrochloride (1c), and 3-nitro-4-flurobenzoic acid with 4-bromo-1-fluoro-2-nitrobenzene the title Compound was recovered as a dark orange solid in near quantitative yield. ¹H NMR: (DMSO-d₆) δ (ppm): 8.22 (d, J=7.6 Hz, 1H), 8.15 (d, J=2.4 Hz, 1H), 7.61 (dd, J=2.4, 9.2 Hz, 1H), 7.30 (d, J=8.8 Hz, 2H), 7.14 (d, J=2.4 Hz, 1H), 7.04-6.99 (m, 2H), 6.89 (t, J=7.4, 1H), 4.92-4.91 (m, 1H), 3.65 (s, 3H), 3.36 (t, J=5.6 Hz, 2H).

### Step 2: (S)-3-((1H-Indol-3-yl)methyl)-7-bromo-3,4-dihydroquinoxalin-2(1H)-one (Compound 64c)

Following the procedure described in Scheme 1, step 3, and Example 16 but substituting Compound 3a for Compound 63c, the title Compound 64c was obtained in 35% yield. ¹H NMR: (DMSO-d₆) δ (ppm): 10.83 (s, 1H), 10.29 (s, 1H), 7.46 (d, J=7.8 Hz, 1H), 7.29 (d, J=8.0 Hz, 1H), 7.07 (d, J=2.0 Hz, 1H), 7.02 (t, J=7.0 Hz, 1H), 6.93 (t, J=7.0 Hz, 1H), 6.82 (dd, J=8.4, 2.3 Hz, 1H), 6.77 (d, J=2.2 Hz, 1H), 6.57 (d, J=8.3 Hz, 1H), 6.03 (s, 1H), 4.06-4.04 (m, 1H), 3.07-2.91 (m, 2H).

### Step 3: (S,E)-3-(2-((1H-Indol-3-yl)methyl)-3-oxo-1,2,3,4-tetrahydroquinoxalin-6-yl)acrylic acid (Compound 65c)

Following the procedure described in Scheme 9, step 2, and Example 66 but substituting Compound 63c for Compound 64c, the title Compound 65c was obtained in 49% yield. ¹H NMR: (DMSO-d₆) δ (ppm): 12.01 (br s, 1H), 10.82 (s, 1H), 10.27 (s, 1H), 7.48 (d, J=8.0 Hz, 1H), 7.33 (d, J=15.8 Hz, 1H), 7.28 (dt, J=8.0, 1.0 Hz, 1H), 7.07 (d, J=2.4 Hz, 1H), 7.04-7.00 (m, 2H), 6.93 (td, J=7.1, 1.0 Hz, 1H), 6.85 (d, J=2.0 Hz, 1H), 6.61 (d, J=8.2 Hz, 1H), 6.48 (d, J=1.8 Hz, 1H), 6.01 (d, J=15.6 Hz, 1H), 4.18-4.15 (m, 1H), 3.09-2.98 (m, 2H). LRMS (ESI): (calc.) 347.1; (found) 348.1 (MH)⁺.

### Step 4: (E)-3-((S)-2-((1H-Indol-3-yl)methyl)-1,2,3,4-tetrahydro-3-oxoquinoxalin-6-yl)-N-hydroxyacrylamide (Compound 66c)

Following the procedure described in Example 1, Compound 4a, step 3, Scheme 1, but substituting acid 3a with acid 65c, the title Compound was recovered with 63% yield (86 mg) (see Scheme 8, Example 43, step1-3, for preparation). ¹H NMR: (DMSO-d₆) δ (ppm): 10.81 (s, 1H), 10.56 (s, 1H), 10.31 (s, 1H), 8.85 (s, 1H), 7.48 (d, J=7.7 Hz, 1H), 7.28 (d, J=8.0 Hz, 1H), 7.20 (d, J=15.7 Hz, 1H), 7.07 (d, J=2.3 Hz, 1H), 7.02 (td, J=7.1, 1.0 Hz, 1H), 6.95-6.88 (m, 2H), 6.82 (s, 1H), 6.61 (d, J=8.2 Hz, 1H), 6.32 (s, 1H), 6.04 (d, J=15.7 Hz, 1H), 4.13 (m, 1H), 3.09-3.02 (m, 2H).

### EXAMPLE 65

Example 65 describes the preparation of Compound 66f using the same procedures as described for Compound 66c in Example 64. Characterization data are presented in Table 7.

TABLE 7

| Ex. | Cpd. | R | Z | Name | Characterization | Scheme |
|---|---|---|---|---|---|---|
| 65 | 66f | 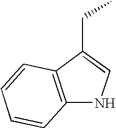 | —NHOH | (E)-3-((R)-2- ((1H-Indol- 3-yl)methyl)- 1,2,3,4- tetrahydro-3- oxoquinoxalin- 6-yl)-N- hydroxyacrylamide | ¹H NMR: (CD₃OD) δ (ppm): 7.52 (d, J=7.8Hz, 1H), 7.38 (d, J=15.6Hz, 1H, 7.29 (d, J=8.0Hz, 1H), 7.08-6.97 (m, 4H), 6.88-6.86 (m, 1H), 6.57 (d, J=8.2Hz, 1H), 6.16 (d, J= | |
| # 15.6Hz, 1H), 4.20-4.17 (m, 1H), 3.21-3.07 (m, 2H). LRMS (ESI): (calc.) 362.1; (found) 363.1 (MH)⁺. | 9 | | | | | |

### EXAMPLE 66

### 3-((S)-2-((1H-indol-3-yl)methyl)-1,2,3,4-tetrahydro-3-oxoquinoxalin-6-yl)-N-hydroxypropanamide (Compound 69)

### Step 2: (E)-3-(4-((S)-1-(methoxycarbonyl)-2-(1H-indol-3-yl)ethylamino)-3-nitrophenyl)acrylic acid (Compound 67)

To a stirred solution of Compound 63c (1.35 g, 3.23 mmol) (see Example 64, step 1, Scheme 9 for preparation) in DMF (20 mL) was added Pd₂(dba)₃ (40 mg, 0.097 mmol), tri-o-toly phosphine (59 mg, 0.195 mmol), triethylamine (1.13 mL, 8.08 mmol), and acrylic acid (0.264 mL, 3.88 mmol). The resulting solution was degassed with nitrogen for 10 min and heated to 100° C. for 16 h. The DMF was removed via rotary evaporation and the resulting oil was diluted with water (30 mL). Aqueous extraction was performed with EtOAc (2×15 mL). Purification was achieved via silica gel chromatography employing a 2:1 hexanes:EtOAc to EtOAc gradient solvent system. This afforded 63 as an orange solid (500 mg, 38%). ¹H NMR: (DMSO-d₆) δ (ppm): 10.20 (s, 1H), 8.59 (d, J=8.0 Hz, 1H), 8.27 (d, J=1.8 Hz, 1H), 7.67 (dd, J=9.0, 1.8 Hz, 1H), 7.54 (d, J=5.9 Hz, 1H), 7.41 (d, J=7.6 Hz, 1H), 7.31 (d, J=8.1 Hz, 1H), 7.11 (s, 1H), 7.09-7.04 (m, 1H), 6.98-6.94 (m, 1H), 6.88 (d, J=9.2 Hz, 1H), 6.32 (d, J=5.9 Hz, 1H), 4.86-4.83 (m, 1H), 3.72 (s, 3H), 3.48-3.43 (m, 2H).

### Step 3: 3-((S)-2-((1H-indol-3-yl)methyl)-1,2,3,4-tetrahydro-3-oxoquinoxalin-6-yl)propanoic acid (Compound 68)

Following the procedure described in Example 1, Compound 4a, step 2, Scheme 1 but substituting acid 2a with acid 67, the title Compound was recovered as a beige solid in near 80% yield. ¹H NMR: (DMSO-d₆) δ (ppm): 10.85 (s, 1H), 10.17 (s, 1H), 7.48 (d, J=7.6 Hz, 1H), 7.31 (d, J=8.0 Hz, 1H), 7.10 (s, 1H), 7.04 (t, J=7.9 Hz, 1H), 6.95 (t, J=7.9 Hz, 1H), 6.57-6.55 (m, 3H), 5.57 (s, 1H), 3.99-3.94 (m, 1H), 3.10-2.87 (m, 2H), 2.63 (t, J=7.4 Hz, 2H), 2.40 (t, J=8.0 Hz, 2H).

### Step 4: 3-((S)-2-((1H-indol-3-yl)methyl)-1,2,3,4-tetrahydro-3-oxoquinoxalin-6-yl)-N-hydroxypropanamide (Compound 69)

Following the procedure described in Example 1, Compound 4a, step 3, Scheme 1, but substituting acid 3a with acid 68, the title Compound was recovered as a white solid in 17% yield. ¹H NMR: (CD₃OD) δ (ppm): 7.52 (d, J=7.8 Hz, 1H), 7.34-7.30 (m, 2H), 7.08 (td, J=6.8, 1.0 Hz, 1H), 7.03-6.97 (m, 2H), 6.69 (dd, J=8.0, 2.0 Hz, 1H), 6.61 (d, J=1.8 Hz, 1H), 6.54 (d, J=7.8 Hz, 1H), 4.02 (dd, J=9.6, 3.7 Hz, 1H), 3.17 (dd, J=14.1, 3.5 Hz, 1H), 3.00-2.94 (m, 1H), 2.79 (t, J=7.2 Hz, 2H), 2.32 (t, J=8.0 Hz, 2H). LRMS (ESI): (calc.) 364.1; (found) 365.1 (MH)⁺.

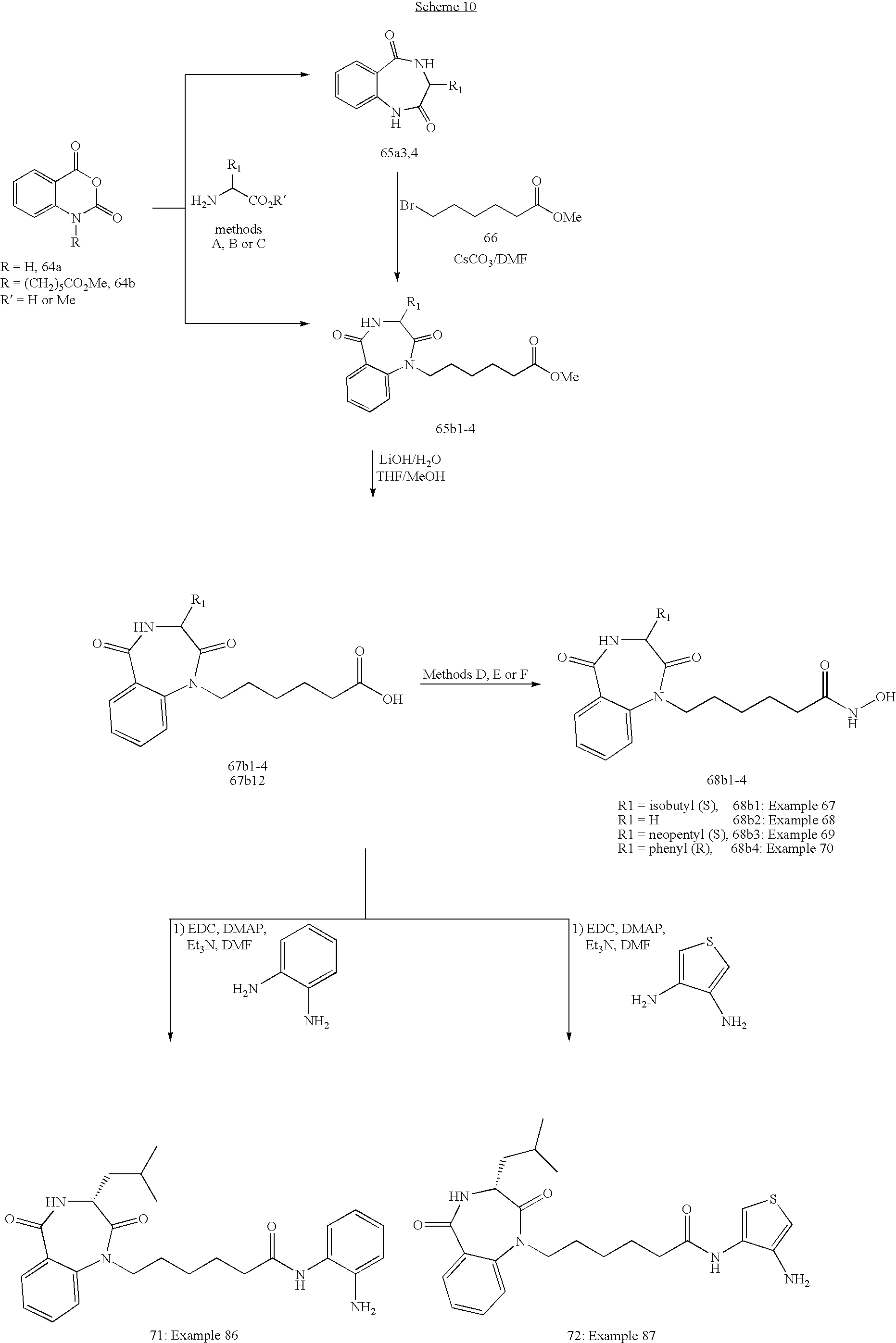

### Synthesis of Intermediate 66: Methyl 7-bromohexanoate

To a solution 6-bromohexanoic acid (10 g, 50 mmol) in MeOH was added few drops of concentrated H₂SO₄. The mixture was then refluxed overnight. Methanol was evaporated and the residue was taken in dichloromethane and washed with saturated solution of sodium bicarbonate and dried over MgSO₄. The solvent was then evaporated to give the desired ester (10.7 g) as slightly yellow oil in quantitative yield. ¹H NMR: (CDCl₃) δ (ppm): 3.6 (s, 3H); 3.3 (m, 2H); 2.4 (m, 2H); 1.5-1.8 (m, 4H); 1.3 (m, 2H).

### Synthesis of Intermediate 64b: Methyl 6-(2,4-dioxo-2H-benzo[d][1,3]oxazin-1(4H)-yl)hexanoate

Sodium hydride (0.15 g; 6 mmol) was added to a solution of isotoic anhydride (1 g; 6 mmol) in dry DMF at 0-5° C. under nitrogen. After 30 min, methyl 6-(2,4-dioxo-2H-benzo[d][1,3]oxazin-1(4H), intermediate 3 (1.26 g; 6 mmol) was added dropwise and the mixture was stirred at room temperature overnight. DMF was evaporated and the residue was taken in EtOAc and the organic layer was washed with water, brine then was dried over MgSO₄. The solvent was evaporated and the residue was purified on silica gel using hexanes/EtOAc (7:3) to give 64b (0.56 g, 32%). ¹H NMR: (CDCl₃) δ (ppm): 7.4 (d, J=6 Hz, 2H); 8.2 (d, J=6 Hz, 2H); 3.6 (s, 3H); 3.3 (m, 2H); 2.4 (m, 2H); 1.5-1.8 (m, 4H); 1.3 (m, 2H)

### EXAMPLE 67

### 6-((S)-2,3,4,5-Tetrahydro-3-isobutyl-2,5-dioxobenzo[e][1,4]diazepin-1-yl)-N-hydroxyhexanamide (Compound 68b₁)

### Step 1: Formation of the benzodiazepine ring: Method A (S)-methyl 6-(3-isobutyl-2,5-dioxo-2,3,4,5-tetrahydrobenzo[e][1,4]diazepin-1-yl)hexanoate (Compound 65b1)

To a solution of isatoic anhydride 64b1 (5 mmol) (or 64a) in acetic acid was added the L-Leucine (5 mmol) and the mixture was refluxed overnight according to the procedure of Reddy et al. (Syn Comm. 33, 237-241, 2003). After cooling NaHCO₃ was added followed by NH₄Cl then the solution was extracted from ethyl acetate. The organic layer was washed with water and brine then dried over MgSO₄. After evaporation of the solvent the residue was purified on silica gel to using EtOAc to give 65b1 (or 65a).

### Step 2: (S)-6-(3-Isobutyl-2,5-dioxo-2,3,4,5-tetrahydrobenzo[e][1,4]diazepin-1-yl)hexanoic acid (Compound 67b1)

To a solution of ester 65b1 (50 mg, 0.14 mmol) in 10 mL MeOH:THF (1:1) was added a solution of lithium hydroxide (0.2 mmol) in water (5 mL). After 90 min the solvent was evaporated and the residue was acidified with HCl 1 N till pH 4 then extracted with EtOAc to give the desired acid 67b1.

### Step 3: 6-((S)-2,3,4,5-tetrahydro-3-isobutyl-2,5-dioxobenzo[e][1,4]diazepin-1-yl)-N-hydroxyhexanamide (Compound 68b₁). Method E

To a solution of the acid 67b1 (0.21 mmol) in DMF was added HOBt (0.21 mmol), EDCI (0.21 mmol), DMAP (0.21 mmol) and the resin bound O-Hydroxylamine (0.07 mmol, purchased commercially from NovaBiochem or prepared according to the procedure of Floyd et al. Tet. Lett., 8045-8048, 1996). The mixture was shacked overnight. The resin was then washed with DMF (3×), DCM (3×) and MeOH (3×) and dried under high vacuum. This resin was treated with TFA:DCM (20%) for 4 h and the liquid was collected and evaporated to a give a residue which was purified on Prep-HPLC to give the hydroxamic acid 68b1 in 15% yield as a white solid (3 mg) after purification on prep-HPLC. ¹H NMR: (CD₃OD) δ (ppm): 7.8 (d, J=8 Hz, 1H); 7.6 (d, J=8 Hz, 1H); 7.2 (m, 2H); 4.2 (m, 2H); 3.6-3.8 (m, 3H); 1.9 (dd, J=14 Hz, 2H); 1.2-1.8(m, 6H); 0.8 (2d, 6H). LRMS (ESI): (calc.) 361; (found) 362 (MH)⁺.

### EXAMPLE 68

### 6-(2,3,4,5-tetrahydro-2,5-dioxobenzo[e][1,4]diazepin-1-yl)-N-hydroxyhexanamide (Compound 68b2)

### Step 1: Formation of the benzodiazepine ring: Method B Methyl 6-(2,5-dioxo-2,3,4,5-tetrahydrobenzo[e][1,4]diazepin-1-yl)hexanoate (Compound 65b2)

Following the procedure described in J. Med. Chem. 1999, 42, 5241, isatoic anhydride 64a or 64b (0.72 mmol) and the Gly-OMe methyl ester (0.80 mmol) in dry pyridine (2.0 mL) were heated at 100° C. for 16 h under nitrogen. The solution was evaporated and diphenyl ether (1.5 mL) was added. The heterogeneous mixture was heated at 180° C. for 1 h. Intermediate 65b2 (or 65a) was obtained after purification on silica gel using EtOAc and hexanes. ¹H NMR: (CDCl₃) δ (ppm): 7.9 (d, J=8 Hz, 1H); 7.6 (dd, J=8 Hz, 1H); 7.4 (dd, J=8 Hz, 1H); 7.3 (d, J=8 Hz, 1H); 6.8 (m, 1H); 4.25 (m, 1H); 3.6-3.8 (m, 3H); 3.6 (s, 3H); 2.2 (dd, J=14 Hz, 2H); 1.2-1.6 (m, 8H). LRMS (ESI): (calc.) 304.3; (found) 305 (MH)⁺.

### Step 2: 6-(2,5-dioxo-2,3,4,5-tetrahydrobenzo[e][1,4]diazepin-1-yl)hexanoic acid (Compound 67b2)

The title Compound 67b2 was prepared following the procedure described in Example 67, Compound 68b1, step 2, Scheme 10, but substituting 65b1 with 65b2. ¹H NMR: (CD₃OD) δ (ppm): 7.9 (d, J=8 Hz, 1H); 7.6 (dd, J=8 Hz, 1H); 7.4 (dd, J=8 Hz, 1H); 7.3 (d, J=8 Hz, 1H); 6.8 (m, 1H); 4.25 (m, 1H); 3.6-3.8 (m, 3H); 2.2 (dd, J=14 Hz, 2H); 1.2-1.6 (m, 8H) LRMS (ESI): (calc.) 290.3; (found) 291 (MH)⁺.

### Step 3: 6-(2,3,4,5-tetrahydro-2,5-dioxobenzo[e][1,4]diazepin-1-yl)-N-hydroxy-hexanamide (Compound 68b₂). Method E

Following the procedure described in Example 67, Compound 68b1, step 3, Scheme 10, but substituting 67b1 with 67b2, the title Compound was obtained as a white solid in 3% yield (5 mg) after purification on prep-HPLC. ¹H NMR: (CD₃OD) δ (ppm): 7.9 (d, J=8 Hz, 1H); 7.6 (dd, J=8 Hz, 1H); 7.4 (dd, J=8 Hz, 1H); 7.3 (d, J=8 Hz, 1H); 6.8 (m, 1H); 4.25 (m, 1H); 3.6-3.8 (m, 3H); 1.9 (dd, J=14 Hz, 2H); 1.2-1.6 (m, 8H). LRMS (ESI): (calc.) 305.3; (found) 306 (MH)⁺.

### EXAMPLE 69

### 6-((S)-2,3,4,5-tetrahydro-3-neopentyl-2,5-dioxobenzo[e][1,4]diazepin-1-yl)-N-hydroxyhexanamide (Compound 68b3)

### Step 1: Formation of the benzodiazepine ring: Method C(S)-3-neopentyl-3,4-dihydro-1H-benzo[e][1,4]diazepine-2,5-dione (65a3)

Following the procedure described in J. Med. Chem. 1999, 42, 5241, Isatoic anhydride 64a (1.2 mmol) and the L-t-butyl leucine (1.2 mmol) in dry pyridine (4.0 mL) were heated at 115° C. for 19 h under nitrogen. The solution was evaporated and diphenyl ether (4 mL) was added. The heterogeneous mixture was heated at 180° C. for 3 h. Intermediate 64a3, was obtained after purification on silica gel using EtOAc and hexanes.

### Step 2: (S)-methyl 6-(3-neopentyl-2,5-dioxo-2,3,4,5-tetrahydrobenzo[e][1,4]diazepin-1-yl)hexanoate (Compound 65b3)

To a solution of 64a3 (0.4 mmol) in DMF (15 mL) was added Intermediate 66 (100 mg, 0.5 mmol) followed by cesium carbonate (162 mg, 0.5 mmol). The solution was stirred for 18 h at rt. After evaporation of the solvent, the crude was purified on silica gel using EtOAc and hexanes to give the desired ester 65b3.

### Step 3: (S)-6-(3-neopentyl-2,5-dioxo-2,3,45-tetrahydrobenzo[e][1,4]diazepin-1-yl)hexanoic acid (Compound 67b3)

The title Compound was obtained following the procedure described in Example 67, Compound 68b1, step 2, Scheme 10, but substituting 65b1 with 65b3.

### Step 4: 6-((S)-2,3,4,5-tetrahydro-3-neopentyl-2,5-dioxobenzo[e][1,4]diazepin-1-yl)-N-hydroxyhexanamide (Compound 68b3) Method D

To a solution of the acid 67b3 (0.15 mmol) in dry DMF (5 mL) was added dry triethylamine (0.3 mmol) followed by BOP (0.22 mmol). The mixture was stirred under nitrogen at room temperature for 30 min. Then hydroxylamine hydrochloride (0.22 mmol) was added followed by triethylamine (0.3 mmol) and the mixture was stirred at room temperature for 16 h. The solvent was evaporated and the residue was purified using prep-HPLC to give the hydroxamic acid 68b3. The title Compound 68b3 was obtained in 22% yield as white solid (29 mg) after purification by preparative TLC. ¹H NMR: (DMSO-d₆) δ (ppm): 10.26 (s, 1H), 8.62 (bs, 1H), 8.55 (d, J=6.4 Hz, 1H), 7.67 (d, J=7.6 Hz, 1H), 7.60 (t, J=7.2 Hz, 1H), 7.49 (d, J=8.0 Hz, 1H), 7.33 (t, J=7.6 Hz, 1H), 4.15 (m, 1H), 3.62 (m, 1H), 3.55 (q, J=5.9 Hz, 1H), 2.01 (dd, J=14.6, 5.0 Hz, 1H), 1.84 (t, J=7.4 Hz, 2H), 1.55 (dd, J=14.6, 6.6 Hz, 1H), 1.27-1.41 (m, 4H), 1.10 (m, 2H). LRMS (ESI): (calc.) 375.5; (found) 376.3 (MH)⁺.

### EXAMPLE 70

### 6-((R)-2,3,4,5-Tetrahydro-2,5-dioxo-3-phenylbenzo[e][1,4]diazepin-1-yl)-N-hydroxyhexanamide (Compound 68b4)

### Step 1: (R)-3-Phenyl-3,4-dihydro-1H-benzo[e][1,4]diazepine-2,5-dione (Compound 65a4)

Method B

The title Compound 65a4, was prepared following the procedure described in Example 68, Compound 68b2, step 1, Scheme 10, but substituting 64b with 64a and the L-t-butyl leucine with Phenyl glycine.

### Step 2: (R)-methyl 6-(2,5-dioxo-3-phenyl-2,3,415-tetrahydrobenzo[e][1,4]diazepin-1-yl)hexanoate (Compound 65b4)

The title Compound 65b4, was prepared following the procedure described in Example 69, Compound 68b3, step 2, Scheme 10, but substituting 64a3 with 64a4

### Step 3: (R)-6-(2,5-Dioxo-3-phenyl-2,3,4,5-tetrahydrobenzo[e][1,4]diazepin-1-yl)hexanoic acid (Compound 67b4)

The title Compound 67b4, was prepared following the procedure described in Example 67, Compound 68b1, step 2, Scheme 10, but substituting 65b1 with 65b4.

### Step 4: 6-((S)-2,3,4,5-tetrahydro-3-neopentyl-2,5-dioxobenzo[e][1,4]diazepin-1-yl)-N-hydroxyhexanamide (Compound 68b3). Method F

To a solution of the acid 67b4 (0.14 mmol) in THF cooled to 0° C. was added triethylamine (0.19 mmol) and trimethylacetyl chloride (0.16 mmol). The resulting suspension was stirred at 0° C. for 15 min. Then hydroxylamine hydrochloride (0.28 mmol) was added followed by triethylamine (0.28 mmol) and the mixture was stirred at 0° C. for 15 min and at room temperature for 24 h. The solvent was evaporated and the residue was taken in ethyl acetate. The organic layer was washed with saturated solutions of ammonium chloride and then sodium bicarbonate and dried over MgSO₄. After concentration the residue was purified on silica gel using 5% methanol in EtOAc with 0.4% of acetic acid to give the hydroxamic acid 68b4. The title Compound 68b4 was obtained in 11% yield as a white solid (22 mg) after purification by preparative TLC. ¹H NMR: (DMSO-d₆) δ (ppm): 10.28 (d, J=5.2 Hz, 1H), 9.21 (d, J=7.6 Hz, 0.5H), 8.89 (d, J=6.0 Hz, 0.5H), 8.63 (s, 1H), 7.73 (dd, J=7.6, 1.6 Hz, 0.5H), 7.64 (t, J=7.6 Hz, 0.5H), 7.55 (d, J=8.0 Hz, 0.5H), 7.43 (m, 1.5H), 7.37 (t, J=7.4 Hz, 0.5H), 7.32 (m, 1.5H), 7.24 (t, J=7.8 Hz, 0.5H), 7.12 (d, J=8.0 Hz, 0.5H), 7.07 (t, J=7.4 Hz, 1H), 7.00 (t, J=7.4 Hz, 1H), 6.91 (d, J=7.2 Hz, 1H), 5.12 (d, J=8.0 Hz, 0.5H), 4.96 (d, J=6.4 Hz, 0.5H), 4.18 (m, J=7.4 Hz, 1H), 3.68 (m, 1H), 1.87 (q, J=7.3 Hz, 2H), 1.35-1.53 (m, 4H), 1.16 (m, 2H). LRMS (ESI): (calc.) 375.5; (found) 376.3 (MH)⁺.

### EXAMPLES 71-85 AND COMPOUND 68b_{6-12, 16-23},

Compounds 68b_{6-12, 16-23}, were prepared using the same procedures as described for Compound 68b₁₋₄ in Examples 67-70. Characterization data are presented in Table 8.

TABLE 8

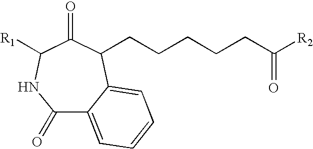

| Ex | Cpd | R1/structure | R2 | Name | Characterization | Scheme | |
|---|---|---|---|---|---|---|---|
| 71 | 68b₆ | 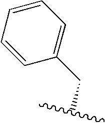 | NHOH | 6-((R)-2,3,4,5- Tetrahydro- 2,5-dioxo-3- ((pyridin-4- yl)methyl)benzo [e][1,4 diazepin- 1-yl)-N- hydroxyhexanamide | ¹H NMR: (MeOD-d₄) δ(ppm): 7.8 (d, J=8 Hz, 1H); 7.6 (d, J=8 Hz, 1H); 7.5 (d, 7.8 Hz, 1H); 7.4 (d, J=8 Hz, 1H); 7-7.3 (m, 5H); 4.7 (t, J=6 Hz, 1H); 3.2 (dd, J=14 Hz, 2H); 2.8-3.2 (m, 2H); 1.9 (m, 2H); 1.2-1.8 (m, 6H). LRMS (ESI): (calc.) 396.4; | | |
| # (found) 397 (MH)⁺. | 10 Method F | | | | | | |
| 72 | 68b₇ | 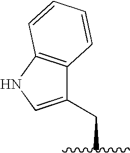 | NHOH | 6-((S)-3-((1H- Indol-3- yl)methyl)- 2,3,4,5- tetrahydro- 2,5- dioxobenzo[e][1,4]diazepin- 1-yl)-N hydroxyhexanamide | ¹H NMR: (MeOD-d₄) δ(ppm): 7.70 (dd, J=1.6, 7.8 Hz, 1H), 7.58 (dt, J=1.8, 7.8 Hz, 1H), 7.44 (d, J=7.8 Hz, 1H), 7.38 (d, J=7.8 Hz, 1H), 7.32 (dt, J=1, 7.2 Hz, 1H), 7.28 (d, J=8 Hz, 1H), 7.11 (s, 1H), 7.03 (dt, J=1, 7 Hz, | | |
| # 1H), 6.90 (dt, J=1, 7.5 Hz, 1H), 4.42 (m, 1H), 4.05 (dd, J=6.5, 8 Hz, 1H), 3.67 (m, 1H), 3.43 (dd, J=6, 15 Hz, 1H), 3.17 (dd, J=7.8, 15 Hz, 1H), 1.99 (t, J=7.4 Hz, 2H), 1.63-1.38 (m, 4H), 1.28-1.13 (m, 2). LRMS (ESI): (calc.) 434.5; (found) 435 (MH)⁺. | 10 Method E | | | | | | |
| 73 | 68b₈ | 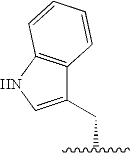 | NHOH | 6-((R)-3-((1H-Hz, indol-3- yl)methyl)- 2,3,4,5- tetrahydro- 2,5- dioxobenzo[e][1,4]diazepin- 1-yl)-N- hydroxyhexanamide | ¹H NMR: (MeOD-d₄) δ(ppm): 7.70 (dd, J=1.6, 7.8 Hz, 1H), 7.58 (dt, J=1.8, 7.8 1H), 7.44 (d, J=7.8 Hz, 1H), 7.38 (d, J=7.8 Hz, 1H), 7.32 (dt, J=1, 7.2 Hz, 1H), 7.28 (d, J=8 Hz, 1H), 7.11 (s, 1H), 7.03 (dt, J=1, 7 Hz, | | |
| # 1H), 6.90 (dt, J=1, 7.5 Hz, 1H), 4.42 (m, 1H), 4.05 (dd, J=6.5, 8 Hz, 1H), 3.67 (m, 1H), 3.43 (dd, J=6, 15 Hz, 1H), 3.17 (dd, J=7.8, 15 Hz, 1H), 1.99 (t, J=7.4 Hz, 2H), 1.63-1.38 (m, 4H), 1.28-1.13 (m, 2). LRMS (ESI): (calc.) 434.5; (found) 435 (MH)⁺. | 10 Method E | | | | | | |
| 74 | 68b₉ | 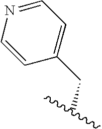 | NHOH | 6-((R)-2,3,4,5- Tetrahydro- 2,5-dioxo-3- ((pyridin-4- yl)methyl)benzo [e][1,4]diazepin- 1-yl)-N- hydroxyhexanamide | ¹H NMR: (MeOD-d₄) δ(ppm): 7.8 (d, J=8 Hz, 1H); 7.6 (d, J=8 Hz, 1H); 7.5 (d, 7.8 Hz, 1H); 7.4 (d, J=8 Hz, 1H); 7-7.3 (m, 4H); 4.7 (t, J=6 Hz, 1H); 3.2( dd, J=14 Hz, 2H); 2.8-3.2 (m, 2H); 1.9 (m, 2H); 1.2-1.8 (m, 6H) LRMS (ESI): (calc.) 396.4; | | |
| # (found) 397 (MH)^{+.} | 10 Method E | | | | | | |
| 76 | 68b₁₁ | 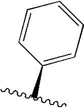 | NHOH | 6-((S)-2,3,4,5- tetrahydro- 2,5-dioxo-3- phenylbenzo[e][1,4]diazepin- 1-yl)-N-hydro- xyhexanamide | ¹H NMR: (MeOD-d₄) δ(ppm): 7.8 (m, 3H); 7.6 | (m, 4H); 7.2 (m, 2H); 5.4 (m, 1H); 1.9 (dd, J=14 Hz, 2H); 1.2-1.8 (m, 6H); LRMS (ESI): (calc.) 381.4; (found) 382 (MH)⁺. | 10 Method E |
| 77 | 68b₁₂ | 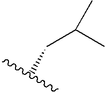 | NHOH | 6-((R)-2,3,4,5- tetrahydro-3- isobutyl-2,5- dioxobenzo[e][1,4]diazepin- 1-yl)-N- amide | ¹H NMR: (MeOD-d₄) δ(ppm). 7.78 (dd, J=1.6, 7.8 Hz, 1H); 7.64 (dt, J=1.8, 8.2 Hz, 1H); 7.49 (d, J=8.4, 1H); 7.39 (dt, J=1, 7.6 Hz, 1H), 4.39 (m, 1H), 3.75 (t, J=8 Hz, 1H), 3.68 (m, 1H), 1.99 (t, J=7.4 Hz, 2H), 1.74 (m, 3H), 1.62-1.38 (m, 4H), 1.3-1.1 (m, 2H), 0.92 (d, | | |
| # J=6.3 Hz, 3H), 0.83 (d, J=6.3 Hz, 3H) LRMS (ESI): (calc.) 361 .4; (found) 362 (MH)⁺. | 10 Method E | | | | | | |
| 78 | 68b₁₆ | 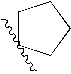 | NHOH | 6-(3,3- Spirocyclopentyl- 2,5-dioxo- 2,3,4,5, tetrahydrobenzo [e][1,4]diazepin-1- yl)-N- hydroxyhexanamide | ¹H NMR: (DMSO-d₆) δ(ppm): 10.27 (s, 1H), 8.62 (d, J=5.2 Hz, 2H), 7.60 (m, 2H), 7.43 (d, J=8.0 Hz, 1H), 7.30 (t, J=7.6 Hz, 1H), 4.15 (m, J=7.2 Hz, 1H), 3.64 (m, 1H), 2.40 (m, 1H), 1.83 (m, 3H), 1.33-1.60 (m, 10H), 1.11 (m, 3H). LRMS (ESI): | | |
| # (calc.) 359.4; (found) 360.3 (MH)⁺. | 10 Method C & D | | | | | | |
| 79 | 68b₁₇ | 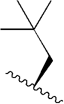 | NHOH | 6-((S)-2,3,4,5- Tetrahydro-3- neopentyl-2,5- dioxobenzo[e][1,4]diazepin- 1-yl)-N- hydroxyhexanamide | ¹H NMR: (MeOD-d₄) δ(ppm): 10.26 (s, 1H), 8.62 (bs, 1H), 8.55 (d, J=6.4 Hz, 1H), 7.67 (d, J=7.6H, 1H), 7.60 (t, J=7.2 Hz, 1H), 7.49 (d, J=8.0 Hz, 1H), 7.33 (t, J=7.6 Hz, 1H), 4.15 (m, 1H), 3.62 (m, 1H), 3.55 (q, J=5.9 Hz, 1H), 2.01 (dd, J=14.6, | | |
| # 5.0 Hz, 1H), 1.84 (t, J=7.4 Hz, 2H), 1.55 (dd, J=14.6, 6.6 Hz, 1H), 1.27-1.41 (m, 4H), 1.10 (m, 2H). LRMS (ESI): (calc.) 375.5; (found) 376.3 (MH)⁺. | 10 Method C & D | | | | | | |
| 80 | 68b₁₈ | 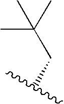 | NHOH | 6-((R)-2,3,4,5- Tetrahydro-3- neopentyl-2,5- dioxobenzo[e][1,4]diazepin- 1-yl)-N- hydroxyhexanamide | ¹H NMR: (MeOD-d₄) δ(ppm): 10.26 (s, 1H), 8.62 (s, 1H), 8.55 (d, J=5.6 Hz, 1H), 7.67 (dd, J=7.6, 1.6 Hz, 1H), 7.60 (td, J=7.6, 1.6 Hz, 1H), 7.50 (d, J=8.4 Hz, 1H), 7.34 (t, J=7.4 Hz, 1H), 4.15 (m, 1H), 3.62 (m, 1H), 3.55 (q, J=5.9 Hz, 1H), 2.01 | | |
| # (dd, J=14.4, 4.8 Hz, 1H), 1.84 (t, J=7.2 Hz, 2H), 1.55 (dd, J=14.4 6.4 Hz, 1H), 1.27-1.41 (m, 4H), 1.10 (m, 2H). LRMS (ESI): (calc.) 375.5; (found) 376.3 (MH)⁺ | 10 Method C & D | | | | | | |
| 81 | 68b₁₉ | 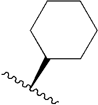 | NHOH | 6-((S)-3- Cyclohexyl- 2,3,4,5- tetrahydro- 2,5- dioxobenzo[e][1,4]diazepin- 1-yl)-N- hydroxyhexanamide | ¹H NMR: (DMSO-d₆) δ(ppm): 10.26 (s, 1H), 8.64 (d, J=6.8 Hz, 1H), 8.62 (s, 1H), 7.64 (d, J=7.6 Hz, 1H), 7.57 (t, J=7.6 Hz, 1H), 7.49 (d, J=8.4 Hz, 1H), 7.31 (t, J=7.4 Hz, 1H), 4.14 (m, 1H), 3.60 (m, 1H), 3.26 (m, 1H), 1.92 (m, 1H), 1.82 (m, 3H), | | |
| # 1.58-1.67 (m, 3H), 1.27-1.41 (m, 4H), 1.04-1.25 (m, 6H), 0.69-0.87 (m, 2H). LRMS (ESI): (calc.) 387.5; (found) 388.4 (MH)⁺. | 10 Method C & D | | | | | | |
| 82 | 68b₂₀ | 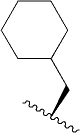 | NHOH | 6-((S)-3- (Cyclohexylmethyl)- 2,3,4,5- tetrahydro- 2,5- dioxobenzo[e][1,4]diazepin- 1-yl)-N- hydroxyhexanamide | ¹H NMR: (DMSO-d₆) δ(ppm): 10.26 (s, 1H), 8.62 (s, 1H), 8.55 (d, J=5.6 Hz, 1H), 7.66 (d, J=7.6 Hz, 1H), 7.58 (t, J=7.8 Hz, 1H), 7.48 (d, J=8.0 Hz, 1H), 7.32 (t, J=7.4 Hz, 1H), 4.18 (m, 1H), 3.60 (m, 2H), 1.83 (t, J=7.4 Hz, 2H), 1.60 (m, 6H), 1.46 | | |
| # (m, 1H), 1.37 (m, 5H), 1.10 (m, 5H), 0.72-0.90 (m, 2H). LRMS (ESI):(calc.) 401.5; (found) 402.4 (MH)⁺. | | | | | | | |
| 83 | 68b₂₁ | 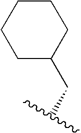 | NHOH | 6-((R)-3- (cyclohexylmethyl)- 2,3,4,5- tetrahydro- 2,5- dioxobenzo[e][1,4]diazepin- 1-yl)-N- hydroxyhexanamide | ¹H NMR: (DMSO-d₆) δ(ppm): 10.26 (s, 1H), 8.62 (s, 1H), 8.55 (d, J=5.6 Hz, 1H), 7.66 (d, J=7.6 Hz, 1H), 7.58 (t, J=7.6 Hz, 1H), 7.48 (d, J=8.0 Hz, 1H), 7.32 (t, J=7.4 Hz, 1H), 4.18 (m, 1H), 3.60 (m, 2H), 1.83 (t, J=7.2 Hz, 2H), 1.60 (m, 6H), 1.46 | | |
| # (m, 1H), 1.37 (m, 5H), 1.10 (m, 5H), 0.72-0.90 (m, 2H). LRMS (ESI): (calc.) 401.5; (found) 402.2 (MH)⁺. | | | | | | | |
| 84 | 68b₂₂ | 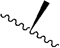 | NHOH | 6-((S)-2,3,4,5- Tetrahydro-3- methyl-2,5- dioxobenzo[e][1,4]diazepin- 1-yl)-N- hydroxyhexanamide | ¹H NMR: (DMSO-d₆) δ(ppm): 10.27 (bs, 1H), 8.63 (bs, 1H), 8.53 (d, J=6.0 Hz, 1H), 7.66 (dd, J=7.6, 1.6 Hz, 1H), 7.58 (td, J=7.8, 1.6 Hz, 1H), 7.47 (d, J=8.0 Hz, 1H), 7.31 (td, J=7.4, 0.8 Hz, 1H), 4.18 (m, 1H), 3.77 (m, 1H), 3.61 (m, 1H), 1.84 (t, | | |
| # J=7.4 Hz, 2H), 1.28-1.41 (m, 4H), 1.22 (d, J=6.8 Hz, 3H), 1.11 (m, 2H). LRMS (ESI): (calc.) 319.4; (found) 320.2 (MH)⁺. | 10 Method B & D | | | | | | |
| 85 | 68b₂₃ | 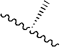 | NHOH | 6-((R)-2,3,4,5- Tetrahydro-3- methyl-2,5- dioxobenzo[e][1,4]diazepin- 1-yl)-N- hydroxyhexanamide | ¹H NMR: (DMSO-d₆) δ(ppm): 10.26 (s, 1H), 8.62 (s, 1H), 8.53 (d, J=5.6 Hz, 1H), 7.66 (dd, J=7.8, 1.4 Hz, 1H), 7.58 (td, J=7.6, 1.6 Hz, 1H), 7.47 (d, J=8.0 Hz, 1H), 7.31 (td, J=7.5, 1.0 Hz, 1H), 4.18 (m, 1H), 3.77 (m, 1H), 3.61 (m, 1H), 1.84 (t, | | |
| # J=7.2 Hz, 2H), 1.27-1.42 (m, 4H), 1.22 (d, J=6.8 Hz, 3H), 1.11 (m, 2H). LRMS (ESI): (calc.) 319.4; (found) 320.2 (MH)⁺. | 10 Method B & F | | | | | | |

### EXAMPLE 86

### N-(2-Aminophenyl)-6-((R)-2,3,4,5-tetrahydro-3-isobutyl-2,5-dioxobenzo[e][1,4]diazepin-1-yl)hexanamide (Compound 71)

### Step 3: N-(2-Aminophenyl)-6-((R)-2,3,45-tetrahydro-3-isobutyl-2,5-dioxobenzo[e][1,4]diazepin-1-yl)hexanamide (Compound 71)

Compound 67b₁₂ (see Scheme 10, Example 67, step 1-2 for preparation) (40 mg, 0.12 mmol) and EDC (44 mg, 0.23 mmol), were stirred in DMF (1.5 mL) under nitrogen at room temperature for 10 min. 1,2-phenylenediamine (19 mg, 0.17 mmol) and DMAP (14 mg, 0.12 mmol) were added and the solution stirred for 16 h. The solvent was then evaporated and the residue dissolved in EtOAc. The solution was washed with saturated NH₄Cl, saturated NaHCO₃, brine, and then dried over MgSO₄. The solvent was evaporated and the residue purified by preparative TLC (EtOAc) to give 71 as a beige solid (20 mg, 40%). ¹H NMR: (DMSO-d₆) δ (ppm): 9.01 (s, 1H), 8.57 (d, J=6.0 Hz, 1H), 7.67 (dd, J=7.8, 1.4 Hz, 1H), 7.58 (t, J=7.6 Hz, 1H), 7.49 (d, J=8.0 Hz, 1H), 7.32 (t, J=7.6 Hz, 1H), 7.10 (d, J=7.6 Hz, 1H), 6.86 (t, J=7.4 Hz, 1H), 6.68 (dd, J=8.0, 1.2 Hz, 1H), 6.50 (t, J=7.6 Hz, 1H), 4.79 (s, 2H), 4.20 (m, 1H), 3.56-3.67 (m, 2H), 2.21 (t, J=7.4 Hz, 2H), 1.57-1.70 (m, 3H), 1.30-1.54 (m, 4H), 1.19 (m, 2H), 0.82 (d, J=6.4 Hz, 3H), 0.73 (d, J=6.4 Hz, 3H). LRMS (ESI): (calc.) 437.26; (found) 437.4 (MH)⁺.

### EXAMPLE 87

### N-(4-Aminothiophen-3-yl)-6-((R)-2,3,4,5-tetrahydro-3-isobutyl-2,5-dioxobenzo[e][1,4]diazepin-1-yl)hexanamide (Compound 72)

### Step 3: N-(4-aminothiophen-3-yl)-6-((R)-2,3,4,5-tetrahydro-3-isobutyl-2,5-dioxobenzo[e][1,4]diazepin-1-yl)hexanamide (Compound 72)

Following the procedure described above (Compound 72, Example 86, Scheme 10), substituting 1,2-Phenylenediamine for 3,4-Diaminothiophene, the title Compound 72 was obtained in 29% yield as a grey solid (15 mg) after a purification on prep-HPLC. ¹H NMR: (DMSO-d₆) δ (ppm): 9.22 (s, 1H), 8.57 (d, J=6.0 Hz, 1H), 7.66 (dd, J=7.6, 1.6 Hz, 1H), 7.58 (td, J=7.6, 1.8 Hz, 1H), 7.49 (d, J=8.4 Hz, 1H), 7.35 (d, J=3.6 Hz, 1H), 7.32 (t, J=7.2 Hz, 1H), 6.02 (d, J=3.6 Hz, 1H), 4.19 (m, 1H), 3.55-3.66 (m, 2H), 2.21 (t, J=7.4 Hz, 2H), 1.56-1.68 (m, 3H), 1.31-1.51 (m, 4H), 1.17 (m, 2H), 0.83 (d, J=6.4 Hz, 3H), 0.73 (d, J=6.4 Hz, 3H). LRMS (ESI): (calc.) 442.4; (found) 443.2 (MH)⁺.

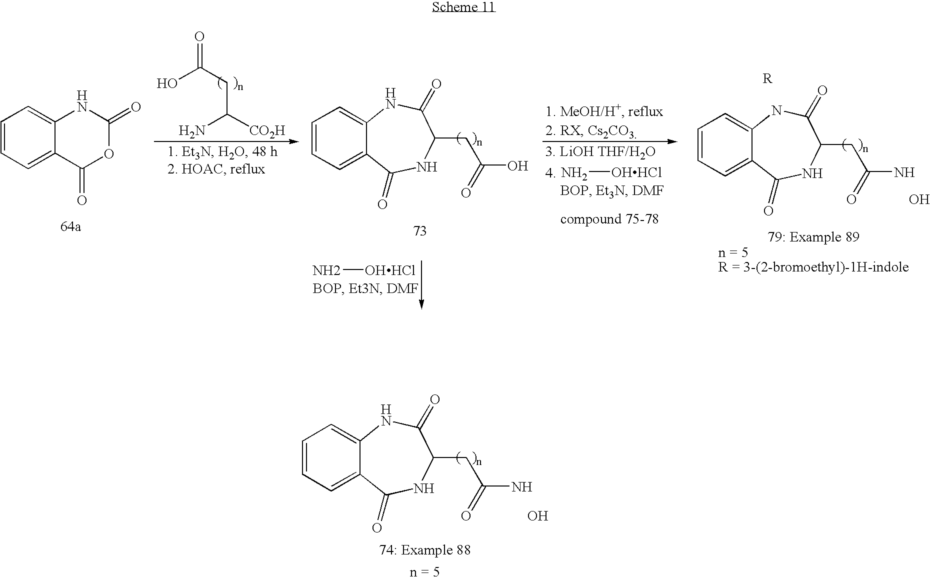

### EXAMPLE 88

### 6-(2,3,4,5-Tetrahydro-2,5-dioxo-1H-benzo[e][1,4]diazepin-3-yl)-N-hydroxyhexanamide (Compound 74)

### Step 1: 6-(2,3,4,5-Tetrahydro-2,5-dioxo-1H-benzo[e][1,4]diazepin-3-yl)hexanoic acid (Compound 73)

Isatoic anhydride 64a (1.63 g, 10 mmol) was reacted with DL-2-aminooctanedioic acid (1.89 g, 10 mmol) and triethylamine (2.17 g, 21.5 mmol) in H₂O (5 mL) according to the procedure of (Synthetic communications 2003, 33 (2), 237-241; Heterocyclic Chemistry 2003, 40, 29). After 48 h at room temperature, the reaction was taken to dryness; acetic acid (10 mL) was added and the mixture was refluxed for 4 h. The reaction was taken to dryness, EtOAc was added and the mixture was extracted with K₂CO₃ solution, then the aqueous layer was acidified to pH 4 with 1 N HCl and the precipitate was filtered and washed with H₂O. The title Compound was obtained in 50% yield as a grey solid (1.45 g). ¹H NMR: (DMSO-d₆) δ (ppm): 12.2 (bs, 1H), 10.32 (bs, 1H), 8.42 (d, J=5.7 Hz, 1H), 7.7 (dd, J=1.6, 7.8 Hz, 1H), 7.47 (ddd, J=1.8, 7.2, 7.4 Hz, 1H), 7.18 (m, 1H), 7.06 (dd, J=1, 8.2 Hz, 1H), 3.57 (m, 1H), 2.16 (t, J=7.2 Hz, 2H), 1.73 (m, 1H), 1.6-1.2 (m, 7H).

### Step 2: 6-(2,3,4,5-Tetrahydro-2,5-dioxo-1H-benzo[e][1,4]diazepin-3-yl)-N-hydroxyhexanamide (Compound 74)

The acid 73 (180 mg, 0.62 mmol) was reacted with polymer supported hydroxyl amine 120 mg, 1.7 mmol/g) prepared according to the procedure of (European Journal of Organic Chemistry 2002, 428-438), EDC (127 mg, 0.62 mmol), HOBt (85 mg, 0.62 mmol), and DMAP (cat. amount) in DMF/CH₂Cl₂ (5/5 mL). After 16 h at room temperature, the resin was filtered out and washed exhaustively with CH₂Cl₂, DMF, CH₂Cl₂, MeOH then CH₂Cl₂ and the resin was allowed to dry then it was treated with 20% TFA in CH₂Cl₂ (8 mL) for 1 h. The resin was filtered and washed with CH₂Cl₂ and all the filtrate was combined and concentrated leaving 55 mg of crude material. Flash chromatography with MeOH/CH₂Cl₂ and a drop of acetic acid, gave the desired hydroxamic acid 74 as a white solid (23 mg, 12%). ¹H NMR: (DMSO-d₆) δ (ppm): 10.25 (bs, 1H), 10.2 (bs, 1H), 8.56 (bs, 1H), 8.34 (d, J=5.7 Hz, 1H), 7.64 (dd, J=1.6, 7.6 Hz, 1H), 7.41 (ddd, J=1.8, 7.2, 7.4 Hz, 1H), 7.12 (dt, J=1.2, 7.8 Hz, 1H), 7.00 (dd, J=0.8, 8.2 Hz, 1H), 3.48 (m, 1H), 1.84 (t, J=7.2 Hz, 2H), 1.68 (m, 2H), 1.54-1.08 (m, 7H).

### EXAMPLE 89

### 6-(1-(2-(1H-Indol-3-yl)ethyl)-2,3,4,5-tetrahydro-2,5-dioxo-1H-benzo[e][1,4]diazepin-3-yl)-N-hydroxyhexanamide (Compound 79)

### Step 2: Methyl 6-(2,5-dioxo-2,3,4,5-tetrahydro-1H-benzo[e][1,4]diazepin-3-yl)hexanoate (Compound 75)

The acid 73 (see step 1, Example 88, Scheme 11 for preparation) (100 mg, 0.34 mmol), MeOH (5 mL) and few drops of H₂SO₄ were refluxed for 10 min, the mixture was taken to dryness, and EtOAc was added and the un-reacted acid was extracted with saturated Na₂CO₃ solution. The organic layer was dried and concentrated leaving the title Compound as white solid (73 mg, 70% yield).

### Step 3: Methyl 6-(1-(2-(1H-indol-3-yl)ethyl)-2,5-dioxo-2,3,4,5-tetrahydro-1H-benzo[e][1,4]diazepin-3-yl)hexanoate (Compound 76)

A mixture of 75 (300 mg, 0.99 mmol), 3-(2-bromoethyl)-1H-indole (242 mg, 1.1 mmol), Cs₂CO₃ (0.965 g, 3 mmol) in dry DMF (10 mL) was stirred at 40° C. for 16 h. H₂O was added and the product was extracted with EtOAc, and the crude product was purified by flash chromatography eluting with 20% EtOAc/hexanes. Ester 76 was obtained in 33% yield (150 mg). ¹H NMR: (DMSO-d₆) δ (ppm): 10.79 (s, 1H), 8.57 (d, J=5.9H, 1H), 7.68 (dd, J=1.8, 7.8 Hz, 1H), 7.59 (dt, J=1.8, 7.2 Hz, 1H), 7.51 (m, 2H), 7.31 (m, 2H), 7.03 (m, 2H), 6.95 (m, 1H), 4.33 (m, 1H), 3.91 (m, 1H), 3.59 (m, 1H), 3.55 (s, 3H), 2.81 (m, 2H), 2.27 (t, J=7.2 Hz, 2H), 1.77 (m, 1H), 1.65 (m, 1H), 1.5 (m, 2H), 1.38-1.2 (m, 4H).

### Step 4: 6-(1-(2-(1H-indol-3-yl)ethyl)-2,5-dioxo-2,3,4,5-tetrahydro-1H-benzo[e][1,4]diazepin-3-yl)hexanoic acid (Compound 77)

The ester methyl 76 (150 mg, 0.34 mmol), was hydrolyzed with LiOH (38 mg, 1.67 mmol) in THF/MeOH/H₂O (1:1:1 mL). After 2 h at room temperature the pH was adjusted to 3 with HCl, the mixture was taken to dryness, then, H₂O was added and the product was extracted with EtOAc, and the organic layer was concentrated giving the title Compound in 88% yield as white solid (130 mg). ¹H NMR: (DMSO-d₆) δ (ppm): 10.79 (s, 1H), 8.56 (d, J=6.1 Hz, 1H), 7.68 (dd, J=1.6, 7.6, 1H), 7.58 (dt, J=1.8, 8.4 Hz, 1H), 7.51 (m, 2H), 7.3 (m, 2H), 7.03 (m, 2H), 6.94 (m, 1H). 4.31 (m, 1H), 3.91 (m, 1H), 3.85 (m, 2H), 2.81 (m, 2H), 2.16 (t, J=7.2 Hz, 2H), 1.76 (m, 1H), 1.63 (m, 1H), 1.46 (m, 2H, 1.35-1.2 (m, 4H).

### Step 5: 6-(1-(2-(1H-Indol-3-yl)ethyl)-2,3,4,5-tetrahydro-2,5-dioxo-1H-benzo[e][1,4]diazepin-3-yl)-N-hydroxyhexanamide (Compound 79)

Acid 77 (110 mg, 0.25 mmol) was converted to the hydroxamic acid 79 in 1% yield as a beige solid using the procedure described in Example 88, step 2, Scheme 11. ¹H NMR: (CD₃OD) δ (ppm): 7.67 (dd, J=1.6, 7.8 Hz, 1H), 7.45 (dt, J=1.8, 8.6 Hz, 1H), 7.41 (d, J=7.6 Hz, 1H), 7.37 (d, J=7.8 Hz, 1H), 7.26 (t, J=7.6 Hz, 1H), 7.18 (d, J=8 Hz, 1H), 6.96 (dt, J=1.2, 6.8 HZ, 1H), 6.87 (t, J=7.8 Hz, 1H), 6.83 (s, 1H), 4.48 (m, 1H), 3.92 (m, 1H), 3.62 (t, J=6.6 Hz, 1H), 2.87 (m, 2H), 2.0 (t, J=7.2 Hz, 2H), 1.85 (m, 1H), 1.67 (m, 1H), 1.55 (m, 2H), 1.28 (m, 4H).

### EXAMPLES 90-93

Examples 90-93 describe the preparation of Compound 80-83 using the same procedures as described for Compound 79 in Example 89, step 1-4 and Example 69, step 4 (method D), Scheme 10. Characterization data are presented in Table 9.

TABLE 9

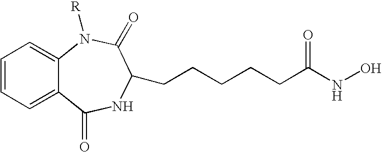

| Ex | Cpd | R | Name | Characterization | Scheme |
|---|---|---|---|---|---|
| 90 | 80 | 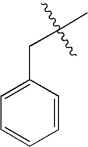 | 6-(1-Benzyl- 2,3,4,5- tetrahydro- 2,5-dioxo-1H- benzo[e][1,4]diazepin-3-yl)- N- hydroxyhexanamide | ¹H NMR: (DMSO-d₆) δ (ppm): 10.26 (d, J=1.8 Hz, 1 H), 8.63 (d, J=1.8 Hz, 1H); 8.62 (d, J=1.8 Hz, 1H), 7.61 (dd, J=7.6, 1.6 Hz, 1H), 7.50 (dt, 1.6, 7 Hz, 1H), 7.43 (d, J=7.4 Hz, 1H), 7.20 (m, 4H), 7.06 (dd, J=8.2, 1.4 Hz, 1H), 5.30 (d, J=16 Hz, 1H), | |
| # 4.96 (d, J=16 Hz, 1H), 3.69 (q, J=6.3 Hz, 1H), 1.9 (t, J=7, 2 H), 1.76 (m, 1H), 1.65 (m, 1H), 1.5-1.2 (m, 6H). LRMS (ESI): (calc.) 395.5; (found) 396 (MH)⁺. | 11 | | | | |
| 91 | 81 | 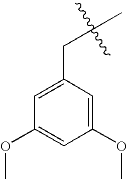 | 6-(1-(3,5- Dimethoxybenzyl)- 2,3,4,5- tetrahydro- 2,5-dioxo-1H- benzo[e][1,4]diazepin-3-yl)- N- hydroxyhexanamide | ¹H NMR: (DMSO-d₆) δ (ppm): 10.25 (s, 1 H), 8.67 (d, J=6.1 Hz, 1H), 8.62 (d, J=1.6 Hz), 7.61 (dd, J=1.4, 7.8 Hz, 1 H), 7.47 (m, 2H), 7.25 (t, J=7.6 Hz, 1H), 6.24 (d, J=2.1 Hz, 1H), 6.18 (d, J=2.1 Hz, 2H), 5.3 (d, J=16 Hz, 1H), 4.85 (d, J=16 Hz, 1H), 3.71 | |
| # (m, 1H), 3.62 (s, 6H), 1.9 (t, J=7 Hz, 2H), 1.77 (m, 1H), 1.65 (m, 1H), 1.5-1.12 (m, 6H). LRMS (ESI): (calc.) 455.5; (found) 456 (MH)⁺. | 11 | | | | |
| 92 | 82 | 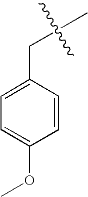 | 6-(1-(4- Methoxybenzyl)- 2,3,4,5- tetrahydro- 2,5-dioxo-1H- benzo[e][1,4]diazepin-3-yl)- N- hydroxyhexanamide | ¹H NMR: (MeOD-d₄) δ (ppm): 7.58 (dd, J=1.4, 7.6 Hz, 1 H), 7.41 (m, 2H), 7.19 (m, 1H), 6.9 (d, J=8.8 Hz, 2H), 6.63 (d, J=8.8 Hz, 2H), 5.31 (d, J=15.1 Hz, 1H), 4.72 (d, J=15.1 Hz, 1H), 3.67 (dd, J=6.5 Hz, 1H), 3.59 (s, 3H), 1.96 (t, J=6.1 Hz, 2H), 1.85 (m, | |
| # 1H), 1.65 (m, 1H), 1.5 (m, 2H), 1.34-1.18 (m, 4H). LRMS (ESI): (calc.) 425.5; (found) 426 (MH)⁺. | 11 | | | | |
| 93 | 83 | 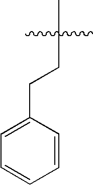 | 6-(2,3,4,5- tetrahydro- 2,5-dioxo-1- phenethyl-1H- benzo[e][1 ,4]diazepin-3-yl)- N- hydroxyhexanamide | ¹H NMR: (DMSO-d₆) δ (ppm): 10.26 (s, 1 H), 8.62 (s, 1 H), 8.51 (d, J=5.5 Hz, 1H), 7.66 (d, J=7.4 Hz, 1H), 7.57 (t, J=7 Hz, 1H), 7.31 (t, J=7.2 Hz, 1H), 7.22-7.08 (m, 5H), 4.31 (m, 1H), 3.88 (m, 1H), 3.53 (m, 1H), 2.69 (m, 2H), 1.89 (t, J=7 Hz, 2H), 1.72 | |
| # (m, 1H), 1.58 (m, 1H), 1.43 (m, 2H), 1.3-1.14 (m 4H). LRMS (ESI): (calc.) 409.5; (found) 410 (MH)⁺. | 11 | | | | |

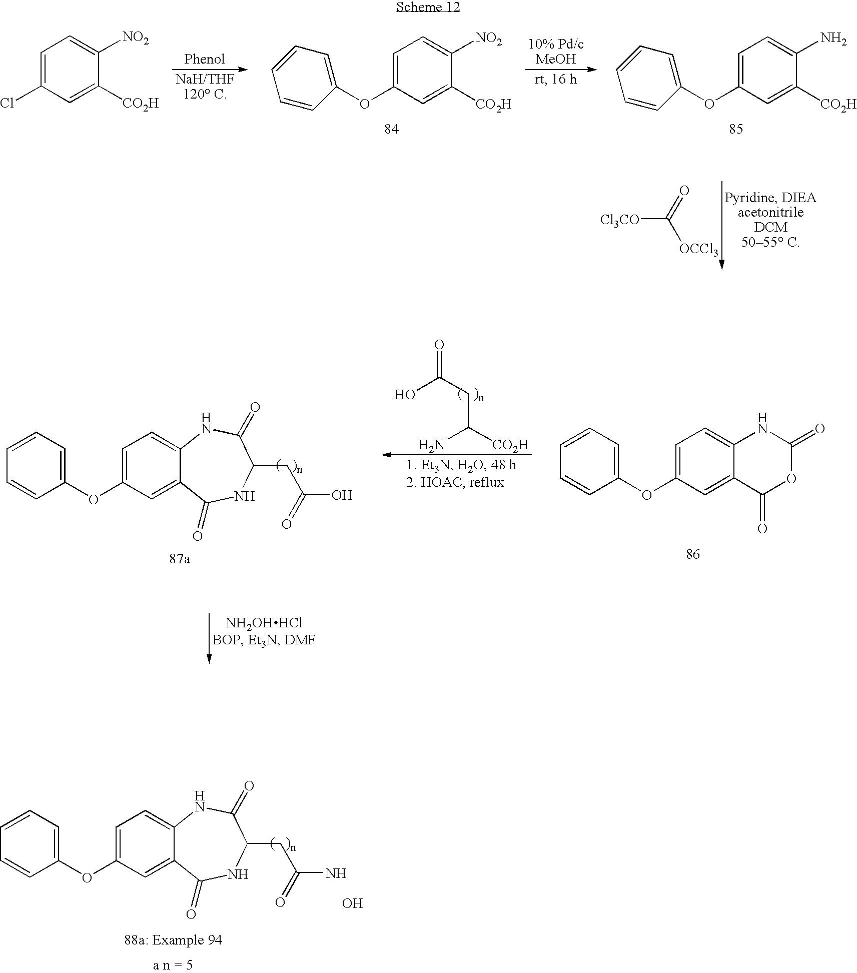

### EXAMPLE 94

### 6-(2,3,4,5-Tetrahydro-2,5-dioxo-7-phenoxy-1H-benzo[e][1,4]diazepin-3-yl)-N-hydroxyhexanamide (Compound 88a)

### Step 1: 2-Nitro-5-phenoxybenzoic acid (Compound 84)

NaH (4 g, 60% in oil, 100 mmol) was added portion wise to a solution of phenol (9.4 g, 100 mmol,) in dry THF (50 mL). After 1 h, 5-chloro-2-nitrobenzoic acid (10 g, 50 mmol) was added and the mixture was heated in a pressure tube at 120° C. for 16 h. After cooling, the mixture was acidified with 1 N HCl and the product was extracted with EtOAc. The organic extracts were washed with brine, dried over MgSO₄, filtered and concentrated. The residue was washed with hexanes to remove the mineral oil giving the title Compound 84 used crude in the next reaction.

### Step 2: 2-Amino-5-phenoxybenzoic acid (Compound 85)

The crude nitro 84 was hydrogenated at 1 atm in MeOH (200 mL) using 10% Pd/C wet catalyst (1 g). After 16 h, the catalyst was filtered through Celite and the filtrate was taken to dryness and the dark residue was treated with a 2 M solution of HCl/ether, and the mixture was stirred for 2 h. The precipitate was filtered out and washed repeatedly with ether and was allowed to air dry giving the title Compound 85 as a light beige solid HCl salt (10.92 g, 82%). ¹H NMR: (DMSO-d₆) δ (ppm): 8.7 (bs), 7.34 (m, 3H), 7.15 (m, 1H), 7.08 (m, 2H), 6.94 (m, 2H).

### Step 3: 6-Phenoxy-1H-benzo[d][1,3]oxazine-2,4-dione (Compound 86)

The isatoic anhydride was prepared according to the method of Huang Jun-Min et al, (Synthetic communication 2002, 14, 2215-2226). 2-Amino-5-phenoxybenzoic acid. HCl salt 85 (500 mg, 1.88 mmol) in acetonitrile (2 mL) was treated with one eq. of DIEA (328 uL, 1.88 mmol) and the mixture was placed in a preheated oil bath at 55° C. Pyridine (304 uL, 3.76 mmol) and a solution of triphosgene (186 mg, 0.627 mmol) DCM (1 mL) were added drop wise over 1 h. After 3.5 h, heating was stopped and the mixture was left at room temperature for 48 h. The reaction was taken to dryness, H₂O added and the precipitate was filtered and washed with H₂O and was allowed to air dry then it was washed repeatedly with ether giving the title Compound 86 as a beige solid in 66% yield (320 mg). LRMS (ESI): (calc.) 255; (found) 254 (M-H)⁻. ¹H NMR: (DMSO-d₆) δ (ppm): 11.74 (s, 1H), 7.49 (dd, J=2.5, 8.8 Hz, 1H), 7.4 (m, 2H), 7.32 (d, J=2.5 Hz, 1H), 7.17 (m, 2H), 7.02 (d, J=7.8 Hz, 2H).

### Step 4: 6-(2,3,4,5-Tetrahydro-2,5-dioxo-7-phenoxy-1H-benzo[e][1,4]diazepin-3-yl)hexanoic acid (Compound 87a)

6-Phenoxy-1H-benzo[d][1,3]oxazine-2,4-dione 86 (320 mg, 1.25 mmol) was reacted with DL-2-aminooctanedioic acid (237 mg, 1.25 mmol) and triethylamine (383 uL, 2.75 mmol) in H₂O (10 mL) in a manner similar to Scheme 11, step 1, Example 84. After Cyclization in acetic acid and work-up the title Compound 87a was obtained in 25% yield as a brown solid. ¹H NMR: (DMSO-d₆) δ (ppm): 11.93 (s, 1H), 10.3 (s, 1H), 8.47 (d, J=5.7 Hz, 1H), 7.4 (m, 2H), 7.24-7.01 (m, 6H), 3.6 (m, 1H), 2.18 (t, J=7.2 Hz, 2H), 1.72 (m, 1H), 1.6-1.2 (m, 7H).

### Step 5: 6-(2,3,4,5-Tetrahydro-2,5-dioxo-7-phenoxy-1H-benzo[e][1,4]diazepin-3-yl)-N-hydroxyhexanamide (Compound 88a)

The title Compound 88a was obtained in 33% yield as a white solid according to method D, Scheme 10, step 4, Example 69. ¹H NMR: (DMSO-d₆) δ (ppm): 10.23 (s, 1H), 10.2 (s, 1H), 8.55 (s, 1H), 8.39 (d, J=5.7 Hz, 1H), 7.32 (t, J=8.4, 2H), 7.16-6.94 (m, 6H), 3.51 (m, 1H), 1.83 (t, J=7.4 Hz, 2H), 1.65 (m, 1H), 1.52-1.1(m, 7H). LRMS (ESI): (calc.) 397.1; (found) 398.3.

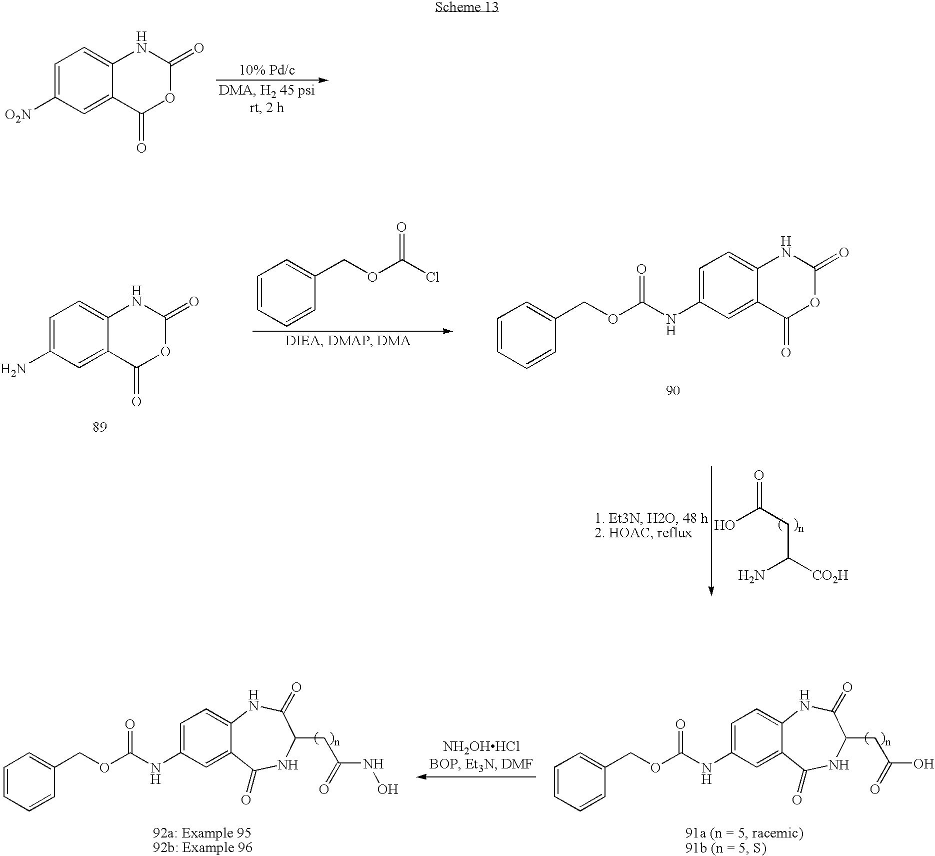

### EXAMPLE 95

### 6-(7-Benzyloxycarbonylamino-2,3,4,5-tetrahydro-2,5-dioxo-1H-benzo[e][1,4]diazepin-3-yl)-N-hydroxyhexanamide (Compound 92a)

### Step 1: 6-Amino-1H-benzo[d][1,3]oxazine-2,4-dione1 (Compound 89)

5-Nitroisatoic anhydride (500 mg, 2.4 mmol), 10% Pd/C (20 mg) in DMA (10 mL) was reduced with H₂ gas (45 psi). After 24 h, the starting material was consumed and the catalyst was filtered over a pad of Celite and crude 89 was used for the next step.

### Step 2: Benzyl 2,4-dihydro-2,4-dioxo-1H-benzo[d][1,3]oxazin-6-ylcarbamate (Compound 90)

DIEA (452 uL, 2.59 mmol) and DMAP (10 mg, 0.0816 mmol) were added to crude 89 in DMA and the mixture was cooled to −10° C. in a salt-ice bath. Benzoylchloroformate (357 μL, 2.5 mmol) was added dropwise and the mix was allowed to warm-up to room temperature o/n. The solvent was removed under reduced pressure, and the crude was loaded on flash silica and eluted with 1:1 EtOAc/hex to 100% EtOAc. The desired fractions were concentrated and the residue was triturated from minimum amount of EtOAc/ether to give the title Compound 90 as beige solid (298 mg, 40%). ¹H NMR: (DMSO-d₆) δ (ppm): 11.61 (bs, 1H), 10.0 (bs, 1H), 8.05 (bs, 1H), 7.73 (dd, J=8.6, 2.2 Hz, 1H), 7.36 (m, 5H), 7.08 (d, J=8.8 Hz, 1H), 5.15 (s, 2H).

### Step 3: 6-(7-Benzyloxycarbonylamino-2,3,4,5-tetrahydro-2,5-dioxo-1H-benzo[e][1,4]diazepin-3-yl)hexanoic acid (Compound 91a)

Compound 90 (120 mg, 0.38 mmol) was reacted with DL-2-aminooctanedioic acid (79.6 mg, 0.38 mmol) and triethylamine (115 uL, 0.83 mmol) in H₂O (1.5 mL) in a manner similar to Scheme 11, Example 88, step 1. After Cyclization in acetic acid and work-up and chromatography over flash Silica eluting with EtOAc, the title Compound 91a was obtained in 30% yield as a beige solid. ¹H NMR: (DMSO-d₆) δ (ppm): 11.93 (bs, 1H), 10.17 (s, 1H), 9.87 (bs, 1H), 8.39 (d, J=5.5 Hz, 1H), 7.8 (s, 1H), 7.55 (dd, J=2, 6.5 Hz, 1H), 7.41-7.3 (m, 4H), 6.98 (d, J=8.8 Hz, 1H), 5.13 (s, 2H), 3.53 (m, 1H), 2.16 (t, J=7.2 Hz, 2H), 1.69 (m, 1H), 1.6-1.1 (m, 7H).

### Step 4: 6-(7-Benzyloxycarbonylamino-2,3,4,5-tetrahydro-2,5-dioxo-1H-benzo[e][1,4]diazepin-3-yl)-N-hydroxyhexanamide (Compound 92a)

Compound 91 (38 mg, 0.087 mmol), was stirred with BOP ((42 mg, 0.095 mmol), DIEA (60.3 uL, 0.35 mmol), and hydroxylamine hydrochloride (6.6 mg, 0.095 mmol) in DMF (2 mL) following method D, Example 69, Scheme 10, step 4. The title Compound was purified on flash Silica eluting with 75% EtOAc/hexanes with few drops of acetic acid. The fractions were combined and concentrated and the residue was triturated from acetonitrile, then from ether to remove the last traces of acetic acid. The desired hydroxamic acid 92a was obtained as an off-white solid (17.5 mg, 44%). ¹H NMR: (DMSO-d₆) δ (ppm): 10.26 (bs, 1H), 10.18 (s, 1H), 9.87 (s, 1H), 8.62 (bs, 1H), 8.38 (d, J=5.9 Hz, 1H), 7.8 (d, J=2.3 Hz, 1H), 7.55 (dd, J=2.5, 8.8 Hz, 1H), 7.41-7.3 (m, 4H), 6.98 (d, J=8.8 Hz, 1H), 5.13 (s, 2H), 3.52 (m, 1H), 1.9 (t, J=7.4 Hz, 2H), 1.7 (m, 1H), 1.6-1.2 (m, 7H). LRMS (ESI): (calc.) 454.5; (found) 455.3.

### EXAMPLE 96

### (S)-Benzyl 3-(6-(hydroxyamino)-6-oxohexyl)-2,5-dioxo-2,3,4,5-tetrahydro-1H-benzo[e][1,4]diazepin-7-ylcarbamate (Compound 92b)

Following the procedure described above for Compound 92a, the title Compound 92b was obtained. ¹H NMR: (DMSO-d₆) δ (ppm): 10.3 (s, 1H), 10.2 (s, 1H), 9.90 (s, 1H), 8.65 (s, 1H), 8.41 (d, J=5.6 Hz, 1H), 7.82 (d, J=2.4 Hz, 1H), 7.56 (dd, J=2.8, 8.8 Hz, 1H), 7.42-7.32 (m, 5H), 6.99 (d, J=8.8 Hz, 1H), 5.14 (s, 2H), 3.52 (dq, J=2.0, 8.0 Hz, 1H), 1.89 (t, J=7.6 Hz, 2H), 1.70 (m, 1H), 1.60-1.10 (m, 7H). LRMS (ESI): (calc.) 454.19; (found) 455.1 (MH)⁺.

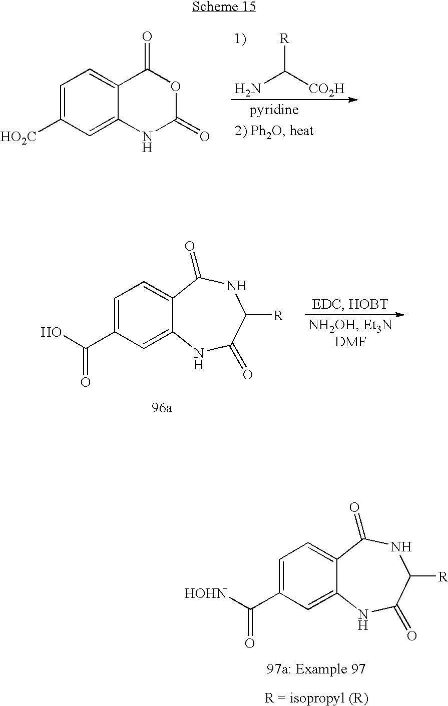

### EXAMPLE 97

(R)—N-Hydroxy-3-isopropyl-2,5-dioxo-2,3,4,5-tetrahydro-1H-benzo[e][1,4]diazepine-8-carboxamide (Compound 97a)

### Step 1: (R)-3-Isopropyl-2,5-dioxo-2,3,4,5-tetrahydro-1H-benzo[e][1,4]diazepine-8-carboxylic acid (Compound 96a)

According to the procedure of Clark et al. (Clark, A. S.; Deans, B.; Stevens, M. F. G.; Tisdale, M. J.; Whelihouse, R. T.; Denny, B. J.; Hartley, J. A. J. Med. Chem. 1995, 38, 1493-1504), 7-Carboxyisatoic anhydride (150 mg, 0.72 mmol) and D-Valine (94 mg, 0.80 mmol) in dry pyridine (2.0 mL) were heated at 100° C. for 16 h under nitrogen. The solution was evaporated and phenyl ether (1.5 mL) was added. The heterogenous mixture was heated at 180° C. for 1 h. The acid 96a, was precipitated by the addition of hexane, filtered, and washed with hexane. The crude acid was dried and used without further purification.

### Step 2: (R)—N-Hydroxy-3-isopropyl-2,5-dioxo-2,3,4,5-tetrahydro-1H-benzo[e][1,4]diazepine-8-carboxamide (Compound 97a)

To the acid 96a (184 mg, 0.66 mmol) in DMF (12.1 mL) was added 1-hydroxybenzotriazole hydrate (225 mg, 1.67 mmol) and 1-[3-(dimethylamino)propyl]-3-ethylcarbodiimide hydrochloride (357 mg, 1.86 mmol). The solution was stirred for 1 h at rt. Hydroxylamine hydrochloride (364 mg, 5.31 mmol) and triethylamine (0.93 mL) were added and the mixture was stirred for 16 h at rt. The solution was evaporated and the crude residue was purified by flash chromatography on silica gel, eluting with 0-20% MeOH in CH₂Cl₂. The partially purified hydroxamic acid was then purified by preparative HPLC to afford Compound 97a (9 mg, 5% yield over three steps):

### EXAMPLES 98-107

Examples 98-107 were prepared using the same procedures as described for Compound 97, Scheme 15, Example 97. Characterization data are presented in Table 10.

TABLE 10

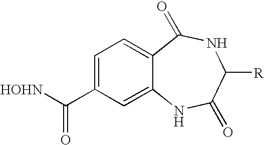

| Ex | Cpd | R | Name | Characterization | Scheme |
|---|---|---|---|---|---|
| 98 | 97b | 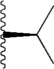 | (S)-N-Hydroxy- 3-isopropyl-2,5- dioxo-2,3,4,5- tetrahydro-1H- benzo[e][1,4]diazepine-8- carboxamide | ¹H NMR: (DMSO-d₆) δ (ppm): 11.29 (bs, 1H), 10.45 (s, 1H), 9.18 (bs, 1H), 8.64 (d, J=6.5 Hz, 1H), 7.75 (d, J=8.0 Hz, 1H), 7.47 (d, J=8.8 Hz, 2H), 3.33-3.22 (m, 1H), 1.94 (bs, 1H), 0.93 (t, J=6.5 Hz, 3H), 0.89 (t, J=6.5 Hz, 3H). LRMS (ESI): (calc.) 277.1; (found) 278.2 (MH)⁺. | 15 |
| 99 | 97c | 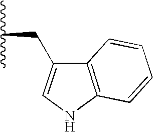 | (S)-3-((1H-Indol- 3-yl)methyl)-N- hydroxy-2,5- dioxo-2,3,4,5- tetrahydro-1H- benzo[e][1,4]diazepine-8- carboxamide | ¹H NMR: (DMSO-d₆) δ (ppm): 11.29 (bs, 1H), 10.82 (s, 1H), 10.51 (s, 1H), 9.15 (bs, 1H), 8.51 (d, J=5.9 Hz, 1H), 7.66 (d, J=8.0 Hz, 1H), 7.47-7.43 (m, 3H), 7.29 (d, J=8.0 Hz, 1H), 7.21 (s, 1H), 7.00 (t, J=7.4 Hz, 1H), 6.87 (t, J=7.4 Hz, 1H), 3.91-3.87 (m, 1H), 3.22 | |
| # (dd, J=14.9, 4.9 Hz, 1H), 2.98 (dd, J=14.7, 9.4 Hz, 1H). LRMS (ESI): (calc.) 364.1; (found) 365.2 (MH)⁺. | 15 | | | | |
| 100 | 97d | 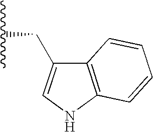 | (R)-3-((1H- Indol-3- yl)methyl)-N- hydroxy-2,5- dioxo-2,3,4,5- tetrahydro-1H- benzo[e][1,4]diazepine-8- carboxamide | ¹H NMR: (DMSO-d₆) δ (ppm): 11.29 (bs, 1H), 10.82 (s, 1H), 10.51 (s, 1H), 9.15 (bs, 1H), 8.51 (d, J=5.9 Hz, 1H), 7.66 (d, J=8.0 Hz, 1H), 7.47-7.43 (m, 3H), 7.29 (d, J=8.0 Hz, 1H), 7.21 (s, 1H), 7.00 (t, J=7.4 Hz, 1H), 6.87 (t, J=7.4 Hz, 1H), 3.91-3.87 (m, 1H), 3.22 | |
| # (dd, J=14.9, 4.9 Hz, 1H), 2.98 (dd, J=14.7, 9.4 Hz, 1H). LRMS (ESI): (calc.) 364.1; (found) 365.1 (MH)⁺. | 15 | | | | |
| 102 | 97f | 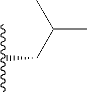 | (R)-N-Hydroxy- 3-isobutyl-2,5- dioxo-2,3,4,5- tetrahydro-1H- benzo[e][1,4]diazepine-8- carboxamide | ¹H NMR: (DMSO-d₆) δ (ppm): 11.31 (bs, 1H), 10.45 (s, 1H), 9.16 (bs, 1H), 8.52 (d, J=5.5 Hz, 1H), 7.75 (d, J=8.2 Hz, 1H), 7.48 (d, J=7.4 Hz, 2H), 3.61 (q, J=6.7 Hz, 1H), 1.71-1.66 (m, 1H), 1.55 (t, J=6.8 Hz, 2H), 0.85 (d, J=6.5 Hz, 3H), 0.77 (d, J=6.7 Hz, 3H). | |
| # LRMS (ESI): (calc.) 291.1; (found) 292.1 (MH)⁺. | 15 | | | | |
| 103 | 97g | 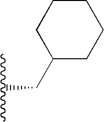 | (R)-3- (Cyclohexylmethyl)- N-hydroxy- 2,5-dioxo- 2,3,4,5- tetrahydro-1H- benzo[e][1,4]diazepine-8- carboxamide | ¹H NMR: (DMSO-d₆) δ (ppm): 11.31 (bs, 1H), 10.45 (s, 1H), 9.17 (bs, 1H), 8.51 (d, J=5.5 Hz, 1H), 7.75 (d, J=8.0 Hz, 1H), 7.48 (d, J=8.2 Hz, 2H), 3.66 (q, J=8.2 Hz, 1H), 1.61-1.55 (m, 7H), 1.41-1.33 (m, 1H), 1.18-1.06 (m, 3H), 0.92-0.76 (m, 2H). LRMS (ESI): | |
| # (calc.) 331.2; (found) 332.2 (MH)⁺. | 15 | | | | |
| 104 | 97h | 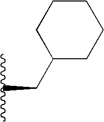 | (S)-3- (Cycbohexylmethyl)- N-hydroxy- 2,5-dioxo- 2,3,4,5- tetrahydro-1H- benzo[e][1,4]diazepine-8- carboxamide | ¹H NMR: (DMSO-d₆) δ (ppm): 11.31 (bs, 1H), 10.45 (s, 1H), 9.17 (bs, 1H), 8.51 (d, J=5.5 Hz, 1H), 7.75 (d, J=8.0 Hz, 1H), 7.48 (d, J=8.2 Hz, 2H), 3.66 (q, J=8.2 Hz, 1H), 1.61-1.55 (m, 7H), 1.41-1.33 (m, 1H), 1.18-1.06 (m, 3H), 0.92-0.76 (m, 2H). LRMS (ESI): | |
| # (calc.) 331.2; (found) 332.1 (MH)⁺. | 15 | | | | |
| 105 | 97i | 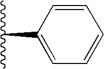 | (S)-N-Hydroxy- 2,5-dioxo-3- phenyl-2,3,4,5- tetrahydro-1H- benzo[e][1,4]diazepine-8- carboxamide | ¹H NMR: (MeOD-d₄) δ (ppm): 7.78 (d, J=8.4 Hz, 1H), 7.40-7.38 (m, 1H), 7.38 (s, 1H), 7.25-7.18 (m, 5H), 5.13 (s, 1H). LRMS (ESI): (calc.) 311.1; (found) 312.2 (MH)⁺. | 15 |
| 106 | 97j | 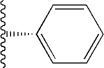 | (R)-N-Hydroxy- 2,5-dioxo-3- phenyl-2,3,4,5- tetrahydro-1H- benzo[e][1,4]diazepine-8- carboxamide | ¹H NMR: (MeOD-d₄) δ (ppm): 7.78 (d, J=8.4 Hz, 1H), 7.40-7.38 (m, 1H), 7.38 (s, 1H), 7.25-7.18 (m, 5H), 5.13 (s, 1H). LRMS (ESI): (calc.) 311.1; (found) 312.2 (MH)⁺. | 15 |
| 107 | 97k | H | N-Hydroxy-2,5- dioxo-2,3,4,5- tetrahydro-1H- benzo[e][1,4]diazepine-8- carboxamide | ¹H NMR: (D₂O) δ (ppm): 7.90 (d, J=8.2 Hz, 1H), 7.58 (d, J=8.2 Hz, 1H), 7.45 (s, 1H), 3.84 (s, 2H). LRMS (ESI): (calc.) 235.1; (found) 236.1 (MH)⁺. | 15 |

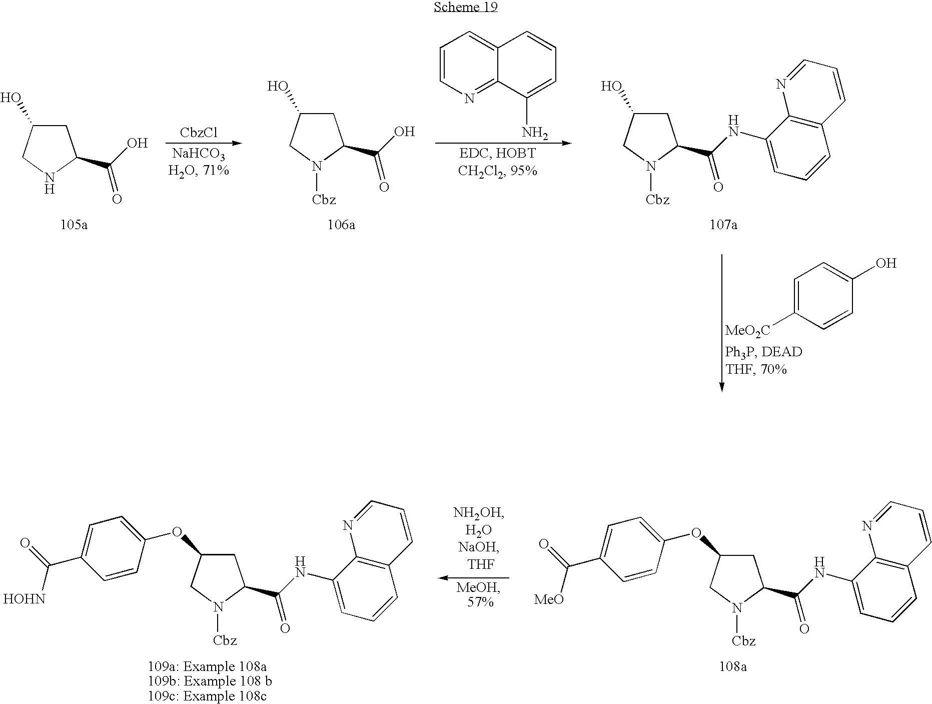

### EXAMPLE 108a

(2S,4S)-Benzyl 4-(4-(hydroxycarbamoyl)phenoxy)-2-(quinolin-8-ylcarbamoyl)pyrrolidine-1-carboxylate (Compound 109a)

### Step 1: (2S,4R)-1-(Benzyloxycarbonyl)-4-hydroxypyrrolidine-2-carboxylic acid (Compound 106a)

Sodium hydrogencarbonate (16.0 g, 191 mmol) was added to a stirred solution of trans-4-hydroxy-L-proline (105a) (10.0 g, 76 mmol) in water (8.7 mL) at 0° C. Benzyl chloroformate (12 mL, 84 mmol) was added and the mixture was stirred for 1 h at 0° C., followed by 1.5 h at rt. The mixture cooled to 0° C. was acidified to pH 2 with concentrated HCl, and extracted with EtOAc three times. The organic layer were washed with brine, dried with MgSO₄, and filtered. The solution was evaporated and the crude residue was purified by flash column chromatography on silica gel, using gradient from 0-20% MeOH/CH₂Cl₂ to afford the title Compound 106a (14.4 g, 71% yield): LRMS (ESI): (calc.) 265.3; (found) 288.1 (M+Na)⁺.

### Step 2: (2S,4R)-benzyl 4-hydroxy-2-(quinolin-8-ylcarbamoyl)pyrrolidine-1-carboxylate (Compound 107a)

8-Aminoquinoline (681 mg, 4.73 mmol) was added to a stirred solution of Compound 106a (835 mg, 3.15 mmol) in CH₂Cl₂ (6.8 mL). The solution was cooled to 0° C., and 1-hydroxybenzotriazole hydrate (468 mg, 3.47 mmol) was added. The mixture was stirred 5 min, then 1-[3-(dimethylamino)propyl]-3-ethylcarbodiimide hydrochloride (665 mg, 3.47 mmol) was added. The mixture was stirred 10 min, followed by 16 h at rt. The solvent was evaporated, EtOAc and sat. aq. NaHCO₃ were added. The aqueous layer was extracted with EtOAc two times. The organic phases were washed with brine, dried with MgSO₄, and filtered. The solution was evaporated and the crude residue was purified by flash column chromatography on silica gel, using gradient from 40% to 80% EtOAc in hexane to afford the title Compound 107a (1.18 g, 95% yield): ¹H NMR: (DMSO-d₆) δ (ppm): 10.36 (d, J=9.6 Hz, 1H), 8.86 (bs, 1H), 8.58 (t, J=6.4 Hz, 1H), 8.40 (d, J=7.2 Hz, 1H), 7.68 (d, J=8.0 Hz, 1H), 7.64-7.55 (m, 2H), 7.35-7.28 (m, 2H), 7.08 (d, J=7.6 Hz, 1H), 6.79 (t, J=7.2 Hz, 1H), 5.15-5.07 (m, 2H), 4.72 (dt, J=31.2, 7.2 Hz, 1H), 4.35 (bs, 1H), 3.63-3.57 (m, 1H), 3.51 (d, J=11.2 Hz, 1H), 2.38-2.22 (m, 1H), 2.13-2.08 (m, 1H). LRMS (ESI): (calc.) 391.2; (found) 392.2 (MH)⁺.

### Step 3: (2S,4S)-Benzyl 4-(4-(methoxycarbonyl)phenoxy)-2-(quinolin-8-ylcarbamoyl)pyrrolidine-1-carboxylate (Compound 108a)

Triphenylphosphine (144 mg, 0.55 mmol) was added to a stirred solution of Compound 107a (196 mg, 0.50 mmol) in THF (5 mL) at 0° C. Methyl 4-hydroxybenzoate (80 mg, 0.53 mmol) was added, followed by diethyl azodicarboxylate (86 μL, 0.55 mmol). The mixture was allowed to warm-up to room temperature slowly and stirred 16 h at rt. The solvent was evaporated, and the crude residue was purified by flash column chromatography on silica gel, using gradient from 20% to 60% EtOAc in hexane to afford the title Compound 108a (184 mg, 70% yield): LRMS (ESI): (calc.) 525.2; (found) 526.2 (MH)⁺.

### Step 4: (2S,4S)-Bbenzyl 4-(4-(hydroxycarbamoyl)phenoxy)-2-(quinolin-8-ylcarbamoyl)pyrrolidine-1-carboxylate (Compound 109a)

Hydroxylamine (0.5 mL, 50% in water) was added to a stirred solution of Compound 108a (42 mg, 0.08 mmol) in THF (0.25 mL) and methanol (0.25 mL). Sodium hydroxide (26 mg, 0.64 mmol) was added, and the mixture was stirred for 1.25 h. The solvent was evaporated, and the residue purified by preparative reverse phase HPLC (aquasil C-18, 100×4.6, 5 uM) with MeOH/H₂O to afford the title Compound 109a (24 mg, 57% yield). LRMS (ESI): (calc.) 526.2; (found); 527.7 (MH)⁺. ¹H NMR: (DMSO-d₆) δ (ppm): 10.94 (bs, 1H), 10.54 (d, J=14.5 Hz, 1H), 8.86 (s, 1H), 8.76 (d, J=19.4 Hz, 1H), 8.60 (d, J=7.6 Hz, 1H), 8.42 (d, J=8.0 Hz, 1H), 7.69 (d, J=8.0 Hz, 1H), 7.63-7.56 (m, 2H), 7.49 (bs, 2H), 7.39 (s, 1H), 7.30 (s, 1H), 7.09 (s, 1H), 6.90-6.83 (m, 1H), 6.68-6.64 (m, 2H), 5.27-5.21 (m, 2H), 4.74-4.61 (m, 1H), 3.97-3.78 (m, 2H), 2.78-2.68 (m, 1H), 2.50-2.33 (m, 2H).

### EXAMPLES 108b AND 108c

Examples 108b and 108c describe the preparation of Compound 109b and 109c using the same procedures as described for Compound 109a in Example 108a. Characterization data are presented in Table 11.

TABLE 11

| Ex | Cpd | Structure | Name | Characterizaiton |
|---|---|---|---|---|
| 108b | 109b | 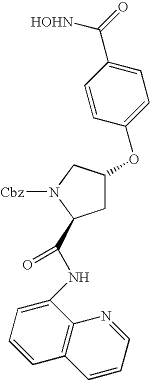 | (2S,4R)- Benzyl 4-(4- (hydroxycarbamoyl) phenoxy)- 2-(quinolin-8- ylcarbamoyl) pyrrolidine-1- carboxylate | ¹H NMR: (DMSO-d₆) δ (ppm): 11.08 (bs, 1H), 10.49 (s, 1H), 8.97-8.89 (m, 1H), 8.87 (d, J=9.6 Hz, 1H), 8.61 (t, J=6.7 Hz, 1H), 8.40 (d, J=9.0 Hz, 1H), 7.73 (d, J=8.4 Hz, 2H), 7.63-7.56 (m, 1H), 7.59 (d, J=8.0 Hz, 1H), 7.30 (s, 2H), 7.11 (d, J=7.3 Hz, |
| # 1H), 7.03 (d, J=8.2 Hz, 2H), 6.81 (t, J=7.2 Hz, 1H), 5.17 (s, 1H), 5.09 (s, 1H), 4.96-4.93 (m, 1H), 3.89-3.82 (m, 2H), 2.70-2.59 (m, 1H), 2.45-2.37 (m, 2H). LRMS (ESI): (calc.) 526.2; (found) 527.6 (MH)⁺. | | | | |
| 108c | 109c | 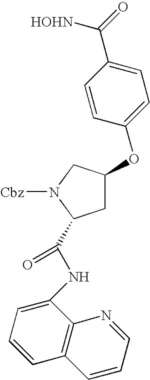 | (2R,4S)- Benzyl 4-(4-(hydroxycarbamoyl) phenoxy)- 2-(quinolin-8- ylcarbainoyl) pyrrolidine-1- carboxylate | ¹H NMR: (DMSO-d₆) δ (ppm): 11.08 (bs, 1H), 10.49 (s, 1H), 8.97-8.89 (m, 1H), 8.87 (d, J=9.6 Hz, 1H), 8.61 (t, J=6.7 Hz, 1H), 8.40 (d, J=9.0 Hz, 1H), 7.73 (d, J=8.4 Hz, 2H), 7.63-7.56 (m, 1H), 7.59 (d, J=8.0 Hz, 1H), 7.30 (s, 2H), 7.11 (d, J=7.3 Hz, |
| # 1H), 7.03 (d, J=8.2 Hz, 2H), 6.81 (t, J=7.2 Hz, 1H), 5.17 (s, 1H), 5.09 (s, 1H), 4.96-4.93 (m, 1H), 3.89-3.82 (m, 2H), 2.70-2.59 (m, 1H), 2.45-2.37 (m, 2H). LRMS (ESI): (calc.) 526.2; (found) 527.6 (MH)⁺. | | | | |

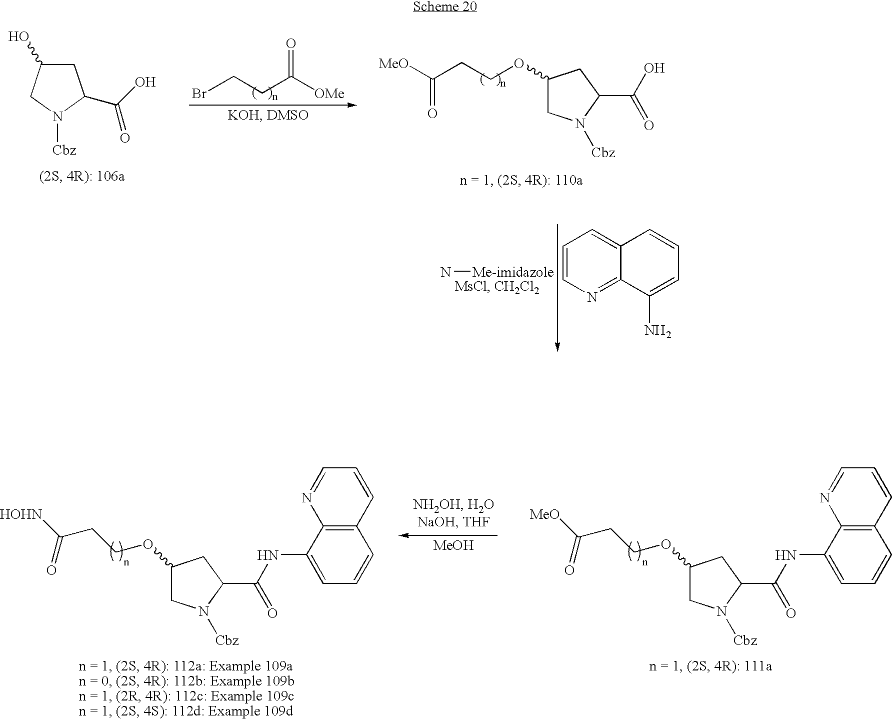

### EXAMPLE 109a

(2S,4R)-benzyl 4-(3-(hydroxyamino)-3-oxopropoxy)-2-(quinolin-8-ylcarbamoyl)pyrrolidine-1-carboxylate (Compound 112a)

### Step 1: (2S,4R)-1-(Benzyloxycarbonyl)-4-(3-methoxy-3-oxopropoxy)pyrrolidine-2-carboxylic acid (Compound 110a)

Potassium hydroxide (1.69 g, 30.2 mmol) was added to a stirred solution of Compound 106a (2.00 g, 7.54 mmol) in DMSO (10 mL). Methyl 3-bromopropionate (1.65 mL, 15.1 mmol) was added dropwise to the mixture, and then stirred for 16 h. Water and EtOAc were added, and concentrated HCl was added up to pH-3. The aqueous layer was extracted with EtOAc two times. The organic phases were dried with MgSO₄ and filtered. The solution was evaporated and the crude residue was purified by flash column chromatography on silica gel, using 60% EtOAc in hexane to afford the title Compound 110a (376 mg, 14% yield). LRMS (ESI): (calc.) 351.1; (found); 352.3 (MH)⁺.

### Step 2: (2S,4R)-Benzyl 4-(3-methoxy-3-oxopropoxy)-2-(quinolin-8-ylcarbamoyl)pyrrolidine-1-carboxylate (Compound 111a)

1-Methylimidazole (88 μL, 1.10 mmol) was added to a stirred solution of Compound 110 (176 mg, 0.50 mmol) in CH₂Cl₂ (2.5 mL). The solution was cooled to 0° C. and methanesulfonyl chloride (39 μL, 0.50 mmol) was added drop-wise. The reaction was allowed to reach room temperature, then 8-aminoquinoline (65 mg, 0.45 mmol) was added. The mixture was stirred at 45° C. for 16 h. The reaction was quenched with sat. aq. NH₄Cl, and extracted three times with CH₂Cl₂. The organic phases were dried with MgSO₄ and filtered. The solution was evaporated and the crude residue was purified by flash column chromatography on silica gel, using gradient from 10% to 60% EtOAc in hexane to afford the title Compound 111a (120 mg, 56% yield). LRMS (ESI): (calc.) 477.2; (found) 478.0 (MH)⁺.

### Step 3: (2S,4R)-Benzyl 4-(3-(hydroxyamino)-3-oxopropoxy)-2-(quinolin-8-ylcarbamoyl)pyrrolidine-1-carboxylate (Compound 112a)

Hydroxylamine (1.1 mL, 50% in water) was added to a stirred solution of Compound 111a (106 mg, 0.22 mmol) in THF (0.6 mL) and methanol (0.6 mL). Sodium hydroxide (89 mg, 2.2 mmol) was added, and the mixture was stirred for 1.5 h. The solvent was evaporated, and the residue purified by preparative reverse phase HPLC (aquasil C-18, 100×4.6, 5 uM) with MeOH in H₂O to afford the title Compound 112a (44 mg, 42% yield): ¹H NMR: (DMSO-d₆) δ (ppm): 10.39 (s, 1H), 8.90-8.84 (m, 1H), 8.77 (s, 1H), 8.62-8.58 (m, 1H), 8.40 (d, J=8.0 Hz, 1H), 7.70-7.54 (m, 3H), 7.37-7.28 (m, 2H), 7.08 (d, J=7.2 Hz, 1H), 6.78 (t, J=7.2 Hz, 1H), 5.12-5.05 (m, 2H), 4.71 (dt, J=29.6, 7.6 Hz, 1H), 4.15 (s, 1H), 3.70-3.56 (m, 4H), 2.21-2.10 (m, 4H). LRMS (ESI): (calc.) 478.2; (found) 479.3 (MH)⁺.

### EXAMPLES 109b, 109c AND 109d

Examples 109b, 109c and 109d describe the preparation of Compound 112b, 112c and 112d using the same procedure as described for Compound 112a in Example 108a. Characterization data are presented in Table 12.

TABLE 12

| Ex | Cpd | Structure | Name | Characterization |
|---|---|---|---|---|
| 109b | 112b | 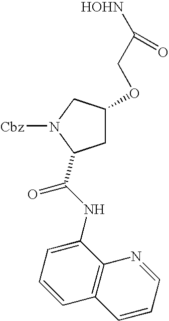 | (2S,4R)-benzyl 4-(2- (hydroxyamino)- 2-oxoethoxy)- 2-(quinolin-8- ylcarbainoyl) pyrrolidine-1- carboxylate | ¹H NMR: (CDCl₃) δ (ppm): 10.71 (s, 1H), 8.73 (s, 1H), 8.69 (t, J=4.5 Hz, 1H), 8.18 (d, J=8.0 Hz, 1H), 7.55 (d, J=3.7 Hz, 2H), 7.45-7.41 (m, 1H), 7.30-7.24 (m, 5H), 5.08-5.00 (m, 2H), 4.69-4.64 (m, 1H), 4.38-4.32 (m, 1H), 4.22 (d, J=5.3 Hz, 2H), 3.95 (d, |
| # J=11.7 Hz, 1H), 3.63 (dd, J=11.5, 4.8 Hz, 1H), 2.68-2.58 (m, 1H), 2.57-2.43 (m, 1H). LRMS (ESI): (calc.) 464.2; (found) 465.2 (MH)⁺. | | | | |
| 109c | 112c | 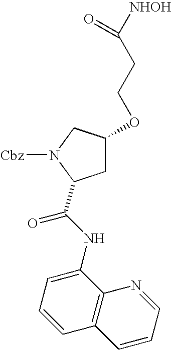 | (2R,4R)-benzyl 4-(3- (hydroxyamino)- 3- oxopropoxy)-2- (quinolin-8- ylcarbamoyl) pyrrolidine-1- carboxylate | ¹H NMR: (DMSO-d₆) δ (ppm): 10.45 (d, J=5.6 Hz, 1H), 10.24-10.22 (m, 1H), 8.85-8.82 (m, 1H), 8.66 (s, 1H), 1H), 7.29 (s, 1H), 7.07 (d, J=6.5 Hz, 8.61 (d, J=7.4 Hz, 1H), 8.39 (d, J=8.0 Hz, 1H), 7.66-7.54 (m, 3H), 7.37 (s, 1H), 6.83-6.78 (m, 1H), 5.23-4.94 (m, |
| # 2H), 4.54 (dd, J=26, 9.6 Hz, 1H), 4.16 (s, 1H), 3.68-3.56 (m, 5H), 2.22-2.18 (m, 1H), 2.05-1.98 (m, 2H). LRMS (ESI): (calc.) 478.2; (found) 479.3 (MH)⁺. | | | | |
| 190d | 112d | 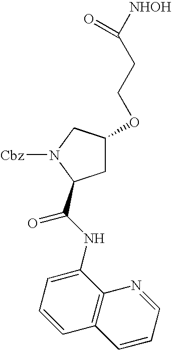 | (2S,4S)-benzyl 4-(3- (hydroxyamino)- 3- oxopropoxy)-2- (quinolin-8- ylcarbamoyl) pyrrolidine-1- carboxylate | ¹H NMR: (DMSO-d₆) δ (ppm): 10.45 (d, J=5.6 Hz, 1H), 10.24-10.22 (m, 1H), 8.85-8.82 (m, 1H), 8.66 (s, 1H), 8.61 (d, J=7.4 Hz, 1H), 8.39 (d, J=8.0 Hz, 1H), 7.66-7.54 (m, 3H), 7.37 (s, 1H), 7.29 (s, 1H), 7.07 (d, J=6.5 Hz, 1H), 6.83-6.78 (m, 1H), 5.23-4.94 (m, |
| # 2H), 4.54 (dd, J=26, 9.6 Hz, 1H), 4.16 (s, 1H), 3.68-3.56 (m, 5H), 2.22-2.18 (m, 1H), 2.05-1.98 (m, 2H). LRMS (ESI): (calc.) 478.2; (found) 479.3 (MH)⁺. | | | | |

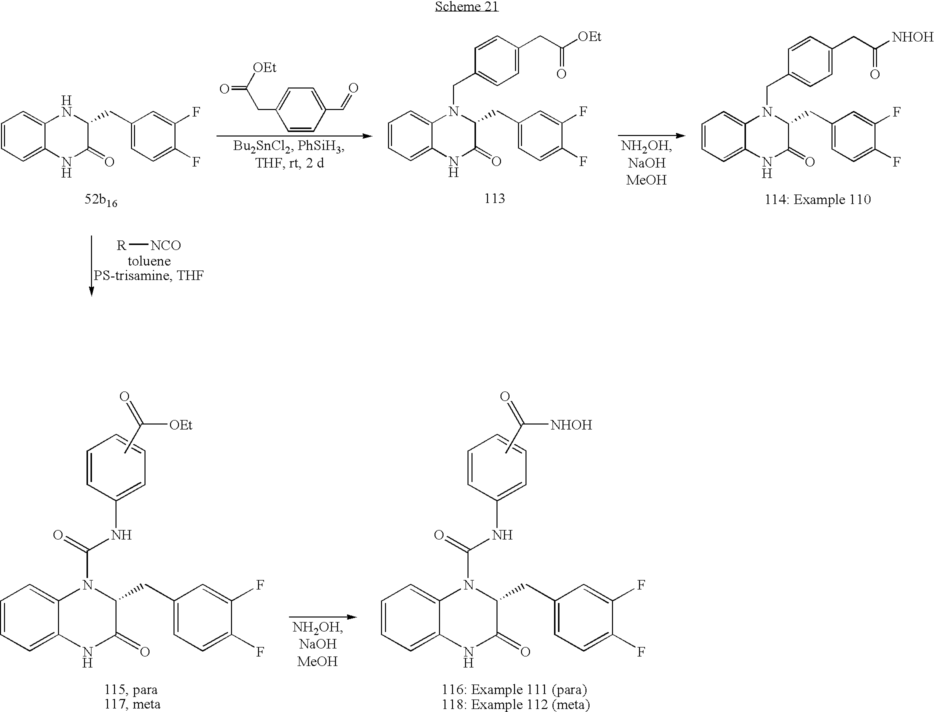

### EXAMPLE 110

(R)-2-(4-((2-(3,4-Difluorobenzyl)-3-oxo-3,4-dihydroquinoxalin-1(2H)-yl)methyl)phenyl)-N-hydroxyacetamide (Compound 114)

### Step 1: (R)-Ethyl 2-(4-((2-(3,4-difluorobenzyl)-3-oxo-3,4-dihydroquinoxalin-1(2H)-yl)methyl)phenyl)acetate (Compound 113)

Following the same procedure described in Example 16, step 3, Scheme 1, but substituting Compound 52b₁₆ for acid 3a, and ethyl 2-(4-formylphenyl)acetate (prepared according to the method of L. G. Goossen, Chem. Commun. 2001, 7, 669-670) for 4-methoxybenzaldehyde, compound 113 was isolated in 91% yield. LRMS (ESI): (calc.) 450.2; (found) 451.2 (MH)⁺.

### Step 2: (R)-2-(4-((2-(3,4-Difluorobenzyl)-3-oxo-3,4-dihydroquinoxalin-1(2H)-yl)methyl)phenyl)-N-hydroxyacetamide (Compound 114)

To a solution of 113 (260 mg, 0.58 mmol) in 1:1 THF/methanol (2.8 mL) was added a 50% wt solution of hydroxylamine in water (3 mL). Sodium hydroxide powder (185 mg, 4.6 mmol) was then added to the mixture. After stirring at room temperature for 15 min the reaction was concentrated under vacuum. The product was purified by flash chromatography eluting with 10% MeOH/CH₂Cl₂. Compound 114 was isolated as beige solid (42 mg, 17%). ¹H NMR: (DMSO-d₆) δ (ppm): 10.62 (s, 1H), 10.46 (s, 1H), 8.79 (s, 1H), 7.24-7.08 (m, 6H), 6.87-6.84 (m, 1H), 6.76 (t, J=6.3 Hz, 1H), 6.69 (d, J=6.5 Hz, 1H), 6.60 (t, J=6.8 Hz, 2H), 4.51 (d, J=15.3 Hz, 1H), 4.20 (d, J=15.5 Hz, 1H), 4.07 (t, J=6.5 Hz, 1H), 3.21 (s, 2H), 2.81 (dd, J=13.7, 6.5 Hz, 1H), 2.73 (dd, J=13.7, 6.6 Hz, 1H). LRMS (ESI): (calc.) 437.2; (found) 438.2 (MH)⁺.

### EXAMPLE 111

(R)-2-(3,4-Difluorobenzyl)-N-(4-(hydroxycarbamoyl)phenyl)-3-oxo-3,4-dihydroquinoxaline-1(2H)-carboxamide (Compound 116)

### Step 1: (R)-Ethyl 4-(2-(3,4-difluorobenzyl)-3-oxo-1,2,3,4-tetrahydroquinoxaline-1-carboxamido)benzoate (Compound 115)

To a solution of 52b₁₆ (137 mg, 0.50 mmol) in toluene (5 mL) was added ethyl 4-isocyanatobenzoate (478 mg, 2.5 mmol). The reaction was stirred at 80° C. for 16 h, then THF (5 mL) was added and the reaction was stir at 70° C. for 30 min. Polymer supported-trisamine (1.11 g, 4.0 mmol) was added and the mixture was stirred for 30 min at room temperature, then it was filtrated, and concentrated. The product was then purified by flash chromatography eluting with 60% AcOEt/hexane. Compound 115 was isolated (165 mg, 71%). LRMS (ESI): (calc.) 465.1; (found) 466.1 (MH)⁺.

### Step 2: (R)-2-(3,4-Difluorobenzyl)-N-(4-(hydroxycarbamoyl)phenyl)-3-oxo-3,4-dihydroquinoxaline-1(2H)-carboxamide (Compound 116)

Following the same procedure described in Example 110, step 2, Scheme 21, but substituting compound 115 for compound 113, compound 116 was isolated as a white solid in 34% yield. ¹H NMR: (DMSO-d₆) δ (ppm): 11.04 (bs, 1H), 10.83 (s, 1H), 9.07 (s, 1H), 8.91 (bs, 1H), 7.62 (d, J=8.8 Hz, 2H), 7.36 (d, J=8.8 Hz, 2H), 7.37-7.32 (m, 1H), 7.26 (dt, J=11, 8.4 Hz, 1H), 7.19-7.13 (m, 1H), 7.13 (t, J=6.1 Hz, 1H), 7.05 (t, J=7.8 Hz, 1H), 6.98 (d, J=7.8 Hz, 1H), 6.90-6.87 (m, 1H), 4.99 (dd, J=8.8, 5.7 Hz, 1H), 2.87 (dd, J=13.7, 5.5 Hz, 1H), 2.64 (dd, J=13.8, 9.0 Hz, 1H). LRMS (ESI): (calc.) 452.1; (found) 453.1 (MH)⁺.

### EXAMPLE 112

(R)-2-(3,4-difluorobenzyl)-N-(3-(hydroxycarbamoyl)phenyl)-3-oxo-3,4-dihydroquinoxaline-1(2H)-carboxamide (Compound 118)

### Step 1: (R)-Ethyl 3-(2-(3,4-difluorobenzyl)-3-oxo-1,2,3,4-tetrahydroquinoxaline-1-carboxamido)benzoate (compound 117)

Following the same procedure described in Example 111, step 1, Scheme 21, but substituting ethyl 3-isocyanatobenzoate for ethyl 4-isocyanatobenzoate, compound 117 was isolated in 60% yield. LRMS (ESI): (calc.) 465.1; (found) 466.1 (MH)⁺.

### Step 2: (R)-2-(3,4-difluorobenzyl)-N-(3-(hydroxycarbamoyl)phenyl)-3-oxo-3,4-dihydroquinoxaline-1(2H)-carboxamide (Compound 118)

Following the same procedure described in Example 110, step 2, Scheme 21, but substituting compound 117 for compound 114, compound 118 was isolated in 7% yield. ¹H NMR: (DMSO-d₆) δ (ppm): 11.13 (bs, 1H), 10.81 (s, 1H), 9.00 (s, 2H), 7.73 (s, 1H), 7.47 (d, J=7.2 Hz, 1H), 7.35 (d, J=7.8 Hz, 1H), 7.31-7.22 (m, 3H), 7.19-7.16 (m, 1H), 7.13 (t, J=7.6 Hz, 1H), 7.06 (t, J=7.6 Hz, 1H), 6.97 (d, J=7.8 Hz, 1H), 6.92-6.88 (m, 1H), 5.00 (dd, J=8.6, 5.7 Hz, 1H), 2.86 (dd, J=13.7, 5.9 Hz, 1H), 2.65 (dd, J=13.7, 8.6 Hz, 1H). LRMS (ESI): (calc.) 452.1; (found) 453.1 (MH)⁺.

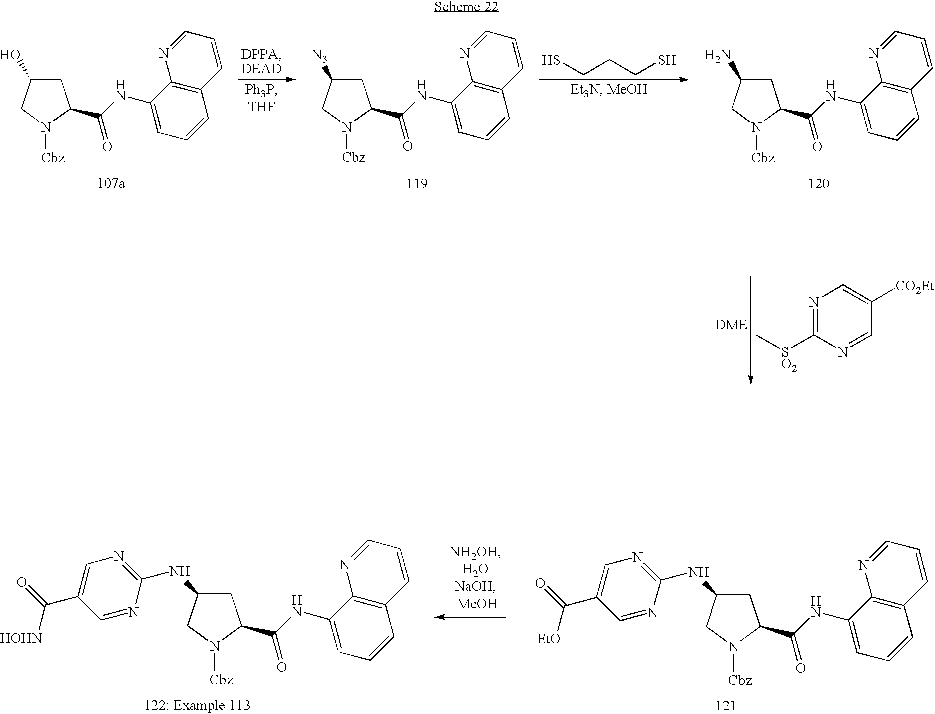

### EXAMPLE 113

(2S,4S)-Benzyl 4-(5-(hydroxycarbamoyl)pyrimidin-2-ylamino)-2-(quinolin-8-ylcarbamoyl)pyrrolidine-1-carboxylate (Compound 122)

### Step 1: (2S,4S)-Benzyl 4-azido-2-(quinolin-8-ylcarbamoyl)pyrrolidine-1-carboxylate (Compound 119)

To a solution of compound 107a (500 mg, 1.28 mmol) in THF (13 mL) was added triphenylphosphine (402 mg, 1.54 mmol). The solution was cooled to 0° C., and diethyl azodicarboxylate (0.26 mL, 1.66 mmol) was added, followed by diphenylphosphoryl azide (0.33 mL, 1.54 mmol). The reaction was allowed to warm slowly to room temperature over 1 h, and it was stirred for an additional 16 h. The solution was evaporated and the crude residue was purified by flash column chromatography on silica gel, eluting with a gradient of 20-40% AcOEt/hexane to afford compound 119 (409 mg, 77%): LRMS (ESI): (calc.) 416.2; (found) 417.2 (MH)⁺.

### Step 2: (2S,4S)-Benzyl 4-amino-2-(quinolin-8-ylcarbamoyl)pyrrolidine-1-carboxylate (Compound 120)

Triethylamine (0.69 mL, 4.92 mmol) and 1,3-propanedithiol (0.49 mL, 4.92 mmol) were added to a stirred solution of Compound 119 (409 mg, 0.98 mmol) in MeOH (6.8 mL) at 0° C. The mixture was stirred for 16 h at room temperature. The solvent was evaporated, EtOAc and sat. aq. NaHCO₃ were added. The aqueous layer was extracted twice with EtOAc. The organic extracts were washed with brine, dried with MgSO₄, and filtered. The solution was evaporated and the crude residue was purified by flash column chromatography on silica gel, using a gradient of 0-10% MeOH/CH₂Cl₂ to afford compound 120 (250 mg, 65%): LRMS (ESI): (calc.) 390.2; (found) 391.2 (MH)⁺.

### Step 3: (2S,4S)-Benzyl 4-(4-(methoxycarbonyl)phenoxy)-2-(quinolin-8-ylcarbamoyl)pyrrolidine-1-carboxylate (Compound 121)

Ethyl 2-(methylsulfonyl)pyrimidine-5-carboxylate (49 mg, 0.21 mmol) was added to a solution of compound 120 (83 mg, 0.21 mmol) in ethylene glycol dimethyl ether (1.0 mL). The mixture was stirred at 80° C. for 16 h. The reaction was quenched with sat. aq. NaHCO₃ and the aqueous layer was extracted with EtOAc three times. The organic extracts were dried with MgSO₄, filtered, and evaporated to give crude 121. LRMS (ESI): (calc.) 540.2; (found) 541.3 (MH)⁺.

### Step 4: (2S,4S)-Benzyl 4-(5-(hydroxycarbamoyl)pyrimidin-2-ylamino)-2-(quinolin-8-ylcarbamoyl)pyrrolidine-1-carboxylate (Compound 122)

Following the same procedure described in Example 108a, step 4, Scheme 19, but substituting compound 121 for compound 108a, compound was isolated as a beige solid in 15% yield. ¹H NMR: (DMSO-d₆) δ (ppm): 11.04 (bs, 1H), 10.46 (d, J=14 Hz, 1H), 9.02 (bs, 1H), 8.88 (bs, 1H), 8.72-8.58 (m, 3H), 8.42 (dd, J=8.2, 1.6 Hz, 1H), 7.98 (t, J=7.0 Hz, 1H), 7.70 (d, J=7.8 Hz, 1H), 7.67-7.58 (m, 2H), 7.42-7.28 (m, 2H), 7.11 (d, J=7.4 Hz, 1H), 6.96 (t, J=7.2 Hz, 1H), 6.81 (t, J=7.4 Hz, 1H), 5.10 (s, 1H), 5.09-4.93 (m, 1H), 4.85-4.73 (m, 1H), 4.60-4.49 (m, 1H), 4.00 (t, J=8.4 Hz, 1H), 3.38-3.33 (m, 1H), 2.77-2.66 (m, 1H), 2.15-2.08 (m, 1H). LRMS (ESI): (calc.) 527.2; (found) 528.3 (MH)⁺.

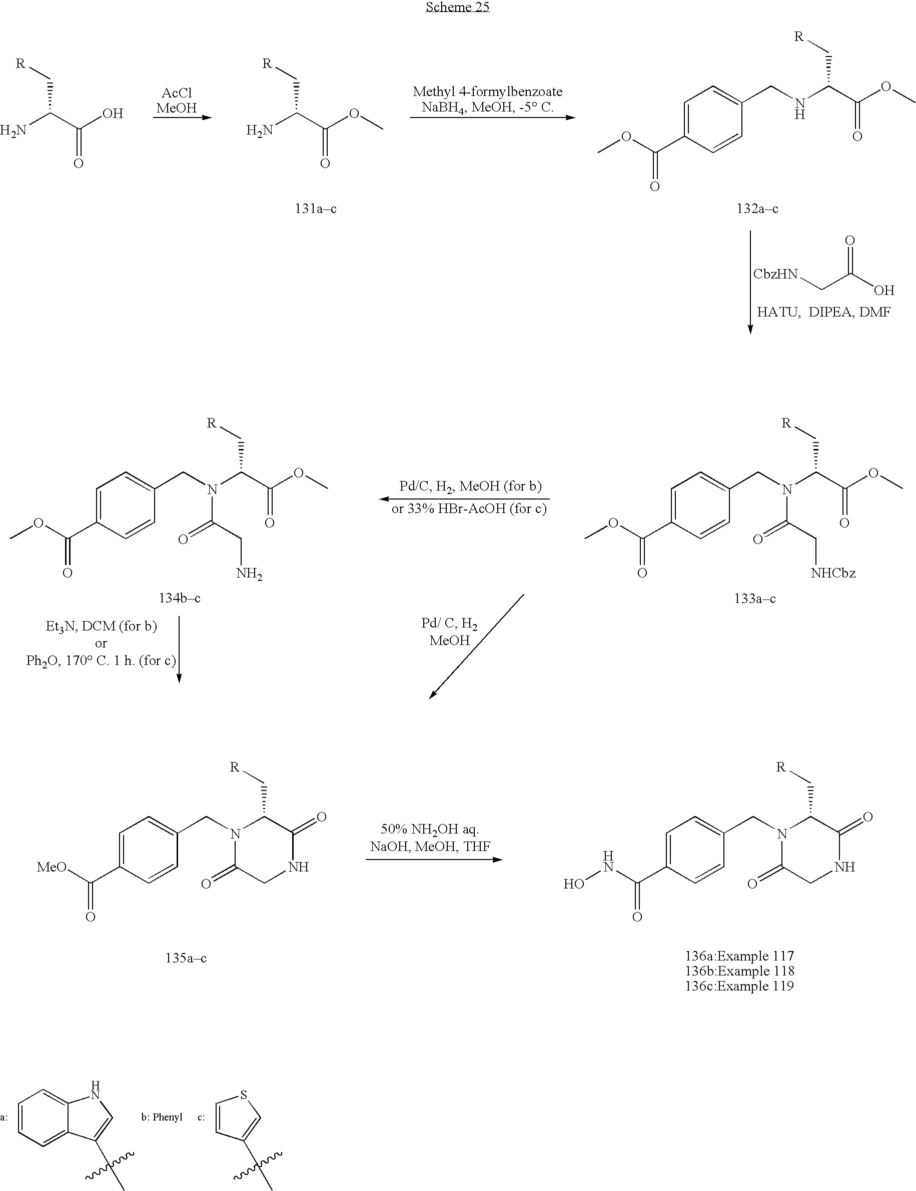

### EXAMPLE 117

(R)-4-((2-((1H-Indol-3-yl)methyl)-3,6-dioxopiperazin-1-yl)methyl)-N-hydroxybenzamide (Compound 136a)

### Step 1: (R)-Methyl 4-((3-(1H-indol-3-yl)-1-methoxy-1-oxorpropan-2-ylamino)methyl)-benzoate (Compound 132a)

(R)-Methyl 2-amino-3-(1H-indol-3-yl)propanoate hydrochloride (2.4 g, 9.4 mmol) was dissolved in DCM, washed with 10% NH₄OH_{(aq)}, dried over sodium sulfate and concentrated. The free amine 131a obtained was dissolved in MeOH (10 mL) with methyl 4-formylbenzoate (1.2 g, 11.3 mmol) and stirred for 3 h at room temperature. NaBH₄ (1.1 g, 10.3 mmol) was then added at −5° C. and the reaction mixture was stirred over night in a −20° C. freezer. The reaction was quenched by adding ice and water, the MeOH was evaporated and the product was extracted with DCM (3×), dried over sodium sulfate and concentrated to afford the title compound 132a (1.4 g, 41%). LRMS (ESI): (calc.) 366.2; (found) 367.3 (MH)⁺.

### Step 2: (R)-Methyl 4-((N-(3-(1H-indol-3-yl)-1-methoxy-1-oxopropan-2-yl)-2-(benzyl-oxycarbonylamino)acetamido) methyl)benzoate (Compound 133a)

DIPEA (0.28 mL, 1.62 mmol) was added to a solution of HATU (0.62 g, 1.62 mmol) and 2-(benzyloxycarbonylamino)acetic acid (0.34 g, 1.62 mmol) in DMF (3 mL) at 0° C. The solution was stirred 10 min then the amine 132a (0.54 g, 1.47 mmol) was added and stirred over night at room temperature. The solution was diluted with AcOEt, washed with water, 1N HCl (×2), NaHCO_{3(aq)} and brine, dried over sodium sulfate and concentrated to afford the title compound 133a as a foam (0.8 g, 97%). LRMS (ESI): (calc.) 557.2; (found) 558.4 (MH)⁺.

### Step 3: (R)-Methyl 4-((2-((1H-indol-3-yl)methyl)-3,6-dioxopiperazin-1-yl)methyl)-benzoate (Compound 135a)

Following the procedure described in Example 1, 4a, step 2 (Scheme 1) but substituting Compound 133a for 2a, the title Compound 135a was obtained as a beige solid (0.23 g, 39%). LRMS (ESI): (calc.) 391.2; (found) 392.2 (MH)⁺.

### Step 4: (R)-4-((2-((1H-Indol-3-yl)methyl)-3,6-dioxopiperazin-1-yl)methyl)-N-hydroxy-benzamide (Compound 136a)

Following the same procedure described in Example 108a, step 4, Scheme 19, but substituting compound 135a for compound 108a, the title compound 136a was isolated as white powder (1 mg, 0.1%). LRMS: (calc) 392.2 (found) 393.1 (MH)⁺. ¹H NMR (MeOD-d₄) δ (ppm): 11.16 (s, 1H), 10.99 (s, 1H), 9.02 (s, 1H), 7.88 (d, J=3.3 Hz, 1H), 7.69 (d, J=8.0 Hz, 2H), 7.45 (d, J=7.8 Hz, 1H), 7.33 (d, J=8.0 Hz, 1H), 7.29 (d, J=8.0 Hz, 2H), 7.08-7.01 (m, 2H), 6.95 (t, J=7.5 Hz, 1H), 5.15 (d, J=15.1 Hz, 1H), 4.15-4.02 (m, 2H), 3.91 (t, J=4.3 Hz, 1H), 3.3-3.2 (m, 2H).

### EXAMPLE 118

(R)-4-((2-Benzyl-3,6-dioxopiperazin-1-yl)methyl)-N-hydroxybenzamide (Compound 136b)

### Step 1: (R)-Methyl 4-((1-methoxy-1-oxo-3-phenylpropan-2-ylamino)methyl)benzoate (Compound 132b)

Following the same procedure described in Example 117, step 1, Scheme 25, but substituting compound 131b for compound 131a, the title compound 132b was isolated as an oil (0.85 g, 49%). ¹H NMR (CDCl₃) δ (ppm): 8.00-7.88 (m, 2H), 7.39-7.05 (m, 7H), 4.01-3.75 (m, 2H), 3.90 (s, 3H), 3.66 (s, 3H), 3.65-3.55 (m, 1H), 3.10-3.00 (m, 2H).

### Step 2: (R)-Methyl 4-((2-(benzyloxycarbonylamino)-N-(1-methoxy-1-oxo-3-phenyl-propan-2-yl)acetamido)methyl)benzoate (Compound 133b)

Following the same procedure described in Example 117, step 2, Scheme 25, but substituting compound 132b, HATU and DIPEA for compound 132a, EDCI and HOBt, the title compound 133b was isolated as an oil (0.7 g, 54%). LRMS (ESI): (calc.) 518.2; (found) 519.6 (MH)⁺.

### Step 3: (R)-Methyl 4-((2-amino-N-(1-methoxy-1-oxo-3-phenylpropan-2-yl)-acetamido)methyl)benzoate (Compound 134b)

Following the same procedure described in Example 117, step 3, Scheme 25, but substituting compound 133b for compound 133a, the title compound 134b was obtained and used without purification. LRMS (ESI): (calc.) 384.2; (found) 385.1 (MH)⁺.

### Step 4: (R)-Methyl 4-((2-benzyl-3,6-dioxopiperazin-1-yl)methyl)benzoate (Compound 135b)

A solution of compound 134b (1.35 mmol) and Et₃N (1 mL, 7.3 mmol) in DCM (10 mL) was stirred at room temperature over night. The solution was diluted in DCM, washed with saturated NaHCO_{3(aq)}, dried over sodium sulfate and concentrated to afford the title compound 135b (120 mg, 25%, yield for two steps). LRMS (ESI): (calc.) 352.1; (found) 353.2 (MH)⁺.

### Step 5: (R)-4-((2-Benzyl-3,6-dioxopiperazin-1-yl)methyl)-N-hydroxybenzamide (Compound 136b)

Following the same procedure described in Example 108a, step 4, Scheme 19, but substituting compound 135b for compound 108a, the title compound 136b was isolated as white powder (6 mg, 12%). LRMS: (calc) 353.1 (found) 353.1 (MH)⁺. ¹H NMR (DMSO-d₆) δ (ppm): 11.16 (s, 1H), 9.01 (s, 1H), 8.07 (d, J=6.5 Hz, 1H), 7.70 (d, J=8.2 Hz, 2H), 7.31 (d, J=8.4 Hz, 2H), 7.30-7.28 (m, 3H), 7.12-7.10 (m, 1H), 5.13 (d, J=15 Hz, 1H), 4.07 (d, J=15 Hz, 1H), 3.95 (t, J=4.6 Hz, 1H), 3.4-3.3 (m, 2H), 3.23 (dd, J=12.9, 5.5 Hz, 1H), 3.04 (dd, J=4.1, 3.7 Hz, 1H).

### EXAMPLE 119

(R)-4-((3,6-Dioxo-2-(thien-2-ylmethyl)piperazin-1-yl)methyl)-N-hydroxybenzamide (Compound 136c)

### Step 1: (R)-Methyl 2-amino-3-(thien-2-yl)propanoate (Compound 131c)

AcCI (4.15 mL, 58.5 mmol) was added dropwise in MeOH (50 mL) at 0° C. The solution was stirred 15 min then (R)-2-amino-3-(thiophen-2-yl)propanoic acid (2.0 g, 11.7 mmol) was added and stirred over night. The solvent was concentrated and the residue was dissolved in DCM, washed with NaHCO_{3(aq)}, dryed with sodium sulfate and concentrated under vacuum to afford the title compound 131c (1.5 g, 69%). LRMS (ESI): (calc.) 185.4; (found) 186.1 (MH)⁺.

### Step 2: (R)-Methyl 4-((1-methoxy-1-oxo-3-(thien-2-yl)propan-2-ylamino)methyl)-benzoate (Compound 132c)

Following the same procedure described in Example 117, step 1, Scheme 25, but substituting compound 131c for compound (R)-Methyl 2-amino-3-(1H-indol-3-yl)propanoate, the title compound 132c was isolated (0.81 g, 45%). LRMS (ESI): (calc.) 333.1; (found) 334.1 (MH)⁺.

### Step 3: (R)-Methyl 4-((2-(benzyloxycarbonylamino)-N-(1-methoxy-1-oxo-3-(thien-2-yl)propan-2-yl)acetamido)methyl)benzoate (Compound 133c)

Following the same procedure described in Example 117, step 2, Scheme 25, but substituting compound 132c for compound 132a, the title compound 133c was isolated as (0.64 g, 81%). LRMS (ESI): (calc.) 524.6; (found) 525.7 (MH)⁺.

### Step 4: (R)-Methyl 4-((2-amino-N-(1-methoxy-1-oxo-3-(thien-2-yl)propan-2-yl)acetamido)methyl)benzoate hydrobromide (Compound 134c)

A solution of compound 133c (0.68 g, 2.7 mmol) and 33% HBr in AcOH (5 mL) was prepared at 0° C. and stirred 1 h at room temperature. The solution was concentrated and triturated in Et₂O (3×) to afford title compound 134c (0.8 g, 63%). LRMS (ESI): (calc.) 390.1; (found) 391.3 (MH)⁺.

### Step 5: (R)-Methyl 4-((3,6-dioxo-2-(thien-2-ylmethyl)piperazin-1-yl)methyl)-benzoate (Compound 135c)

Compound 134c (0.8 g, 1.7 mmol) was dissolved in DCM, washed with 10% NH₄OH_{(aq)}, dried over sodium sulfate and concentrated. The free amine obtained was dissolved in phenyl ether (5 mL) and stirred at 170° C. for 1 h. The solution was cooled down, diluted with hexanes and stirred 20 min. The precipitate obtain was filtered, rinsed with hexanes to afford the title compound as a beige solid (0.8 g, 100%). LRMS: (calc) 358.1 (found) 359.5 (MH)⁺.

### Step 6: (R)-4-((3,6-Dioxo-2-(thien-2-ylmethyl)piperazin-1-yl)methyl)-N-hydroxy-benzamide (Compound 136c)

Following the same procedure described in Example 108a, step 4, Scheme 19, but substituting compound 135c for compound 108a, the title compound 136c was isolated as a beige solid (130 mg, 100%). LRMS: (calc) 359.1 (found) 360.1 (MH)⁺. ¹H NMR (DMSO-d₆) δ (ppm): 11.16 (s, 1H), 9.01 (s, 1H), 8.14 (s, 1H), 7.69 (d, J=7.8 Hz, 2H), 7.46 (d, J=5.1 Hz, 1H), 7.32 (d, J=8.1 Hz, 2H), 6.98 (t, J=3.7 Hz, 1H), 6.79 (s, 1H), 5.11 (d, J=15.4 Hz, 1H), 4.14 (d, J=14.8 Hz, 1H), 4.03 (s, 1H), 3.95 (s, 1H), 3.46 (d, J=15.1 Hz, 1H), 3.3-3.2 (m, 1H), 2.72 (d, J=17.4 Hz, 1H).

Compositions

In the third aspect, the invention provides compositions comprising an inhibitor of histone deacetylase according to the invention and a pharmaceutically acceptable carrier, excipient, or diluent. Compounds of the invention may be formulated by any method known in the art and may be prepared for administration by any route, including, without limitation, parenteral, oral, sublingual, transdermal, topical, intranasal, intratracheal, or intrarectal. In certain preferred embodiments, compounds of the invention are administered intravenously in a hospital setting. In certain other preferred embodiments, administration may preferably be by the oral route. The compositions may be in any form, including but not limited to liquid solutions or suspensions; for oral administration, formulations may be in the form of tablets or capsules; and for intranasal formulations, in the form of powders, nasal drops or aerosols. The compositions may be administered locally or systemically.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, or other materials well known in the art. The preparation of pharmaceutically acceptable formulations is described in, e.g., Remington's Pharmaceutical Sciences, 18^{th} Edition, ed. A. Gennaro, Mack Publishing Co., Easton, Pa., 1990.

As used herein, the term pharmaceutically acceptable salts refer to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z-, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate). As used herein, the term “salt” is also meant to encompass complexes, such as with an alkaline metal or an alkaline earth metal.

The active compound is included in the pharmaceutically acceptable carrier or diluent in an amount sufficient to deliver an HDAC inhibiting effective amount without causing serious toxic effects. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

In certain preferred embodiments of the second aspect of the invention, the composition further comprises an antisense oligonucleotide that inhibits the expression of a histone deacetylase gene. The combined use of a nucleic acid level inhibitor (e.g., antisense oligonucleotide) and a protein level inhibitor (i.e., inhibitor of histone deacetylase enzyme activity) results in an improved inhibitory effect, thereby reducing the amounts of the inhibitors required to obtain a given inhibitory effect as compared to the amounts necessary when either is used individually. The antisense oligonucleotide according to this aspect of the invention is complementary to regions of RNA or double-stranded DNA that encode one or more of HDAC-1, HDAC-2, HDAC-3, HDAC-4, HDAC-5, HDAC-6, HDAC-7, HDAC-8, HDAC-9, HDAC-10, HDAC-11, SirT1, SirT2, SirT3, SirT4, SirT5, SirT6 and SirT7 (see e.g., GenBank Accession Number U50079 for HDAC-1, GenBank Accession Number U31814 for HDAC-2, and GenBank Accession Number U75697 for HDAC-3).

Inhibition of Histone Deacetylase

In the fourth aspect, the present invention provides a method of inhibiting histone deacetylase, comprising contacting the histone deacetylase with an inhibition effective amount of an inhibitor of histone deacetylase of the present invention.

In a preferred embodiment of the fourth aspect of the invention, the invention provides a method of inhibiting histone deacetylase in a cell, comprising contacting a cell in which inhibition of histone deacetylase is desired with an inhibitor of histone deacetylase according to the invention or a composition comprising an inhibitor of histone decetylase according to the invention. Because compounds of the invention inhibit histone deacetylase, they are useful research tools for the study of histone deacetylases and their role in biological processes.

Measurement of the enzymatic activity of a histone deacetylase can be achieved using known methodologies. For example, Yoshida et al., J. Biol. Chem., 265: 17174-17179 (1990), describes the assessment of histone deacetylase enzymatic activity by the detection of acetylated histones in trichostatin A treated cells. Taunton et al., Science, 272: 408-411 (1996), similarly describes methods to measure histone deacetylase enzymatic activity using endogenous and recombinant HDAC-1.

In some preferred embodiments, the histone deacetylase inhibitor interacts with and reduces the activity of all histone deacetylases in a cell. In some other preferred embodiments according to this aspect of the invention, the histone deacetylase inhibitor interacts with and reduces the activity of fewer than all histone deacetylases in the cell. In certain preferred embodiments, the inhibitor interacts with and reduces the activity of one histone deacetylase (e.g., HDAC-1), but does not interact with or reduce the activities of other histone deacetylases (e.g., HDAC-2, HDAC-3, HDAC-4, HDAC-5, HDAC-6, HDAC-7, HDAC-8, HDAC-9, HDAC-10, HDAC-11, SirT1, SirT2, SirT3, SirT4, SirT5, SirT6 and SirT7).

For purposes of the invention, the term “oligonucleotide” includes polymers of two or more deoxyribonucleosides, ribonucleosides, or 2′-substituted ribonucleoside residues, or any combination thereof. Preferably, such oligonucleotides have from about 6 to about 100 nucleoside residues, more preferably from about 8 to about 50 nucleoside residues, and most preferably from about 12 to about 30 nucleoside residues. The nucleoside residues may be coupled to each other by any of the numerous known internucleoside linkages. Such internucleoside linkages include without limitation phosphorothioate, phosphorodithioate, alkylphosphonate, alkylphosphonothioate, phosphotriester, phosphoramidate, siloxane, carbonate, carboxymethylester, acetamidate, carbamate, thioether, bridged phosphoramidate, bridged methylene phosphonate, bridged phosphorothioate and sulfone internucleoside linkages. In certain preferred embodiments, these internucleoside linkages may be phosphodiester, phosphotriester, phosphorothioate, or phosphoramidate linkages, or combinations thereof. The term oligonucleotide also encompasses such polymers having chemically modified bases or sugars and/or having additional substituents, including without limitation lipophilic groups, intercalating agents, diamines and adamantane.

For purposes of the invention the term “2′-substituted ribonucleoside” includes ribonucleosides in which the hydroxyl group at the 2′ position of the pentose moiety is substituted to produce a 2′-O-substituted ribonucleoside. Preferably, such substitution is with a lower alkyl group containing 1-6 saturated or unsaturated carbon atoms, or with an aryl or allyl group having 2-6 carbon atoms, wherein such alkyl, aryl or allyl group may be unsubstituted or may be substituted, e.g., with halo, hydroxy, trifluoromethyl, cyano, nitro, acyl, acyloxy, alkoxy, carboxyl, carbalkoxyl, or amino groups. The term “2′-substituted ribonucleoside” also includes ribonucleosides in which the 2′-hydroxyl group is replaced with an amino group or with a halo group, preferably fluoro.

Particularly preferred antisense oligonucleotides utilized in this aspect of the invention include chimeric oligonucleotides and hybrid oligonucleotides.

For purposes of the invention, a “chimeric oligonucleotide” refers to an oligonucleotide having more than one type of internucleoside linkage. One preferred example of such a chimeric oligonucleotide is a chimeric oligonucleotide comprising a phosphorothioate, phosphodiester or phosphorodithioate region, preferably comprising from about 2 to about 12 nucleotides, and an alkylphosphonate or alkylphosphonothioate region (see e.g., Pederson et al. U.S. Pat. Nos. 5,635,377 and 5,366,878). Preferably, such chimeric oligonucleotides contain at least three consecutive internucleoside linkages selected from phosphodiester and phosphorothioate linkages, or combinations thereof.

For purposes of the invention, a “hybrid oligonucleotide” refers to an oligonucleotide having more than one type of nucleoside. One preferred example of such a hybrid oligonucleotide comprises a ribonucleotide or 2′-substituted ribonucleotide region, preferably comprising from about 2 to about 12 2′-substituted nucleotides, and a deoxyribonucleotide region. Preferably, such a hybrid oligonucleotide contains at least three consecutive deoxyribonucleosides and also contains ribonucleosides, 2′-substituted ribonucleosides, preferably 2′-O-substituted ribonucleosides, or combinations thereof (see e.g., Metelev and Agrawal, U.S. Pat. No. 5,652,355).

The exact nucleotide sequence and chemical structure of an antisense oligonucleotide utilized in the invention can be varied, so long as the oligonucleotide retains its ability to inhibit expression of the gene of interest. This is readily determined by testing whether the particular antisense oligonucleotide is active. Useful assays for this purpose include quantitating the mRNA encoding a product of the gene, a Western blotting analysis assay for the product of the gene, an activity assay for an enzymatically active gene product, or a soft agar growth assay, or a reporter gene construct assay, or an in vivo tumor growth assay, all of which are known in the art, or are as described in detail in this specification or in, for example, Ramchandani et al. (1997) Proc. Natl. Acad. Sci. USA 94: 684-689.

Antisense oligonucleotides utilized in the invention may conveniently be synthesized on a suitable solid support using well known chemical approaches, including H-phosphonate chemistry, phosphoramidite chemistry, or a combination of H-phosphonate chemistry and phosphoramidite chemistry (i.e., H-phosphonate chemistry for some cycles and phosphoramidite chemistry for other cycles). Suitable solid supports include any of the standard solid supports used for solid phase oligonucleotide synthesis, such as controlled-pore glass (CPG) (see, e.g., Pon, R. T. (1993) Methods in Molec. Biol. 20: 465-496).

Particularly preferred oligonucleotides have nucleotide sequences of from about 13 to about 35 nucleotides which include the nucleotide sequences shown in Table 13. Yet additional particularly preferred oligonucleotides have nucleotide sequences of from about 15 to about 26 nucleotides and comprise the nucleotide sequences shown in Table 13.

TABLE 13

position

| | | |
|---|---|---|
| Accession | Nucleotide | within |

| | | | | | | |
|---|---|---|---|---|---|---|
| Oligo | Target | Number | Position | Sequence | Gene | Seq ID No. |

| | | | | | | |
|---|---|---|---|---|---|---|
| HDAC1 AS1 | Human | U50079 | 1585-1604 | 5′-GAAACGTGAGGGACTCAGCA-3′ | 3′-UTR | Seq ID No:1 |
| | HDAC1 | | | | | |
| HDAC1 AS2 | Human | U50079 | 1565-1584 | 5′-GGAAGCCAGAGCTGGAGAGG-3′ | 3′-UTR | Seq ID No:2 |
| | HDAC1 | | | | | |
| HDAC2 AS | Human | U31814 | 1643-1622 | 5′-GCTGAGCTGTTCTGATTTGG-3′ | 3′-UTR | Seq ID No:3 |
| | HDAC2 | | | | | |
| HDAC3 AS | Human | AF039703 | 1276-1295 | 5′-CGCTTTCCTTGTCATTGACA-3′ | 3′-UTR | Seq ID No:4 |
| | HDAC3 | | | | | |
| HDAC4 AS1 | Human | AB006626 | 514-33 | 5-GCTGCCTGCCGTGCCCACCC-3′ | 5′-UTR | Seq ID No:5 |
| | HDAC4 | | | | | |
| HDAC4 AS2 | Human | AB006626 | 7710-29 | 5′-TACAGTCCATGCAACCTCCA-3′ | 3′-UTR | Seq ID No:6 |
| | HDAC4 | | | | | |
| HDAC5 AS | Human | AF039691 | 2663-2682 | 5′-CTTCGGTCTCACCTGCTTGG-3′ | 3′-UTR | Seq ID No:7 |
| | HDAC5 | | | | | |
| HDAC6 AS | Human | AJ011972 | 3791-3810 | 5′-CAGGCTGGAATGAGCTACAG-3′ | 3′-UTR | Seq ID No:8 |
| | HDAC6 | | | | | |
| HDAC7 AS | Human | AF239243 | 2896-2915 | 5′-CTTCAGCCAGGATGCCCACA-3′ | 3′-UTR | Seq ID No:9 |
| | HDAC7 | | | | | |
| HDAC8 AS1 | Human | AF230097 | 51-70 | 5′-CTCCGGCTCCTCCATCTTCC-3′ | 5′-UTR | Seq ID No:10 |
| | HDAC8 | | | | | |
| HDAC8 AS2 | Human | AF230097 | 1328-1347 | 5′-AGCCAGCTGCCACTTGATGC-3′ | 3′-UTR | Seq ID No:11 |
| | HDAC8 | | | | | |

In certain preferred embodiments of the invention, the antisense oligonucleotide and the HDAC inhibitor of the present invention are administered separately to a mammal, preferably a human. For example, the antisense oligonucleotide may be administered to the mammal prior to administration to the mammal of the HDAC inhibitor of the present invention. The mammal may receive one or more dosages of antisense oligonucleotide prior to receiving one or more dosages of the HDAC inhibitor of the present invention.

In another example, the HDAC inhibitor of the present invention may be administered to the mammal prior to administration of the antisense oligonucleotide. The mammal may receive one or more dosages of the HDAC inhibitor of the present invention prior to receiving one or more dosages of antisense oligonucleotide.

In certain preferred embodiments of the present invention, the HDAC inhibitor of the present invention may be administered together with other HDAC inhibitors known in the art or which will be discovered. Administration of such HDAC inhibitors may be done sequentially or concurrently. In certain preferred embodiments of the present invention the compositions comprise HDAC inhibitors of the present invention and/or an antisense oligonucleotide and/or another HDAC inhibitor known in the art or which will be discovered. The active ingredients of such compositions may act synergistically to inhibit histone deacetylase.

In certain embodiments, the known HDAC inhibitor is selected from the group consisting of, but not limited to, trichostatin A, depudecin, trapoxin, suberoylanilide hydroxamic acid, FR901228, MS-27-275, CI-994 sodium butyrate, MGCD0103, and those compounds found in WO 2003/024448, WO 2004/069823, WO 2001/038322, U.S. Pat. No. 6,541,661, WO 01/70675, WO 2004/035525 and WO 2005/030705.

The following Examples are intended to further illustrate certain preferred embodiments of the invention, and are not intended to limit the scope of the invention.

### ASSAY EXAMPLES

### Assay Example 1

### Inhibition of Histone Deacetylase Enzymatic Activity

The following protocol is used to assay the compounds of the invention. In the assay, the buffer used is 25 mM HEPES, pH 8.0, 137 mM NaCl, 2.7 mM KCl, 1 mM MgCl₂ and the substrate is Boc-Lys(Ac)-AMC in a 50 mM stock solution in DMSO. The enzyme stock solution is 4.08 μg/mL in buffer.

The compounds are pre-incubated (2 μl in DMSO diluted to 13 μl in buffer for transfer to assay plate) with enzyme (20 μl of 4.08 μg/mL) for 10 min at room temperature (35 μl pre-incubation volume). The mixture is pre-incubated for 5 min at room temperature. The reaction is started by bringing the temperature to 37° C. and adding 16 μl substrate. Total reaction volume is 50 μl. The reaction is stopped after 20 min by addition of 50 μl developer, prepared as directed by Biomol (Fluor-de-Lys developer, Cat. #KI-105). A plate is incubated in the dark for 10 min at room temperature before reading (λ_{EX}=360 nm, λ_{Em}=470 nm, Cutoff filter at 435 nm).

Table 14 shows that HDAC inhibitors of the present invention have HDAC inhibitor activity (IC₅₀) against one or more of HDAC-1, HDAC-2, HDAC-3, HDAC-4, HDAC-5, HDAC-6, HDAC-7, HDAC-8, HDAC-9, HDAC-10, HDAC-11, SirT1, SirT2, SirT3, SirT4, SirT5, SirT6 and SirT7. In the table, “A” indicates inhibitory activity at a concentration of <0.05 μM; “B” indicates inhibitory activity at a concentration >0.05 μM but <0.5 μM, “C” indicates inhibitory activity at >0.5 μM but <2 μM and “D” indicates inhibitory activity at a concentration of >2 μM but <10 μM.

TABLE 14

| | HDAC | | HDAC | | HDAC | | HDAC |
|---|---|---|---|---|---|---|---|
| | Inhibitory | | Inhibitory | | Inhibitory | | Inhibitory |
| Cpd | Activity (IC₅₀) | Cpd | Activity (IC₅₀) | Cpd | Activity (IC₅₀) | Cpd | Activity (IC₅₀) |
| 4k | D | 80 | B | 97k | D | 54b13 | A |
| 66f | B | 81 | C | 4e | D | 54b14 | B |
| 4h | D | 13 | B | 68b16 | D | 68b23 | B |
| 6h | D | 24f | A | 68b18 | B | 54b7 | A |
| 5c | D | 8 | C | 68b3 | B | 54b15 | A |
| 23a | D | 4i | D | 68b19 | C | 54b16 | A |
| 47b | D | 66c | C | 68b20 | C | 54b17 | B |
| 47a | B | 92a | B | 68b21 | B | 97c | C |
| 4c | C | 83 | C | 18c | D | 97h | C |
| 24a | B | 88a | B | 18e | C | 54b21 | A |
| 4b | D | 26e | B | 18f | C | 72 | D |
| 62 | B | 82 | C | 109a | B | 54b18 | A |
| 49 | B | 68b8 | B | 112a | C | 92b | B |
| 97a | D | 4g | D | 16h | C | 97b | D |
| 54b19 | B | 69 | C | 16b | C | 97d | C |
| 54b20 | A | 43 | D | 16e | D | 97f | D |
| 54c | B | 16f | B | 4l | C | 97g | D |
| 68b7 | D | 18a | D | 109c | B | 97i | D |
| 24e | A | 18b | C | 68b4 | B | 97j | D |
| 29a | B | 16a | D | 71 | D | 112d | B |
| 68b9 | C | 16c | C | 54b1 | A | 136b | A |
| 68b6 | C | 54a1 | B | 54b5 | A | 136a | A |
| 68b1 | C | 54a2 | A | 54b3 | A | 136c | B |
| 68b11 | B | 109b | B | 54b4 | A | 110 | C |
| 79 | B | 112c | C | 54b2 | A | 116 | A |
| 74 | B | 112b | C | 54b8 | B | 118 | B |
| 68b12 | B | 68b22 | C | 56a | C | 122 | A |
| 4f | D | 54b10 | A | 54b9 | A | | |
| 4a | D | 16g | D | 54b11 | A | | |
| 4d | D | 16d | D | 54b12 | A | | |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A compound of the formula (I): 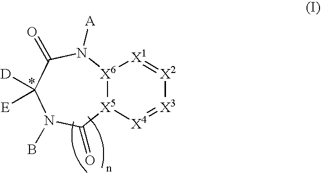
and N-oxides, hydrates, solvates, pharmaceutically acceptable salts, prodrugs and complexes thereof, wherein
n is 0 or 1;
X¹, X², X³ and X⁴ are independently selected from the group consisting of CH, C-Z and N, wherein no more than two of X¹, X², X³ and X⁴ are N and no more than one of X¹, X², X³ and X⁴ are C-Z;
X⁵-X⁶ is C═C;
or
X¹, X², X³ and X⁴ are absent, X⁵ is a covalent bond and X⁶ is independently selected from the group consisting of CH₂ and CH(Z), with the provisio that an N, O or S(O)₀₋₁ in Z is separated from the CH of X⁶ by at least two carbon atoms;
Z is independently selected from the group consisting of halo, —CF₃, —NO₂, —CN, —(C₀-C₆)alkyl-OR¹, —(C₀-C₆)alkyl-N(R¹)₂, —(C₁-C₆)alkyl, —N(R¹)—C(O)—(C₁-C₆)alkyl, —N(R¹)—S(O)₂—(C₁-C₆)alkyl, —O—(C₂-C₆)alkyl-N(R¹)(R¹), —S—R¹, —(C₀-C₆)alkyl-C(O)—OR¹, —N(R¹)—C(O)—CF₃, —N(R¹)—(C₂-C₆)alkyl-N(R¹)(R¹), —(C₀-C₇)alkyl-W, —(C₂-C₇)alkenyl-W, —(C₂-C₇)alkynyl-W, —(C₀-C₅)alkyl-CH═CH—W, —C(O)—(C₁-C₇)alkyl-W, —(C₀-C₃)alkyl-N(R¹)—C(O)—(C₁-C₆)alkyl-W, —(C₀-C₃)alkyl-N(R¹)—C(S)—(C₁-C₆)alkyl-W, —C(O)—N(R¹)—(C₁-C₆)alkyl-W, —(C₀-C₃)alkyl-N(R¹)—(C₁-C₆)alkyl-W, —(C₀-C₃)alkyl-N(R¹)—C(O)—N(R¹)—(C₁-C₆)alkyl-W, —(C₀-C₃)alkyl-N(R¹)—C(O)—O—(C₁-C₆)alkyl-W, —S(O)₂—N(R¹)—(C₁-C₆)alkyl-W, —(C₀-C₃)alkyl-N(R¹)—S(O)₂—(C₁-C₆)alkyl-W, —C(O)—N(R¹)₂, —(C₀-C₃)alkyl-O—C(O)—N(R¹)—(C₁-C₆)alkyl-W, —(C₀-C₃)alkyl-O—(C₁-C₆)alkyl-W, —(C₀-C₃)alkyl-S—(C₁-C₆)alkyl-W, —N(R¹)—C(O)—OR¹, —S(O)₂—N(R¹)₂, —N(R¹)—S(O)₂R¹, —(C₀-C₇)alkyl-aryl-W, —(C₀-C₇)alkyl-heteroaryl-W, —(C₀-C₃)alkyl-O—(C₀-C₃)alkyl-aryl, —(C₀-C₃)alkyl-O—(C₀-C₃)alkyl-heteroaryl, -aryl, —(C₁-C₆)alkylaryl-, -heteroaryl, —(C₁-C₆)alkylheteroaryl-, —(C₁-C₈) heteroalkyl, —(C₃-C₆)cycloalkyl, —(C₃-C₆)heterocycloalkyl, —(C₀-C₃)alkyl-N(R¹)—(C₀-C₃)alkyl-aryl-(CH═CH)₀₋₁—W, —(C₀-C₃)alkyl-O—(C₀-C₃)alkyl-aryl-(CH═CH)₀₋₁—W, —(C₀-C₃)alkyl-S—(C₀-C₃)alkyl-aryl-(CH═CH)₀₋₁—W, —(C₀-C₃)alkyl-N(R¹)C(O)—O—(C₀-C₃)alkyl-aryl-(CH═CH)₀₋₁—W, —(C₀-C₃)alkyl-O—C(O)N(R¹)—(C₀-C₃)alkyl-aryl-(CH═CH)₀₋₁—W, —(C₀-C₃)alkyl-S(O)₂N(R¹)—(C₀-C₃)alkyl-aryl-(CH═CH)₀₋₁—W, —(C₀-C₃)alkyl-N(R¹)S(O)₂—(C₀-C₃)alkyl-aryl-(CH═CH)₀₋₁—W, —(C₀-C₃)alkyl-C(O)N(R¹)—(C₀-C₃)alkyl-aryl-(CH═CH)₀₋₁—W, —(C₀-C₃)alkyl-N(R¹)C(O)—(C_{O-}C₃)alkyl-aryl-(CH═CH)₀₋₁—W, —(C₀-C₃)alkyl-N(R¹)C(O)N(R¹)—(C₀-C₃)alkylaryl(CH═CH)₀₋₁—W, —(C₀-C₃)alkyl-(CH═CH)—(C₀-C₃)alkyl-aryl-(CH═CH)₀₋₁—W, —(C₀-C₃)alkyl-N(R¹)—(C₀-C₃)alkyl-heteroaryl-(CH═CH)₀₋₁—W, —(C₀-C₃)alkyl-O—(C₀-C₃)alkyl-heteroaryl-(CH═CH)₀₋₁—W, —(C₀-C₃)alkyl-S—(C₀-C₃)alkyl-heteroaryl-(CH═CH)₀₋₁—W, —(C₀-C₃)alkyl-N(R¹)C(O)—O—(C₀-C₃)alkyl-heteroaryl-(CH═CH)O, —W, —(C₀-C₃)alkyl-OC(O)N(R¹)—(C₀-C₃)alkyl-heteroaryl-(CH═CH)₀₋₁—W, —(C₀-C₃)alkyl-S(O)₂N(R¹)—(C₀-C₃)alkyl-heteroaryl-(CH═CH)₀₋₁—W, —(C₀-C₃)alkyl-N(R¹)S(O)₂—(C₀-C₃)alkyl-heteroaryl-(CH═CH)₀₋₁—W, —(C₀-C₃)alkyl-C(O)N(R¹)—(C₀-C₃)alkyl-heteroaryl-(CH═CH)₀₋₁—W, —(C₀-C₃)alkyl-N(R¹)C(O)—(C₀-C₃)alkyl-heteroaryl-(CH═CH)₀₋₁—W, —(C₀-C₃)alkyl-N(R¹)C(O)N(R¹)— (C₀-C₃)alkyl-heteroaryl-(CH═CH)₀₋₁—W and —(C₀-C₃)alkyl-(CH═CH)—(C₀-C₃)alkyl-heteroaryl-(CH═CH)₀₋₁—W, —(C₀-C₅)alkyl-C≡C—W, —(C₀-C₃)alkyl-O—(C₀-C₃)alkyl-aryl-(C{cube root}C)₀₋₁—W, —(C₀-C₃)alkyl-S—(C₀-C₃)alkyl-aryl-(C≡C)₀₋₁—W, —(C₀-C₃)alkyl-N(R¹)C(O)—O—(C₀-C₃)alkyl-aryl-(C≡C)₀₋₁—W, —(C₀-C₃)alkyl-O—C(O)N(R¹)—(C₀-C₃)alkyl-aryl-(C≡C)₀₋₁—, —W, —(C₀-C₃)alkyl-S(O)₂N(R¹)—(C₀-C₃)alkyl-aryl-(C≡C)₀₋₁—W, —(C₀-C₃)alkyl-N(R¹)S(O)₂—(C₀-C₃)alkyl-aryl-(C≡C)₀₋₁—W, —(C₀-C₃)alkyl-C(O)N(R¹)—(C_{O-}C₃)alkyl-aryl-(C≡C)₀₋₁—W, —(C₀-C₃)alkyl-N(R¹)C(O)—(C₀-C₃)alkyl-aryl-(C≡C)₀₋₁—W, —(C₀-C₃)alkyl-N(R¹)C(O)N(R¹)—(C_{O-}C₃)alkyl-aryl-(C≡C)₀₋₁—W, —(C₀-C₃)alkyl-(C≡C)—(C₀-C₃)alkyl-aryl-(CH═CH)₀₋₁—W, —(C₀-C₃)alkyl-(CH═CH)—(C₀-C₃)alkyl-aryl-(C≡C)₀₋₁—W, —(C₀-C₃)alkyl-(C≡C)—(C₀-C₃)alkyl-aryl-(C≡C)₀₋₁—W, —(C₀-C₃)alkyl-N(R¹)—(C₀-C₃)alkyl-heteroaryl-(C≡C)₀₋₁—W, —(C₀-C₃)alkyl-O—(C₀-C₃)alkyl-heteroaryl-(C≡C)₀₋₁—W, —(C₀-C₃)alkyl-S—(C₀-C₃)alkyl-heteroaryl-(C≡C)₀₋₁—W, —(C₀-C₃)alkyl-N(R¹)C(O)—O—(C₀-C₃)alkyl-heteroaryl-(C≡C)₀₋₁—W, —(C₀-C₃)alkyl-OC(O)N(R¹)—(C₀-C₃)alkyl-heteroaryl-(C≡C)₀₋₁—W, —(C₀-C₃)alkyl-S(O)₂N(R₁)—(C₀-C₃)alkyl-heteroaryl-(C≡C)₀₋₁—W, —(C₀-C₃)alkyl-N(R¹)S(O)₂—(C₀-C₃)alkyl-heteroaryl-(C≡C)₀₋₁—W, —(C₀-C₃)alkyl-C(O)N(R¹)—(C₀-C₃)alkyl-heteroaryl-(C≡C)₀₋₁—W, —(C₀-C₃)alkyl-N(R¹)C(O)—(C₀-C₃)alkyl-heteroaryl-(C≡C)₀₋₁—W, —(C₀-C₃)alkyl-N(R¹)C(O)N(R¹)—(C₀-C₃)alkyl-heteroaryl-(C≡C)₀₋₁ —W, —(C₀-C₃)alkyl-(C≡C)—(C₀-C₃)alkyl-heteroaryl-(CH═CH)₀₋₁—W, —(C₀-C₃)alkyl-(CH═CH)—(C₀-C₃)alkyl-heteroaryl-(C≡C)₀₋₁—W, —(C₀-C₃)alkyl-(C≡C)—(C₀-C₃)alkyl-heteroaryl-(C≡C)₀₋₁—W, (C₀-C₃)alkyl-C(O)—N(R¹)—(C₁-C₆)alkyl-W, (C₀-C₃)alkyl-C(S)—N(R¹)—(C₁-C₆)alkyl-W, —(C₀-C₃)alkyl-N(R₁)—C(O)—(C₁-C₆)alkyl-C(O)-aryl, —(C₀-C₃)alkyl-N(R¹)—C(O)—(C₁-C₆)alkyl-C(O)-heteroaryl, —(C₀-C₃)alkyl-N(R₁)—C(O)—(C₁-C₆)alkyl-C(O)—N(R₁)-aryl, and —(C₀-C₃)alkyl-N(R₁)—C(O)—(C₁-C₆)alkyl-C(O)—N(R₁)-heteroaryl, wherein each of the aryl, heteroaryl, cycloalkyl and heterocyclyl moieties of the above-mentioned Z is optionally substituted with one or more substituents selected from the group consisting of oxo, —OH, —CN, —(C₁-C₆)alkyl, —(C₁-C₆)alkoxy, —NO₂, —N(R¹)₂, halo, —SH, mono- to per-halogenated —(C₁-C₆)alkyl, and —(C₂-C₄)alkyl-N(R¹)₂, wherein two R¹ groups, together with the nitrogen atom to which they are attached optionally form a heterocyclyl group;
R¹ is independently selected from the group consisting of —H, —(C₁-C₆)alkyl, —(C₁-C₆)heteroalkyl, —(C₃-C₆)cycloalkyl, heterocyclyl, —(C₀-C₆)alkyl-aryl, —(C₀-C₆)alkyl-heteroaryl and —(C₂-C₄)alkyl-N(R¹)₂, wherein each aryl, heteroaryl, cycloalkyl and heterocyclyl moiety of said —(C₃-C₆)cycloalkyl, heterocyclyl, —(C₀-C₆)alkyl-aryl and —(C₀-C₆)alkyl-heteroaryl is optionally substituted with one or more substituents selected from the group consisting of oxo, —OH, —CN, —(C₁-C₆)alkyl, —(C₁-C₆)alkoxy, —NO₂, —N(R¹)₂, halo, aryl, heteroaryl, mono- to per-halogenated-(C₁-C₆)alkyl and —(C₂-C₄)alkyl-N(R¹)₂, wherein two R¹ groups, together with the nitrogen atom to which they are attached optionally form a heterocyclyl group;
W is selected from the group consisting of —C(O)—NH—OH, —C(O)—C₁-C₄ alkyl, —C(O)—N(R¹)₂, —(C₁-C₆)alkyl-N(OH)—C(O)H—, —(C₁-C₆)alkyl-SR¹, —(C₁-C₆)alkyl-S—C(O)—(C₁-C₄)alkyl, —C(O)—OR¹, 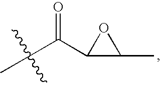
—C(O)—(C₁-C₄)alkyl-SH, —C(O)—(C₁-C₄)alkyl-S—C(O)R¹, —C(O)—(C₁-C₄)alkyl-5-heteroaryl, —(C₁-C₆)alkyl-NH—C(O)—(C₁-C₆) alkyl-halo, —(C₁-C₆)alkyl-NH—C(O)—(C₁-C₆)alkyl-SH, —(C₁-C₆)alkyl-NH—C(O)—(C₁-C₆)alkyl-SC(O)R¹, —C(O)—NH—(C₂-C₆)alkyl-SH, —C(O)—N(R¹)—(C₀-C₆)alkyl-SR¹, —C(O)-cycloalkyl, —C(O)-heterocyclyl, —C(O)—N(R¹)-aryl-Q, —C(O)—N(R¹)— heteroaryl-Q, —C(O)-aryl, —C(O)-heteroaryl and —C(O)—(C₁-C₆) alkyl wherein the alkyl is optionally substituted with one or more substituents selected from the group consisting of halo, mono to per-halogenated-(C₁-C₆)alkyl, —C(O)-heteroaryl, —C(O)—NH-heteroaryl and —C(O)—NH-aryl, wherein each aryl and heteroaryl moiety of the afore-mentioned W group is optionally substituted with one or more substituents selected from the group consisting of —NH₂, —OH, —SH, —CN, —NO₂, —N(R¹)₂, halo, mono- to per-halogenated —(C₁-C₆)alkyl, aryl and heteroaryl;
E and D are independently selected from the group consisting of —H, —(C₁-C₆)alkyl, —(C₁-C₆)heteroalkyl, —(C₀-C₆)alkyl-(C₃-C₆)cycloalkyl, —(C₀-C₆) heteroalkyl-(C₃-C₆)cycloalkyl, —(C₀-C₆)alkyl-(C₃-C₆) heterocyclyl, —(C₀-C₆)heteroalkyl-(C₃-C₆)heterocyclyl, —(C₀-C₆)alkyl-aryl, —(C₀-C₆)alkyl-heteroaryl, —(C₀-C₆)alkyl-heteroaryl-(C₀-C₃)alkyl-aryl, —(C₀-C₆)alkyl-aryl-(C₀-C₃)alkyl-aryl, —(C₀-C₆)alkyl-heteroaryl-(C₀-C₃)alkyl-heteroaryl, —(C₀-C₆)alkyl-aryl-(C₀-C₃)alkyl-heteroaryl, heterocyclyl, —(C₁-C₆)alkyl-S—R¹, —(C₁-C₆)heteroalkyl-S—R¹, —(C₁-C₆)alkyl-O—R¹, —(C₁-C₆)heteroalkyl-O—R¹, —C₁-C₆ alkyl-W, —(C₁-C₆)heteroalkyl-W, —(C₁-C₆)alkyl-M-(C₁-C₃)alkyl-W, —(C₁-C₆)heteroalkyl-M-(C₁-C₃)alkyl-W, —(C₁-C₆)alkyl-N(R¹)₂, —(C₁-C₆)heteroalkyl-N(R¹)₂, —(C₁-C₆)alkyl-N(R¹)—C(O)—OR¹, —(C₀-C₆)alkyl-C(O)—O—(C₁-C₆)alkyl, —(C₀-C₆)alkyl-C(O)—O—(C₁-C₆) heteroalkyl, —(C₀-C₆)heteroalkyl-C(O)—O—(C₁-C₆)alkyl, —(C₀-C₆)heteroalkyl-C(O)—O—(C₁-C₆)heteroalkyl, —(C₀-C₆)alkyl-C(O)—O—(C₁-C₆)cycloalkyl, —(C₀-C₆) heteroalkyl-C(O)—O—(C₁-C₆)cycloalkyl, —(C₀-C₆)alkyl-C(O)—O—(C₁-C₆)heterocyclyl, —(C₀-C₆) heteroalkyl-C(O)—O—(C₁-C₆)heterocyclyl, —(C₀-C₆)alkyl-C(O)—N(R¹)₂, —(C₀-C₆)heteroalkyl-C(O)—N(R¹)₂ and —C(O)—N(R¹)—C₂-C₆alkyl-W, wherein each aryl, heteroaryl, cycloalkyl or heterocyclyl moiety is optionally substituted with one or more groups selected from R²; wherein
M is selected from the group consisting of CH₂, O, S, S(O), S(O)₂ and N(R¹); or
C and D together with the carbon atom to which they are attached form a (C₃-C₆)cycloalkyl, wherein the cycloalkyl is optionally substituted;
R² is independently selected from the group consisting of —H, —(C₁-C₆)alkyl, —(C₁-C₆)heteroalkyl, —(C₀-C₆)alkyl-OR¹, —(C₀-C₆)heteroalkyl-OR¹, —(C₀-C₆)alkyl-C(O)—OR¹, —(C₀-C₆)heteroalkyl-C(O)—OR¹, —CH═CH—C(O)—OR¹, —C≡C—C(O)—OR¹, —CH═CH—C(O)—N(R¹)₂, —C≡C—C(O)—N(R¹)₂, —N(R¹)—C(O)—CF₃, —C(O)—N(R¹)—CF₃, —N(R¹)—(C₁-C₆)alkyl-N(R¹)₂, —N(R¹)—(C₁-C₆)heteroalkyl-N(R¹)₂, —(C₀-C₆)alkyl-N(R¹)₂, —(C₀-C₆)heteroalkyl-N(R¹)₂, —N(R¹)—C(O)—(C₁-C₆)alkyl, —C(O)—N(R¹)—(C₁-C₆)alkyl, —N(R¹)—C(O)—(C₁-C₆)heteroalkyl, —C(O)—N(R¹)—(C₁-C₆)heteroalkyl, —N(R¹)—S(O)₂—(C₁-C₆)alkyl, —N(R¹)—S(O)₂—(C₁-C₆)heteroalkyl, —S(O)₂—N(R¹)—(C₁-C₆)alkyl, —S(O)₂—N(R¹)—(C₁-C₆)heteroalkyl, —O—(C₁-C₆)alkyl-N(R¹)₂, —O—(C₁-C₆)heteroalkyl-N(R¹)₂, —S—(C₁-C₆)alkyl-N(R¹)₂, —S—(C₁-C₆)heteroalkyl-N(R¹)₂, —S—R¹, —S(O)—(C₁-C₆)alkyl, —S(O)—(C₁-C₆)heteroalkyl, —S(O)₂—(C₁-C₆)alkyl, —S(O)₂—(C₁-C₆)heteroalkyl, —(C₃-C₆)cycloalkyl, heterocyclyl, halo, —CF₃, —OCF₃, —C(Ph)₃, —CN, —(C₁-C₆)alkylaryl, aryl, heteroaryl, —(C₁-C₆)alkylheteroaryl, —(C₁-C₆) heteroalkylaryl, —(C₁-C₆)heteroalkylheteroaryl, and —(C₁-C₆)alkyl substituted with a moiety selected from the group consisting of halo, —OH, —NO₂, —(C₀-C₆)alkyl-C(O)—N(R¹)₂ and —(C₀-C₆)heteroalkyl-C(O)—N(R¹)₂;
A and B are independently selected from the group consisting of —H, —(C₁-C₆)alkyl, —(C₁-C₆)heteroalkyl, —(C₃-C₆)cycloalkyl, heterocyclyl, —(C₀-C₆)alkyl-aryl, —(C₀-C₆)alkyl-heteroaryl, —(C₀-C₆)heteroalkyl-aryl, —(C₀-C₆)hetroalkyl-heteroaryl, —S(O)₂—(C₀-C₆)alkyl-aryl, —S(O)₂—(C₀-C₆)alkyl-heteroaryl, —S(O)₂—(C₀-C₆)heteroalkyl-aryl, —S(O)₂—(C₀-C₆)heteroalkyl-heteroaryl, —C(O)—(C₁-C₆)alkyl-aryl, —C(O)—(C₁-C₆)alkyl-heteroaryl, —C(O)—(C₁-C₆)heteoralkyl-aryl, —C(O)—(C₁-C₆)heteroalkyl-heteroaryl, —C(O)O—(C₀-C₆)alkyl-aryl, —C(O)O—(C₁-C₆)alkyl-heteroaryl, —C(O)O—(C₁-C₆)heteroalkyl-aryl, —C(O)O—(C₁-C₆)heteroalkyl-heteroaryl, —C(O)N(R¹)—(C₁-C₆)alkyl-aryl, —C(O)N(R¹)—(C₁-C₆)heteroalkyl-aryl, —C(O)N(R¹)—(C₁-C₆)alkyl-heteroaryl, —C(O)N(R¹)—(C₁-C₆)heteroalkyl-heteroaryl, —(C₂-C₆)alkyl-N(R¹)₂, —(C₂-C₆)heteroalkyl-N(R¹)₂, —(C₂-C₆)alkyl-O(R¹), —(C₂-C₆)heteroalkyl-O(R¹), —(C₁-C₇)alkyl-W, —(C₁-C₇)heteoralkyl-W, —(C₂-C₅)alkyl-(CH═CH)₀₋₁—W, —(C₂-C₅)heteroalkyl-(CH═CH)₀₋₁—W, —(C₂-C₅)alkyl-(C≡C)₀₋₁—W, —(C₂-C₅)heteroalkyl-C≡C—W, —C(O)—(C₁-C₇)alkyl-W, —C(O)—(C₁-C₇)heteroalkyl-W, —S(O)₂—(C₁-C₆)alkyl-W, —S(O)₂—(C₁-C₆)heteroalkyl-W, —(C₀-C₇)alkyl-aryl-(CH═CH)₀₋₁—W, —(C₀-C₇)heteroalkyl-aryl-(CH═CH)₀₋₁—W, —(C₀-C₇)alkyl-aryl-(C≡C)₀₋₁—W, —(C₀-C₇)heteroalkyl-aryl-(C≡C)₀₋₁—W, —(C₀-C₇)alkyl-heteroaryl-(CH═CH)₀₋₁—W, —(C₀-C₇)heteroalkyl-heteroaryl-(CH═CH)₀₋₁—W, —(C₀-C₇)alkyl-heteroaryl-(C≡C)₀₋₁—W, —(C₀-C₇)heteroalkyl-heteroaryl-(C≡C)₀₋₁—W, —(C₀-C₇)alkyl-aryl-(C₀-C₄)alkyl-W, —(C₀-C₇)heteroalkyl-aryl-(C₀-C₄)alkyl-W, —(C₀-C₇)alkyl-aryl-(C₀-C₄)heteroalkyl-W, —(C₀-C₇) heteroalkyl-aryl-(C₀-C₄)heteroalkyl-W, —(C₀-C₇)alkyl-heteroaryl-(C₀-C₄)alkyl-W, —(C₀-C₇) heteroalkyl-heteroaryl-(C₀-C₄)alkyl-W, —(C₀-C₇)alkyl-heteroaryl-(C₀-C₄) heteroalkyl-W, —(C₀-C₇) heteroalkyl-heteroaryl-(C₀-C₄) heteroalkyl-W, —S(O)₂—(C₁-C₆)alkyl-aryl-(C₀-C₄)alkyl-(CH═CH)₀₋₁—W, —S(O)₂—(C₁-C₆)heteroalkyl-aryl-(C₀-C₄)alkyl-(CH═CH)₀₋₁—W, —S(O)₂—(C₁-C₆)alkyl-aryl-(C₀-C₄) heteroalkyl-(CH═CH)₀₋₁—W, —S(O)₂—(C₁-C₆)heteroalkyl-aryl-(C₀-C₄)heteroalkyl-(CH═CH)₀₋₁—W, —S(O)₂—(C₁-C₆)alkyl-aryl-(C₀-C₄)alkyl-(C≡C)₀₋₁—W, —S(O)₂—(C₁-C₆)heteroalkyl-aryl-(C₀-C₄)alkyl-(C≡C)₀₋₁—W, —S(O)₂—(C₁-C₆)alkyl-aryl-(C₀-C₄)heteroalkyl-(C≡C)₀₋₁—W, —S(O)₂—(C₁-C₆)heteroalkyl-aryl-(C₀-C₄)heteroalkyl-(C≡C)₀₋₁—W, —S(O)₂—(C₁-C₆)alkyl-heteroaryl-(C₀-C₄)alkyl-(CH═CH)₀₋₁—W, —S(O)₂—(C₁-C₆)heteroalkyl-heteroaryl-(C₀-C₄)alkyl-(CH═CH)₀₋₁—W, —S(O)₂—(C₁-C₆)alkyl-heteroaryl-(C₀-C₄)heteroalkyl-(CH═CH)₀₋₁—W, —S(O)₂—(C₁-C₆)heteroalkyl-heteroaryl-(C₀-C₄)heteroalkyl-(CH═CH)₀₋₁—W, —S(O)₂—(C₁-C₆)alkyl-heteroaryl-(C₀-C₄)alkyl-(C≡C)₀₋₁—W, —S(O)₂—(C₁-C₆)heteroalkyl-heteroaryl-(C₀-C₄)alkyl-(C≡C)₀₋₁—W, —S(O)₂—(C₁-C₆)alkyl-heteroaryl-(C₀-C₄)heteroalkyl-(C≡C)₀₋₁—W, —S(O)₂—(C₁-C₆)heteroalkyl-heteroaryl-(C₀-C₄)heteroalkyl-(C≡C)₀₋₁—W, —C(O)—(C₁-C₆)alkyl-aryl-(C₀-C₄)alkyl-(CH═CH)₀₋₁—W, —C(O)—(C₁-C₆)heteroalkyl-aryl-(C₀-C₄)alkyl-(CH═CH)₀₋₁—W, —C(O)—(C₁-C₆)alkyl-aryl-(C₀-C₄)heteroalkyl-(CH═CH)₀₋₁—W, —C(O)—(C₁-C₆)heteroalkyl-aryl-(C₀-C₄) heteroalkyl-(CH═CH)₀₋₁—W, —C(O)—(C₁-C₆)alkyl-aryl-(C₀-C₄)alkyl-(C≡C)₀₋₁—W, —C(O)—(C₁-C₆)heteroalkyl-aryl-(C₀-C₄)alkyl-(C≡C)₀₋₁—W, —C(O)—(C₁-C₆)alkyl-aryl-(C₀-C₄) heteroalkyl-(C≡C)₀₋₁—W, —C(O)—(C₁-C₆) heteroalkyl-aryl-(C₀-C₄)heteroalkyl-(C≡C)₀₋₁—W, —C(O)—(C₁-C₆)alkyl-heteroaryl-(C₀-C₄)alkyl-(CH═CH)₀₋₁—W, —C(O)—(C₁-C₆)heteroalkyl-heteroaryl-(C₀-C₄)alkyl-(CH═CH)₀₋₁—W, —C(O)—(C₁-C₆)alkyl-heteroaryl-(C₀-C₄)heteroalkyl-(CH═CH)₀₋₁—W, —C(O)—(C₁-C₆)heteroalkyl-heteroaryl-(C₀-C₄)heteroalkyl-(CH═CH)₀₋₁—W, —C(O)—(C₁-C₆)alkyl-heteroaryl-(C₀-C₄)alkyl-(C≡C)₀₋₁—W, —C(O)—(C₁-C₆)heteroalkyl-heteroaryl-(C₀-C₄)alkyl-(C≡C)₀₋₁—W, —C(O)—(C₁-C₆)alkyl-heteroaryl-(C₀-C₄)heteroalkyl-(C≡C)₀₋₁—W, —C(O)—(C₁-C₆) heteroalkyl-heteroaryl-(C₀-C₄)heteroalkyl-(C≡C)₀₋₁—W, —C(O)O—(C₀-C₆)alkyl-aryl-(C₀-C₄)alkyl-(CH═CH)₀₋₁—W, —C(O)O—(C₀-C₆)heteroalkyl-aryl-(C₀-C₄)alkyl-(CH═CH)₀₋₁—W, —C(O)O—(C₀-C₆)alkyl-aryl-(C₀-C₄)heteroalkyl-(CH═CH)₀₋₁—W, —C(O)O—(C₀-C₆)heteroalkyl-aryl-(C₀-C₄) heteroalkyl-(CH═CH)₀₋₁—W, —C(O)O—(C₀-C₆)alkyl-aryl-(C₀-C₄)alkyl-(C≡C)₀₋₁—W, —C(O)O—(C₀-C₆)heteroalkyl-aryl-(C₀-C₄)alkyl-(C≡C)₀₋₁—W, —C(O)O—(C₀-C₆)alkyl-aryl-(C₀-C₄) heteroalkyl-(C≡C)₀₋₁—W, —C(O)O—(C₀-C₆)heteroalkyl-aryl-(C₀-C₄)heteroalkyl-(C≡C)₀₋₁—W, —C(O)O—(C₀-C₆)alkyl-heteroaryl-(C₀-C₄)alkyl-(CH═CH)₀₋₁—W, —C(O)O—(C₀-C₆)heteroalkyl-heteroaryl-(C₀-C₄)alkyl-(CH═CH)₀₋₁—W, —C(O)O—(C₁-C₆)alkyl-heteroaryl-(C₀-C₄)heteroalkyl-(CH═CH)₀₋₁—W, —C(O)O—(C₁-C₆)heteroalkyl-heteroaryl-(C₀-C₄)heteroalkyl-(CH═CH)₀₋₁—W, —C(O)O—(C₁-C₆)alkyl-heteroaryl-(C₀-C₄)alkyl-(C≡C)₀₋₁—W, —C(O)O—(C₁-C₆)heteroalkyl-heteroaryl-(C₀-C₄)alkyl-(C≡C)₀₋₁—W, —C(O)O—(C₁-C₆)alkyl-heteroaryl-(C₀-C₄)heteroalkyl-(C≡C)₀₋₁—W, —C(O)O—(C₁-C₆)heteroalkyl-heteroaryl-(C₀-C₄)heteroalkyl-(C≡C)₀₋₁—W, —C(O)N(R¹)—(C₀-C₆)alkyl-aryl-(C₀-C₄)alkyl-(CH═CH)₀₋₁—W, —C(O)N(R₁)—(C₀-C₆)heteroalkyl-aryl-(C₀-C₄)alkyl-(CH═CH)₀₋₁—W, —C(O)N(R₁)—(C₀-C₆)alkyl-aryl-(C₀-C₄)heteroalkyl-(CH═CH)₀₋₁—W, —C(O)N(R₁)—(C₁-C₆)heteroalkyl-aryl-(C₀-C₄)heteroalkyl-(CH═CH)₀₋₁—W, —C(O)N(R₁)—(C₁-C₆)alkyl-aryl-(C₀-C₄)alkyl-(C≡C)₀₋₁—W, —C(O)N(R₁)—(C₁-C₆)heteroalkyl-aryl-(C₀-C₄)alkyl-(C≡C)₀₋₁—W, —C(O)N(R₁)—(C₁-C₆)alkyl-aryl-(C₀-C₄)heteroalkyl-(C≡C)₀₋₁—W, —C(O)N(R₁)—(C₁-C₆)heteroalkyl-aryl-(C₀-C₄) heteroalkyl-(C≡C)₀₋₁—W, —C(O)N(R¹)—(C₁-C₆)alkyl-heteroaryl-(C₀-C₄)alkyl-(CH═CH)₀₋₁—W, —C(O)N(R¹)—(C₁-C₆)heteroalkyl-heteroaryl-(C₀-C₄)alkyl-(CH═CH)₀₋₁—W, —C(O)N(R¹)—(C₁-C₆)alkyl-heteroaryl-(C₀-C₄)heteroalkyl-(CH═CH)₀₋₁—W, —C(O)N(R¹)—(C₁-C₆)heteroalkyl-heteroaryl-(C₀-C₄)heteroalkyl-(CH═CH)₀₋₁—W, —C(O)N(R¹)—(C₁-C₆)alkyl-heteroaryl-(C₀-C₄)alkyl-(C≡C)₀₋₁—W, —C(O)N(R¹)—(C₁-C₆)heteroalkyl-heteroaryl-(C₀-C₄)alkyl-(C≡C)₀₋₁—W, —C(O)N(R¹)—(C₁-C₆)alkyl-heteroaryl-(C₀-C₄)heteroalkyl-(C≡C)₀₋₁—W and —C(O)N(R¹)—(C₁-C₆)heteroalkyl-heteroaryl-(C₀-C₄)heteroalkyl-(C≡C)₀₋₁—W;
wherein each of the alkyl and heteroalkyl moieties is optionally substituted; and
wherein each of the aryl, heteroaryl, cycloalkyl or heterocyclyl moieties is optionally substituted with one or more groups selected from R²; and
the asterisk mark * indicates a chiral carbon atom,
with the proviso that no more than two of Z, A, B, D and E end with the moiety W.

2. The compound according to claim 1, wherein n is 0.

3. The compound according to claim 1, wherein n is 1.

4. The compound according to claim 1, wherein X¹, X², X³ and X⁴ are independently selected from the group consisting of CH and C-Z, wherein no more than one of X¹, X², X³ and X⁴ are C-Z.

5. The compound according to claim 1, wherein X¹, X², X³ and X⁴ are independently selected from the group consisting of CH, N and C-Z, wherein no more than two of X¹, X², X³ and X⁴ are N and no more than one of X¹, X², X³ and X⁴ are C-Z, wherein Z is selected from the group consisting of —H, halo, —CF₃, —NO₂, —CN, —(C₀-C₆)alkyl-OR¹, —(C₀-C₆)alkyl-N(R¹)₂, —(C₁-C₆)alkyl, —N(R¹)—C(O)—(C₁-C₆)alkyl, —N(R¹)—S(O)₂—(C₁-C₆)alkyl, —O—(C₂-C₆)alkyl-N(R¹)(R¹), S—R¹, —(C₀-C₆)alkyl-C(O)—OR¹, —N(R¹)—C(O)—CF₃ or —N(R¹)—(C₂-C₆)alkyl-N(R¹)(R¹), —(C₀-C₇)alkyl-W, —C(O)—N(R¹)—(C₁-C₆)alkyl-W, —(C₀-C₃)alkyl-N(R¹)—C(O)—(C₁-C₆)alkyl-W, —(C₀-C₃)alkyl-N(R¹)—C(O)—(C₁-C₆)alkyl-C(O)-aryl, —(C₀-C₃)alkyl-N(R¹)—C(O)—(C₁-C₆)alkyl-C(O)-heteroaryl, —(C₀-C₃)alkyl-N(R¹)—C(O)—(C₁-C₆)alkyl-C(O)—N(R¹)-aryl, —(C₀-C₃)alkyl-N(R¹)—C(O)−(C₁-C₆)alkyl-C(O)—N(R¹)-heteroaryl, —(C₀-C₇)alkyl-aryl-W, —(C₀-C₆)alkyl-OR¹, —N(R¹)—C(O)—OR¹, wherein each of the aryl, heteroaryl, cycloalkyl and heterocyclyl moieties of the above-mentioned Z is optionally substituted with one or more substituents selected from the group consisting of oxo, —OH, —CN, —(C₁-C₆)alkyl, —(C₁-C₆)alkoxy, —NO₂, —N(R¹)₂, halo, —SH, mono- to per-halogenated-(C₁-C₆)alkyl and —(C₂-C₄)alkyl-N(R¹)₂, wherein two R¹ groups, together with the nitrogen atom to which they are attached optionally form a heterocyclyl group.

6. The compound according to claim 1, wherein X¹, X², X³ and X⁴ are independently selected from the group consisting of CH, C-Z and N, wherein no more than two of X¹, X², X³ and X⁴ are N and no more than one of X¹, X², X³ and X⁴ are C-Z, wherein Z is selected from the group consisting of —F, —Cl, —Br, CF₃, NO₂, —CN, —OR¹, —NR¹R¹, —(CH₂)₀₋₄OR¹, —(CH₂)₀₋₄N(R¹)₂, —CH₂OH, —CH₃, —N(R¹)C(O)CH₃, —N(R¹)SO₂CH₃, —O(CH₂)₂₋₄N(R¹)(R¹), —SR¹, —(CH₂)₀₋₄C(O)OR¹, —N(R¹)C(O)CF₃ and —N(R¹)(CH₂)₂N(R¹)(R¹), wherein two R¹ groups, together with the nitrogen atom to which they are attached, optionally form a heterocyclyl group.

7. The compound according to claim 1, wherein X¹, X², X³ and X⁴ are independently selected from the group consisting of CH and C-Z, wherein only one of X¹, X², X³ and X⁴ are C-Z, and wherein Z is selected from the group consisting of —H, —(C₀-C₇)alkyl-W, —(C₀-C₅)alkyl-CH═CH—W, —(C₀-C₅)alkyl-C≡C—W, —C(O)—(C₁-C₇)alkyl-W, —(C₀-C₃)alkyl-N(R¹)—C(O)—(C₁-C₆)alkyl-W, —(C₀-C₃)alkyl-N(R¹)—C(S)—(C₁-C₆)alkyl-W, —C(O)—N(R¹)—(C₁-C₆)alkyl-W, —C(S)—N(R¹)—(C₁-C₆)alkyl-W, —(C₀-C₃)alkyl-N(R¹)—(C₁-C₆)alkyl-W, —(C₀-C₃)alkyl-N(R¹)—C(O)—N(R¹)—(C₁-C₆)alkyl-W, —(C₀-C₃)alkyl-N(R¹)—C(S)—N(R¹)—(C₁-C₆)alkyl-W, —(C₀-C₃)alkyl-N(R¹)—C(O)—O—(C₁-C₆)alkyl-W, —S(O)₂—N(R¹)—(C₁-C₆)alkyl-W, —(C₀-C₃)alkyl-N(R¹)—S(O)₂—(C₁-C₆)alkyl-W, —O—C(O)—N(R¹)₂, —(C₀-C₆)alkyl-O—C(O)—N(R¹)—(C₁-C₆)alkyl-W, —(C₀-C₃)alkyl-O—(C₁-C₆)alkyl-W, —(C₀-C₃)alkyl-S—(C₁-C₆)alkyl-W, —N(R¹)—C(O)—O—S(O)₂—N(R¹)₂, —N(R¹)—S(O)₂—R¹, —(C₀-C₇)alkyl-aryl-W, —(C₀-C₇)alkyl-heteroaryl-W, —(C₀-C₃)alkyl-O—(C₀-C₃)alkyl-aryl, —(C₀-C₃)alkyl-O—(C₀-C₃)alkyl-heteroaryl, -aryl, —(C₁-C₆)alkylaryl, -heteroaryl, —(C₁-C₆)alkylheteroaryl, —(C₁-C₈)heteroalkyl, —(C₃-C₆)cycloalkyl, —(C₃-C₆)heterocycloalkyl, —(C₀-C₃)alkyl-N(R¹)—C(O)—(C₁-C₆)alkyl-C(O)-aryl, —(C₀-C₃)alkyl-N(R¹)—C(O)—(C₁-C₆)alkyl-C(O)-heteroaryl, —(C₀-C₃)alkyl-N(R¹)—C(O)—(C₁-C₆)alkyl-C(O)—N(R¹)-aryl and —(C₀-C₃)alkyl-N(R¹)—C(O)—(C₁-C₆)alkyl-C(O)—N(R¹)-heteroaryl, wherein two R¹ groups, together with the nitrogen atom to which they are attached, optionally form a heterocyclyl group.

8. The compound according to claim 7, wherein R¹ is independently —(C₀-C₆)alkyl-aryl or —(C₁-C₄)alkyl.

9. The compound according to claim 8, wherein R¹ is independently selected from the group consisting of phenyl, benzyl, methyl, ethyl, t-butyl and i-propyl.

10. The compound according to claim 7, wherein when Z is —(C₂-C₄)alkyl-N(R¹)₂ the two R¹ groups, together with the nitrogen atom to which they are attached, optionally form a heterocyclyl selected from the group consisting of morpholinyl, piperazinyl, piperidinyl, pyrrolydinyl, and azetidinyl.

11. The compound according to claim 1, wherein W is selected from the group consisting of 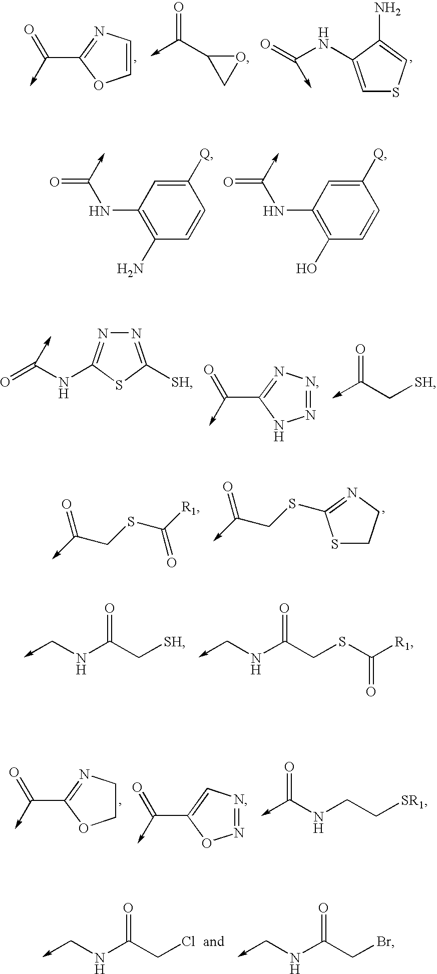
wherein Q is selected from the group consisting of —H, —(C₁-C₆)alkyl, —(C₀-C₆)alkyl-OR¹, heterocyclyl, —N(R¹)₂, halo, aryl and heteroaryl;

12. The compound according to claim 1, wherein W is selected from the group consisting of —C(O)—NH—OH, —COCF₃, —COCHF₂, —COCH₂F, —C(O)CH₃, —C(O)C₂H₅, —(CH₂)₁₋₆—N(OH)C(O)H and —CON(R¹)₂.

13. The compound according to claim 1, wherein Q is independently selected from the group consisting of heterocyclyl, aryl and heteroaryl.

14. The compound according to claim 13, wherein Q is independently selected from the group consisting of thiophenyl, furanyl, tetrazolyl, imidazolyl, pyridinyl, pyrimidinyl, pyrrolyl, thiazolyl, oxazolyl and isooxazolyl.

15. The compound according to claim 1, wherein E and D are independently selected from the group consisting of —H, —(C₁-C₆)alkyl, —(C₁-C₆)heteroalkyl, —(C₁-C₆)alkyl-OR¹, —(C₁-C₆)alkyl-C(O)—N(R¹)₂, —(C₁-C₆)alkyl-C(O)—O—(C₁-C₆)alkyl, 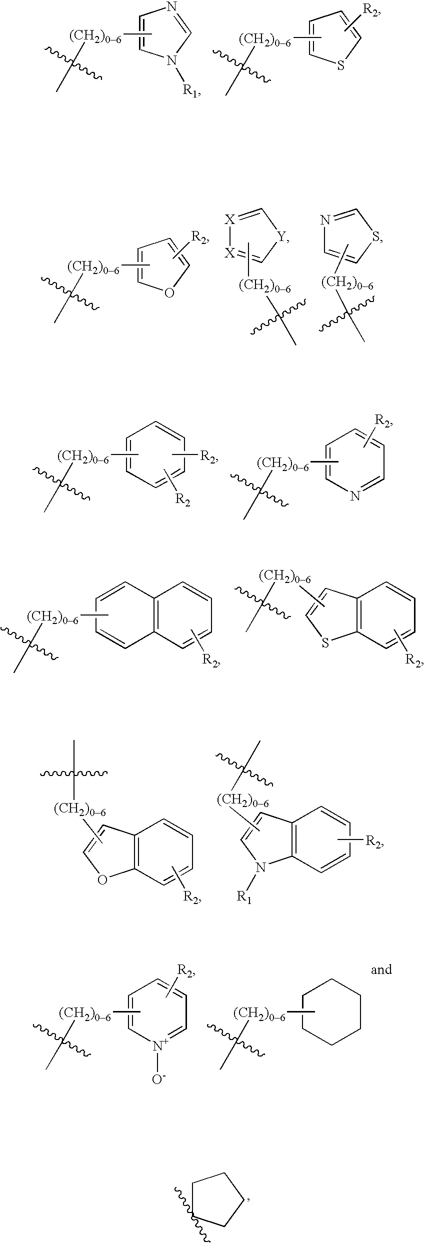
wherein Y is selected from the group consisting of —O—, —NR¹—, and —S—, and X is —CH— or —N—.

16. The compound according to claim 1, wherein E and D together with the carbon atom to which they are attached form a 3- to 6-membered cycloalkyl wherein the cycloalkyl is optionally substituted.

17. The compound according to claim 1, wherein R² is independently selected from the group consisting of —H, —CH₃, —OR₁, —(CH₂)₀₋₄N(R₁)₂, —F, —Cl, —Br, —OCF₃, —CF₃, —C(Ph)₃, NO₂, alkyl, aryl, heteroaryl, SR₁ and —CN.

18. The compound according to claim 1, wherein A and B are independently selected from the group consisting of —H, —(C₁-C₆)alkyl, heteroalkyl, —(C₃-C₆)cycloalkyl, heterocycle, —(C₀-C₃)alkyl-aryl, —(C₀-C₃)alkyl-heteroaryl, —(CH₂)₁₋₅—W, —S(O)₂—(CH₂)₀₋₅-aryl, —S(O)₂—(CH₂)₀₋₅—heteroaryl and —C(O)—R²;
wherein each of the alkyl and heteroalkyl moieties is optionally substituted; and
wherein each of the aryl and heteroaryl moieties is optionally substituted with one or more moieties selected from the group consisting of —(C₀-C₆)alkyl-aryl, —(C₀-C₆)alkyl-heteroaryl, —(C₁-C₆)alkyl, halo, —OH, —O—(C₁-C₆)alkyl, —C(O)OH, —C(O)—NH—OH.

19. The compound according to claim 1, wherein A and B are independently selected from the group consisting of —H, 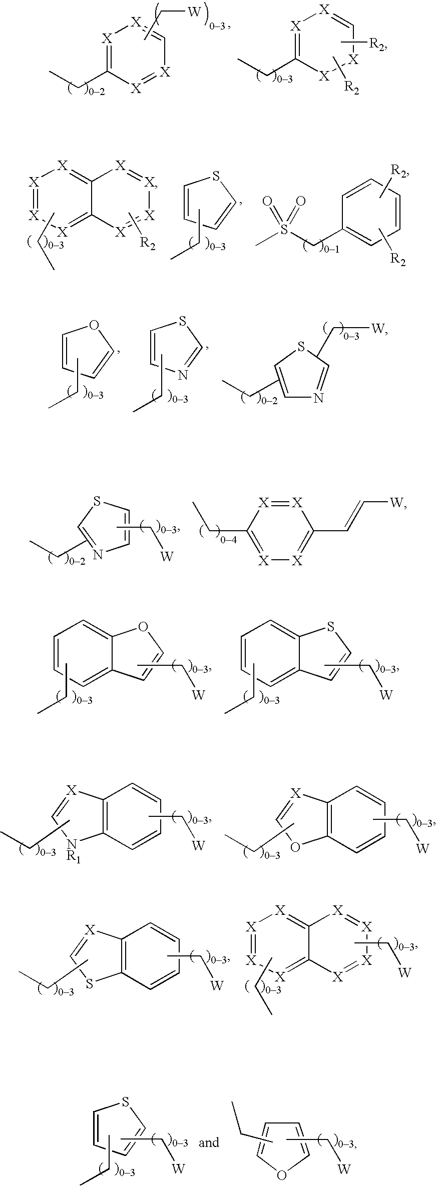
wherein X is —CH— or —N—.

20. The compound according to claim 1, wherein
X¹, X³ and X⁴ are CH;
X² is C-Z;
n is 0; and
A is —H,
with the proviso that one of E or D is H.

21. The compound according to claim 20, of the formula (II) 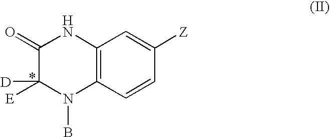
and N-oxides, hydrates, solvates, pharmaceutically acceptable salts, prodrugs and complexes thereof, wherein
Z is selected from the group consisting of —H, —C(O)—N(R¹)₂, —C(O)—N(R¹)—(C₁-C₆)alkyl-W, —(C₀-C₇)alkyl-W, —(C₂-C₇)alkenyl-W, —(C₀-C₃)alkyl-N(R¹)—C(O)—(C₁-C₆)alkyl-W and —(C₀-C₇)alkyl-aryl-W;
B is selected from the group consisting of —H, —S(O)₂—(C₀-C₆)alkyl-aryl, —(C₀-C₆)alkyl-aryl, —(C₀-C₆)alkyl-heteroaryl and —(C₀-C₇)alkyl-aryl-(CH═CH)₀₋₁—W; and
E and D are independently selected from the group consisting of —H, —(C₁-C₆)alkyl, —(C₁-C₆)heteroalkyl, —(C₀-C₆)alkyl-aryl, —(C₀-C₆)alkyl-heteroaryl, —(C₀-C₆)alkyl-W, —(C₀-C₆)alkyl-C(O)—N(R¹)₂, wherein each of the aryl and heteroaryl is optionally substituted with one or more groups selected from R²,
with the proviso that one of E and D is —H.

22. The compound according to claim 21, or a pharmaceutically acceptable salt thereof, wherein
Z is selected from the group consisting of —C(O)—N(R¹)₂, —C(O)—N(R¹)—(C₁-C₆)alkyl-W; and
B is —H.

23. The compound according to claim 22, of the formula (III) 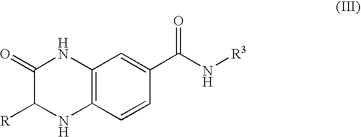
and N-oxides, hydrates, solvates, pharmaceutically acceptable salts, prodrugs and complexes thereof, wherein R and R³ are a combination selected from the group consisting of:

| R | R₃ |
|---|---|
| 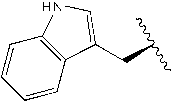 | 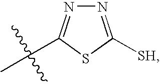 |
| 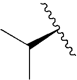 | 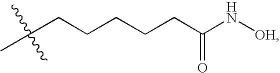 |
| 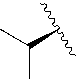 | —OH, |
| 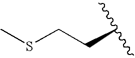 | —OH, |
| 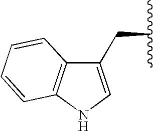 | —OH, |
| 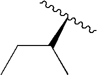 | —OH, |
| 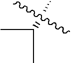 | —OH, |
| 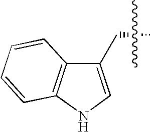 | —OH, |
| 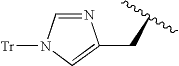 | —OH, |
| 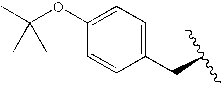 | —OH, |
| 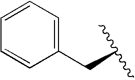 | —OH, |
| H | —OH, |
| 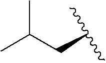 | —OH, |
| 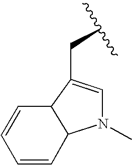 | —OH, |
| 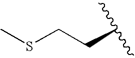 | 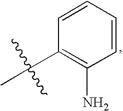 |
| 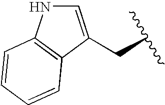 | 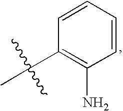 |
| 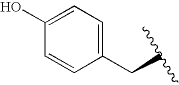 | —OH, |
| 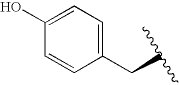 | 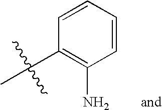 |
| 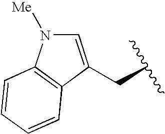 | —OH, |

24. The compound according to claim 21, or a pharmaceutically acceptable salt thereof, wherein
Z is —C(O)—NH—OH;
B is selected from the group consisting of —S(O)₂—(C₀-C₆)alkyl-aryl, —(C₀-C₆)alkyl-aryl, —(C₀-C₆)alkyl-heteroaryl, each of which is optionally substituted and —(C₀-C₇)alkyl-aryl-(CH═CH)₀₋₁—W; and
E and D are independently selected from the group consisting of —H and —(C₁-C₆)alkyl,
wherein the alkyl moiety is optionally substituted,
with the proviso that one of C and D is —H.

25. The compound according to claim 21, of the formula (IV) 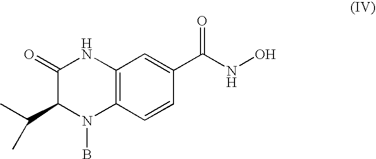
and N-oxides, hydrates, solvates, pharmaceutically acceptable salts, prodrugs and complexes thereof, wherein B is selected from the group consisting of

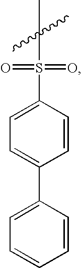

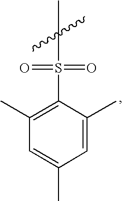

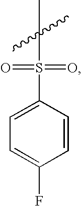

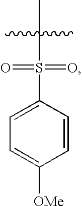

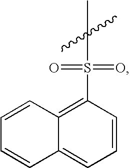

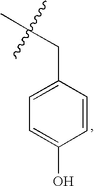

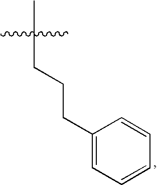

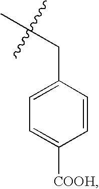

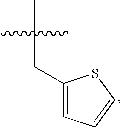

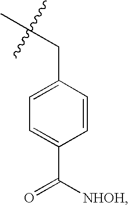

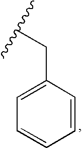

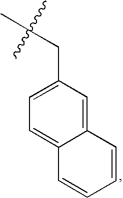

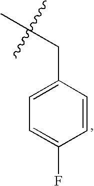

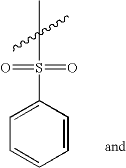

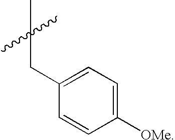

26. The compound according to claim 1, wherein
n is 0;
X¹, X³ and X⁴ are CH;
X² is C-Z;
Z is —(C₀-C₃)alkyl-N(R¹)—C(O)—(C₁-C₆)alkyl-W;
W is selected from the group consisting of —C(O)—NH—OH, —C(O)-heteroaryl, —C(O)-aryl, —C(O)—OR¹, —C(O)—N(R¹)₂ and —C(O)-alkyl, wherein the aryl and heteroaryl moieties of said W are optionally substituted;
A is —H;
B is —H or —(C₀-C₆)alkyl-aryl, wherein the aryl moiety is optionally substituted with one or more groups selected from R²; and
E and D are independently selected from the group consisting of —H, —(C₁-C₆)alkyl and —(C₀-C₆)alkyl-heteroaryl, wherein the heteroaryl moiety is optionally substituted, with the proviso that at least one of E and D are —H.

27. The compound according to claim 25, of the formula (V) 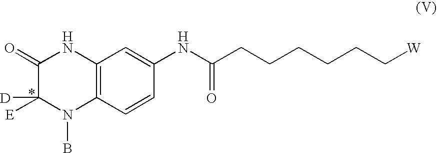
and N-oxides, hydrates, solvates, pharmaceutically acceptable salts, prodrugs and complexes thereof, wherein the combination of R and R⁴ is selected from the group consisting of

| B | E | D | W |
|---|---|---|---|
| H | 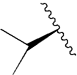 | H | 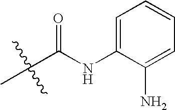 |
| H | 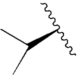 | H | 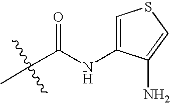 |
| H | 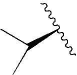 | H | 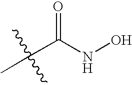 |
| H | H | 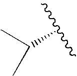 | 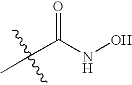 |
| H | H | 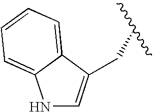 | 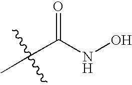 |
| 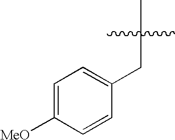 | H | 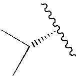 | 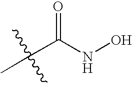 |
| H | 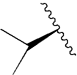 | H | 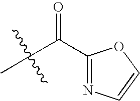 |
| H | 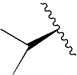 | H | 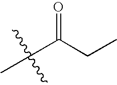 |

28. The compound according to claim 1, wherein
n is 0;
X¹, X², X³ and X⁴ are CH;
A is H;
B is selected from the group consisting of —(C₀-C₆)alkyl-aryl and —(C₀-C₆)alkyl-aryl-(CH═CH)₀₋₁—W, wherein the W moiety is optionally meta or para to the —(C₀-C₆)alkyl moiety, and wherein the aryl moiety of each of the aforementioned B is optionally substituted with one or more substitutents selected from R²;
W is selected from the group consisting of —C(O)—NH—OH, —C(O)—NH-aryl, wherein the aryl is optionally substituted,
E and D are independently selected from the group consisting of —H, —(C₁-C₆)alkyl-M-(C₁-C₃)alkyl-W, —(C₀-C₆)alkyl-C(O)—N(R¹)₂, —(C₀-C₆)alkyl-heteroaryl, —(C₀-C₆)alkyl-aryl and —(C₁-C₆)alkyl-N(R¹)—C(O)—OR¹; and
R¹ is independently selected from the group consisting of —H and —(C₁-C₆)alkyl.

29. The compound according to claim 28, of the formula (VI) 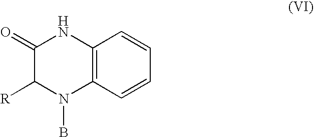
and N-oxides, hydrates, solvates, pharmaceutically acceptable salts, prodrugs and complexes thereof, wherein B and R are a combination selected from the group consisting of

| R | B |
|---|---|
| 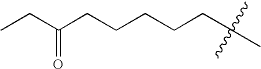 | 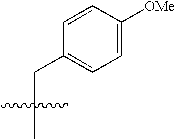 |
| 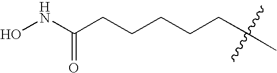 | 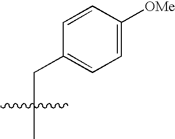 |
| 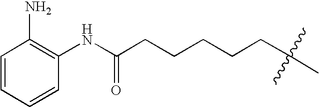 | 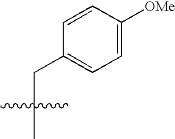 |
| 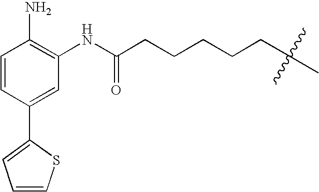 | 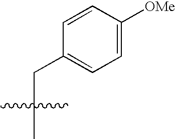 |

30. The compound according to claim 28, of the formula (VII) 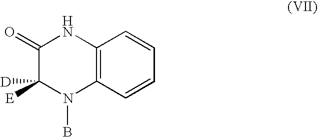
and N-oxides, hydrates, solvates, pharmaceutically acceptable salts, prodrugs and complexes thereof, wherein Bu D and E are a combination selected from the group consisting of

| D | E | B |
|---|---|---|
| 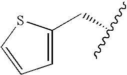 | H | 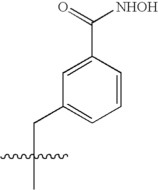 |
| 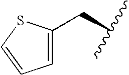 | H | 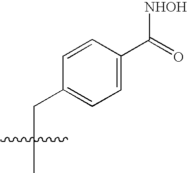 |
| H | 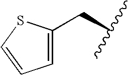 | 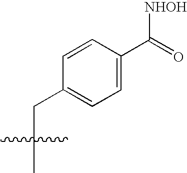 |
| 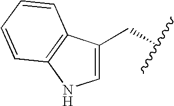 | H | 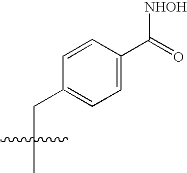 |
| H | 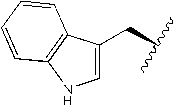 | 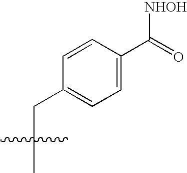 |
| H | 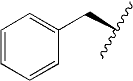 | 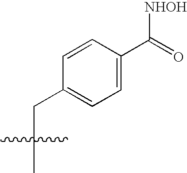 |
| 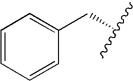 | H | 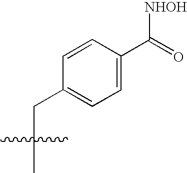 |
| H | H | 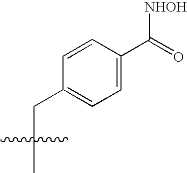 |
| 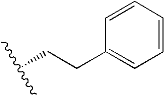 | H | 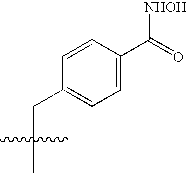 |
| 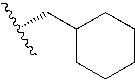 | H | 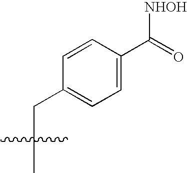 |
| 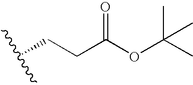 | H | 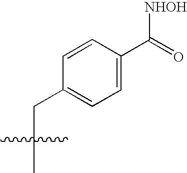 |
| 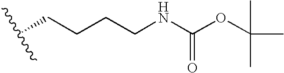 | H | 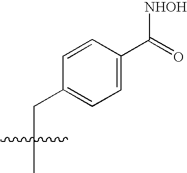 |
| 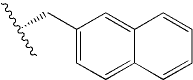 | H | 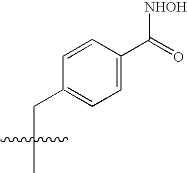 |
| 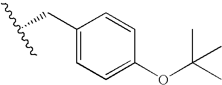 | H | 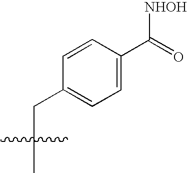 |
| 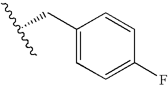 | H | 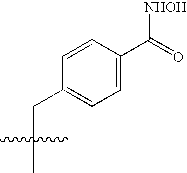 |
| 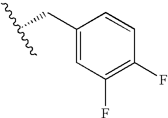 | H | 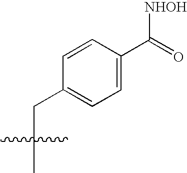 |
| 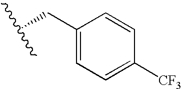 | H | 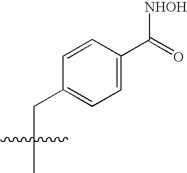 |
| 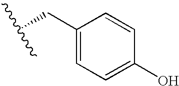 | H | 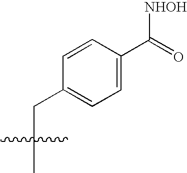 |
| H | 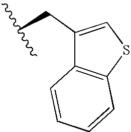 | 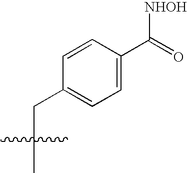 |
| 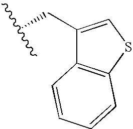 | H | 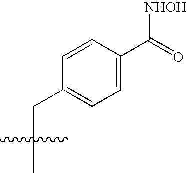 |
| 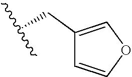 | H | 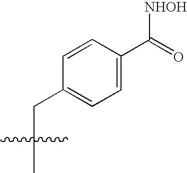 |
| 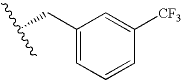 | H | 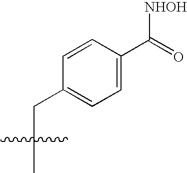 |
| H | 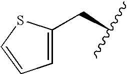 | 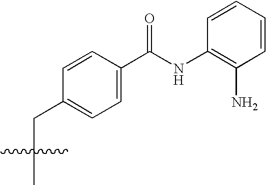 |

31. The compound according to claim 1, wherein
n is 0;
X¹, X² and X³ are CH;
X⁴ is C-Z;
Z is —(C₀-C₇)alkyl-aryl-W;
W is —C(O)—N(R₁)₂;
A and B are —H; and
E and D are independently selected from the group consisting of —H and —(C₁-C₆)alkyl-heteroaryl, with the proviso that one of C and D is —H.

32. The compound according to claim 30, of the formula (VIII) 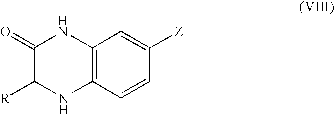
and N-oxides, hydrates, solvates, pharmaceutically acceptable salts, prodrugs and complexes thereof, wherein the combination of R and Z is selected from the group consisting of

| R | Z |
|---|---|
| 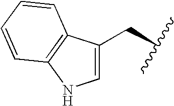 | 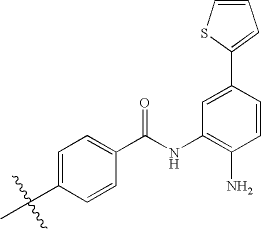 |

33. The compound according to claim 1, wherein
n is 0;
X¹, X² and X³ are CH;
X⁴ is C-Z;
Z is —(C₀-C₇)alkyl-W or —(C₂-C₇)alkenyl-W;
W is —C(O)—NH—OH;
A and B are —H; and
E and D are independently selected from the group consisting of —H and —(C₁-C₆)alkyl-heteroaryl, with the proviso that one of C and D is —H.

34. The compound according to claim 32, of the formula (IX) 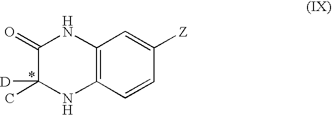
and N-oxides, hydrates, solvates, pharmaceutically acceptable salts, prodrugs and complexes thereof, wherein the combination of
E, D and Z is selected from the group consisting of

| E | D | Z |
|---|---|---|
| 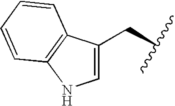 | H | 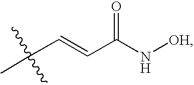 |
| 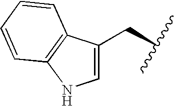 | H | 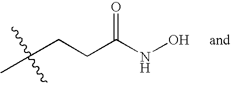 |
| H | 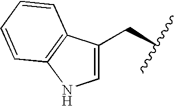 | 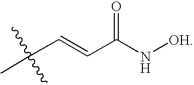 |

35. The compound according to claim 1, wherein
n is 1;
X¹ and X⁴ are CH;
X² and X³ are C-Z;
Z is selected from the group consisting of —H, —(C₀-C₇)alkyl-W, —(C₀-C₆)alkyl-OR¹, —N(R¹)—C(O)—OR¹ and —(C₀-C₃)alkyl-N(R¹)—C(O)—(C₁-C₆)alkyl-W;
A is selected from the group consisting of —H and —(C₁-C₇)alkyl-W, —(C₀-C₆)alkyl-aryl, —(C₀-C₆)alkyl-heteroaryl, wherein the aryl and heteoraryl moiety are optionally substituted with one or more substituents selected from the group consisting of R²;
B is —H;
D and E are independently selected from the group consisting of —H, —(C₁-C₆)alkyl, —(C₀-C₆)alkyl-(C₃-C₆)cylcoalkyl, —(C₀-C₆)alkyl-aryl, —(C₁-C₆)alkyl-heteroaryl, —(C₁-C₆)alkyl-W,
wherein each of the cylcoalkyl, aryl and heteroaryl moieties is optionally substituted with one or more groups selected from R²;
W is independently selected from the group consisting of —C(O)—NH—OH, —C(O)—OR¹, —C(O)—N(R¹)₂;
R¹ is independently selected from the group consisting of —H and —(C₀-C₆)-alkyl-aryl, —(C₀-C₆)alkyl-heteroaryl, —(C₁-C₆)-alkyl wherein each of the aryl and heteroaryl moieties is optionally substituted; and
R² is selected from the group consisting of —(C₀-C₆)alkyl substituted with halo, —(C₀-C₆)alkyl-OR₁, —(C₁-C₇)alkyl-W.

36. The compound according to claim 35, wherein
X¹, X², X³ and X⁴ are CH;
A is selected from the group consisting of —(C₁-C₇)alkyl-W;
D and E are independently selected from the group consisting of —H, —(C₁-C₆)alkyl, —(C₀-C₆)alkyl-(C₃-C₆)cylcoalkyl, —(C₀-C₆)alkyl-aryl, —(C₁-C₆)alkyl-heteroaryl, wherein each of the cylcoalkyl, aryl and heteroaryl moieties is optionally substituted with one or more groups selected from R²;
W is independently selected from the group consisting of —C(O)—NH—OH, —C(O)—OR¹, —C(O)—N(R¹)₂;
R¹ is independently selected from the group consisting of —H, —(C₀-C₆)-alkyl-aryl and —(C₀-C₆)alkyl-heteroaryl, wherein each of the aryl and heteroaryl moieties is optionally substituted; and
R² is selected from the group consisting of —(C₀-C₆)alkyl-OR₁.

37. The compound according to claim 36, of the formula (X) 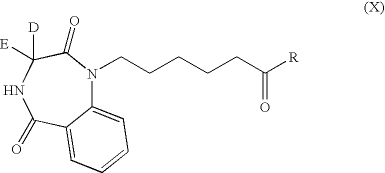
and N-oxides, hydrates, solvates, pharmaceutically acceptable salts, prodrugs and complexes thereof, wherein D, E and R are a combination selected from the group consisting of

| D | E | R |
|---|---|---|
| 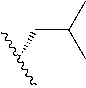 | H | 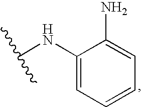 |
| H | 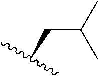 | 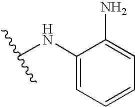 |
| 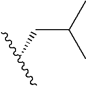 | H | 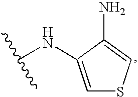 |
| H | 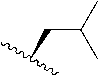 | 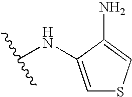 |
| 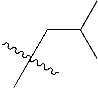 | H | 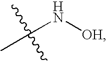 |
| H | H | 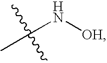 |
| 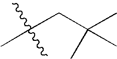 | H | 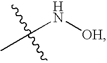 |
| 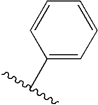 | H | 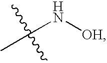 |
| H | 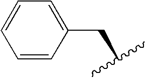 | —OH, |
| H | 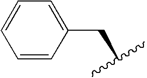 | —NH—OH |
| 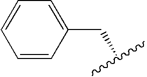 | H | 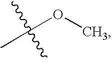 |
| 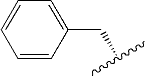 | H | —OH, |
| 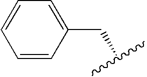 | H | —NH—OH, |
| H | 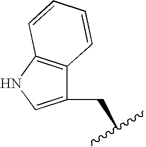 | 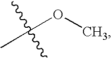 |
| H | 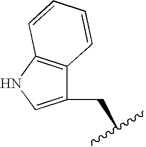 | —OH, |
| H | 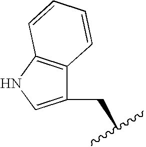 | —NH—OH, |
| 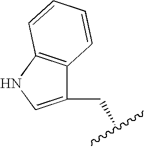 | H | —OH, |
| 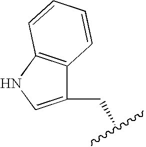 | H | —NH—OH, |
| 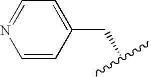 | H | —OH, |
| 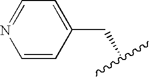 | H | —NH—OH, |
| H | 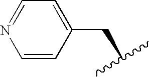 | —OH, |
| H | 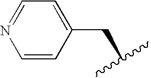 | —NH—OH, |
| H | 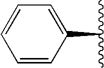 | —OH, |
| H | 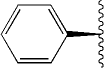 | —NH—OH, |
| 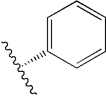 | H | —NH—OH, |
| 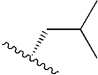 | H | —OH, |
| 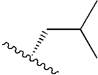 | H | —NH—OH, |
| H | 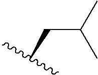 | —NH—OH, |
| H | 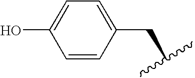 | —OH, |
| 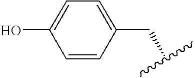 | H | —OH, |
| H | 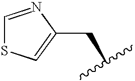 | —OH, |
| 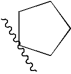 | | —NH—OH, |
| H | 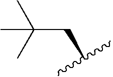 | —NH—OH, |
| 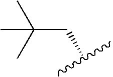 | H | —NH—OH, |
| H | 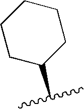 | —NH—OH, |
| 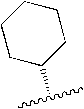 | H | —NH—OH, |
| H | 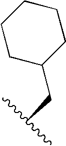 | —NH—OH, |
| 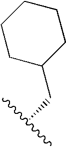 | H | —NH—OH, |
| H | 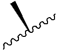 | —NH—OH and |
| 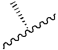 | H | —NH—OH. |

38. The compound according to claim 36, of the formula (XI) 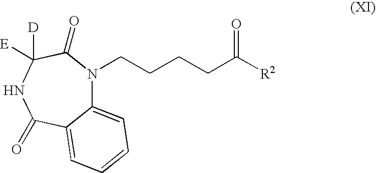
and N-oxides, hydrates, solvates, pharmaceutically acceptable salts, prodrugs and complexes thereof, wherein D, E and R are a combination selected from the group consisting of

| D | E | R |
|---|---|---|
| H | 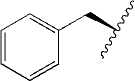 | 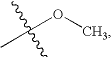 |
| H | 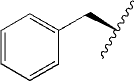 | —OH, |
| 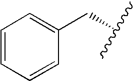 | H | 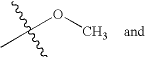 |
| 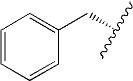 | H | —OH. |

39. The compound according to claim 35, wherein
X¹, X², X³ and X⁴ are CH;
A is selected from the group consisting of —H, —(C₀-C₆)alkyl-aryl, —(C₀-C₆)alkyl-heteroaryl,
wherein the aryl and heteoraryl moiety are optionally substituted with one or more substituents selected from the group consisting of R²;
B is —H;
D and E are independently selected from the group consisting of —H, —(C₁-C₆)alkyl-W;
W is —C(O)—NH—OH; and
R² is selected from the group consisting of —(C₀-C₆)alkyl substituted with halo and —(C₀-C₆)alkyl-OR₁.

40. The compound according to claim 39, of the formula (XII) 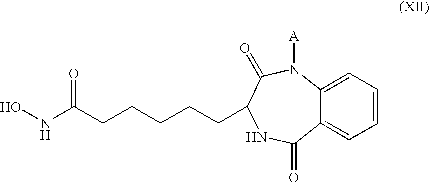
and N-oxides, hydrates, solvates, pharmaceutically acceptable salts, prodrugs and complexes thereof, wherein A is selected from the group consisting of 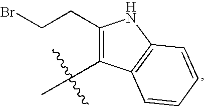
H,

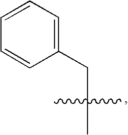

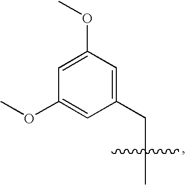

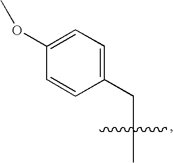

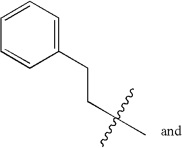

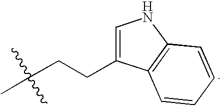

41. The compound according to claim 35, wherein
X¹, X² and X³ are CH;
X³ is C-Z;
Z is —(C₀-C₆)alkyl-OR¹;
R¹ is —(C₀-C₆)alkyl-aryl;
A is —H;
D and E are independently selected from the group consisting of —H and —(C₁-C₆)alkyl-W; and
W is —C(O)—NH—OH and —OR¹.

42. The compound according to claim 41, of the formula (XIII) 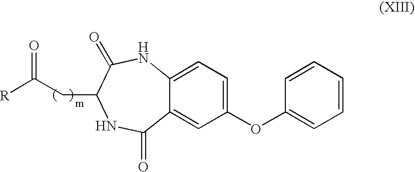
and N-oxides, hydrates, solvates, pharmaceutically acceptable salts, prodrugs and complexes thereof, wherein the combination of m and R is selected from the group consisting of

| m | R |
|---|---|
| 1 | —NH—OH |
| 2 | —NH—OH |
| 5 | —NH—OH |
| 1 | —OH |
| 2 | —OH |
| 4 | —OH |

43. The compound according to claim 35, wherein
X¹, X² and X⁴ are CH;
X³ is C-Z;
Z is selected from the group consisting of —N(R¹)—C(O)—OR¹ and —(C₀-C₃)alkyl-N(R¹)—C(O)—(C₁-C₆)alkyl-W;
A and B are —H;
D and E are independently selected from the group consisting of —H, —(C₁-C₆)alkyl and —(C₁-C₆)alkyl-W;
W is independently selected from the group consisting of —C(O)—NH—OH and —C(O)—OR¹; and
R¹ is independently selected from the group consisting of —H and —(C₀-C₆)-alkyl-aryl, wherein the aryl moiety is optionally substituted.

44. The compound according to claim 43, of the formula (XIV) 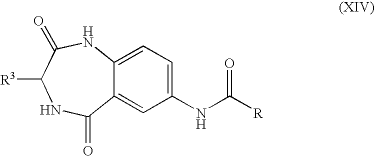
and N-oxides, hydrates, solvates, pharmaceutically acceptable salts, prodrugs and complexes thereof, wherein the combination of D, E and R is selected from the group consisting of

| R³ | R |
|---|---|
| 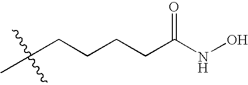 | 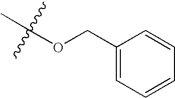 |
| 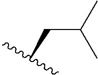 | 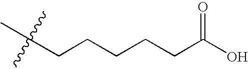 |
| 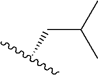 | 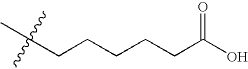 |

45. The compound according to claim 35, wherein
X¹, X³ and X⁴ are CH;
X² is C-Z;
Z is —(C₀-C₇)alkyl-W;
A and B are —H;
D and E are independently selected from the group consisting of —H, —(C₁-C₆)alkyl, —(C₀-C₆)alkyl-(C₃-C₆)cylcoalkyl, —(C₀-C₆)alkyl-aryl and —(C₁-C₆)alkyl-heteroaryl, wherein each of the cylcoalkyl, aryl and heteroaryl moieties is optionally substituted with one or more groups selected from R²; and
W is —C(O)—NH—OH.

46. The compound according to claim 45, of the formula (XV) 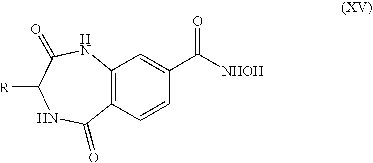
and N-oxides, hydrates, solvates, pharmaceutically acceptable salts, prodrugs and complexes thereof, wherein R is selected from the group consisting of 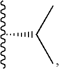 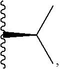 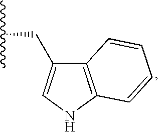 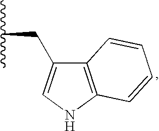 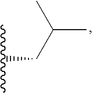 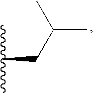 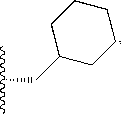 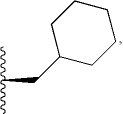 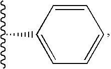 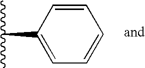
H.

47. The compound of claim 1, or a pharmaceutically acceptable salt thereof, selected from the group consisting of
(S)-1,2,3,4-Tetrahydro-N-hydroxy-2-isopropyl-3-oxoquinoxaline-6-carboxamide,
(S)-1,2,3,4-tetrahydro-N-hydroxy-2-(2-(methylthio)ethyl)-3-oxoquinoxaline-6-carboxamide,
(S)-2-((1H-indol-3-yl)methyl)-1,2,3,4-tetrahydro-N-hydroxy-3-oxoquinoxaline-6-
carboxamide,
(S)-2-sec-butyl-1,2,3,4-tetrahydro-N-hydroxy-3-oxoquinoxaline-6-carboxamide,
(R)-1,2,3,4-tetrahydro-N-hydroxy-2-isopropyl-3-oxoquinoxaline-6-carboxamide,
(R)-2-((1H-indol-3-yl)methyl)-1,2,3,4-tetrahydro-N-hydroxy-3-oxoquinoxaline-6-
carboxamide,
(S)-1,2,3,4-tetrahydro-N-hydroxy-3-oxo-2-((1-trityl-1H-imidazol-4-yl)methyl)quinoxaline-6-
carboxamide,
(S)-2-(4-tert-butoxybenzyl)-1,2,3,4-tetrahydro-N-hydroxy-3-oxoquinoxaline-6-carboxamide,
(S)-2-Benzyl-1,2,3,4-tetrahydro-N-hydroxy-3-oxoquinoxaline-6-carboxamide,
(S)-1,2,3,4-Tetrahydro-N-hydroxy-2-isobutyl-3-oxoquinoxaline-6-carboxamide,
(S)-1,2,3,4-tetrahydro-N-hydroxy-2-((1-methyl-1-H-indol-3-yl)methyl)-3-oxoquinoxaline-6-
carboxamide,
(S)-2-((1H-indol-3-yl)methyl)-N-(2-aminophenyl)-1,2,3,4-tetrahydro-3-oxoquinoxaline-6-
carboxamide,
(S)-2-(4-hydroxybenzyl)-1,2,3,4-tetrahydro-N-hydroxy-3-oxoquinoxaline-6-carboxamide,
(S)-1-(4-Methoxybenzyl)-1,2,3,4-tetrahydro-N-hydroxy-2-isopropyl-3 oxoquinoxaline-6-
carboxamide,
(S)—N-(5-(Hydroxycarbamoyl)pentyl)-1,2,3,4-tetrahydro-2-isopropyl-3-oxoquinoxaline-6-
carboxamide,
(S)-1-(4-fluorobenzyl)-N-hydroxy-2-isopropyl-3-oxo-1,2,3,4-tetrahydroquinoxaline-6-
carboxamide,
(S)-1-(4-hydroxybenzyl)-1,2,3,4-tetrahydro-N-hydroxy-2-isopropyl-3-oxoquinoxaline-6-
carboxamide,
(S)-1,2,3,4-tetrahydro-N-hydroxy-2-isopropyl-3-oxo-1-(3-phenylpropyl)quinoxaline-6-
carboxamide,
4-(((S)-7-(hydroxycarbamoyl)-2,3-dihydro-3-isopropyl-2-oxoquinoxalin-4(1H)-
yl)methyl)benzoic acid,
(S)-1,2,3,4-tetrahydro-N-hydroxy-2-isopropyl-3-oxo-1-((thiophen-2-yl)methyl)quinoxaline-6-
carboxamide,
(S)—N-hydroxy-1-(4-(N-hydroxycarbamimidoyl)benzyl)-2-isopropyl-3-oxo-1,2,3,4-
tetrahydroquinoxaline-6-carboxamide,
(S)-1-benzyl-1,2,3,4-tetrahydro-N-hydroxy-2-isopropyl-3-oxoquinoxaline-6-carboxamide,
(S)-1,2,3,4-tetrahydro-N-hydroxy-2-isopropyl-1-((naphthalen-3-yl)methyl)-3-oxoquinoxaline-
6-carboxamide,
(S)—N-hydroxy-2-isopropyl-3-oxo-1-(phenylsulfonyl)-1,2,3,4-tetrahydroquinoxaline-6-
carboxamide,
(S)—N-hydroxy-1-(biphenyl-4-sulfonyl)-2-isopropyl-3-oxo-1,2,3,4-tetrahydroquinoxaline-6-
carboxamide,
(S)—N-hydroxy-2-isopropyl-3-oxo-1-(2,4,6-trimethybenzenesulfonyl)-1,2,3,4-
tetrahydroquinoxaline-6-carboxamide,
(S)—N-hydroxy-1-(4-methoxybenzenesulfonyl-2-isopropyl-3-oxo-1,2,3,4-
tetrahydroquinoxaline-6-carboxamide,
(S)—N-hydroxy-1-(1-naphthalenesulfonyl-2-isopropyl-3-oxo-1,2,3,4-tetrahydroquinoxaline-6-
carboxamide,
N1-(2-Aminophenyl)-N8—((S)-1,2,3,4-tetrahydro-2-isopropyl-3-oxoquinoxalin-6-
yl)octanediamide,
N1—((S)-1,2,3,4-Tetrahydro-2-isopropyl-3-oxoquinoxalin-6-yl)-N8-hydroxyoctanediamide,
N1—((R)-1,2,3,4-Tetrahydro-2-isopropyl-3-oxoquinoxalin-6-yl)-N8-hydroxyoctanediamide,
N1—((R)-2-((1H-Indol-3-yl)methyl)-1,2,3,4-tetrahydro-3-oxoquinoxalin-6-yl)-N8-
hydroxyoctanediamide,
N1—((R)-1-(4-Methoxybenzyl)-1,2,3,4-tetrahydro-2-isopropyl-3-oxoquinoxalin-6-yl)-N8-
hydroxyoctanediamide,
N—((S)-1,2,3,4-Tetrahydro-2-isopropyl-3-oxoquinoxalin-6-yl)-8-(oxazol-2-yl)-8-
oxooctanamide,
6-((R)-1-(4-methoxybenzyl)-1,2,3,4-tetrahydro-3-oxoquinoxalin-2-yl)-N-hydroxy-4-oxy-
hexanamide,
6-(1-(4-methoxybenzyl)-1,2,3,4-tetrahydro-3-oxoquinoxalin-2-yl)-N-hydroxyhexanamide,
6-(1-(4-methoxybenzyl)-1,2,3,4-tetrahydro-3-oxoquinoxalin-2-yl)-N-(2-
aminophenyl)hexanamide,
6-(1-(4-methoxybenzyl)-1,2,3,4-tetrahydro-3-oxoquinoxalin-2-yl)-N-(2-amino-5-(thiophen-2-
yl)phenyl)hexanamide,
4-(((S)-2,3-Dihydro-2-oxo-3-((thiophen-2-yl)methyl)quinoxalin-4(1H)-yl)methyl)-N-
hydroxybenzamide,
4-(((R)-2,3-Dihydro-2-oxo-3-((thiophen-2-yl)methyl)quinoxalin-4(1H)-yl)methyl)-N-
hydroxybenzamide,
N-hydroxy-4-((3-oxo-3,4-dihydroquinoxalin-1(2H)-yl)methyl)-benzamide,
(R)-4-((2-((1H-indol-3-yl)methyl)-3-oxo-3,4-dihydroquinoxalin-1(2H)-yl)methyl)-N-
hydroxybenzamide,
(S)-4-((2-((1H-indol-3-yl)methyl)-3-oxo-3,4-dihydroquinoxalin-1(2H)-yl)methyl)-N-
hydroxybenzamide,
(S)-4-((2-benzyl-3-oxo-3,4-dihydroquinoxalin-1(2H)-yl)methyl)-N-hydroxybenzamide,
(R)-4-((2-benzyl-3-oxo-3,4-dihydroquinoxalin-1(2H)-yl)methyl)-N-hydroxybenzamide,
N-hydroxy-4-((3-oxo-3,4-dihydroquinoxalin-1(2H)-yl)methyl)benzamide,
(R)—N-hydroxy-4-((3-oxo-2-phenethyl-3,4-dihydroquinoxalin-1(2H)-yl)methyl)benzamide,
(R)-4-((2-(cyclohexylmethyl)-3-oxo-3,4-dihydroquinoxalin-1(2H)-yl)methyl)-N-
hydroxybenzamide,
(R)-tert-butyl 4-(1-(4-(hydroxycarbamoyl)benzyl)-3-oxo-1,2,3,4-tetrahydroquinoxalin-2-
yl)butylcarbamate,
(R)—N-hydroxy-4-((2-(naphthalen-2-ylmethyl)-3-oxo-3,4-dihydroquinoxalin-1(2H)-
yl)methyl)benzamide,
(R)-4-((2-(4-tert-butoxybenzyl)-3-oxo-3,4-dihydroquinoxalin-1(2H)-yl)methyl)-N-
hydroxybenzamide,
(R)-tert-butyl 3-(1-(4-(hydroxycarbamoyl)benzyl)-3-oxo-1,2,3,4-tetrahydroquinoxalin-2-
yl)propanoate,
(R)-4-((2-(benzo[b]thiophen-3-ylmethyl)-3-oxo-3,4-dihydroquinoxalin-1(2H)-yl)methyl)-N-
hydroxybenzamide,
(S)-4-((2-(benzo[b]thiophen-3-ylmethyl)-3-oxo-3,4-dihydroquinoxalin-1(2H)-yl)methyl)-N-
hydroxybenzamide,
(R)-4-((2-(4-fluorobenzyl)-3-oxo-3,4-dihydroquinoxalin-1(2H)-yl)methyl)-N-
hydroxybenzamide,
(R)-4-((2-(3,4-difluorobenzyl)-3-oxo-3,4-dihydroquinoxalin-1(2H)-yl)methyl)-N-
hydroxybenzamide,
(R)-4-((2-(4-trifluoromethylbenzyl)-3-oxo-3,4-dihydroquinoxalin-1(2H)-yl)methyl)-N-
hydroxybenzamide,
(R)—N-hydroxy-4-((2-(4-hydroxybenzyl)-3-oxo-3,4-dihydroquinoxalin-1(2H)-
yl)methyl)benzamide,
(R)—N-hydroxy-4-((2-(3-(trifluoromethyl)benzyl)-3-oxo-3,4-dihydroquinoxalin-1(2H)-
yl)methyl)benzamide,
(R)-4-((2-(furan-3-ylmethyl)-3-oxo-3,4-dihydroquinoxalin-1(2H)-yl)methyl)-N-
hydroxybenzamide,
(R)—N-hydroxy-4-((3-oxo-2-(pyridin-3-ylmethyl)-3,4-dihydroquinoxalin-1(2H)-
yl)methyl)benzamide,
(S)—N-(2-aminophenyl)-4-((3-oxo-2-(thiophen-2-ylmethyl)-3,4-dihydroquinoxalin-1(2H)-
yl)methyl)benzamide,
4-(((R)-2,3-Dihydro-2-oxo-3-((thiophen-2-yl)methyl)quinoxalin-4(1H)-yl)methyl)-N-
hydroxybenzamide,
4-((S)-2-((1H-indol-3-yl)methyl)-1,2,3,4-tetrahydro-3-oxoquinoxalin-6-yl)-N-(2-amino-5-
(thiophen-2-yl)phenyl)benzamide,
(E)-3-((S)-2-((1H-indol-3-yl)methyl)-1,2,3,4-tetrahydro-3-oxoquinoxalin-6-yl)-N-
hydroxyacrylamide,
(E)-3-((R)-2-((1H-indol-3-yl)methyl)-1,2,3,4-tetrahydro-3-oxoquinoxalin-6-yl)-N-
hydroxyacrylamide,
6-((S)-2,3,4,5-tetrahydro-3-isobutyl-2,5-dioxobenzo[e][1,4]diazepin-1-yl)-N-
hydroxyhexanamide,
6-((S)-2,3,4,5-tetrahydro-3-neopentyl-2,5-dioxobenzo[e][1,4]diazepin-1-yl)-N-
hydroxyhexanamide,
6-((R)-2,3,4,5-tetrahydro-2,5-dioxo-3-phenylbenzo[e][1,4]diazepin-1-yl)-N-
hydroxyhexanamide,
6-((R)-2,3,4,5-tetrahydro-2,5-dioxo-3-((pyridin-4-yl)methyl)benzo[e][1,4]diazepin-1-yl)-N-
hydroxyhexanamide,
6-((S)-3-((1H-indol-3-yl)methyl)-2,3,4,5-tetrahydro-2,5-dioxobenzo[e][1,4]diazepin-1-yl)-N-
hydroxyhexanamide,
6-((R)-3-((1H-indol-3-yl)methyl)-2,3,4,5-tetrahydro-2,5-dioxobenzo[e][1,4]diazepin-1-yl)-N-
hydroxyhexanamide,
6-((R)-2,3,4,5-tetrahydro-2,5-dioxo-3-((pyridin-4-yl)methyl)benzo[e][1,4]diazepin-1-yl)-N-
hydroxyhexanamide,
6-((S)-2,3,4,5-tetrahydro-2,5-dioxo-3-phenylbenzo[e][1,4]diazepin-1-yl)-N-
hydroxyhexanamide,
6-((R)-2,3,4,5-tetrahydro-3-isobutyl-2,5-dioxobenzo[e][1,4]diazepin-1-yl)-N-
hydroxyhexanamide,
6-(3,3-spirocyclopentyl-2,5-dioxo-2,3,4,5,tetrahydrobenzo[e][1,4]diazepin-1-yl)-N-
hydroxyhexanamide,
6-((S)-2,3,4,5-tetrahydro-3-neopentyl-2,5-dioxobenzo[e][1,4]diazepin-1-yl)-N-
hydroxyhexanamide,
6-((R)-2,3,4,5-tetrahydro-3-neopentyl-2,5-dioxobenzo[e][1,4]diazepin-1-yl)-N-
hydroxyhexanamide,
6-((S)-3-cyclohexyl-2,3,4,5-tetrahydro-2,5-dioxobenzo[e][1,4]diazepin-1-yl)-N-
hydroxyhexanamide,
6-((S)-3-(cyclohexylmethyl)-2,3,4,5-tetrahydro-2,5-dioxobenzo[e][1,4]diazepin-1-yl)-N-
hydroxyhexanamide,
6-((R)-3-(cyclohexylmethyl)-2,3,4,5-tetrahydro-2,5-dioxobenzo[e][1,4]diazepin-1-yl)-N-
hydroxyhexanamide,
6-((S)-2,3,4,5-tetrahydro-3-methyl-2,5-dioxobenzo[e][1,4]diazepin-1-yl)-N-
hydroxyhexanamide,
6-((R)-2,3,4,5-tetrahydro-3-methyl-2,5-dioxobenzo[e][1,4]diazepin-1-yl)-N-
hydroxyhexanamide,
3-((S)-2-((1H-indol-3-yl)methyl)-1,2,3,4-tetrahydro-3-oxoquinoxalin-6-yl)-N-
hydroxypropanamide,
N-(2-aminophenyl)-6-((R)-2,3,4,5-tetrahydro-3-isobutyl-2,5-dioxobenzo[e][1,4]diazepin-1-
yl)hexanamide,
N-(4-aminothiophen-3-yl)-6-((R)-2,3,4,5-tetrahydro-3-isobutyl-2,5-
dioxobenzo[e][1,4]diazepin-1-yl)hexanamide,
6-(2,3,4,5-tetrahydro-2,5-dioxo-1H-benzo[e][1,4]diazepin-3-yl)-N-hydroxyhexanamide,
6-(1-(2-(1H-indol-3-yl)ethyl)-2,3,4,5-tetrahydro-2,5-dioxo-1H-benzo[e][1,4]diazepin-3-yl)-N-
hydroxyhexanamide,
6-(1-benzyl-2,3,4,5-tetrahydro-2,5-dioxo-1H-benzo[e][1,4]diazepin-3-yl)-N-
hydroxyhexanamide,
6-(1-(3,5-dimethoxybenzyl)-2,3,4,5-tetrahydro-2,5-dioxo-1H-benzo[e][1,4]diazepin-3-yl)-N-
hydroxyhexanamide,
6-(1-(4-methoxybenzyl)-2,3,4,5-tetrahydro-2,5-dioxo-1H-benzo[e][1,4]diazepin-3-yl)-N-
hydroxyhexanamide,
6-(2,3,4,5-tetrahydro-2,5-dioxo-1-phenethyl-1H-benzo[e][1,4]diazepin-3-yl)-N-
hydroxyhexanamide,
6-(2,3,4,5-tetrahydro-2,5-dioxo-7-phenoxy-1H-benzo[e][1,4]diazepin-3-yl)-N-
hydroxyhexanamide,
6-(7-Benzyloxycarbonylamino-2,3,4,5-tetrahydro-2,5-dioxo-1H-benzo[e][1,4]diazepin-3-yl)-
N-hydroxyhexanamide,
(S)-benzyl 3-(6-(hydroxyamino)-6-oxohexyl)-2,5-dioxo-2,3,4,5-tetrahydro-1H-
benzo[e][1,4]diazepin-7-ylcarbamate,
(R)—N-hydroxy-3-isopropyl-2,5-dioxo-2,3,4,5-tetrahydro-1H-benzo[e][1,4]diazepine-8-
carboxamide,
(S)—N-hydroxy-3-isopropyl-2,5-dioxo-2,3,4,5-tetrahydro-1H-benzo[e][1,4]diazepine-8-
carboxamide,
(S)-3-((1H-indol-3-yl)methyl)-N-hydroxy-2,5-dioxo-2,3,4,5-tetrahydro-1H-
benzo[e][1,4]diazepine-8-carboxamide,
(R)-3-((1H-indol-3-yl)methyl)-N-hydroxy-2,5-dioxo-2,3,4,5-tetrahydro-1H-
benzo[e][1,4]diazepine-8-carboxamide,
(R)—N-hydroxy-3-isobutyl-2,5-dioxo-2,3,4,5-tetrahydro-1H-benzo[e][1,4]diazepine-8-
carboxamide,
(R)-3-(cyclohexylmethyl)-N-hydroxy-2,5-dioxo-2,3,4,5-tetrahydro-1H-
benzo[e][1,4]diazepine-8-carboxamide,
(S)-3-(cyclohexylmethyl)-N-hydroxy-2,5-dioxo-2,3,4,5-tetrahydro-1H-
benzo[e][1,4]diazepine-8-carboxamide,
(S)—N-hydroxy-2,5-dioxo-3-phenyl-2,3,4,5-tetrahydro-1H-benzo[e][1,4]diazepine-8-
carboxamide,
(R)—N-hydroxy-2,5-dioxo-3-phenyl-2,3,4,5-tetrahydro-1H-benzo[e][1,4]diazepine-8-
carboxamide,
N-hydroxy-2,5-dioxo-2,3,4,5-tetrahydro-1H-benzo[e][1,4]diazepine-8-carboxamide,
(R)-2-(4-((2-(3,4-Difluorobenzyl)-3-oxo-3,4-dihydroquinoxalin-1(2H)-yl)methyl)phenyl)-N-
hydroxyacetamide,
(R)-2-(3,4-Difluorobenzyl)-N-(4-(hydroxycarbamoyl)phenyl)-3-oxo-3,4-dihydroquinoxaline-
1(2H)-carboxamide,
(R)-2-(3,4-difluorobenzyl)-N-(3-(hydroxycarbamoyl)phenyl)-3-oxo-3,4-dihydroquinoxaline-
1(2H)-carboxamide,
(2S,4S)-Benzyl 4-(5-(hydroxycarbamoyl)pyrimidin-2-ylamino)-2-(quinolin-8-
ylcarbamoyl)pyrrolidine-1-carboxylate,
(R)-4-((2-((1H-Indol-3-yl)methyl)-3,6-dioxopiperazin-1-yl)methyl)-N-hydroxybenzamide,
(R)-4-((2-Benzyl-3,6-dioxopiperazin-1-yl)methyl)-N-hydroxybenzamide,
(R)-4-((3,6-Dioxo-2-(thien-2-ylmethyl)piperazin-1-yl)methyl)-N-hydroxybenzamide and
6-(2,3,4,5-tetrahydro-2,5-dioxobenzo[e][1,4]diazepin-1-yl)-N-hydroxyhexanamide.

48. A compound of the formula XVI, 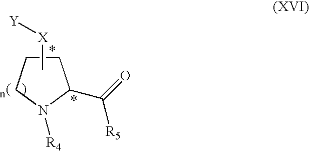
and N-oxides, hydrates, solvates, pharmaceutically acceptable salts, prodrugs and complexes thereof, wherein
n is 1 or 2;
X is selected from the group consisting of —O—, —S—, —N(R¹)— and —CH(R¹)—;
Y is selected from the group consisting of —(C₀-C₇)alkyl-heteroaryl-W, —(C₁-C₇)alkyl-W, —(C₀-C₇)alkyl-aryl-W and —C(O)—(C₁-C₇)alkyl-W;
W is selected from the group consisting of —C(O)—NH—OH, —C(O)—(C₁-C₄)alkyl, —C(O)—N(R¹)₂, —(C₂-C₆)alkyl-N(OH)—C(O)H—, —(C₁-C₆)alkyl-SR¹, —(C₁-C₆)alkyl-SC(O)—(C₁-C₄)alkyl, —C(O)—OR¹, —C(O)—(C₁-C₄)alkylepoxide, —C(O)—(C₁-C₄)alkyl-SH, —C(O)—(C₁-C₄)alkyl-SC(O)R¹, —C(O)—(C₁-C₄)alkyl-5-heteroaryl, —(C₁-C₆)alkyl-NH—C(O)—(C₁-C₆)alkyl-halo, —(C₁-C₆)alkyl-NH—C(O)—(C₁-C₆)alkyl-SH, —(C₁-C₆)alkyl-NH—C(O)—(C₁-C₆)alkyl-SC(O)R¹, —C(O)—NH—(C₂-C₆)alkyl-SH and —C(O)—(C₁-C₆)alkyl,
wherein the alkyl of said —C(O)—(C₁-C₆)alkyl is optionally substituted with one or more substituents selected from the group consisting of mono to per-halogenated —(C₁-C₆)alkyl, —C(O)-heteroaryl, —C(O)—NH-heteroaryl and —C(O)—NH-aryl;
wherein each aryl and heteroaryl is optionally substituted with one or more substituents selected from the group consisting of —NH₂, —OH, SH, —CN, —NO₂, —N(R¹)₂, halo, mono- to per-halogenated-(C₁-C₆)alkyl, aryl, heteroaryl, 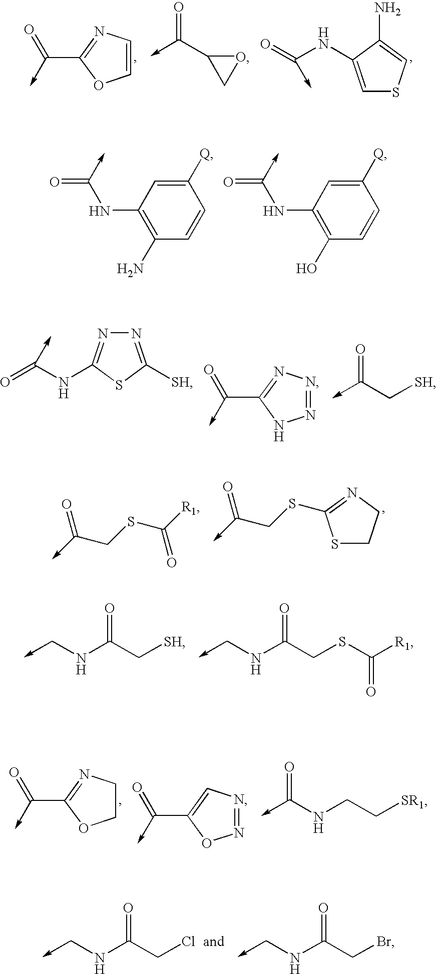
wherein Q is selected from the group consisting of heterocyclic, aryl and heteroaryl;
R¹ is independently selected from the group consisting of —H, —(C₁-C₆)alkyl, —(C₁-C₆)heteroalkyl, —(C₃-C₆)cycloalkyl, -heterocyclyl, —(C₀-C₆)alkyl-aryl and —(C₀-C₆)alkyl-heteroaryl,
wherein each afore-mentioned R¹ aryl, heteroaryl, cycloalkyl and heterocyclyl moiety is optionally substituted with one or more substituents selected from the group consisting of oxo, —OH, —CN, —(C₁-C₆)alkyl, —(C₁-C₆)alkoxy, —NO₂, —N(R¹)₂, halo, —SH, mono- to per-halogenated-(C₁-C₆)alkyl and —(C₂-C₄)alkyl-N(R¹)₂,
wherein optionally the R¹ together with the nitrogen atom to which they are attached form a heterocyclyl group;
R⁴ is selected from the group consisting of —S(O)₂—(C₁-C₆)alkyl, —S(O)₂—(C₁-C₆)heteroalkyl, —S(O)₂—(C₁-C₆)aryl, —S(O)₂—(C₁-C₆)alkylaryl, —S(O)₂—(C₁-C₆)heteroaryl, —S(O)₂—(C₁-C₆)arylalkyl, —S(O)₂—(C₁-C₆)heterocyclic, —C(O)—(C₁-C₆)alkyl, —C(O)—(C₁-C₆)heteroalkyl, —C(O)—(C₁-C₆)aryl, —C(O)—(C₁-C₆)alkylaryl, —C(O)—(C₁-C₆)heteroaryl, —C(O)—(C₁-C₆)arylalkyl, —C(O)—(C₁-C₆)heterocyclic and —C(O)—OR¹;
R⁵ is selected from the group consisting of —OR¹ and —N(R¹)₂; and
the asterisk mark * indicates a chiral carbon atom.
with the proviso that when X is NR¹, Y is —C(O)—(C₁-C₇)alkyl-W or —S(O)₂—(C₁-C₆)alkyl-W.

49. The compound according to claim 48, wherein Q is selected from the group consisting of thiopheneyl, furanyl, tetrazolyl, imidazolyl, pyridinyl and pyrimidinyl.

50. The compound according to claim 49, wherein
n is 1;
X is —O—;
Y is selected from the group consisting of —(C₁-C₇)alkyl-W, —(C₀-C₇)alkyl-aryl-W and —C(O)—(C₁-C₇)alkyl-W;
W is —C(O)—NH—OH;
R⁴ is —C(O)—OR¹; and
R⁵ is —N(R¹)₂.

51. The compound according to claim 48, of the formula XVII 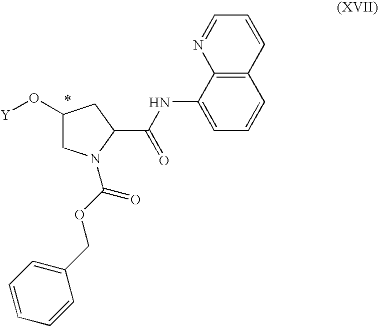
and N-oxides, hydrates, solvates, pharmaceutically acceptable salts, prodrugs and complexes thereof, wherein Y is selected from the group consisting of

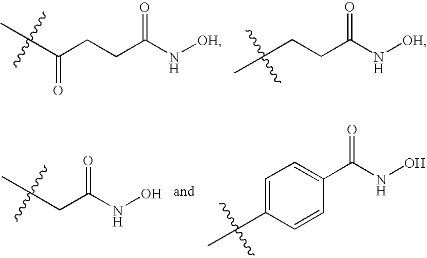

52. The compound according to claim 48, or a pharmaceutically acceptable salt thereof, selected from the group consisting of
(2S,4R)-benzyl 4-(3-(hydroxyamino)-3-oxopropoxy)-2-
(quinolin-8-ylcarbamoyl)pyrrolidine-1-carboxylate,
(2S,4R)-benzyl 4-(2-(hydroxyamino)-2-oxoethoxy)-2-
(quinolin-8-ylcarbamoyl)pyrrolidine-1-carboxylate,
(2R,4R)-benzyl 4-(3-(hydroxyamino)-3-oxopropoxy)-2-
(quinolin-8-ylcarbamoyl)pyrrolidine-1-carboxylate,
(2S,4S)-benzyl 4-(3-(hydroxyamino)-3-oxopropoxy)-2-
(quinolin-8-ylcarbamoyl)pyrrolidine-1-carboxylate,
(2S,4S)-benzyl 4-(4-(hydroxycarbamoyl)phenoxy)-2-
(quinolin-8-ylcarbamoyl)pyrrolidine-1-carboxylate,
(2R,4S)-benzyl 4-(4-(hydroxycarbamoyl)phenoxy)-2-
(quinolin-8-ylcarbamoyl)pyrrolidine-1-carboxylate and
(2S,4R)-benzyl 4-(4-(hydroxycarbamoyl)phenoxy)-2-
(quinolin-8-ylcarbamoyl)pyrrolidine-1-carboxylate.

53. The compound according to claim 1, wherein
X¹, X², X³ and X⁴ are absent;
X⁵ is a covalent bond;
X⁵ is CH₂;
n is 1;
B is —(C₀-C₇)alkyl-aryl-(C₀-C₄)alkyl-W;
W is —C(O)NHOH;
A is H; and
E and D are independently selected from a group consisting of —H, —(C₀-C₆)alkyl-aryl- and —(C₀-C₆)alkyl-heteroaryl-, wherein each aryl and heteroaryl moiety is optionally substituted with one or more R².

54. The compound according to claim 53, of the formula XVIII 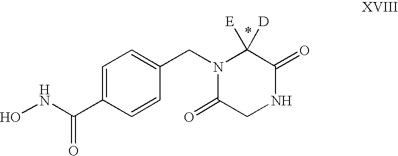
and N-oxides, hydrates, solvates, pharmaceutically acceptable salts, prodrugs and complexes thereof.

55. The compound according to claim 54, wherein one of D and E is H and the other is selected from the group consisting of 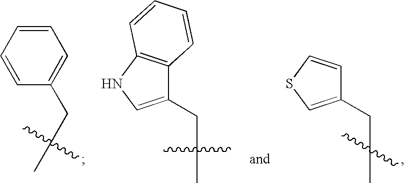
wherein each aryl or heteroaryl moiety is optionally substituted with one or more groups selected from R².

56. A composition comprising a compound according to claim 1 and a pharmaceutically acceptable carrier.

57. A composition comprising a compound according to claim 48 and a pharmaceutically acceptable carrier.

58. A method of inhibiting histone deacetylase, the method comprising contacting the histone deacetylase with an inhibiting effective amount of a compound according to claim 1.

59. A method of inhibiting histone deacetylase, the method comprising contacting the histone deacetylase with an inhibiting effective amount of a compound according to claim 48.

60. A method of inhibiting histone deacetylase in a cell, the method comprising contacting the cell with a compound according to claim 1, in an amount sufficient to inhibit histone decetylase.

61. A method of inhibiting histone deacetylase in a cell, the method comprising contacting the cell with a compound according to claim 48, in an amount sufficient to inhibit histone decetylase.

62. A method of inhibiting histone deacetylase in a cell, the method comprising contacting the cell with a composition according to claim 56, in an amount sufficient to inhibit histone decetylase.

63. A method of inhibiting histone deacetylase in a cell, the method comprising contacting the cell with a composition according to claim 57, in an amount sufficient to inhibit histone decetylase.

64. The composition according to claim 56, further comprising an additional histone deacetylase inhibitor.

65. The composition according to claim 57, further comprising an additional histone deacetylase inhibitor.

66. The method according to claim 58, further comprising contacting the cell with an additional histone deacetylase inhibitor in an amount sufficient to inhibit histone decetylase.

67. The method according to claim 59, further comprising contacting the cell with an additional histone deacetylase inhibitor in an amount sufficient to inhibit histone decetylase.

68. A method of inhibiting histone deacetylase in a cell, the method comprising contacting the cell with a composition according to claim 64, in an amount sufficient to inhibit histone decetylase.

69. A method of inhibiting histone deacetylase in a cell, the method comprising contacting the cell with a composition according to claim 65, in an amount sufficient to inhibit histone decetylase.

70. The compound according to claim 1, with the proviso that only one of Z, A, B, D and E end in with the moiety W.


SEQUENCE LISTING (11 sequences)

SEQ ID NO: 1 - DNA, 20 bp, Artificial
  Synthetic
gaaacgtgagggactcagca

SEQ ID NO: 2 - DNA, 20 bp, Artificial
  Synthetic
ggaagccagagctggagagg

SEQ ID NO: 3 - DNA, 20 bp, Artificial
  Synthetic
gctgagctgttctgatttgg

SEQ ID NO: 4 - DNA, 20 bp, Artificial
  Synthetic
cgctttccttgtcattgaca

SEQ ID NO: 5 - DNA, 20 bp, Artificial
  Synthetic
gctgcctgccgtgcccaccc

SEQ ID NO: 6 - DNA, 20 bp, Artificial
  Synthetic
tacagtccatgcaacctcca

SEQ ID NO: 7 - DNA, 20 bp, Artificial
  Synthetic
cttcggtctcacctgcttgg

SEQ ID NO: 8 - DNA, 20 bp, Artificial
  Synthetic
caggctggaatgagctacag

SEQ ID NO: 9 - DNA, 20 bp, Artificial
  Synthetic
cttcagccaggatgcccaca

SEQ ID NO: 10 - DNA, 20 bp, Artificial
  Synthetic
ctccggctcctccatcttcc

SEQ ID NO: 11 - DNA, 20 bp, Artificial
  Synthetic
agccagctgccacttgatgc